(19) United States
(12) Patent Application Publication
(10) Pub. No.: US 2024/0300942 A1
(43) Pub. Date: Sep. 12, 2024

(54) SMALL MOLECULES FOR TREATEMENT OF CANCER

(71) Applicant: VRise Therapeutics Inc., Cambridge, MA (US)

(72) Inventors: Prashant Kashinath BHAVAR, Cambridge, MA (US); Anuj Ramesh KSHIRSAGAR, Hyderabad (IN); Partha SARMA, Hyderabad (IN); Adilakshmi GANDHAM, Hyderabad (IN); Uday Kumar SURAMPUDI, Cambridge, MA (US)

(73) Assignee: VRise Therapeutics Inc., Cambridge, MA (US)

(21) Appl. No.: 18/260,928

(22) PCT Filed: Oct. 7, 2022
(86) PCT No.: PCT/IB2022/059630

§ 371 Date: Jul. 10, 2023

Related U.S. Application Data
(60) Provisional application No. 63/308,727, filed on Feb. 10, 2022.

(30) Foreign Application Priority Data
Oct. 8, 2021 (IN) 202141046053

(51) Int. Cl. C07D 471/04; A61K 31/496; A61K 31/5377; A61K 31/541; A61P 35/00

(52) U.S. Cl. CPC C07D 471/04; A61K 31/496; A61K 31/5377; A61K 31/541; A61P 35/00

(57) ABSTRACT

The present application relates to novel compounds described herein, the method of preparing the same, its pharmaceutical composition and method for use thereof. In particular the invention relates to compounds of formula (A) or their pharmaceutically acceptable salts thereof as inhibitors of KRAS protein and useful in treatment, prevention and/or amelioration of diseases or disorders associated with KRAS especially the Cancer.

This application claims the benefit of priority to Indian provisional patent application number 202141046053 filed dated 8 Oct. 2021 and U.S. provisional patent application No. 63/308,727 filed dated 10 Feb. 2022, the disclosures of both of which are incorporated herein by reference in their entirety.

### FIELD OF THE INVENTION

The present application relates to novel compounds described herein, the method of preparing the same, its pharmaceutical composition and method for use thereof. In particular, the invention relates to compounds of formula (A) or their pharmaceutically acceptable salts thereof as inhibitors of KRAS protein and useful in treatment, prevention and/or amelioration of diseases or disorders associated with KRAS especially the Cancer.

### BACKGROUND OF THE INVENTION

Genes are in the DNA of each cell of human body that control how the cell functions, including: how quickly it grows, how often it divides or how long it lives. Genes control how your cells work by making proteins. The proteins have specific functions and act as messengers for the cell and each gene must have the correct instructions for making its protein. This allows the protein to perform the correct function for the cell. All cancers begin when one or more genes in a cell mutate. A mutation is a change. It creates an abnormal protein. Or it may prevent a protein's formation. An abnormal protein provides different information than a normal protein. This can cause cells to multiply uncontrollably and become cancerous.

Broadly the two basic types of genetic mutations that are referred to by researchers are (a) Acquired mutations and factors that cause these mutations include use of Tobacco, exposure to Ultraviolet (UV) radiation. Viruses or age. Cancer that occurs because of acquired mutations is called sporadic cancer. (b) Germline mutations: A germline mutations are less common and occurs in a sperm cell or egg cell. Because the mutation affects reproductive cells, it can pass from generation to generation. Cancer caused by germline mutations is called inherited cancer and accounts for about 5% to 20% of all cancers.

Many of the genes that contribute to cancer development fall into broad categories:

DNA repair genes. These fix mistakes made when DNA is copied. Many of them function as tumor suppressor genes. BRCA1, BRCA2, and p53 are all DNA repair genes. If a person has an error in a DNA repair gene, mistakes remain uncorrected and the mistakes may lead to mutations. These mutations may eventually lead to cancer, particularly is the said mutations occurs in tumor suppressor genes or oncogenes.

Tumor suppressor genes. These are protective genes. Normally, they limit cell growth by monitoring how quickly cells divide into new cells, repairing mismatched DNA and controlling cell death. When a tumor suppressor gene mutates, cells grow uncontrollably, and they may eventually form a tumor. Examples of tumor suppressor genes include BRCA1, BRCA2, and p53 or TP53

Oncogenes. These turn a healthy cell into a cancerous cell. Mutations in these genes are not known to be inherited. The two most common oncogenes are HER2, a specialized protein that controls cancer growth and spread. It is found in some cancer cells. For example, breast and ovarian cancer cells and RAS, the gene of RAS family, which makes proteins involved in cell communication pathways, cell growth, and cell death.

One such mutation that has been reported, is the mutation in RAS family of genes. RAS has been known to acts as a molecular switch and is a monomeric globular protein that is associated with the plasma membrane. RAS can either bind to guanosine 5′-diphosphate (GDP) (known as a Resting or in inactive state) or guanosine-5-triphosphate (GTP) and converts GDP to GTP (known as a “switched on” or in active state). It is the growth promoting stimuli that causes the induction of RAS wherein the exchange of GDP to GTP occurs there by allowing the active state of RAS to interact with and activate other proteins. This activation of RAS provides a signal to the cell to continue to grow and divide.

It's noteworthy to mention, the intrinsic ability of RAS protein to turn off by switching back to GDP from GTP is very low and for RAS to turn off it requires GTPase-activating proteins (GAPs) which interact with RAS and greatly accelerate the conversion of GTP to GDP. Any mutation in RAS which affects its ability to interact with GAP or to convert GTP back to GDP result in a prolonged activation of the protein and consequently a prolonged and overactive RAS signalling ultimately lead to cancer.

RAS family is further divided in several members such as HRAS; KRAS; DIRAS1; DIRAS2; DIRAS3; ERAS; GEM; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS however the most notable RAS members associated with cancers are Harvey rat sarcoma viral oncogene homolog (HRAS) Kirsten rat sarcoma viral oncogene homolog (KRAS) and Neuroblastoma rat sarcoma viral oncogene homolog (NRAS).

With the three HRAS. KRAS and NRAS members, majority of mutations of around 25-30% in tumors are detected in KRAS gene with around 30% of all human tumors been reported to have some mutation to RAS gene.

Mutation of KRAS gene are more common in pancreatic cancer, lung adenocarcinoma, colorectal cancer, gall bladder cancer, thyroid cancer, and bile duct cancer. KRAS mutations have also been seen in about 25% of patients with NSCLC, and some studies have indicated that KRAS mutations are a negative prognostic factor in patients with NSCLC. Recently, KRAS mutations have been found to confer resistance to epidermal growth factor receptor (EGFR) targeted therapies in colorectal cancer; Understanding the status of KRAS mutation seems to be gaining importance prior to use of tyrosine kinase inhibitors (TKI).

The most common KRAS mutations are found in the P-loop at residue G12 and G13 and at residue Q61 with G12C been a frequent mutation of KRAS gene. G12C mutation have been reported to be around 14-15% across several cancers with majority been reported in Lung and colon cancer.

Researchers have learned a lot about how cancer genes work. But many cancers are not linked with a specific gene. Cancer likely involves multiple gene mutations. Moreover, some evidence suggests that genes interact with their environment. This further complicates our understanding of the role genes play in cancer. Researchers continue to study how genetic changes affect cancer development. This knowledge has led to improvements in cancer care, including early detection, risk reduction, the use of targeted therapy, and survival.

We believe, Cancer in general have very few options of treatment, especially when the cancer is a result of metastasis and is unresectable. On one end there have been several advancements in options of treatment for example use of chemotherapy either alone or in combination with radiation and/or surgery however on the other side there has been a significant amount of challenges with prognosis especially for the cancers such as the lung cancers, pancreatic cancer, prostate cancer, gastric cancer, endometrial cancer, ovarian cancer, colorectal cancer.

Accordingly, there do exists an unmet medical need for treatments for such cancers and the present invention aims to address the same.

### SUMMARY OF THE INVENTION

The present invention relates to compounds of formula (A), or pharmaceutically acceptable salts or compositions and methods of treatment with them, In particular the present invention relates to compounds of formula (A) and their pharmaceutically acceptable salts thereof useful in the treatment of RAS mediated cancer.

In one embodiment of the invention, the substituted naphthyridine compounds represented by structural formula (A)

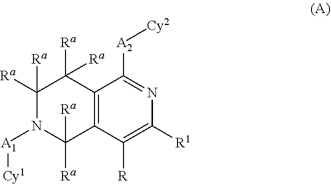

or a tautomer thereof, isotope thereof, prodrug thereof. N-oxide thereof, a pharmaceutically acceptable ester thereof, a pharmaceutically acceptable salt thereof, or a stereoisomer thereof, wherein

A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—.

A₂ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O), —, —C(═O)—, —NR^{x}—, —CO—NR^{x}— and —NR^{x}—CO—.

Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl.

Cy² is selected from cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl which is optionally substituted by E.

E is a group capable of forming a covalent bond or an “Electrophile” or “electrophilic moiety capable of forming a covalent bond.

R at each occurrence is independently selected from CN (Cyano), COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R, —SR^{b}, —SOR^{c}, —SO₂R^{b}, —CR^{b}R^{c}C(═O)R^{b} or —CR^{b}R^{c}C(═S)R^{z}.

R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NRS(═O)R^{c}, —NRS(═O)₂R^{c}, —NR^{b}—OR^{c}, ═N—NR^{b}R^{c}, —NR^{b}C(═O)OR^{c}, —NR^{b}C(═O)R, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}(═O)R^{b} or —CR^{b}R^{c}C(═S)R^{z}.

R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which may be same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S; each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{x} and R^{y} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z)—R^{a}, substituted or unsubstituted alkyl group, substituted or unsubstituted alkenyl, substituted or unsubstituted alkynyl, and substituted or unsubstituted Cycloalkyl;
Z is selected from O or S; and
each occurrence of p is independently 0, 1 or 2.

In one embodiment of the invention, the substituted naphthyridine compounds represented by structural formula (A-I)

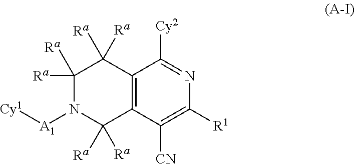

or a tautomer thereof, isotope thereof, prodrug thereof, N-oxide thereof, a pharmaceutically acceptable ester thereof, a pharmaceutically acceptable salt thereof, or a stereoisomer thereof, wherein A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—.

Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl.

Cy² is selected from cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl which is optionally substituted by E.

E is a group capable of forming a covalent bond or an “Electrophile” or “electrophilic moiety capable of forming a covalent bond.

R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR, —SOR^{c}, —SO₂R^{b}, —CR^{b}R^{c}(═O)R^{b} or —CR^{b}R^{c}C(═S)R^{z}.

R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NRS(═O)R^{c}, —NR^{b}S(═O)₂R, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which may be same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S; each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{x} and R^{y} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group; and
each occurrence of p is independently 0, 1 or 2.

In one embodiment of the invention, the substituted naphthyridine compounds represented by structural formula (A-II)

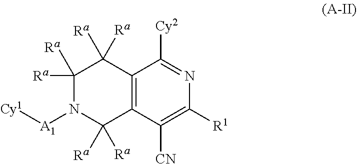

or a tautomer thereof, isotope thereof, prodrug thereof, N-oxide thereof, a pharmaceutically acceptable ester thereof, a pharmaceutically acceptable salt thereof, or a stereoisomer thereof, wherein
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—.

Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl.

Cy² is substituted or unsubstituted heterocyclyl, which is optionally substituted by E.

E is a group capable of forming a covalent bond or an “Electrophile” or “electrophilic moiety capable of forming a covalent bond.

R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}. —R^{b}NR^{c}C(═O)R^{b}, —R^{b}OR^{c}, —R^{b}C(═O)OR^{c}, —R^{b}C(═O)NR^{b}R^{c}, —R^{b}C(═O)R, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}.

R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which may be same or different and are selected from O, NR^{a} or S or; any two R^{a} attached to the same carbon atom may be joined to a form a Oxo (C═O). Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{x} and R^{y} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group; and
each occurrence of p is independently 0, 1 or 2.

Further preferred are compounds having the formula (A), (A-I) or (A-II), wherein A₁ is absent or is —CR^{b}R^{c}—; wherein each of R^{b} and R^{c} is independently selected from hydrogen or substituted or unsubstituted alkyl.

Further preferred are compounds having the formula (A), (A-I) or (A-II) wherein A₁ is absent or is —CR^{b}R^{c}—; wherein R^{b} is independently methyl or ethyl and R^{c} is hydrogen.

Further preferred are compounds having the formula (A), (A-I) or (A-II) wherein A₁ is absent or is —CR^{b}R^{c}—; wherein R^{b} is hydrogen and R^{c} is hydrogen.

Further preferred are compounds having the formula (A), (A-I) or (A-II) wherein A₁ is absent or —CH₂ or —(C═O)

Further preferred are compounds having the formula (A), (A-I) or (A-II) wherein A₂ is absent or is —CR^{b}R^{c}—; wherein each of R^{b} and R^{c} is independently selected from hydrogen, substituted or unsubstituted alkyl

Further preferred are compounds having the formula (A), (A-I) or (A-II) wherein A₂ is absent or is —CR^{b}R^{c}—; where in R^{b} is independently methyl or ethyl and R^{c} is hydrogen.

Further preferred are compounds having the formula (A), (A-I) or (A-II) wherein A₂ is absent.

Further preferred are compounds having the formula (A), (A-I) or (A-II), wherein Cy¹ is selected form a cyclic group selected from substituted or unsubstituted heterocyclyl, substituted or unsubstituted aryl, substituted or unsubstituted arylalkyl, substituted or unsubstituted heteroaryl, substituted or unsubstituted heteroarylalkyl.

Further preferred are compounds having the formula (A), (A-I) or (A-II), wherein Cy¹ is selected form a cyclic group selected from substituted or unsubstituted aryl and substituted or unsubstituted heteroaryl.

Further preferred are compounds having the formula (A), (A-I) or (A-II), wherein Cy¹ is selected form

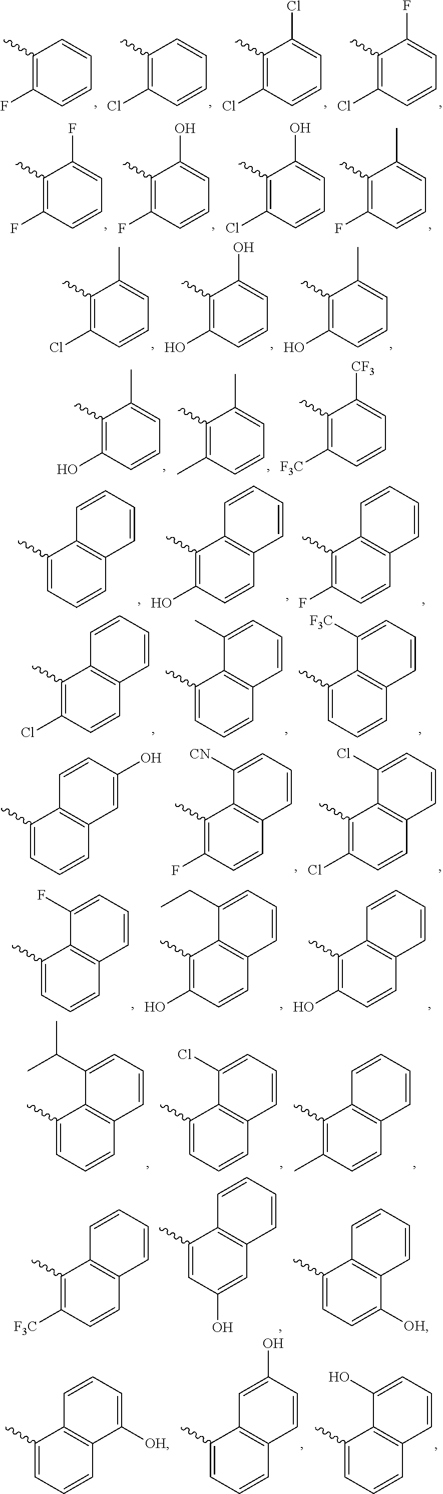

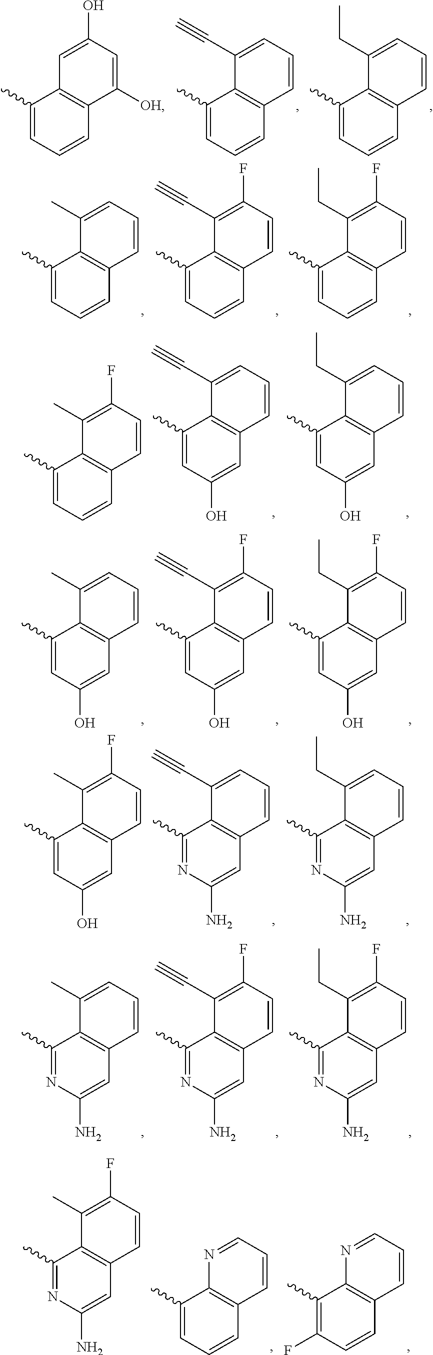

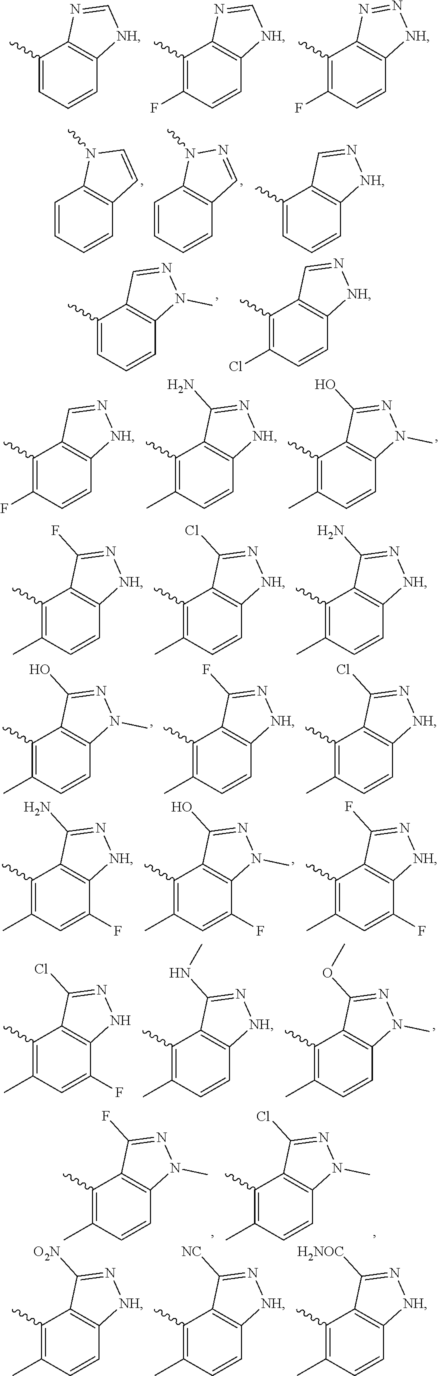

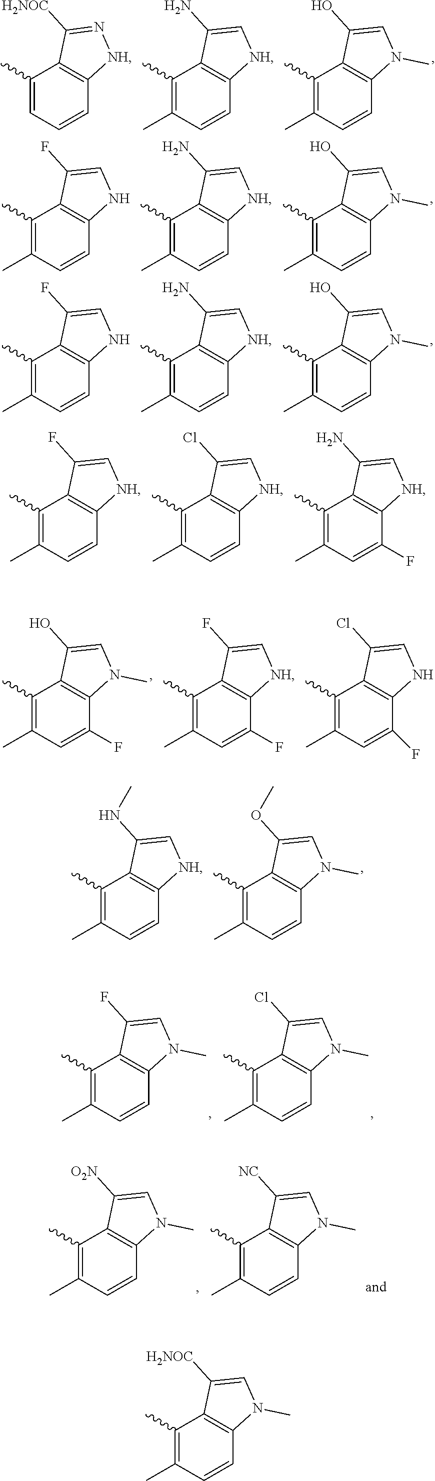

Further preferred are compounds having the formula (A), (A-I) or (A-II), wherein Cy² is selected from cyclic group selected from substituted or unsubstituted cycloalkyl, substituted or unsubstituted cycloalkylalkyl, substituted or unsubstituted heterocyclyl and substituted or unsubstituted heterocyclylalkyl.

Further preferred are compounds having the formula (A), (A-I) or (A-II), wherein Cy² is selected from cyclic group selected from substituted or unsubstituted cycloalkyl, substituted or unsubstituted cycloalkylalkyl, substituted or unsubstituted heterocyclyl and substituted or unsubstituted heterocyclylalkyl, wherein each group is optionally further substituted with group E.

Further preferred are compounds having the formula (A), (A-I) or (A-II), wherein Cy² is selected form

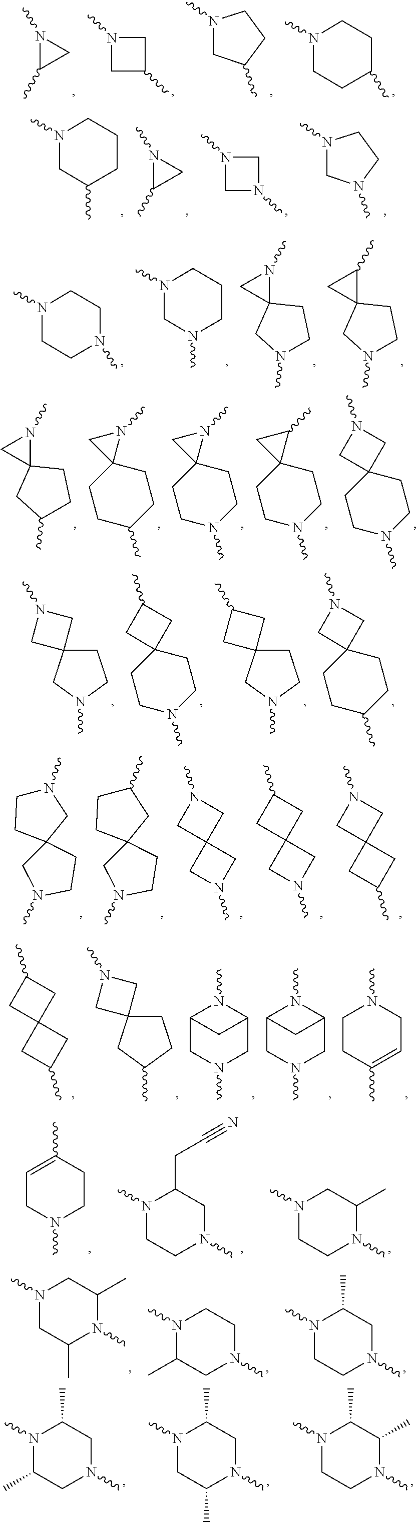

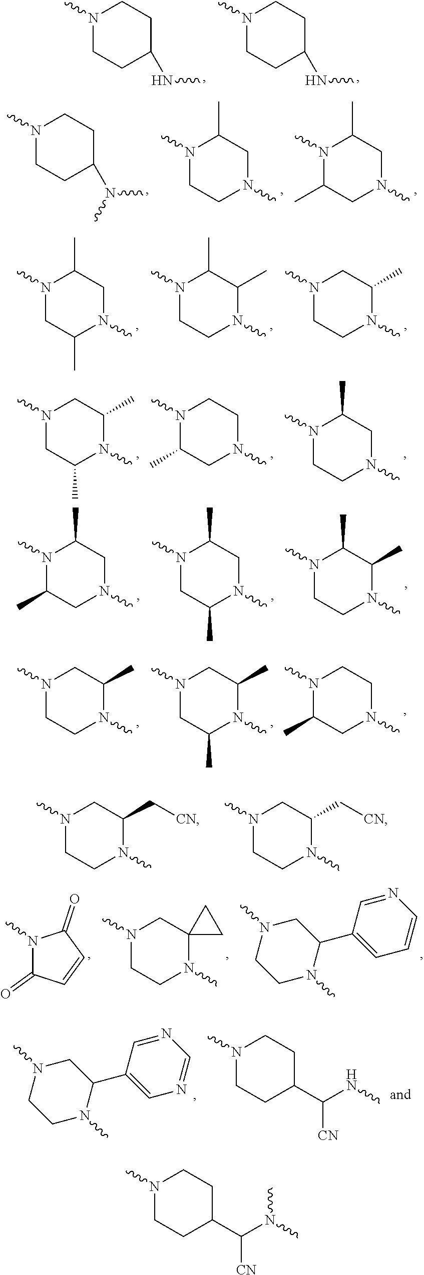

substituted with E a group capable of forming a covalent bond.

Further preferred are compounds having the formula (A), (A-I) or (A-II), wherein E is selected from

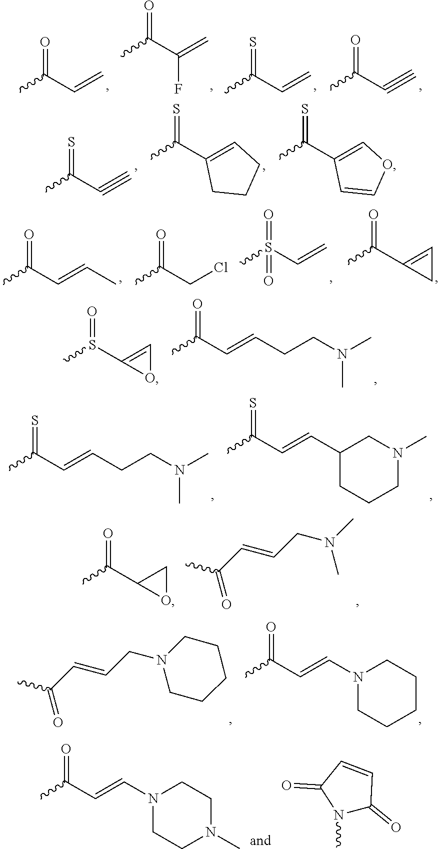

Further preferred are compounds having the formula (A), (A-I) or (A-II), wherein E is selected from

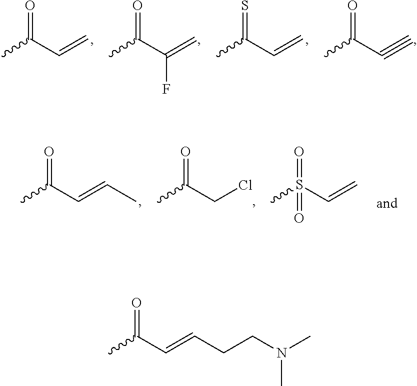

Further preferred are compounds having the formula (A), (A-I) or (A-II), wherein A₂-Cy²-E is selected from

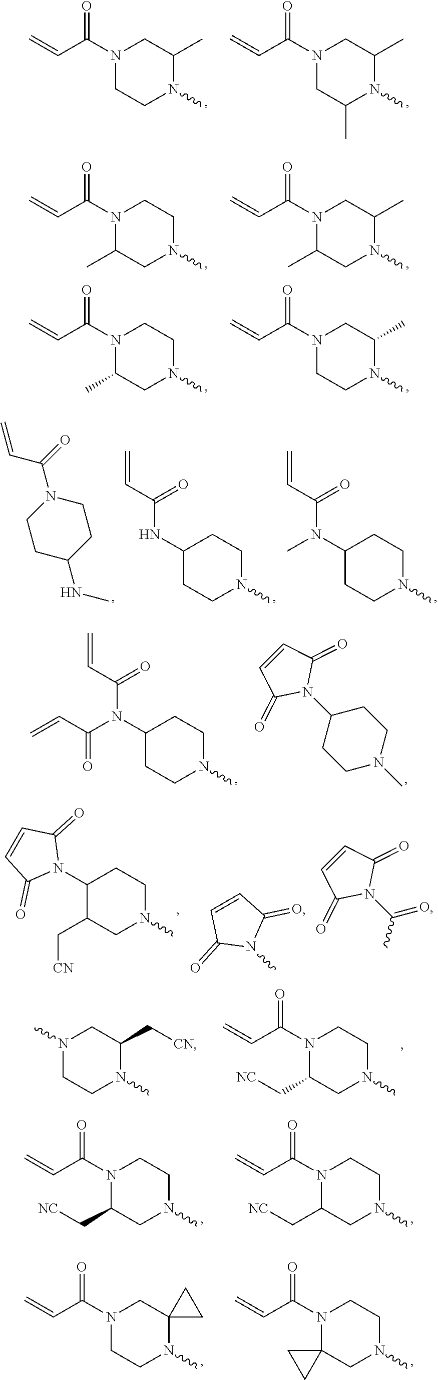

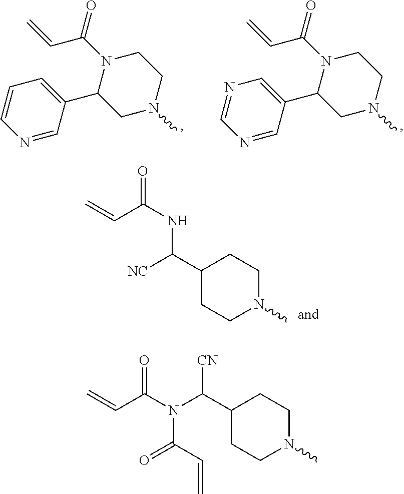

Further preferred are compounds having the formula (A), (A-I) or (A-II), wherein A₂-Cy²-E is selected from

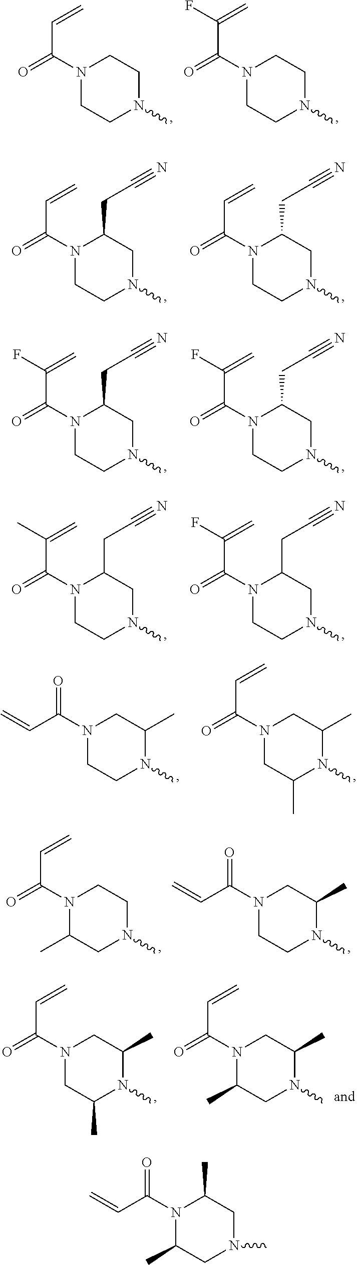

Further preferred are compounds having the formula (A), (A-I) or (A-II), wherein R is Cyano (CN).

Further preferred are compounds having the formula (A), (A-I) or (A-II), wherein R¹ is selected from hydrogen, halogen, OR^{b}, S—R^{b}, —S(═O)pR^{b}—, —C(═O)—R^{b}, —NR^{b}R^{c}, —CO—NR^{b}R^{c}— and —NR^{b}—CO—R^{c};

Further preferred are compounds having the formula (A), (A-I) or (A-II), wherein R¹ is —O—R^{b}

Further preferred are compounds having the formula (A), (A-I) or (A-II), wherein R¹ is —NR^{b}R^{c}

Further preferred are compounds having the formula (A), (A-I) or (A-II), wherein R¹ is independently selected from, substituted or unsubstituted alkyl, substituted or unsubstituted alkoxy, substituted or unsubstituted heterocyclyl, substituted or unsubstituted heterocyclylalkyl, substituted or unsubstituted heteroaryl, substituted or unsubstituted heteroarylalkyl.

Further preferred are compounds having the formula (A), (A-I) or (A-II), wherein R¹ is independently selected from substituted or unsubstituted alkyl, substituted or unsubstituted heterocyclyl, substituted or unsubstituted heterocyclylalkyl and substituted or unsubstituted heteroaryl

Further preferred are compounds having the formula (A), (A-I) or (A-II), wherein R¹ is independently selected from

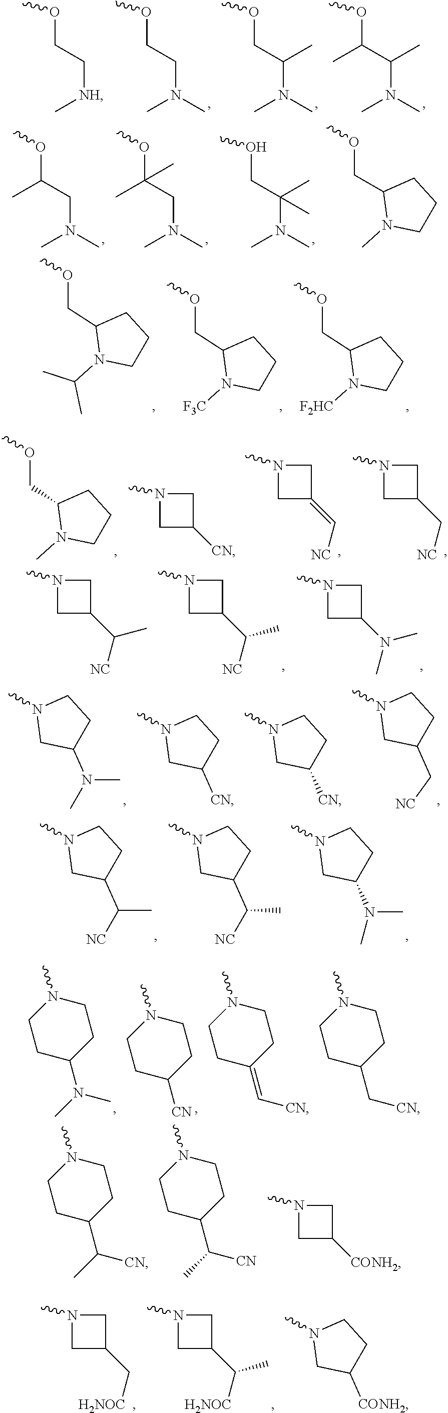

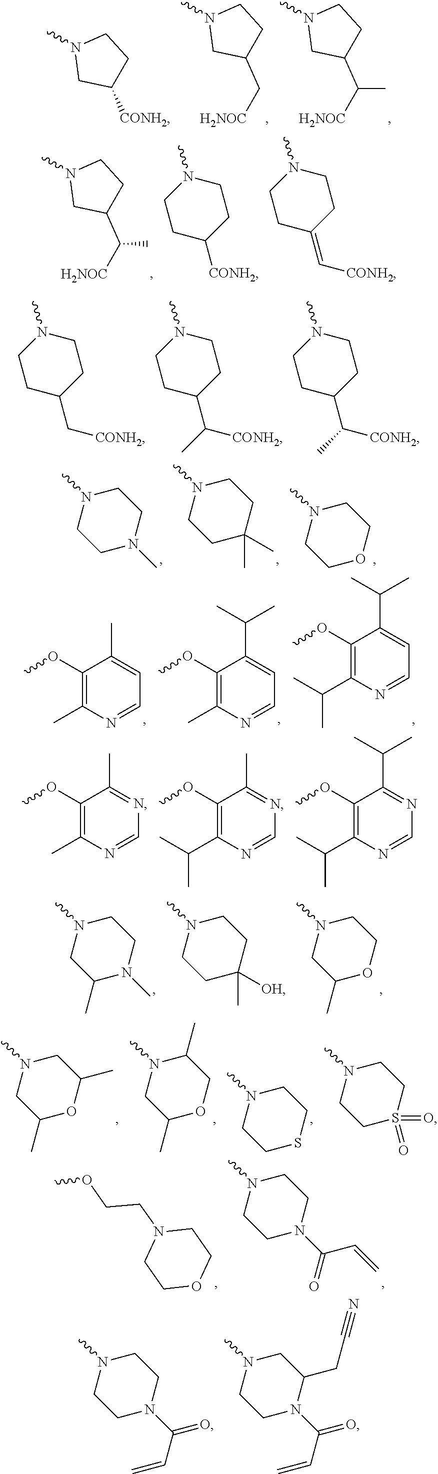

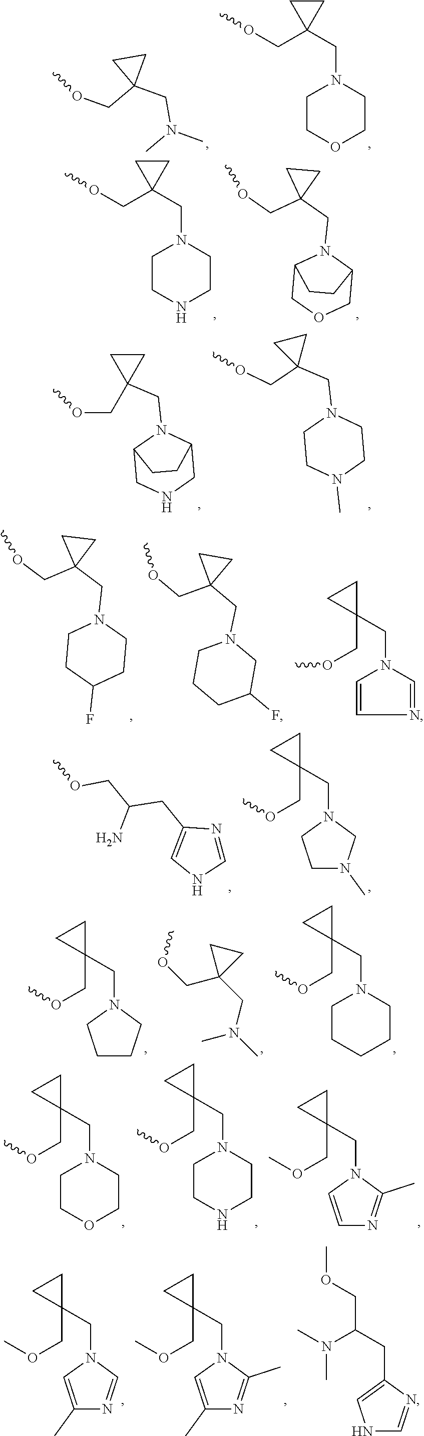

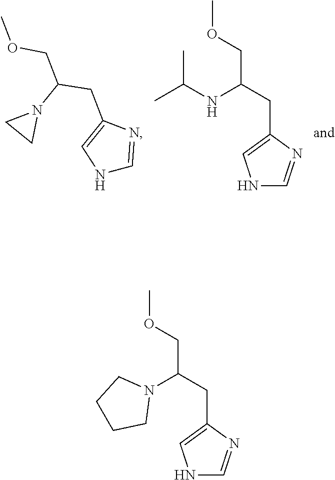

Further preferred are compounds having the formula (A), (A-I) or (A-II), wherein R^{a} is Hydrogen or substituted or unsubstituted alkyl

Further preferred are compounds having the formula (A), (A-I) or (A-II), wherein two R^{a} attached to same carbon atom form a C═O (Oxo) group.

Further preferred are compounds having the formula (A), (A-I) or (A-II), wherein A₁ is
(i) absent or is —CR^{b}R^{c}—; wherein each of R^{b} and R^{c} are independently selected from hydrogen or substituted or unsubstituted alkyl and/or
(ii) absent or is —CR^{b}R^{c}—; wherein R^{b} is independently methyl or ethyl and R^{c} is hydrogen and/or
(iii) absent or is —CR^{b}R^{c}—; wherein R^{b} is hydrogen and R^{c} is hydrogen and/or
(iv) absent or —CH₂ or —(C═O) and/or any combinations thereof

A₂ is
(i) absent or is —CR^{b}R^{c}—; wherein each of R^{b} and R^{c} are independently selected from hydrogen, substituted or unsubstituted alkyl; and/or
(ii) absent or is —CR^{b}R^{c}—; where in R^{b} is independently methyl or ethyl and R^{c} is hydrogen and/or
(iii) absent and/or any combinations thereof; Cy¹ is selected form
(i) a cyclic group selected from substituted or unsubstituted heterocyclyl, substituted or unsubstituted aryl, substituted or unsubstituted arylalkyl, substituted or unsubstituted heteroaryl, substituted or unsubstituted heteroarylalkyl and/or
(ii) a cyclic group selected from substituted or unsubstituted aryl and substituted or unsubstituted heteroaryl and/or combinations thereof Cy² is selected from
(i) cyclic group selected from substituted or unsubstituted cycloalkyl, substituted or unsubstituted cycloalkylalkyl, substituted or unsubstituted heterocyclyl and substituted or unsubstituted heterocyclylalkyl and/or
(ii) cyclic group selected from substituted or unsubstituted cycloalkyl, substituted or unsubstituted cycloalkylalkyl, substituted or unsubstituted heterocyclyl and substituted or unsubstituted heterocyclylalkyl, wherein each group is optionally further substituted with group E and/or combinations thereof; R is Cyano (CN); and

R¹ is selected from
(i)hydrogen, halogen, OR^{b}, S—R^{b}, —S(═O)pR^{b}—, —C(═O)—R^{b}, —NR^{b}R^{c}, —CO—NR^{b}R^{c}— and —NR^{b}—CO—R^{c}; and/or
(ii) —O—R^{b} and/or
(iii) —NR^{b}R^{c} and/or
(iv) substituted or unsubstituted alkyl, substituted or unsubstituted alkoxy, substituted or unsubstituted heterocyclyl, substituted or unsubstituted heterocyclylalkyl, substituted or unsubstituted heteroaryl, substituted or unsubstituted heteroarylalkyl and/or
(v) substituted or unsubstituted alkyl, substituted or unsubstituted heterocyclyl, substituted or unsubstituted heterocyclylalkyl and substituted or unsubstituted heteroaryl and/or combinations thereof.

Further preferred are compounds having the formula (A), (A-I) or (A-II), wherein

R at each occurrence is independently selected from CN (Cyano), COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}.

A₁ is absent or substituted or unsubstituted C₁₋₄ alkyl;

A₂ is absent or substituted or unsubstituted C₁₋₄ alkyl

Cy¹ is selected form substituted or unsubstituted aryl or substituted or unsubstituted heteroaryl;

Cy² is selected from substituted or unsubstituted heterocyclyl, which is optionally substituted by E.

E is a group capable of forming a covalent bond or an “Electrophile” or “electrophilic moiety capable of forming a covalent bond.

R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR, —NR^{b}C(═O)R, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}.

R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NRS(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R or —CR^{b}R^{c}C(═S)R^{z} or any two R^{a} may be joined to a form a substituted or unsubstituted saturated or unsaturated 3-6 member ring, which may optionally include heteroatoms which may be same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S.
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{x} and R^{y} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z)—R^{a}, substituted or unsubstituted alkyl group, substituted or unsubstituted alkenyl, substituted or unsubstituted alkynyl, and substituted or unsubstituted Cycloalkyl;
Z is selected from O or S; and
each occurrence of p is independently 0, 1 or 2.

Further preferred are compounds having the formula (A), (A-I) or (A-II), wherein
A₁ is absent or substituted or unsubstituted alkyl;
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl.
Cy² is selected from cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl which is optionally substituted by E.
E is a group capable of forming a covalent bond or an “Electrophile” or “electrophilic moiety capable of forming a covalent bond;
R at each occurrence is independently selected from CN (Cyano), COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —ROC(═O)R^{c}, —SR, —SOR^{c}, —SO₂R^{b}, —CR^{b}R^{c}C(═O)R^{b} or —CR^{b}R^{c}C(═S)R^{z}.
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NRS(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which may be same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{x} and R^{y} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z)—R^{a}, substituted or unsubstituted alkyl group, substituted or unsubstituted alkenyl, substituted or unsubstituted alkynyl, and substituted or unsubstituted Cycloalkyl;
Z is selected from O or S; and
each occurrence of p is independently 0, 1 or 2.

Further preferred are compounds having the formula (A), (A-I) or (A-II), wherein
Cy¹ is selected from substituted or unsubstituted aryl or substituted or unsubstituted heteroaryl;
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ, —O—, —S—, —S(═O)ₚ—, —C(═O)—, —NR^{x}—, —CO—NR^{x}— and —NR^{x}—CO—.
Cy² is selected from cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl which is optionally substituted by E.
E is a group capable of forming a covalent bond or an “Electrophile” or “electrophilic moiety capable of forming a covalent bond.
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}.
   R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which may be same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S.
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{x} and R^{y} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z)—R^{a}, substituted or unsubstituted alkyl group, substituted or unsubstituted alkenyl, substituted or unsubstituted alkynyl, and substituted or unsubstituted Cycloalkyl;
Z is selected from O or S; and
each occurrence of p is independently 0, 1 or 2.

Further preferred are compounds having the formula (A), (A-I) or (A-II), wherein
Cy² is substituted or unsubstituted heterocyclyl, which is optionally substituted by E;
E is a group capable of forming a covalent bond or an “Electrophile” or “electrophilic moiety capable of forming a covalent bond.
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—.
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl.
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{c}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}.
R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{b}C, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which may be same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S.
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{x} and R^{y} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group,
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z)—R^{a}, substituted or unsubstituted alkyl group, substituted or unsubstituted alkenyl, substituted or unsubstituted alkynyl, and substituted or unsubstituted Cycloalkyl;
Z is selected from O or S; and
each occurrence of p is independently 0, 1 or 2.

Further preferred are compounds having the formula (A), (A-I) or (A-II), wherein
R¹ is selected from hydrogen, halogen, —NR^{b}R^{c} or —OR^{b};
   A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—.
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl.
Cy² is selected from cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl which is optionally substituted by E.
E is a group capable of forming a covalent bond or an “Electrophile” or “electrophilic moiety capable of forming a covalent bond.
R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, ═NR—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}C, —R^{b}C(═O)OR, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which may be same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S, each occurrence of R^{b} and R^{c} are independently selected from hydrogen, substituted or unsubstituted alkyl, or variables of R^{b} and R^{c} together with the nitrogen which they attached can form a substituted or unsubstituted heterocyclic ring;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z)—R^{a}, substituted or unsubstituted alkyl group, substituted or unsubstituted alkenyl, substituted or unsubstituted alkynyl, and substituted or unsubstituted Cycloalkyl;
Z is selected from O or S; and
each occurrence of p is independently 0, 1 or 2.

In another embodiment of the invention, the substituted naphthyridine compounds represented by structural formula (A-III)

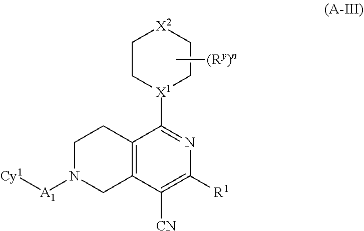

or a tautomer thereof, isotope thereof, prodrug thereof, N-oxide thereof, a pharmaceutically acceptable ester thereof, a pharmaceutically acceptable salt thereof, or a stereoisomer thereof, wherein
A₁ is absent or substituted or unsubstituted alkyl;
Cy¹ is selected from substituted or unsubstituted aryl or substituted or unsubstituted heteroaryl;
R¹ is selected from hydrogen, halogen, substituted or unsubstituted alkyl, —NR^{b}R^{c} or —OR^{b};
each occurrence of R^{b} and R^{c} are independently selected from hydrogen, substituted or unsubstituted alkyl, or variables of R^{b} and R^{c} together with the nitrogen which they attached can form a substituted or unsubstituted heterocyclic ring;
X¹ is C or N;
X² is selected from —N-E or CH₂-E, O or S;
   E is a group capable of forming a covalent bond or an “Electrophile” or “electrophilic moiety capable of forming a covalent bond.
R^{y} is selected from hydrogen, halogen, substituted or unsubstituted alkyl; and
n is 0, 1, 2, 3, 4, 5, 6, 7 or 8.

Further preferred are compounds having the formula (A-III), wherein A₁ is absent or methyl.

Further preferred are compounds having the formula (A-III), wherein A₁ is absent

Further preferred are compounds having the formula (A-III), wherein Cy¹ is selected from substituted or unsubstituted aryl;

Further preferred are compounds having the formula (A-III), wherein Cy¹ is selected from phenyl or naphthalene, optionally substituted with halogen, hydroxy or substituted or unsubstituted alkyl:

Further preferred are compounds having the formula (A-III), wherein Cy¹ is selected from quinoline or quinazoline, optionally substituted with halogen, hydroxy or substituted or unsubstituted alkyl;

Further preferred are compounds having the formula (A-III), wherein R¹ is selected from hydrogen, halogen,

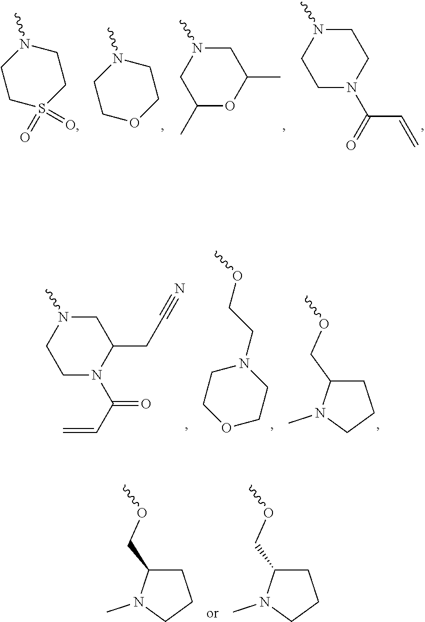

Further preferred are compounds having the formula (A-III), wherein X¹ is N.

Further preferred are compounds having the formula (A-III), wherein X² is selected from —N-E or CH₂-E or O, wherein E is selected from

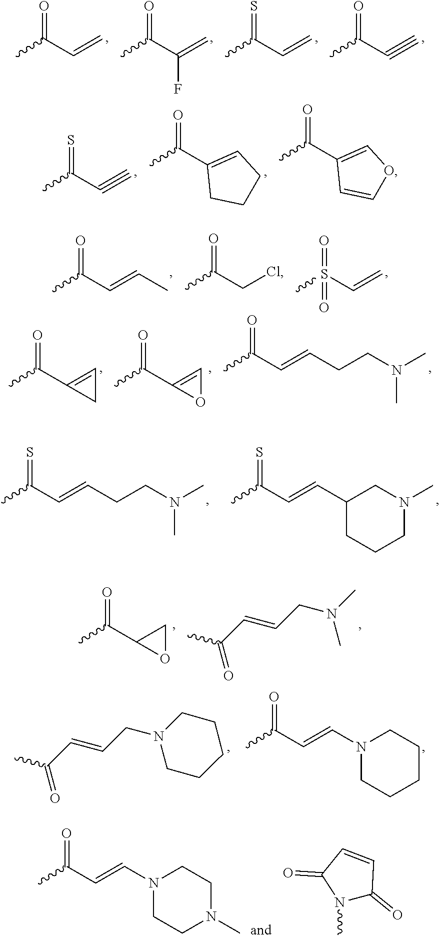

Further preferred are compounds having the formula (A-III), wherein X² is selected from —N-E or CH₂-E or O, wherein E is selected from

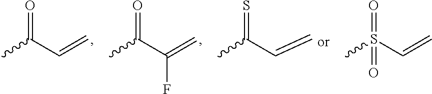

Further preferred are compounds having the formula (A-III), wherein
A₁ is absent or methyl;
Cy¹ is selected from substituted or unsubstituted aryl;
R¹ is selected from hydrogen, halogen,

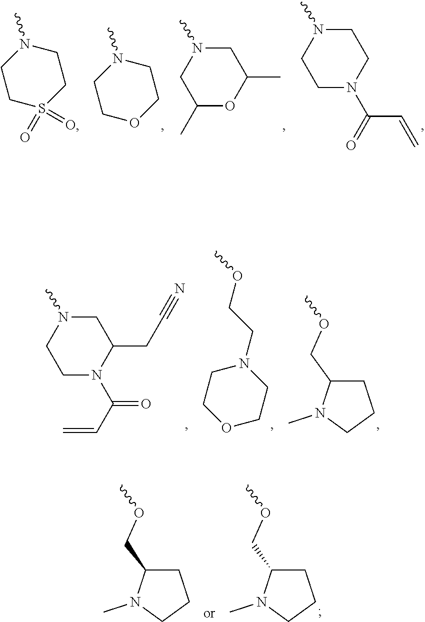

X¹ is N; and
X² is selected from —N-E or CH₂-E or O, wherein E is selected from

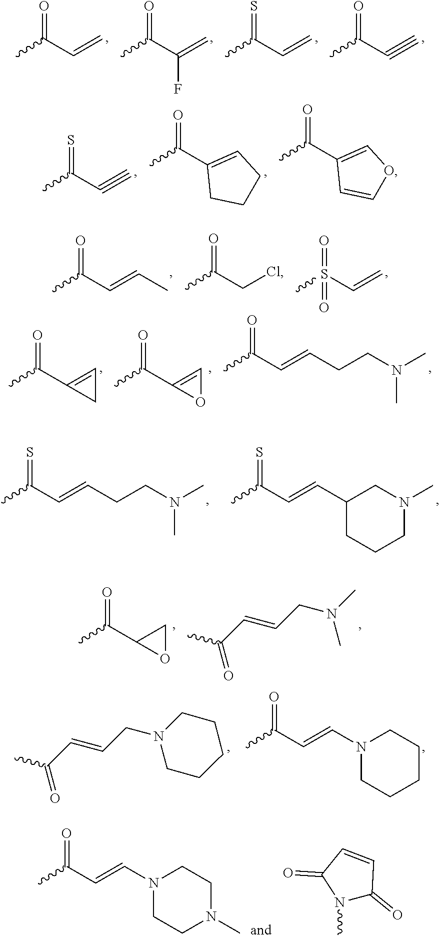

The non-limiting representative compounds of the present invention are as listed herein below and pharmaceutically acceptable salts thereof. The present invention should not be construed to be limited to these compounds.
1. 1-(4-acryloylpiperazin-1-yl)-3-(1,1-dioxidothiomorpholino)-6-(naphthalen-1-yl)-5,6,7,8-tetrahydro-2,6-naphthyridine-4-carbonitrile;
2. 1-(4-acryloylpiperazin-1-yl)-6-(8-chloronaphthalen-1-yl)-3-morpholino-5,6,7,8-tetrahydro-2,6-naphthyridine-4-carbonitrile;
3. 1-(4-acryloylpiperazin-1-yl)-3-(2-morpholinoethoxy)-6-(naphthalen-1-yl)-5,6,7,8-tetrahydro-2,6-naphthyridine-4-carbonitrile;
4. 1-(4-acryloylpiperazin-1-yl)-6-(8-chloronaphthalen-1-yl)-3-(2-orpholinoethoxy)-5,6,7,8-tetrahydro-2,6-naphthyridine-4-carbonitrile;
5. 3-(4-acryloylpiperazin-1-yl)-1-(2,6-dimethylmorpholino)-6-(naphthalen-1-yl)-5,6,7,8-tetrahydro-2,6-naphthyridine-4-carbonitrile;
6. 3-(4-acryloylpiperazin-1-yl)-6-(8-chloronaphthalen-1-yl)-1-(2,6-dimethylmorpholino)-5,6,7,8-tetrahydro-2,6-naphthyridine-4-carbonitrile;
7. 3-(4-acryloyl-3-(cyanomethyl)piperazin-1-yl)-1-(2,6-dimethylmorpholino)-6-(naphthalen-1-yl)-5,6,7,8-tetrahydro-2,6-naphthyridine-4-carbonitrile;
8. (S)-1-(4-acryloylpiperazin-1-yl)-6-(8-chloronaphthalen-1-yl)-3-((1-methylpyrrolidin-2-yl)methoxy)-5,6,7,8-tetrahydro-2,6-naphthyridine-4-carbonitrile;
9. (S)-1-(4-acryloylpiperazin-1-yl)-6-(3-hydroxynaphthalen-1-yl)-3-((1-methylpyrrolidin-2-yl)methoxy)-5,6,7,8-tetrahydro-2,6-naphthyridine-4-carbonitrile;
10. (S)-1-(4-(2-fluoroacryloyl)piperazin-1-yl)-3-((1-methylpyrrolidin-2-yl)methoxy)-6-(quinazolin-4-yl)-5,6,7,8-tetrahydro-2,6-naphthyridine-4-carbonitrile;
11. (S)-6-(8-chloronaphthalen-1-yl)-3-((1-methylpyrrolidin-2-yl)methoxy)-1-(4-(vinylsulfonyl)piperazin-1-yl)-5,6,7,8-tetrahydro-2,6-naphthyridine-4-carbonitrile;
12. 6-(8-chloronaphthalen-1-yl)-1-(4-(2-fluoroacryloyl)-3-methylpiperazin-1-yl)-3-(((S)-1-methylpyrrolidin-2-yl)methoxy)-5,6,7,8-tetrahydro-2,6-naphthyridine-4-carbonitrile. 13. 1-(4-acryloyl-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile;
14. 1—((S)-4-acryloyl-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile;
15. 1—((R)-4-acryloyl-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile;
16. 1-(4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile;
17. 1—((S)-4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile;
18. 1—((R)-4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile;
19. 1-(4-acryloyl-3-(cyanomethyl)piperazin-1-yl)-6-(3-hydroxynaphthalen-1-yl)-3-(((S)-1-methylpyrrolidin-2-yl)methoxy)-5,6,7,8-tetrahydro-2,6-naphthyridine-4-carbonitrile;
20. 1-(3-(cyanomethyl)-4-(2-fluoroacryloyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile;
21. (S)-1-(4-acryloylpiperazin-1-yl)-6-(isoquinolin-4-yl)-3-((1-methylpyrrolidin-2-yl)methoxy)-5,6,7,8-tetrahydro-2,6-naphthyridine-4-carbonitrile;
22. (S)-1-(4-acryloylpiperazin-1-yl)-3-((1-methylpyrrolidin-2-yl)methoxy)-6-(quinolin-8-yl)-5,6,7,8-tetrahydro-2,6-naphthyridine-4-carbonitrile;
23. 1-(4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
24. 1—((R)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
25. 1—((S)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
26. 6-(8-chloronaphthalen-1-yl)-1-(3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile;
27. 6-(8-chloronaphthalen-1-yl)-1-((S)-3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile;
28. 6-(8-chloronaphthalen-1-yl)-1-((R)-3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile;
29. 1-(4-acryloylpiperazin-1-yl)-6-benzyl-3-chloro-5,6,7,8-tetrahydro-2,6-naphthyridine-4-carbonitrile;
30. N-(1-(6-benzyl-3-chloro-4-cyano-5,6,7,8-tetrahydro-2,6-naphthyridin-1-yl)piperidin-4-yl)acrylamide;
31. N-(1-(6-benzyl-4-cyano-5,6,7,8-tetrahydro-2,6-naphthyridin-1-yl)piperidin-4-yl)acrylamide;
32. 1,3-bis(4-acryloylpiperazin-1-yl)-6-benzyl-5,6,7,8-tetrahydro-2,6-naphthyridine-4-carbonitrile;
33. 1-(4-acryloylpiperazin-1-yl)-6-benzyl-4-cyano-5,6,7,8-tetrahydro-2,6-naphthyridin-3-yl acrylate;
34. 1-(4-acryloylpiperazin-1-yl)-6-benzyl-3-morpholino-5,6,7,8-tetrahydro-2,6-naphthyridine-4-carbonitrile;
35. 1-(4-acryloylpiperazin-1-yl)-6-benzyl-3-(4-methylpiperazin-1-yl)-5,6,7,8-tetrahydro-2,6-naphthyridine-4-carbonitrile;
36. 1-(4-acryloyl-3-(cyanomethyl)piperazin-1-yl)-6-benzyl-3-(4-methylpiperazin-1-yl)-5,6,7,8-tetrahydro-2,6-naphthyridine-4-carbonitrile;
37. 6-(1-naphthoyl)-1-(4-acryloyl-3-(cyanomethyl)piperazin-1-yl)-3-(4-methylpiperazin-1-yl)-5,6,7,8-tetrahydro-2,6-naphthyridine-4-carbonitrile;
38. (S)-1-(4-acryloyl-2-methylpiperazin-1-yl)-6-benzyl-3-(4-methylpiperazin-1-yl)-5,6,7,8-tetrahydro-2,6-naphthyridine-4-carbonitrile;
39. (R)-1-(4-acryloyl-2-methylpiperazin-1-yl)-6-benzyl-3-(4-methylpiperazin-1-yl)-5,6,7,8-tetrahydro-2,6-naphthyridine-4-carbonitrile;
40. 1-(4-acryloylpiperazin-1-yl)-6-benzyl-3-hydroxy-5,6,7,8-tetrahydro-2,6-naphthyridine-4-carbonitrile;
41. 1-(4-acryloylpiperazin-1-yl)-3-(4-methylpiperazin-1-yl)-6-(naphthalen-1-yl)-5,6,7,8-tetrahydro-2,6-naphthyridine-4-carbonitrile;
42. 1-(4-acryloylpiperazin-1-yl)-6-benzyl-3-(piperidin-1-yl)-5,6,7,8-tetrahydro-2,6-naphthyridine-4-carbonitrile;
43. 1-(4-acryloylpiperazin-1-yl)-6-benzyl-3-(2-fluoropyridin-4-yl)-5,6,7,8-tetrahydro-2,6-naphthyridine-4-carbonitrile;
44. 1-(4-acryloylpiperazin-1-yl)-6-(naphthalen-1-yl)-3-(piperidin-1-yl)-5,6,7,8-tetrahydro-2,6-naphthyridine-4-carbonitrile;
45. 1-(4-acryloylpiperazin-1-yl)-6-benzyl-3-((1-methylpyrrolidin-2-yl)methoxy)-5,6,7,8-tetrahydro-2,6-naphthyridine-4-carbonitrile;
46. 1,3-bis(4-acryloylpiperazin-1-yl)-6-(naphthalen-1-yl)-5,6,7,8-tetrahydro-2,6-naphthyridine-4-carbonitrile;
47. 1,3-bis(4-acryloylpiperazin-1-yl)-6-(benzo[b]thiophen-4-yl)-5,6,7,8-tetrahydro-2,6-naphthyridine-4-carbonitrile;
48. 1-(4-acryloylpiperazin-1-yl)-6-(naphthalen-1-yl)-3-(4-propionylpiperazin-1-yl)-5,6,7,8-tetrahydro-2,6-naphthyridine-4-carbonitrile;
49. 3-(4-acryloylpiperazin-1-yl)-6-(naphthalen-1-yl)-1-(4-propionylpiperazin-1-yl)-5,6,7,8-tetrahydro-2,6-naphthyridine-4-carbonitrile;
50. 1-(4-acryloylpiperazin-1-yl)-3-morpholino-6-(naphthalen-1-yl)-5,6,7,8-tetrahydro-2,6-naphthyridine-4-carbonitrile;
51. 3-(4-acetylpiperazin-1-yl)-1-(4-acryloylpiperazin-1-yl)-6-(naphthalen-1-yl)-5,6,7,8-tetrahydro-2,6-naphthyridine-4-carbonitrile;
52. 1-(4-acryloylpiperazin-1-yl)-3-(2,6-dimethylmorpholino)-6-(naphthalen-1-yl)-5,6,7,8-tetrahydro-2,6-naphthyridine-4-carbonitrile;
53. 1-(4-acryloylpiperazin-1-yl)-3-(4-(methylsulfonyl)piperazin-1-yl)-6-(naphthalen-1-yl)-5,6,7,8-tetrahydro-2,6-naphthyridine-4-carbonitrile;
54. 1-(4-acryloylpiperazin-1-yl)-3-(4-ethylpiperazin-1-yl)-6-(naphthalen-1-yl)-5,6,7,8-tetrahydro-2,6-naphthyridine-4-carbonitrile;
55. (S)-1-(4-acryloylpiperazin-1-yl)-3-((1-methylpyrrolidin-2-yl)methoxy)-6-(naphthalen-1-yl)-5,6,7,8-tetrahydro-2,6-naphthyridine-4-carbonitrile;
56. 1-(4-acryloylpiperazin-1-yl)-6-(naphthalen-1-yl)-5,6,7,8-tetrahydro-2,6-naphthyridine-4-carbonitrile;
57. 1-(4-(2-fluoroacryloyl)piperazin-1-yl)-6-(naphthalen-1-yl)-3-(4-propionylpiperazin-1-yl)-5,6,7,8-tetrahydro-2,6-naphthyridine-4-carbonitrile and pharmaceutically acceptable salts thereof.

Further preferred is a compound selected from
1-(4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
1-((R)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
1-((S)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile and pharmaceutically acceptable salts thereof.

Further preferred is a compound
1-(4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile and pharmaceutically acceptable salts thereof.

Further preferred is a compound
1-((R)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile and pharmaceutically acceptable salts thereof.

Further preferred is a compound
1-((S)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile and pharmaceutically acceptable salts thereof.

Further preferred is a compound selected from
6-(8-chloronaphthalen-1-yl)-1-(3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile;
6-(8-chloronaphthalen-1-yl)-1-((S)-3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile;
6-(8-chloronaphthalen-1-yl)-1-((R)-3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile and pharmaceutically acceptable salts thereof.

Further preferred is a compound
6-(8-chloronaphthalen-1-yl)-1-(3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile and pharmaceutically acceptable salts thereof.

Further preferred is a compound
6-(8-chloronaphthalen-1-yl)-1-((S)-3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile and pharmaceutically acceptable salts thereof.

Further preferred is a compound
6-(8-chloronaphthalen-1-yl)-1-((R)-3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile and pharmaceutically acceptable salts thereof.

TABLE-1
Examples

| Ex. | Structure |
|---|---|
| 1 | 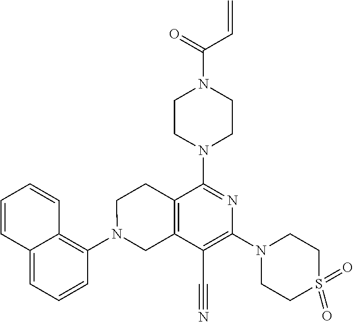 [SMILES: C=CC(=O)N1CCN(c2nc(N3CCS(=O)(=O)CC3)c(C#N)c3c2CCN(c2cccc4ccccc24)C3)CC1] |
| 2 | 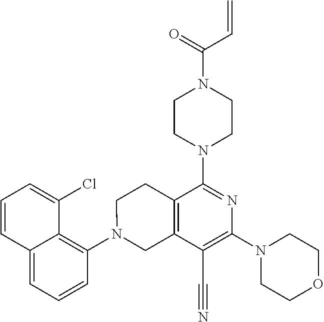 [SMILES: C=CC(=O)N1CCN(c2nc(N3CCOCC3)c(C#N)c3c2CCN(c2cccc4cccc(Cl)c24)C3)CC1] |
| 3 | 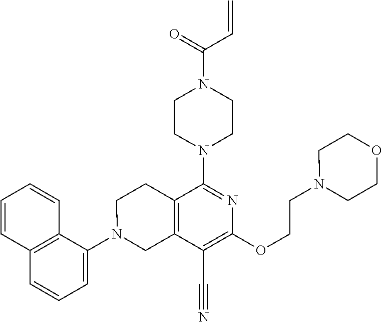 [SMILES: C=CC(=O)N1CCN(c2nc(OCCN3CCOCC3)c(C#N)c3c2CCN(c2cccc4ccccc24)C3)CC1] |
| 4 | 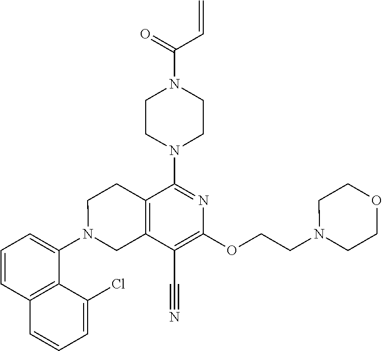 [SMILES: C=CC(=O)N1CCN(c2nc(OCCN3CCOCC3)c(C#N)c3c2CCN(c2cccc4cccc(Cl)c24)C3)CC1] |
| 5 | 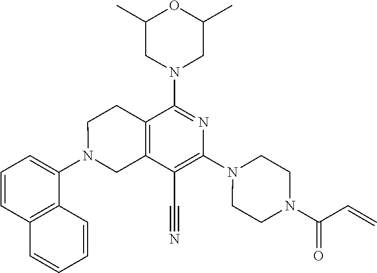 [SMILES: C=CC(=O)N1CCN(c2nc(N3CC(C)OC(C)C3)c3c(c2C#N)CN(c2cccc4ccccc24)CC3)CC1] |
| 6 | 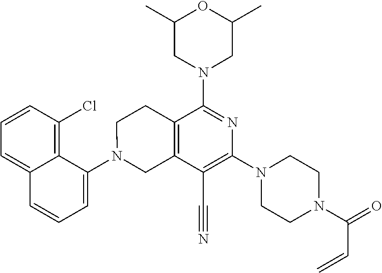 [SMILES: C=CC(=O)N1CCN(c2nc(N3CC(C)OC(C)C3)c3c(c2C#N)CN(c2cccc4cccc(Cl)c24)CC3)CC1] |
| 7 | 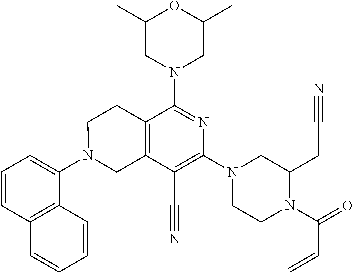 [SMILES: C=CC(=O)N1CCN(c2nc(N3CC(C)OC(C)C3)c3c(c2C#N)CN(c2cccc4ccccc24)CC3)CC1CC#N] |
| 8 | 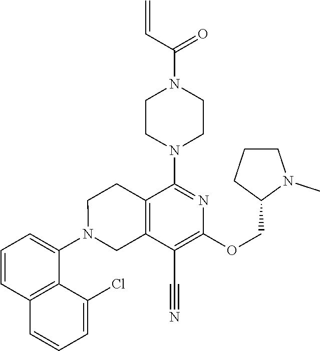 [SMILES: C=CC(=O)N1CCN(c2nc(OC[C@@H]3CCCN3C)c(C#N)c3c2CCN(c2cccc4cccc(Cl)c24)C3)CC1] |
| 9 | 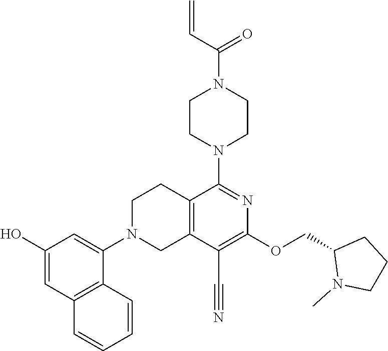 [SMILES: C=CC(=O)N1CCN(c2nc(OC[C@@H]3CCCN3C)c(C#N)c3c2CCN(c2cc(O)cc4ccccc24)C3)CC1] |
| 10 | 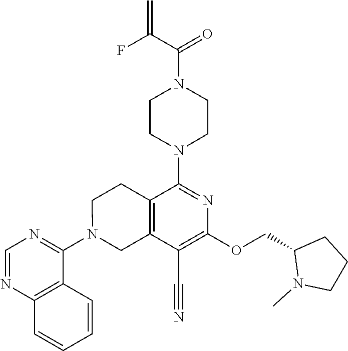 [SMILES: C=C(F)C(=O)N1CCN(c2nc(OC[C@@H]3CCCN3C)c(C#N)c3c2CCN(c2ncnc4ccccc24)C3)CC1] |
| 11 | 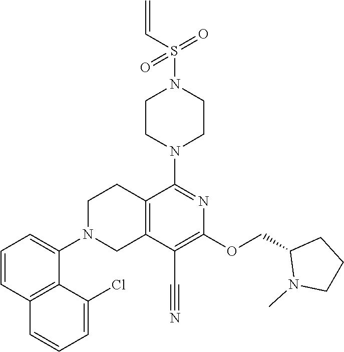 [SMILES: C=CS(=O)(=O)N1CCN(c2nc(OC[C@@H]3CCCN3C)c(C#N)c3c2CCN(c2cccc4cccc(Cl)c24)C3)CC1] |
| 12 | 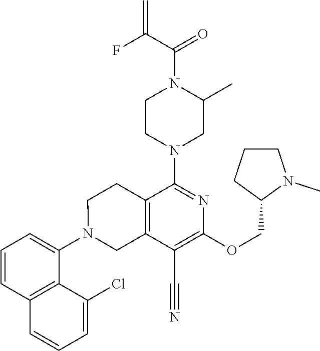 [SMILES: C=C(F)C(=O)N1CCN(c2nc(OC[C@@H]3CCCN3C)c(C#N)c3c2CCN(c2cccc4cccc(Cl)c24)C3)CC1C] |
| 13 | 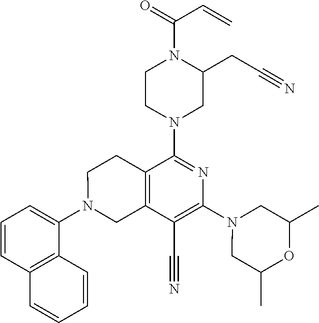 [SMILES: C=CC(=O)N1CCN(c2nc(N3CC(C)OC(C)C3)c(C#N)c3c2CCN(c2cccc4ccccc24)C3)CC1CC#N] |
| 14 | 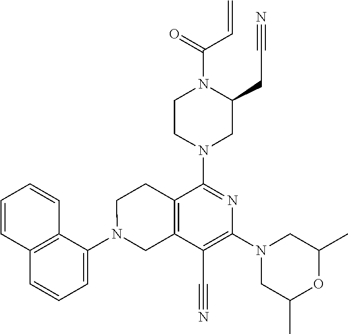 [SMILES: C=CC(=O)N1CCN(c2nc(N3CC(C)OC(C)C3)c(C#N)c3c2CCN(c2cccc4ccccc24)C3)C[C@@H]1CC#N] |
| 15 | 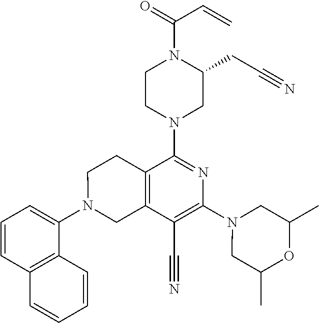 [SMILES: C=CC(=O)N1CCN(c2nc(N3CC(C)OC(C)C3)c(C#N)c3c2CCN(c2cccc4ccccc24)C3)C[C@H]1CC#N] |
| 16 | 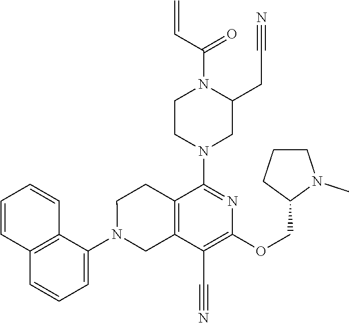 [SMILES: C=CC(=O)N1CCN(c2nc(OC[C@@H]3CCCN3C)c(C#N)c3c2CCN(c2cccc4ccccc24)C3)CC1CC#N] |
| 17 | 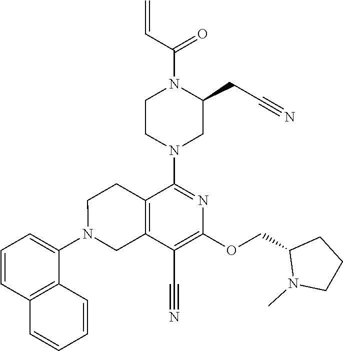 [SMILES: C=CC(=O)N1CCN(c2nc(OC[C@@H]3CCCN3C)c(C#N)c3c2CCN(c2cccc4ccccc24)C3)C[C@@H]1CC#N] |
| 18 | 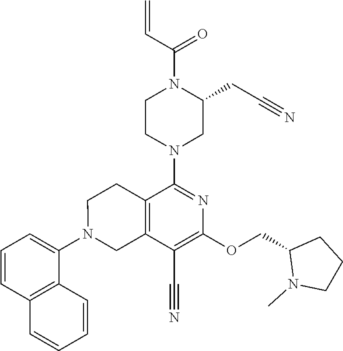 [SMILES: C=CC(=O)N1CCN(c2nc(OC[C@@H]3CCCN3C)c(C#N)c3c2CCN(c2cccc4ccccc24)C3)C[C@H]1CC#N] |
| 19 | 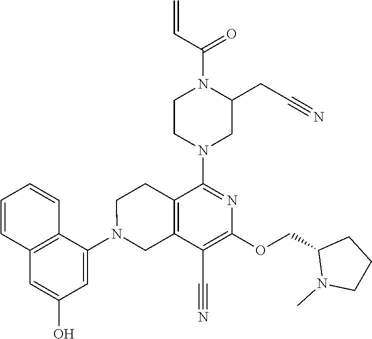 [SMILES: C=CC(=O)N1CCN(c2nc(OC[C@@H]3CCCN3C)c(C#N)c3c2CCN(c2cc(O)cc4ccccc24)C3)CC1CC#N] |
| 20 | 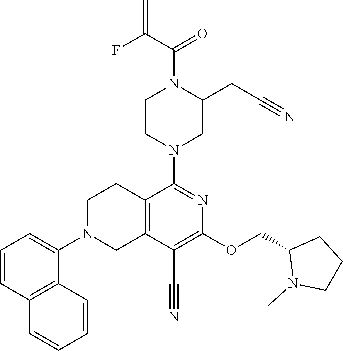 [SMILES: C=C(F)C(=O)N1CCN(c2nc(OC[C@@H]3CCCN3C)c(C#N)c3c2CCN(c2cccc4ccccc24)C3)CC1CC#N] |
| 21 | 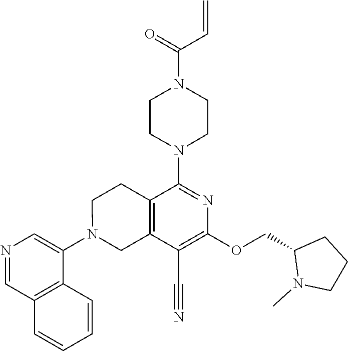 [SMILES: C=CC(=O)N1CCN(c2nc(OC[C@@H]3CCCN3C)c(C#N)c3c2CCN(c2cncc4ccccc24)C3)CC1] |
| 22 | 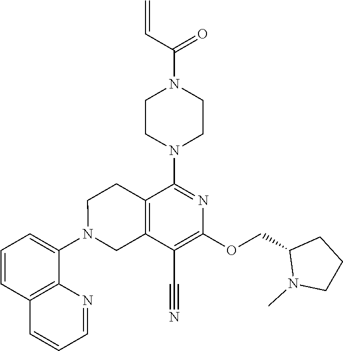 [SMILES: C=CC(=O)N1CCN(c2nc(OC[C@@H]3CCCN3C)c(C#N)c3c2CCN(c2cccc4cccnc24)C3)CC1] |
| 23 | 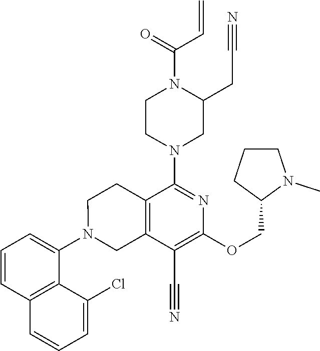 [SMILES: C=CC(=O)N1CCN(c2nc(OC[C@@H]3CCCN3C)c(C#N)c3c2CCN(c2cccc4cccc(Cl)c24)C3)CC1CC#N] |
| 24 | 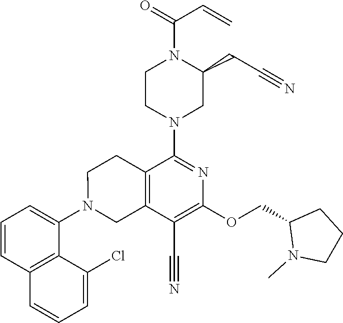 [SMILES: C=CC(=O)N1CCN(c2nc(OC[C@@H]3CCCN3C)c(C#N)c3c2CCN(c2cccc4cccc(Cl)c24)C3)C[C@@H]1CC#N] |
| 25 | 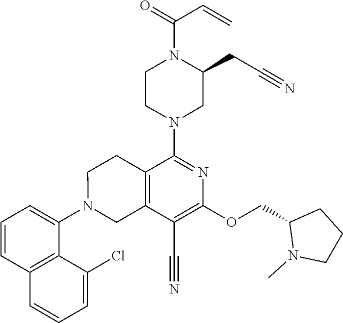 [SMILES: C=CC(=O)N1CCN(c2nc(OC[C@@H]3CCCN3C)c(C#N)c3c2CCN(c2cccc4cccc(Cl)c24)C3)C[C@@H]1CC#N] |
| 26 | 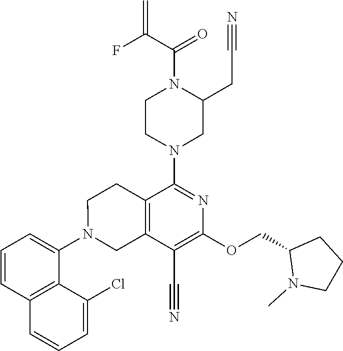 [SMILES: C=C(F)C(=O)N1CCN(c2nc(OC[C@@H]3CCCN3C)c(C#N)c3c2CCN(c2cccc4cccc(Cl)c24)C3)CC1CC#N] |
| 27 | 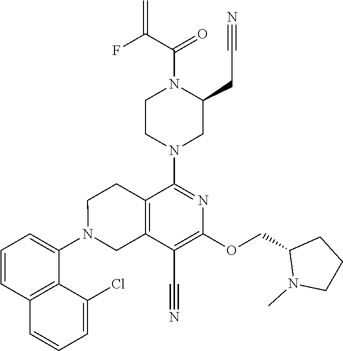 [SMILES: C=C(F)C(=O)N1CCN(c2nc(OC[C@@H]3CCCN3C)c(C#N)c3c2CCN(c2cccc4cccc(Cl)c24)C3)C[C@@H]1CC#N] |
| 28 | 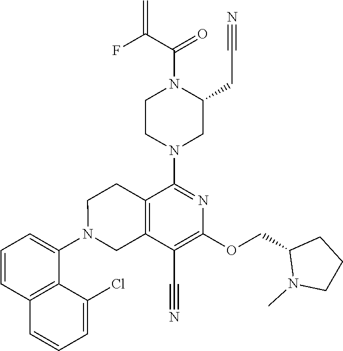 [SMILES: C=C(F)C(=O)N1CCN(c2nc(OC[C@@H]3CCCN3C)c(C#N)c3c2CCN(c2cccc4cccc(Cl)c24)C3)C[C@H]1CC#N] |
| 29 | 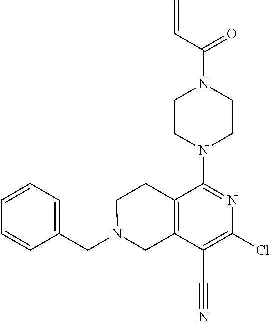 [SMILES: C=CC(=O)N1CCN(c2nc(Cl)c(C#N)c3c2CCN(Cc2ccccc2)C3)CC1] |
| 30 | 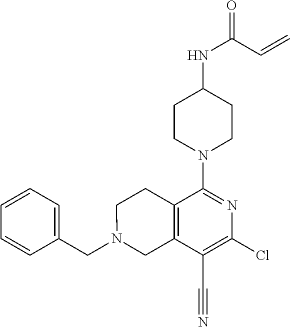 [SMILES: C=CC(=O)NC1CCN(c2nc(Cl)c(C#N)c3c2CCN(Cc2ccccc2)C3)CC1] |
| 31 | 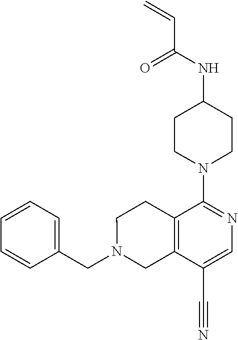 [SMILES: C=CC(=O)NC1CCN(c2ncc(C#N)c3c2CCN(Cc2ccccc2)C3)CC1] |
| 32 | 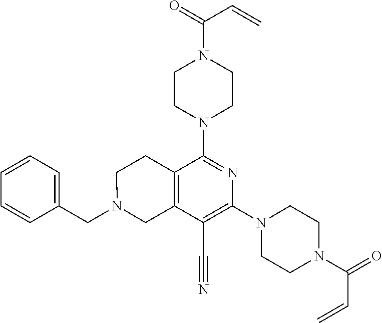 [SMILES: C=CC(=O)N1CCN(c2nc(N3CCN(C(=O)C=C)CC3)c3c(c2C#N)CN(Cc2ccccc2)CC3)CC1] |
| 33 | 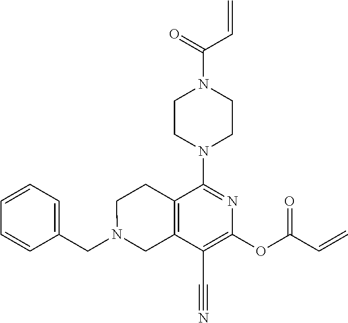 [SMILES: C=CC(=O)Oc1nc(N2CCN(C(=O)C=C)CC2)c2c(c1C#N)CN(Cc1ccccc1)CC2] |
| 34 | 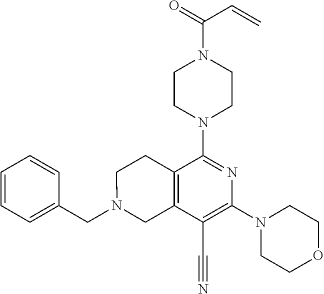 [SMILES: C=CC(=O)N1CCN(c2nc(N3CCOCC3)c(C#N)c3c2CCN(Cc2ccccc2)C3)CC1] |
| 35 | 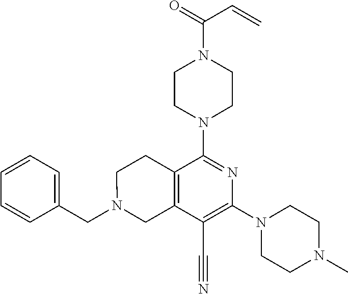 [SMILES: C=CC(=O)N1CCN(c2nc(N3CCN(C)CC3)c(C#N)c3c2CCN(Cc2ccccc2)C3)CC1] |
| 36 | 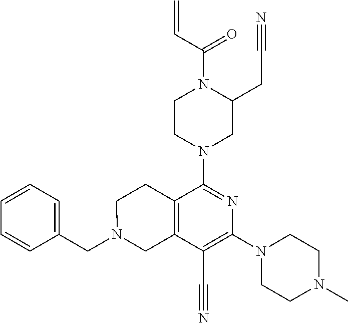 [SMILES: C=CC(=O)N1CCN(c2nc(N3CCN(C)CC3)c(C#N)c3c2CCN(Cc2ccccc2)C3)CC1CC#N] |
| 37 | 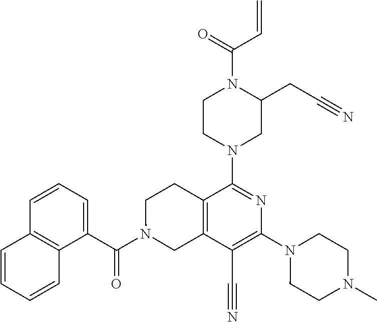 [SMILES: C=CC(=O)N1CCN(c2nc(N3CCN(C)CC3)c(C#N)c3c2CCN(C(=O)c2cccc4ccccc24)C3)CC1CC#N] |
| 38 | 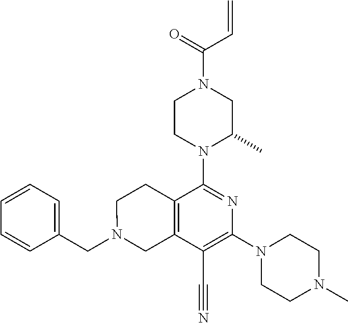 [SMILES: C=CC(=O)N1CCN(c2nc(N3CCN(C)CC3)c(C#N)c3c2CCN(Cc2ccccc2)C3)[C@@H](C)C1] |
| 39 | 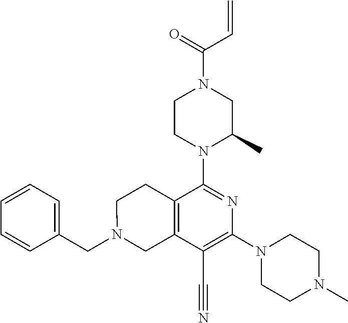 [SMILES: C=CC(=O)N1CCN(c2nc(N3CCN(C)CC3)c(C#N)c3c2CCN(Cc2ccccc2)C3)[C@H](C)C1] |
| 40 | 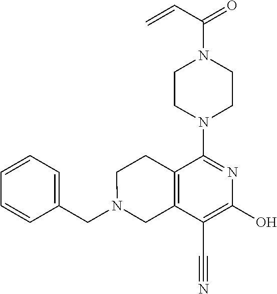 [SMILES: C=CC(=O)N1CCN(c2nc(O)c(C#N)c3c2CCN(Cc2ccccc2)C3)CC1] |
| 41 | 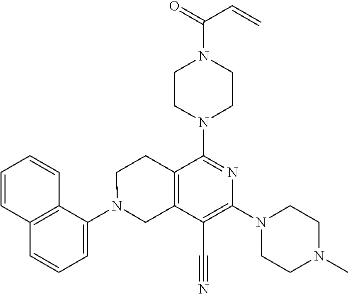 [SMILES: C=CC(=O)N1CCN(c2nc(N3CCN(C)CC3)c(C#N)c3c2CCN(c2cccc4ccccc24)C3)CC1] |
| 42 | 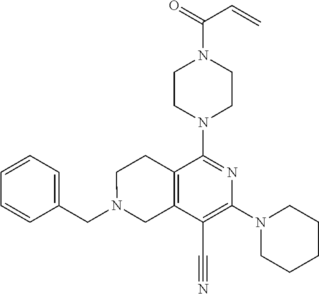 [SMILES: C=CC(=O)N1CCN(c2nc(N3CCCCC3)c(C#N)c3c2CCN(Cc2ccccc2)C3)CC1] |
| 43 | 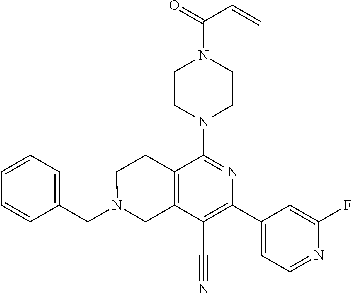 [SMILES: C=CC(=O)N1CCN(c2nc(-c3ccnc(F)c3)c(C#N)c3c2CCN(Cc2ccccc2)C3)CC1] |
| 44 | 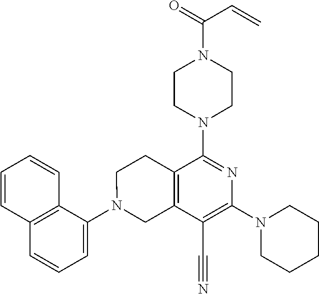 [SMILES: C=CC(=O)N1CCN(c2nc(N3CCCCC3)c(C#N)c3c2CCN(c2cccc4ccccc24)C3)CC1] |
| 45 | 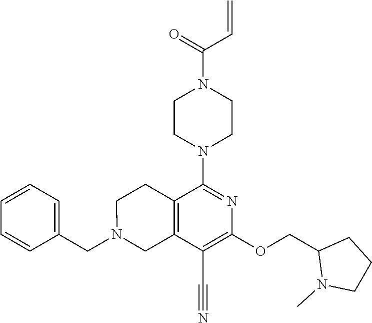 [SMILES: C=CC(=O)N1CCN(c2nc(OCC3CCCN3C)c(C#N)c3c2CCN(Cc2ccccc2)C3)CC1] |
| 46 | 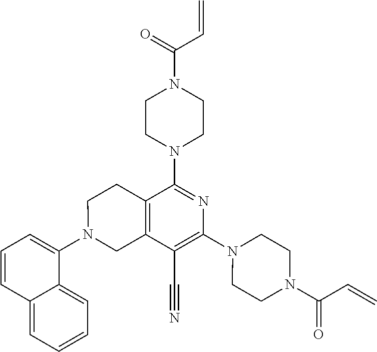 [SMILES: C=CC(=O)N1CCN(c2nc(N3CCN(C(=O)C=C)CC3)c3c(c2C#N)CN(c2cccc4ccccc24)CC3)CC1] |
| 47 | 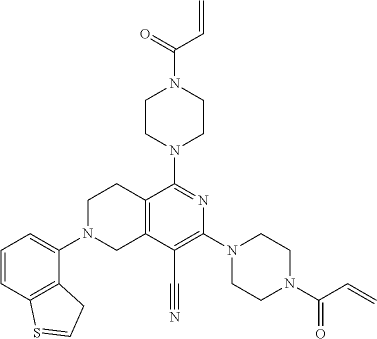 |
| 48 | 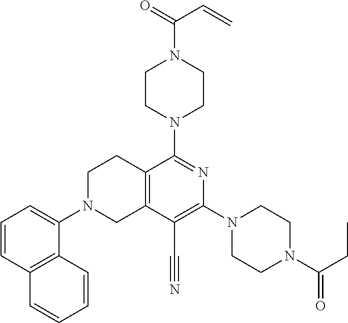 [SMILES: C=CC(=O)N1CCN(c2nc(N3CCN(C(=O)CC)CC3)c(C#N)c3c2CCN(c2cccc4ccccc24)C3)CC1] |
| 49 | 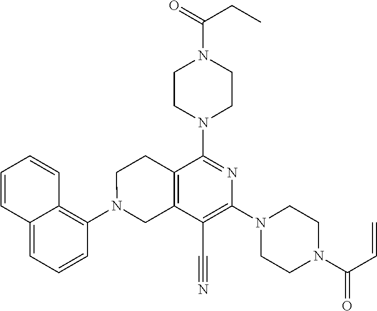 [SMILES: C=CC(=O)N1CCN(c2nc(N3CCN(C(=O)CC)CC3)c3c(c2C#N)CN(c2cccc4ccccc24)CC3)CC1] |
| 50 | 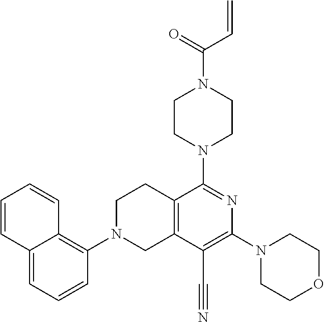 [SMILES: C=CC(=O)N1CCN(c2nc(N3CCOCC3)c(C#N)c3c2CCN(c2cccc4ccccc24)C3)CC1] |
| 51 | 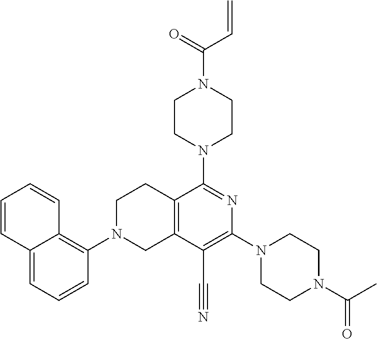 [SMILES: C=CC(=O)N1CCN(c2nc(N3CCN(C(C)=O)CC3)c(C#N)c3c2CCN(c2cccc4ccccc24)C3)CC1] |
| 52 | 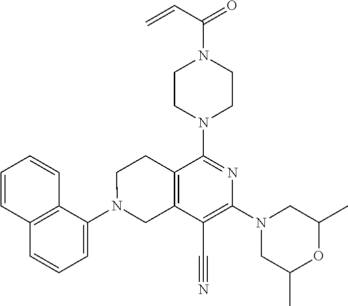 [SMILES: C=CC(=O)N1CCN(c2nc(N3CC(C)OC(C)C3)c(C#N)c3c2CCN(c2cccc4ccccc24)C3)CC1] |
| 53 | 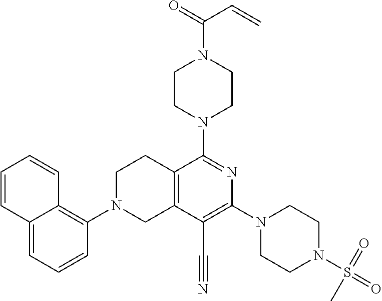 [SMILES: C=CC(=O)N1CCN(c2nc(N3CCN(S(C)(=O)=O)CC3)c(C#N)c3c2CCN(c2cccc4ccccc24)C3)CC1] |
| 54 | 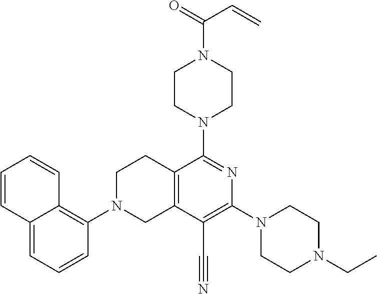 [SMILES: C=CC(=O)N1CCN(c2nc(N3CCN(CC)CC3)c(C#N)c3c2CCN(c2cccc4ccccc24)C3)CC1] |
| 55 | 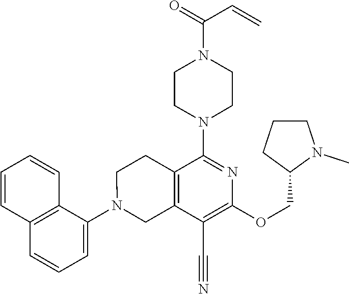 [SMILES: C=CC(=O)N1CCN(c2nc(OC[C@@H]3CCCN3C)c(C#N)c3c2CCN(c2cccc4ccccc24)C3)CC1] |
| 56 | 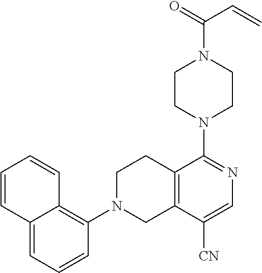 [SMILES: C=CC(=O)N1CCN(c2ncc(C#N)c3c2CCN(c2cccc4ccccc24)C3)CC1] |
| 57 | 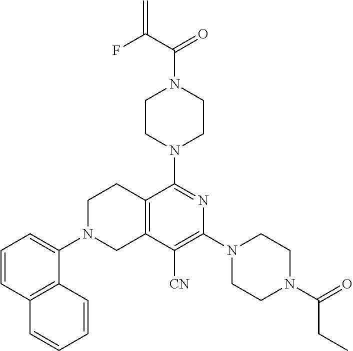 [SMILES: C=C(F)C(=O)N1CCN(c2nc(N3CCN(C(=O)CC)CC3)c(C#N)c3c2CCN(c2cccc4ccccc24)C3)CC1] |

The non-linking representative compounds of the present invention as listed herein below, and pharmaceutically acceptable salts thereof are

TABLE-2

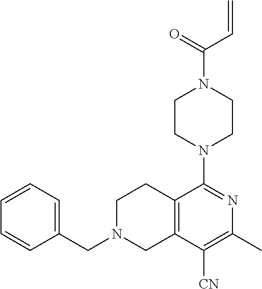 [SMILES: C=CC(=O)N1CCN(c2nc(C)c(C#N)c3c2CCN(Cc2ccccc2)C3)CC1]

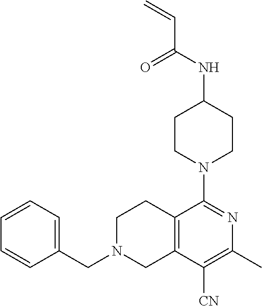 [SMILES: C=CC(=O)NC1CCN(c2nc(C)c(C#N)c3c2CCN(Cc2ccccc2)C3)CC1]

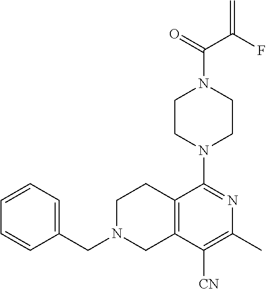 [SMILES: C=C(F)C(=O)N1CCN(c2nc(C)c(C#N)c3c2CCN(Cc2ccccc2)C3)CC1]

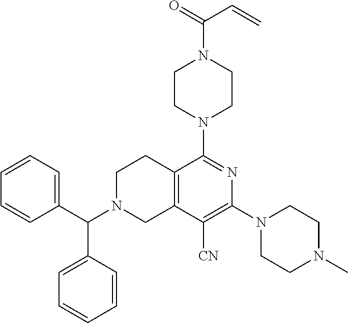 [SMILES: C=CC(=O)N1CCN(c2nc(N3CCN(C)CC3)c(C#N)c3c2CCN(C(c2ccccc2)c2ccccc2)C3)CC1]

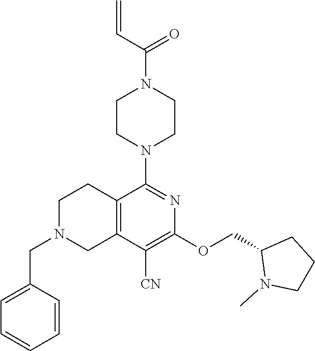 [SMILES: C=CC(=O)N1CCN(c2nc(OC[C@@H]3CCCN3C)c(C#N)c3c2CCN(Cc2ccccc2)C3)CC1]

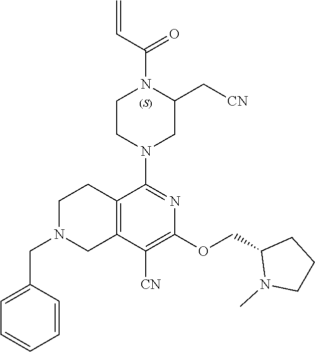

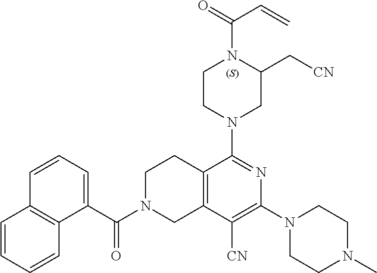

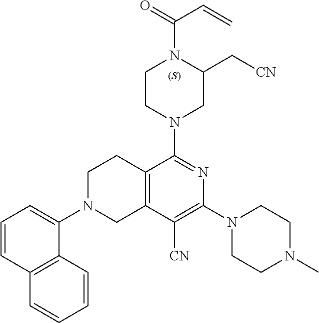

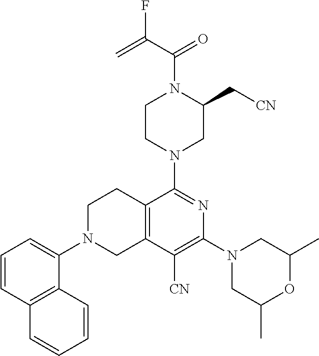 [SMILES: C=C(F)C(=O)N1CCN(c2nc(N3CC(C)OC(C)C3)c(C#N)c3c2CCN(c2cccc4ccccc24)C3)C[C@@H]1CC#N]

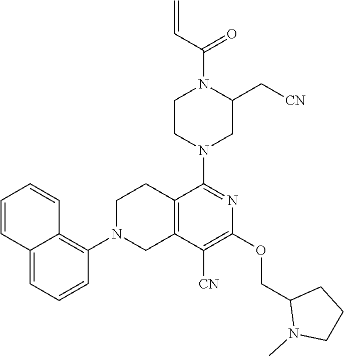 [SMILES: C=CC(=O)N1CCN(c2nc(OCC3CCCN3C)c(C#N)c3c2CCN(c2cccc4ccccc24)C3)CC1CC#N]

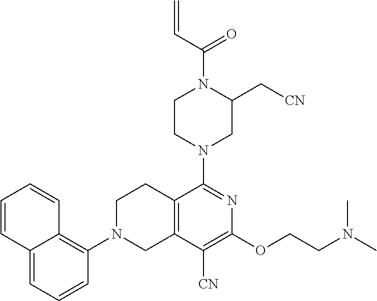 [SMILES: C=CC(=O)N1CCN(c2nc(OCCN(C)C)c(C#N)c3c2CCN(c2cccc4ccccc24)C3)CC1CC#N]

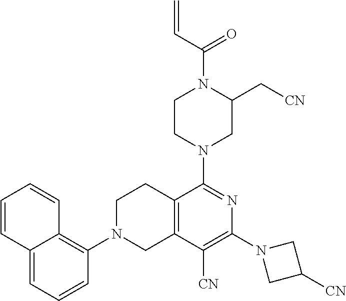 [SMILES: C=CC(=O)N1CCN(c2nc(N3CC(C#N)C3)c(C#N)c3c2CCN(c2cccc4ccccc24)C3)CC1CC#N]

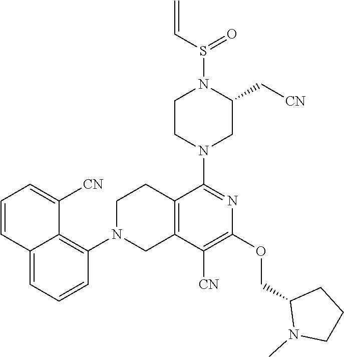 [SMILES: C=CS(=O)N1CCN(c2nc(OC[C@@H]3CCCN3C)c(C#N)c3c2CCN(c2cccc4cccc(C#N)c24)C3)C[C@H]1CC#N]

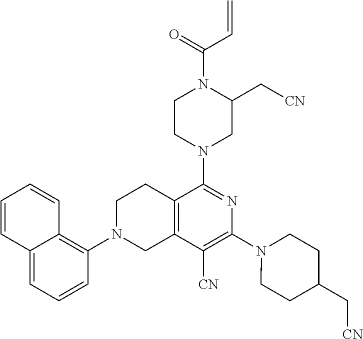 [SMILES: C=CC(=O)N1CCN(c2nc(N3CCC(CC#N)CC3)c(C#N)c3c2CCN(c2cccc4ccccc24)C3)CC1CC#N]

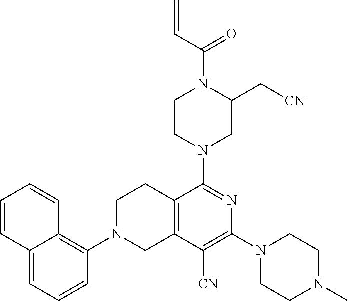 [SMILES: C=CC(=O)N1CCN(c2nc(N3CCN(C)CC3)c(C#N)c3c2CCN(c2cccc4ccccc24)C3)CC1CC#N]

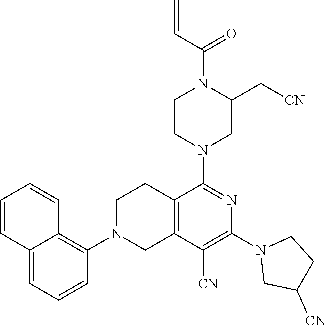 [SMILES: C=CC(=O)N1CCN(c2nc(N3CCC(C#N)C3)c(C#N)c3c2CCN(c2cccc4ccccc24)C3)CC1CC#N]

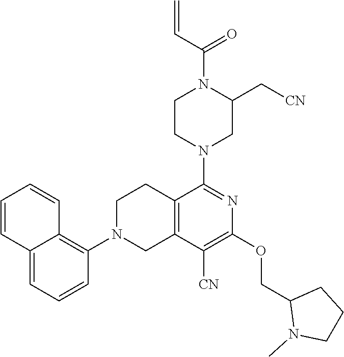 [SMILES: C=CC(=O)N1CCN(c2nc(OCC3CCCN3C)c(C#N)c3c2CCN(c2cccc4ccccc24)C3)CC1CC#N]

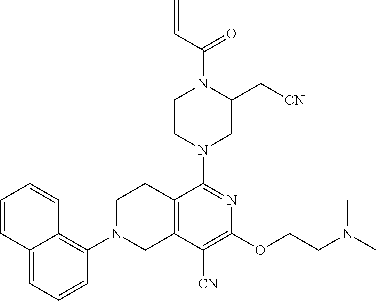 [SMILES: C=CC(=O)N1CCN(c2nc(OCCN(C)C)c(C#N)c3c2CCN(c2cccc4ccccc24)C3)CC1CC#N]

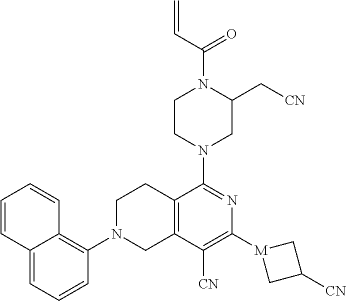

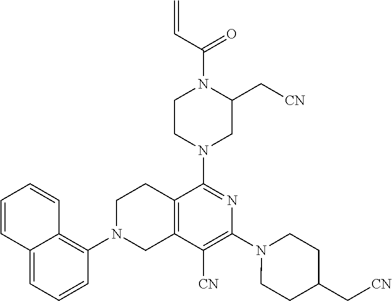 [SMILES: C=CC(=O)N1CCN(c2nc(N3CCC(CC#N)CC3)c(C#N)c3c2CCN(c2cccc4ccccc24)C3)CC1CC#N]

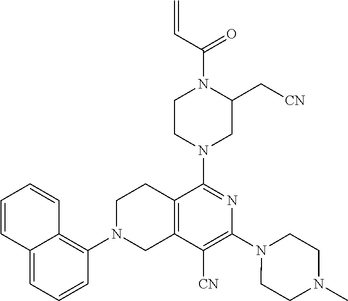 [SMILES: C=CC(=O)N1CCN(c2nc(N3CCN(C)CC3)c(C#N)c3c2CCN(c2cccc4ccccc24)C3)CC1CC#N]

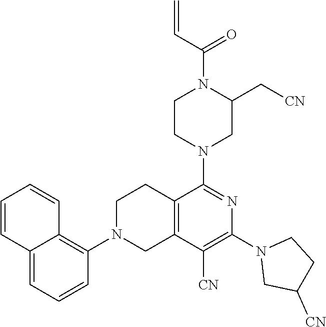 [SMILES: C=CC(=O)N1CCN(c2nc(N3CCC(C#N)C3)c(C#N)c3c2CCN(c2cccc4ccccc24)C3)CC1CC#N]

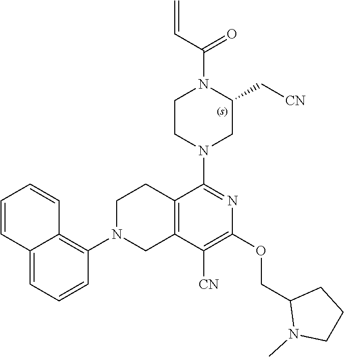 [SMILES: C=CC(=O)N1CCN(c2nc(OCC3CCCN3C)c(C#N)c3c2CCN(c2cccc4ccccc24)C3)C[C@H]1CC#N]

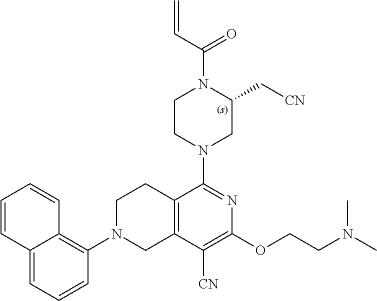 [SMILES: C=CC(=O)N1CCN(c2nc(OCCN(C)C)c(C#N)c3c2CCN(c2cccc4ccccc24)C3)C[C@H]1CC#N]

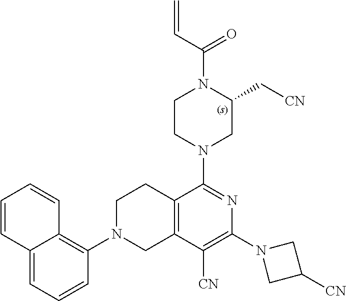 [SMILES: C=CC(=O)N1CCN(c2nc(N3CC(C#N)C3)c(C#N)c3c2CCN(c2cccc4ccccc24)C3)C[C@H]1CC#N]

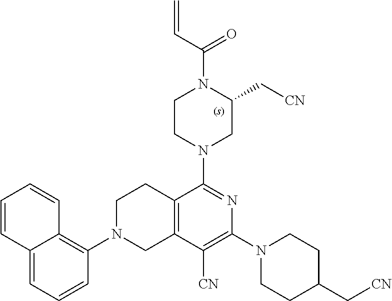 [SMILES: C=CC(=O)N1CCN(c2nc(N3CCC(CC#N)CC3)c(C#N)c3c2CCN(c2cccc4ccccc24)C3)C[C@H]1CC#N]

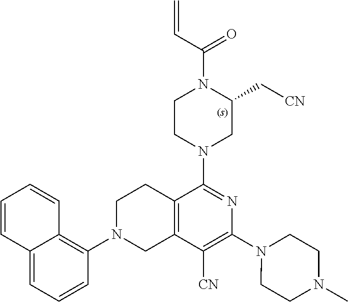 [SMILES: C=CC(=O)N1CCN(c2nc(N3CCN(C)CC3)c(C#N)c3c2CCN(c2cccc4ccccc24)C3)C[C@H]1CC#N]

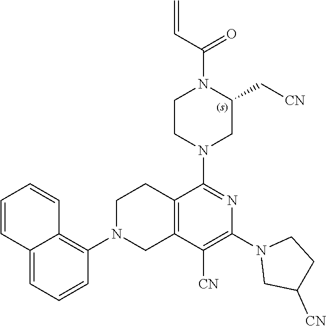 [SMILES: C=CC(=O)N1CCN(c2nc(N3CCC(C#N)C3)c(C#N)c3c2CCN(c2cccc4ccccc24)C3)C[C@H]1CC#N]

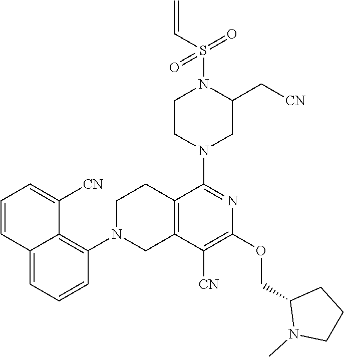 [SMILES: C=CS(=O)(=O)N1CCN(c2nc(OC[C@@H]3CCCN3C)c(C#N)c3c2CCN(c2cccc4cccc(C#N)c24)C3)CC1CC#N]

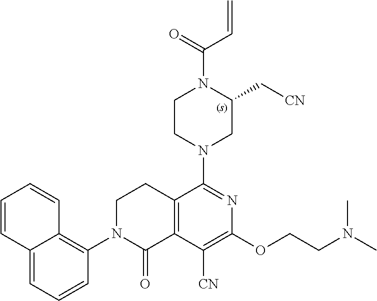 [SMILES: C=CC(=O)N1CCN(c2nc(OCCN(C)C)c(C#N)c3c2CCN(c2cccc4ccccc24)C3=O)C[C@H]1CC#N]

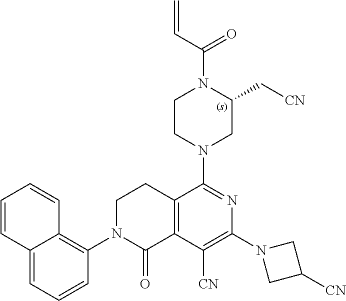 [SMILES: C=CC(=O)N1CCN(c2nc(N3CC(C#N)C3)c(C#N)c3c2CCN(c2cccc4ccccc24)C3=O)C[C@H]1CC#N]

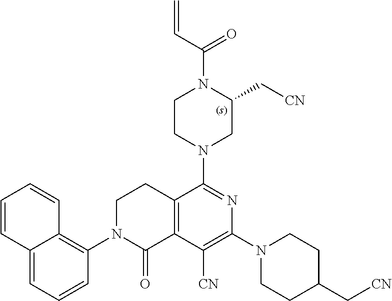 [SMILES: C=CC(=O)N1CCN(c2nc(N3CCC(CC#N)CC3)c(C#N)c3c2CCN(c2cccc4ccccc24)C3=O)C[C@H]1CC#N]

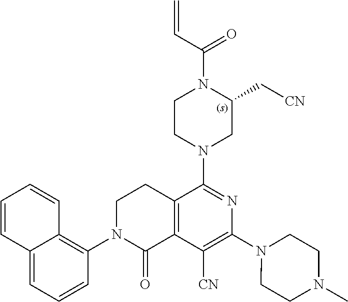 [SMILES: C=CC(=O)N1CCN(c2nc(N3CCN(C)CC3)c(C#N)c3c2CCN(c2cccc4ccccc24)C3=O)C[C@H]1CC#N]

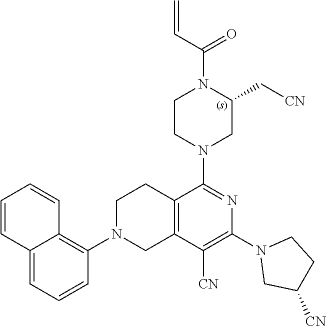 [SMILES: C=CC(=O)N1CCN(c2nc(N3CC[C@H](C#N)C3)c(C#N)c3c2CCN(c2cccc4ccccc24)C3)C[C@H]1CC#N]

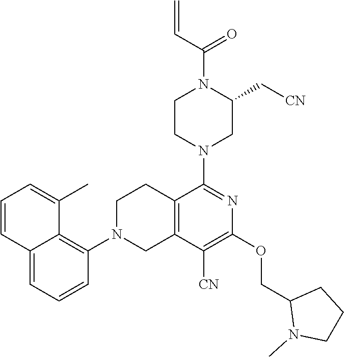 [SMILES: C=CC(=O)N1CCN(c2nc(OCC3CCCN3C)c(C#N)c3c2CCN(c2cccc4cccc(C)c24)C3)C[C@H]1CC#N]

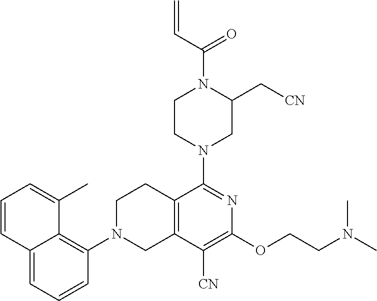 [SMILES: C=CC(=O)N1CCN(c2nc(OCCN(C)C)c(C#N)c3c2CCN(c2cccc4cccc(C)c24)C3)CC1CC#N]

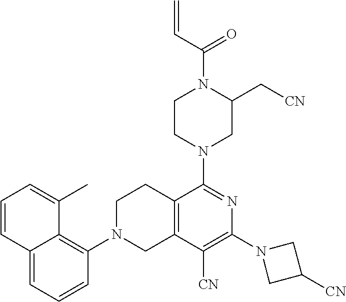 [SMILES: C=CC(=O)N1CCN(c2nc(N3CC(C#N)C3)c(C#N)c3c2CCN(c2cccc4cccc(C)c24)C3)CC1CC#N]

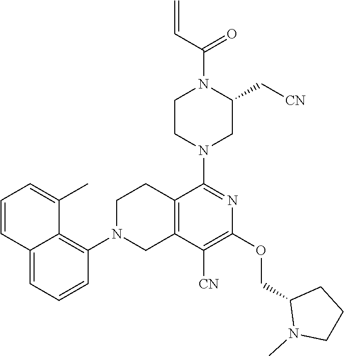 [SMILES: C=CC(=O)N1CCN(c2nc(OC[C@@H]3CCCN3C)c(C#N)c3c2CCN(c2cccc4cccc(C)c24)C3)C[C@H]1CC#N]

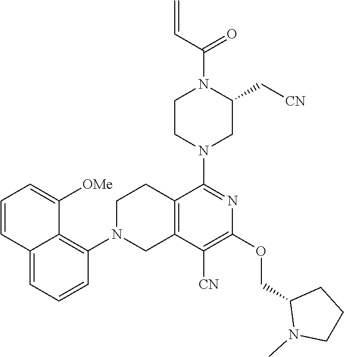 [SMILES: C=CC(=O)N1CCN(c2nc(OC[C@@H]3CCCN3C)c(C#N)c3c2CCN(c2cccc4cccc(OC)c24)C3)C[C@H]1CC#N]

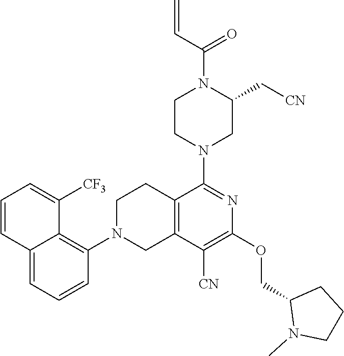 [SMILES: C=CC(=O)N1CCN(c2nc(OC[C@@H]3CCCN3C)c(C#N)c3c2CCN(c2cccc4cccc(C(F)(F)F)c24)C3)C[C@H]1CC#N]

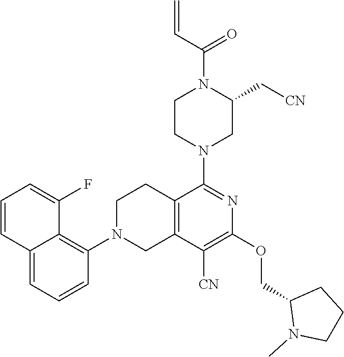 [SMILES: C=CC(=O)N1CCN(c2nc(OC[C@@H]3CCCN3C)c(C#N)c3c2CCN(c2cccc4cccc(F)c24)C3)C[C@H]1CC#N]

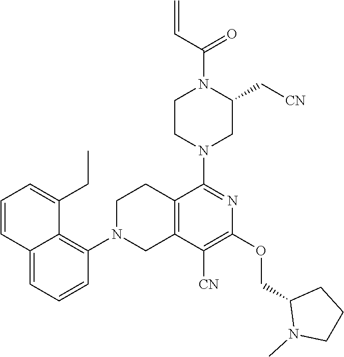 [SMILES: C=CC(=O)N1CCN(c2nc(OC[C@@H]3CCCN3C)c(C#N)c3c2CCN(c2cccc4cccc(CC)c24)C3)C[C@H]1CC#N]

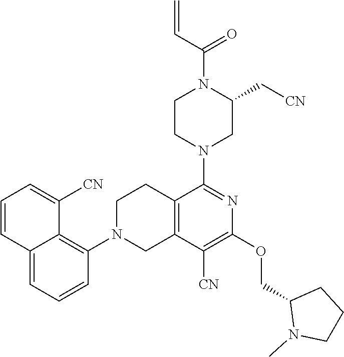 [SMILES: C=CC(=O)N1CCN(c2nc(OC[C@@H]3CCCN3C)c(C#N)c3c2CCN(c2cccc4cccc(C#N)c24)C3)C[C@H]1CC#N]

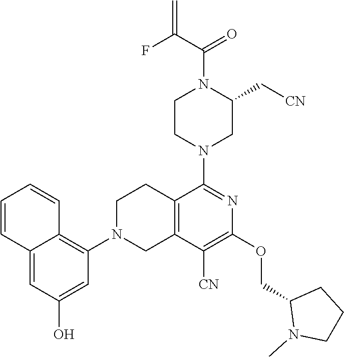 [SMILES: C=C(F)C(=O)N1CCN(c2nc(OC[C@@H]3CCCN3C)c(C#N)c3c2CCN(c2cc(O)cc4ccccc24)C3)C[C@H]1CC#N]

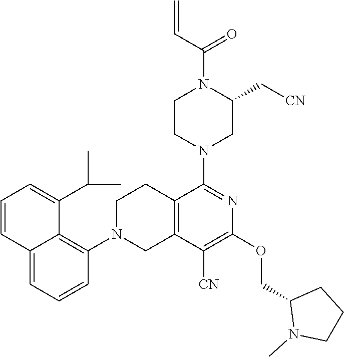 [SMILES: C=CC(=O)N1CCN(c2nc(OC[C@@H]3CCCN3C)c(C#N)c3c2CCN(c2cccc4cccc(C(C)C)c24)C3)C[C@H]1CC#N]

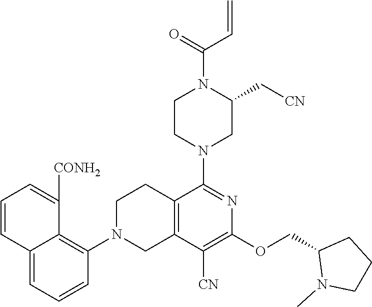 [SMILES: C=CC(=O)N1CCN(c2nc(OC[C@@H]3CCCN3C)c(C#N)c3c2CCN(c2cccc4cccc(C(N)=O)c24)C3)C[C@H]1CC#N]

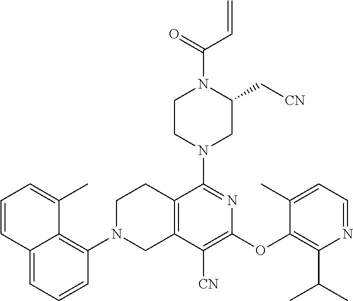 [SMILES: C=CC(=O)N1CCN(c2nc(Oc3c(C)ccnc3C(C)C)c(C#N)c3c2CCN(c2cccc4cccc(C)c24)C3)C[C@H]1CC#N]

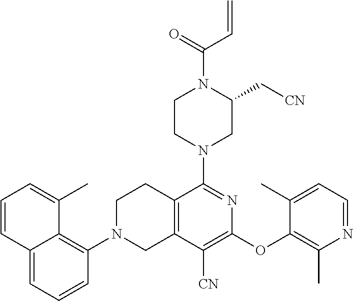 [SMILES: C=CC(=O)N1CCN(c2nc(Oc3c(C)ccnc3C)c(C#N)c3c2CCN(c2cccc4cccc(C)c24)C3)C[C@H]1CC#N]

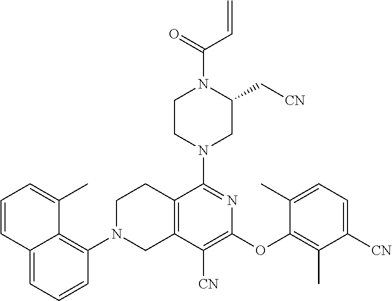 [SMILES: C=CC(=O)N1CCN(c2nc(Oc3c(C)ccc(C#N)c3C)c(C#N)c3c2CCN(c2cccc4cccc(C)c24)C3)C[C@H]1CC#N]

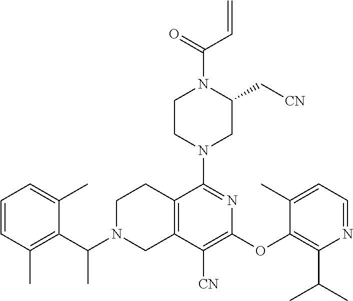 [SMILES: C=CC(=O)N1CCN(c2nc(Oc3c(C)ccnc3C(C)C)c(C#N)c3c2CCN(C(C)c2c(C)cccc2C)C3)C[C@H]1CC#N]

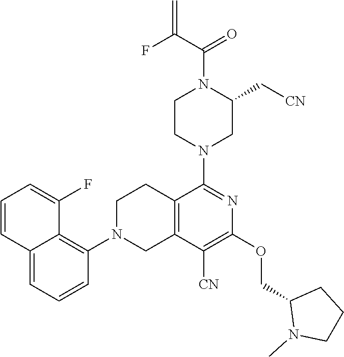 [SMILES: C=C(F)C(=O)N1CCN(c2nc(OC[C@@H]3CCCN3C)c(C#N)c3c2CCN(c2cccc4cccc(F)c24)C3)C[C@H]1CC#N]

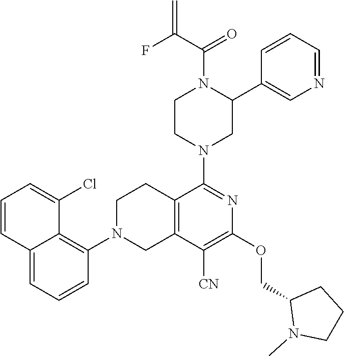 [SMILES: C=C(F)C(=O)N1CCN(c2nc(OC[C@@H]3CCCN3C)c(C#N)c3c2CCN(c2cccc4cccc(Cl)c24)C3)CC1c1cccnc1]

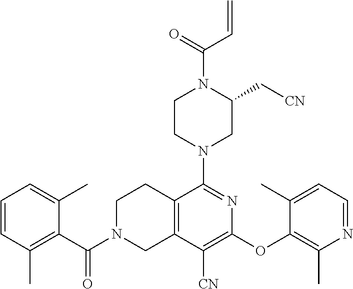 [SMILES: C=CC(=O)N1CCN(c2nc(Oc3c(C)ccnc3C)c(C#N)c3c2CCN(C(=O)c2c(C)cccc2C)C3)C[C@H]1CC#N]

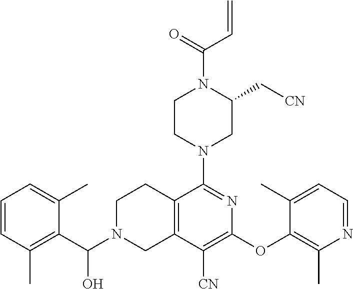 [SMILES: C=CC(=O)N1CCN(c2nc(Oc3c(C)ccnc3C)c(C#N)c3c2CCN(C(O)c2c(C)cccc2C)C3)C[C@H]1CC#N]

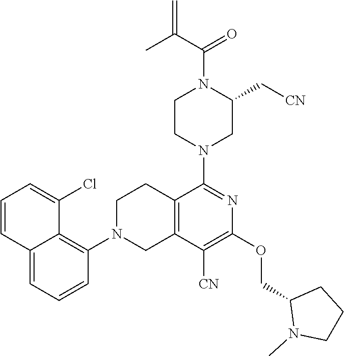 [SMILES: C=C(C)C(=O)N1CCN(c2nc(OC[C@@H]3CCCN3C)c(C#N)c3c2CCN(c2cccc4cccc(Cl)c24)C3)C[C@H]1CC#N]

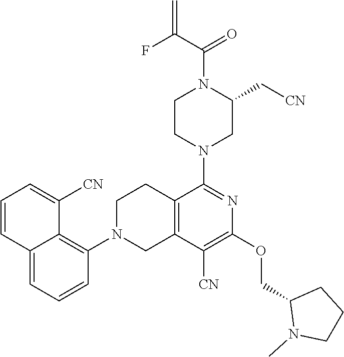 [SMILES: C=C(F)C(=O)N1CCN(c2nc(OC[C@@H]3CCCN3C)c(C#N)c3c2CCN(c2cccc4cccc(C#N)c24)C3)C[C@H]1CC#N]

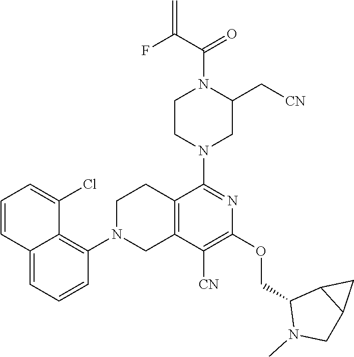 [SMILES: C=C(F)C(=O)N1CCN(c2nc(OC[C@@H]3C4CC4CN3C)c(C#N)c3c2CCN(c2cccc4cccc(Cl)c24)C3)CC1CC#N]

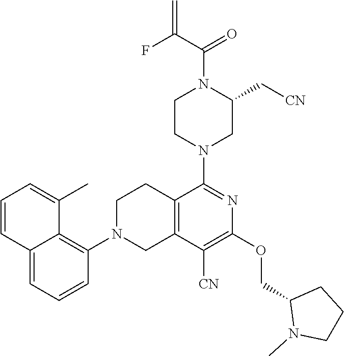 [SMILES: C=C(F)C(=O)N1CCN(c2nc(OC[C@@H]3CCCN3C)c(C#N)c3c2CCN(c2cccc4cccc(C)c24)C3)C[C@H]1CC#N]

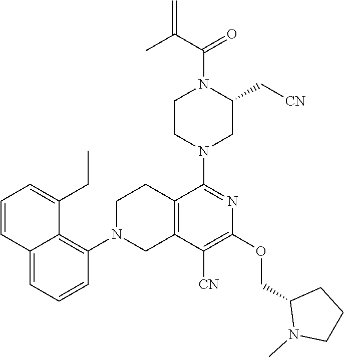 [SMILES: C=C(C)C(=O)N1CCN(c2nc(OC[C@@H]3CCCN3C)c(C#N)c3c2CCN(c2cccc4cccc(CC)c24)C3)C[C@H]1CC#N]

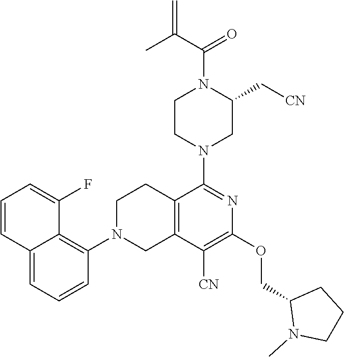 [SMILES: C=C(C)C(=O)N1CCN(c2nc(OC[C@@H]3CCCN3C)c(C#N)c3c2CCN(c2cccc4cccc(F)c24)C3)C[C@H]1CC#N]

Another embodiment of the invention is a composition comprising a compound of the invention, or a pharmaceutically acceptable salt thereof, and a pharmaceutically acceptable carrier.

Yet another embodiment of the invention is a method for treating cancer in a subject in need thereof, the method comprising administering to a subject in need thereof a therapeutically effective amount of a compound of the invention, or a pharmaceutically acceptable salt thereof, or a composition comprising a compound of the invention, or a pharmaceutically acceptable salt thereof.

Without being bound by a particular theory, provided herewith are the compounds that can modulate (e.g., inhibit) one or more members of the KRAS family, for example, one or more of KRAS mutants. More specifically, and without being bound by a particular theory, it is believed that the compounds described herein can bind to KRAS G12C and function as covalent inhibitor of KRAS G12C.

As such, in another embodiment, the invention is a method of treating a KRAS mediated disorder in a subject in need thereof, in particular KRAS G12C mediated disorder comprising administering to the subject in need thereof a therapeutically effective amount of a compound of the invention, or a pharmaceutically acceptable salt thereof, or a pharmaceutical composition comprising a compound of the invention, or a pharmaceutically acceptable salt thereof.

Another embodiment of the invention is use of a compound of the invention for treating cancer or a RAS-mediated disorder, in particular KRAS G12C mediated disorder in a subject.

Another embodiment of the invention is use of a compound of the invention for the manufacture of a medicament for treating cancer or a RAS mediated disorder, in particular KRAS G12C mediated disorder in a subject.

Compounds of the present invention, and pharmaceutically acceptable salts and/or compositions thereof, are useful for treating a variety of cancers, such as solid cancer and, more specifically, solid cancers with KRASG12 mutation.

### BRIEF DESCRIPTION OF THE DRAWINGS

FIG. 1A: Line graph depicting the effect of Cpd A in KRASG12C mutant cell lines in a 2D cell viability assay.

FIG. 1B: Line Graph depicting the effect of Cpd A in KRASG12C mutant cell lines in a 3D cell viability assay.

FIG. 1C: Line Graph depicting the effect of Cpd A on eCT26 cell lines in a 3D cell viability assay.

FIG. 1D: Line Graph depicting the effect of Cpd A on CO-04-0070 a patient derived cell line in a 3D cell viability assay.

FIG. 1E: Line Graph depicting the selectivity of Cpd A on Non-KRASG12C cell line in a 2D cell viability assay.

FIG. 2: Line Graph depicting the antitumor effect of Cpd A in NCI-H358 Xenograft

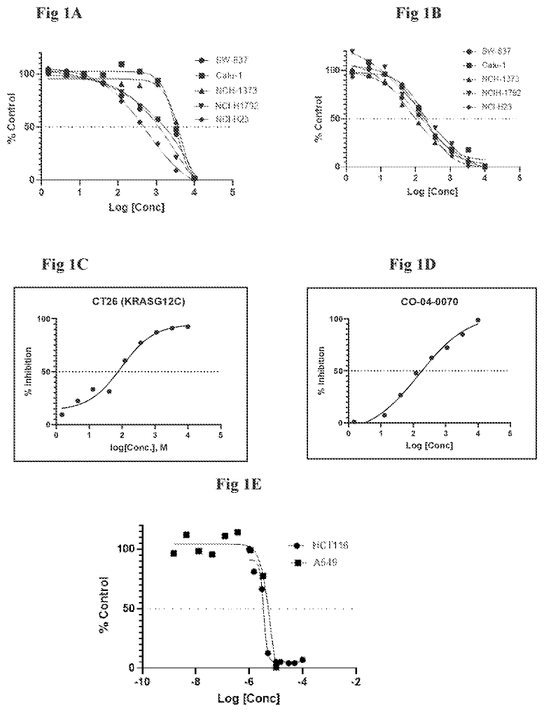

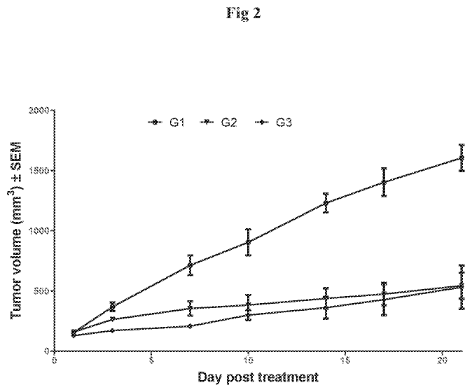

### DETAILED DESCRIPTION

A description of example embodiments of the invention follows. Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

Unless specified otherwise within this specification, the nomenclature used in this specification generally follows the examples and rules stated in Nomenclature of Organic Chemistry, Sections A, B, C, D, E. F, and H, Pergamon Press, Oxford, 1979, which is incorporated by reference herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

Compounds of the present invention may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemates, racemic mixtures, and as individual diastereomers or enantiomers, with all possible isomers and mixtures thereof, including optical isomers, being included in the present invention.

As used herein the following definitions shall apply unless otherwise indicated. Further many of the groups defined herein can be optionally substituted. The listing of substituents in the definition is exemplary and is not to be construed to limit the substituents defined elsewhere in the specification.

The term “alkyl”, unless otherwise specified, refers to a straight or branched hydrocarbon chain radical consisting solely of carbon and hydrogen atoms, containing no unsaturation, having from one to eight carbon atoms, and which is attached to the rest of the molecule by a single bond. e.g., methyl, ethyl, n-propyl, 1-methylethyl (isopropyl), n-butyl, n-pentyl, and 1,1-dimethylethyl (t-butyl). The term “C₁₋₃ alkyl” refers to an alkyl group as defined above having up to 3 carbon atoms. The term “C₁₋₆ alkyl” refers to an alkyl group as defined above having up to 6 carbon atoms. In appropriate circumstances, the term “alkyl” refers to a hydrocarbon chain radical as mentioned above which is bivalent.

The term “alkenyl”, unless otherwise specified, refers to an aliphatic hydrocarbon group containing one or more carbon-carbon double bonds and which may be a straight or branched or branched chain having about 2 to about 10 carbon atoms, e.g., ethenyl. I-propenyl, 2-propenyl (allyl), iso-propenyl, 2-methyl-1-propenyl, 1-butenyl, and 2-butenyl. The term “C₂₋₆ alkenyl” refers to an alkenyl group as defined above having up to 6 carbon atoms. In appropriate circumstances, the term “alkenyl” refers to a hydrocarbon group as mentioned above which is bivalent.

The term “alkynyl”, unless otherwise specified, refers to a straight or branched chain hydrocarbyl radical having at least one carbon-carbon triple bond, and having in the range of 2 to up to 12 carbon atoms (with radicals having in the range of 2 to up to 10 carbon atoms presently being preferred) e.g., ethynyl, propynyl, and butenyl. The term “C₂₋₆ alkynyl” refers to an alkynyl group as defined above having up to 6 carbon atoms. In appropriate circumstances, the term “alkynyl” refers to a hydrocarbyl radical as mentioned above which is bivalent.

The term “alkoxy” unless otherwise specified, denotes an alkyl, cycloalkyl, or cycloalkylalkyl group as defined above attached via an oxygen linkage to the rest of the molecule. The term “substituted alkoxy” refers to an alkoxy group where the alkyl constituent is substituted (i.e., —O-(substituted alkyl). For example “alkoxy” refers to the group —O-alkyl, including from 1 to 8 carbon atoms of a straight, branched, cyclic configuration and combinations thereof attached to the parent structure through an oxygen atom. Examples include methoxy, ethoxy, propoxy, isopropoxy, cyclopropyloxy, and cyclohexyloxy. In appropriate circumstances, the term “alkoxy” refers to a group as mentioned above which is bivalent.

The term “cycloalkyl”, unless otherwise specified, denotes a non-aromatic mono or multicyclic ring system of about 3 to 12 carbon atoms such as cyclopropyl, cyclobutyl, cyclopentyl, and cyclohexyl. Examples of multicyclic cycloalkyl groups include perhydronaphthyl, adamantyl and norbornyl groups, bridged cyclic groups, and spirobicyclic groups, e.g., spiro (4,4) non-2-yl. The term “C₃₋₆ cycloalkyl” refers to a cycloalkyl group as defined above having up to 6 carbon atoms.

The term “cycloalkylalkyl”, unless otherwise specified, refers to a cyclic ring-containing radical containing in the range of about 3 up to 8 carbon atoms directly attached to an alkyl group which is then attached to the main structure at any carbon from the alkyl group, such as cyclopropylmethyl, cyclobutylethyl, and cyclopentylethyl.

The term “cycloalkenyl”, unless otherwise specified, refers to cyclic ring-containing radicals containing in the range of about 3 up to 8 carbon atoms with at least one carbon-carbon double bond such as cyclopropenyl, cyclobutenyl, and cyclopentenyl. The term “cycloalkenylalkyl” refers to a cycloalkenyl group directly attached to an alkyl group which is then attached to the main structure at any carbon from the alkyl group.

The term “aryl”, unless otherwise specified, refers to aromatic radicals having in the range of 6 up to 20 carbon atoms such as phenyl, naphthyl, tetrahydronaphthyl, indanyl, and biphenyl.

The term “arylalkyl”, unless otherwise specified, refers to an aryl group as defined above directly bonded to an alkyl group as defined above, e.g., —CH₂C₆H₅ and —C₂H₅C₆H₅.

The term “heterocyclic ring”, unless otherwise specified, refers to a non-aromatic 3 to 15 member ring radical which consists of carbon atoms and at least one heteroatom selected from nitrogen, phosphorus, oxygen and sulfur. For purposes of this invention, the heterocyclic ring radical may be a mono-, bi-, tri- or tetracyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized. The heterocyclic ring radical may be attached to the main structure at any heteroatom or carbon atom.

The term “heterocyclyl”, unless otherwise specified, refers to a heterocylic ring radical as defined above. The heterocyclyl ring radical may be attached to the main structure at any heteroatom or carbon atom.

The term “heterocyclylalkyl”, unless otherwise specified, refers to a heterocylic ring radical as defined above directly bonded to an alkyl group. The heterocyclylalkyl radical may be attached to the main structure at any carbon atom in the alkyl group. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

The term “heteroaryl”, unless otherwise specified, refers to an optionally substituted 5 to 14 member aromatic ring having one or more heteroatoms selected from N, O, and S as ring atoms. The heteroaryl may be a mono-, bi- or tricyclic ring system. Examples of such “heterocyclic ring” or “heteroaryl” radicals include, but are not limited to, oxazolyl, thiazolyl, imidazolyl, pyrrolyl, furanyl, pyridinyl, pyrimidinyl, pyrazinyl, benzofuranyl, indolyl, benzothiazolyl, benzoxazolyl, carbazolyl, quinolyl, isoquinolyl, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, 4-piperidonyl, pyrrolidinyl, pyridazinyl, oxazolinyl, oxazolidinyl, triazolyl, indanyl, isoxazolyl, isoxazolidinyl, morpholinyl, thiazolinyl, thiazolidinyl, isothiazolyl, quinuclidinyl, isothiazolidinyl, isoindolyl, indolinyl, isoindolinyl, octahydroindolyl, octahydroisoindolyl, decahydroisoquinolyl, benzimidazolyl, thiadiazolyl, benzopyranyl, tetrahydrofuryl, tetrahydropyranyl, thienyl, benzothienyl, thiamorpholinyl, thiamorpholinyl sulfoxide, thiamorpholinyl sulfone, dioxaphospholanyl, oxadiazolyl, chromanyl, and isochromanyl. The heteroaryl ring radical may be attached to the main structure at any heteroatom or carbon atom. The term “substituted heteroaryl” also includes ring systems substituted with one or more oxide (—O—) substituents, such as pyridinyl N-oxides.

The term “heteroarylalkyl”, unless otherwise specified, refers to a heteroaryl ring radical as defined above directly bonded to an alkyl group. The heteroarylalkyl radical may be attached to the main structure at any carbon atom from alkyl group.

The term “cyclic ring” refers to a cyclic ring containing 3 to 10 carbon atoms.

The term “substituted” unless otherwise specified, refers to substitution with any one or any combination of the following substituents which may be the same or different and are independently selected from hydrogen, hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted heteroaryl, substituted or unsubstituted heteroarylalkyl, substituted or unsubstituted heterocyclic ring, substituted heterocyclylalkyl ring, substituted or unsubstituted guanidine, —COOR^{x}, —C(O)R^{x}, —C(S)R^{x}, —C(O)NR^{x}R^{y}, —C(O)ONR^{x}R^{y}, —NR^{y}R^{z}, —NR^{x}CONR^{y}R^{z}, —N(R^{x})SOR^{y}, —N(R^{x})SO₂R^{y}, -(═N—N(R^{x})R^{y}), —NR^{x}C(O)OR^{y}, —NR^{x}R^{y}, —NR^{x}C(O)R^{y}—, —NR^{x}C(S)R^{y}—NR^{x}C(S)NR^{x}R^{y}, —SONR^{x}R^{y}—, —SO₂NR^{x}R^{y}—, —OR^{x}, —OR^{x}C(O)NR^{y}R^{z}, —OR^{x}C(O)OR^{y}—, —OC(O)R^{x}, —OC(O)NR^{x}R^{y}, —R—NR^{y}C(O)R^{z}, —R^{x}OR^{y}, —R^{x}C(O)OR^{y}, —R^{x}C(O)NR^{y}R^{z}, —R^{x}C(O)R^{x}, —R^{x}OC(O)R^{y}, —SR^{x}, —SOR^{x}, —SO₂R^{x}, and —ONO₂, wherein R^{x}, R^{y} and R^{z} in each of the above groups can be hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted amino, substituted or unsubstituted heteroaryl, substituted or unsubstituted heteroarylalkyl, substituted or unsubstituted heterocyclic ring, or substituted heterocyclylalkyl ring, or any two of R^{x}, R^{y} and R^{z} may be joined to form a substituted or unsubstituted saturated or unsaturated 3-10 membered ring, which may optionally include heteroatoms which may be the same or different and are selected from O, NR^{x}(e.g., R^{x} can be hydrogen or C₁₋₆ alkyl) or S. Substitution or the combinations of substituents envisioned by this invention are preferably those that result in the formation of a stable or chemically feasible compound. The term stable as used herein refers to the compounds or the structure that are not substantially altered w % ben subjected to conditions to allow for their production, detection and preferably their recovery, purification and incorporation into a pharmaceutical composition. The substituents in the aforementioned “substituted” groups cannot be further substituted. For example, when the substituent on “substituted alkyl” is “substituted aryl”, the substituent on “substituted aryl” cannot be “substituted alkenyl”.

The term “halo”, “halide”, or, alternatively, “halogen” means fluoro, chloro, bromo or iodo.

The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

The term “protecting group” or “PG” refers to a substituent that is employed to block or protect a particular functionality. Other functional groups on the compound may remain reactive. For example, an “amino-protecting group” is a substituent attached to an amino group that blocks or protects the amino functionality in the compound. Suitable amino-protecting groups include, but are not limited to, acetyl, trifluoroacetyl, tert-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable hydroxy-protecting groups include, but are not limited to, acetyl and silyl. A “carboxy-protecting group” refers to a substituent of the carboxy group that blocks or protects the carboxy functionality. Suitable carboxy-protecting groups include, but are not limited to, —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, and nitroethyl. For a general description of protecting groups and their use, see T. W. Greene, Protective Groups in Organic Synthesis, John Wiley & Sons, New York, 1991.

Certain of the compounds described herein contain one or more asymmetric centers and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures. Non-limiting examples of intermediate mixtures include a mixture of isomers in a ratio of 10:90, 13:87, 17:83, 20:80, or 22:78. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

The term “tautomers” refers to compounds, which are characterized by relatively easy interconversion of isomeric forms in equilibrium. These isomers are intended to be covered by this invention. “Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g. in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4 (1H)-one tautomers.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a 3C- or ¹⁴C-enriched carbon are within the scope of this invention. Such compounds are useful, for example, as analytical tools, as probes in biological assays, or as therapeutic agents in accordance with the present invention. For example, in the case of variable R, the (C₁-C₄)alkyl or the —O—(C₁-C₄)alkyl can be suitably deuterated (e.g., —CD₃, -OCD₃).

The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

The term “stereoisomers” is a general term for all isomers of an individual molecule that differ only in the orientation of their atoms in space. It includes mirror image isomers (enantiomers), geometric (cis/trans) isomers and isomers of compounds with more than one chiral center that are not mirror images of one another (diastereomers).

“Electrophile” or “electrophilic moiety” is any moiety capable of reacting with a nucleophile (e.g., a moiety having a lone pair of electrons, a negative charge, a partial negative charge and/or an excess of electrons, for example a —SH group). Electrophiles typically are electron poor or comprise atoms which are electron poor. In certain embodiments an electrophile contains a positive charge or partial positive charge, has a resonance structure which contains a positive charge or partial positive charge or is a moiety in which delocalization or polarization of electrons results in one or more atom which contains a positive charge or partial positive charge. In some embodiments, the electrophiles comprise conjugated double bonds, for example an α,β-unsaturated carbonyl or α,β-unsaturated thiocarbonyl compound.

A “leaving group or atom” (Lg or Ig) is any group or atom that will, under the reaction conditions, cleave from the starting material, thus promoting reaction at a specified site. Suitable examples of such groups unless otherwise specified are halogen atoms and mesyloxy, p-nitrobenzensulphonyloxy and tosyloxy groups.

“Prodrug” is meant to indicate a compound that may be converted under physiological conditions or by solvolysis to a biologically active compound described herein (e.g., compound of structure (A), (A-I), (A-II) and (A-III)). Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems.” A.C.S. Symposium Series, Vol. 14, and in Bioreversible Carriers in Drug Design, ed. Edward B. Roche, American Pharmaceutical Association and Pergamon Press, 1987, Prodrug design is discussed generally in Hardma, et al. (Eds.). Goodman and Gilman's The Pharmacological Basis of Therapeutics, 9th ed., pp. 11-16 (1996) all of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound and the like.

In some embodiments, prodrugs include compounds of structure (A), (A-I), (A-II) and (A-III) having a phosphate, phosphoalkoxy, ester or boronic ester substituent. Without being bound by theory, it is believed that such substituents are converted to a hydroxyl group under physiological conditions. Accordingly, embodiments include any of the compounds disclosed herein, wherein a hydroxyl group has been replaced with a phosphate, phosphoalkoxy, ester or boronic ester group, for example a phosphate or phosphoalkoxy group. For example, in some embodiments a hydroxyl group on the R¹ moiety is replaced with a phosphate, phosphoalkoxy, ester or boronic ester group, for example a phosphate or alkoxy phosphate group.

The term “ester” refers to a compound, which is formed by reaction between an acid and an alcohol with elimination of water. An ester can be represented by the general formula RCOOR′.

These prodrugs and esters are intended to be covered within the scope of this invention.

Additionally, the instant invention also includes the compounds which differ only in the presence of one or more isotopically enriched atoms for example replacement of hydrogen with deuterium or tritium, or the replacement of a carbon by ¹³C- or ¹⁴C-enriched carbon.

When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range may vary from, for example, between 1% and 15% of the stated number or numerical range.

The term “comprising” (and related terms such as “comprise” or “comprises” or “having” or “including”) includes those embodiments, for example, an embodiment of any composition of matter, composition, method, or process, or the like, that “consist of” or “consist essentially of” the described features.

The following abbreviations and terms have the indicated meanings throughout; Abbreviations used herein have their conventional meaning within the chemical and biological arts.

The term “cell proliferation” refers to a phenomenon by which the cell number has changed as a result of division. This term also encompasses cell growth by which the cell morphology has changed (e.g., increased in size) consistent with a proliferative signal.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried. In one embodiment, the amount of compound administered ranges from about 0.1 mg to 5 g, from about 1 mg to 2.0 g, from about 100 mg to 1.5 g, from about 200 mg to 1.5 g, from about 400 mg to 1.5 g, and from about 400 mg to 1.0 g.

As used herein, “treatment,” “treating,” or “ameliorating” refers to an approach for obtaining beneficial or desired results including but not limited to therapeutic benefit and/or a prophylactic benefit. By therapeutic benefit is meant eradication or amelioration of the underlying disorder being treated. In addition a therapeutic benefit is also achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. These terms are used interchangeably.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, an amount of a compound effective to treat a disorder, or a “therapeutically effective amount” refers to an amount of the compound which is effective, upon single or multiple dose administration to a subject or a cell, in curing, alleviating, relieving or improving one or more symptoms of a disorder.

As used herein, an amount of a compound effective to prevent a disorder, or a “prophylactically effective amount” of the compound refers to an amount effective, upon single- or multiple-dose administration to the subject, in preventing or delaying the onset or recurrence of a disorder or one or more symptoms of the disorder.

As used herein, the term “subject” or “patient” is intended to include human and non-human animals. Exemplary human subjects include a human patient having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, cow, pig, etc., and companion animals (dog, cat, horse, etc.).

The methods described herein can be useful in both human therapeutics and veterinary applications (e.g., dogs, cats, cows, sheep, pigs, horses, goats, chickens, turkeys, ducks, and geese).

In some embodiments, the patient is a mammal, and in some embodiments, the patient is human.

“Radiation therapy” means exposing a patient, using routine methods and compositions known to the practitioner, to radiation emitters such as alpha-particle emitting radionuclides (e.g., actinium and thorium radionuclides), low linear energy transfer (LET) radiation emitters (i.e. beta emitters), conversion electron emitters (e.g. strontium-89 and samarium-153-EDTMP), or high-energy radiation, including without limitation x-rays, gamma rays, and neutrons.

The term “pharmaceutically acceptable carrier” or “pharmaceutically acceptable excipient” includes, but is not limited to, any and all, a non-toxic solvent, dispersant, excipient, adjuvant, fillers, salts, disintegrants, binders, lubricants, glidants, wetting agents, controlled release matrices, colorants/flavoring, carriers, buffers, stabilizers, solubilizers, or other material and combinations thereof which is mixed with the active ingredient in order to permit the formation of a pharmaceutical composition, i.e., a dosage form capable of being administered to a patient. One example of such a carrier is pharmaceutically acceptable oil typically used for parenteral administration. Pharmaceutically acceptable carriers are well known in the art.

It is understood that substituents and substitution patterns on the compounds of the invention can be selected by one of ordinary skill in the art to provide compounds that are chemically stable and that can be readily synthesized by techniques known in the art, as well as those methods set forth below. In general, the term “substituted,” whether preceded by the term “optionally” or not, means that one or more hydrogens of the designated moiety are replaced with a suitable substituent. Unless otherwise indicated, an “optionally substituted group” can have a suitable substituent at each substitutable position of the group and, when more than one position in any given structure may be substituted with more than one substituent selected from a specified group, the substituent can be either the same or different at every position. Alternatively, an “optionally substituted group” can be unsubstituted.

Combinations of substituents envisioned by this invention are preferably those that result in the formation of stable or chemically feasible compounds. If a substituent is itself substituted with more than one group, it is understood that these multiple groups can be on the same carbon atom or on different carbon atoms, as long as a stable structure results. The term “stable,” as used herein, refers to compounds that are not substantially altered when subjected to conditions to allow for their production, detection, and, in certain embodiments, their recovery, purification, and use for one or more of the purposes disclosed herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds of this invention include salts derived from suitable inorganic and organic acids and bases that are compatible with the treatment of patients.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable acid addition salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

In some embodiments, exemplary inorganic acids which form suitable salts include, but are not limited thereto, hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tricarboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2 phenoxybenzoic, p-toluenesulfonic acid and other sulfonic acids such as methanesulfonic acid and 2-hydroxyethanesulfonic acid. Either the mono- or di-acid salts can be formed, or such salts can exist in either a hydrated, solvated or substantially anhydrous form. In general, the acid addition salts of these compounds are more soluble in water and various hydrophilic organic solvents, and generally demonstrate higher melting points in comparison to their free base forms.

In some embodiments, acid addition salts of the compounds of formula A are most suitably formed from pharmaceutically acceptable acids, and include, for example, those formed with inorganic acids, e.g., hydrochloric, sulfuric or phosphoric acids and organic acids e.g. succinic, maleic, acetic or fumaric acid.

Other non-pharmaceutically acceptable salts, e.g., oxalates can be used, for example, in the isolation of compounds of formula (A), (A-I), (A-II) and (A-III) for laboratory use, or for subsequent conversion to a pharmaceutically acceptable acid addition salt. Also included within the scope of the invention are base addition salts (such as sodium, potassium and ammonium salts), solvates and hydrates of compounds of the invention. The conversion of a given compound salt to a desired compound salt is achieved by applying standard techniques, well known to one skilled in the art.

An “anti-cancer agent”. “anti-tumor agent” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository The term “cell proliferation” refers to a phenomenon by which the cell number has changed as a result of division. This term also encompasses cell growth by which the cell morphology has changed (e.g., increased in size) consistent with a proliferative signal

The term “selective inhibition” or “selectively inhibit” refers to a biologically active agent refers to the agent's ability to preferentially reduce the target signaling activity as compared to off-target signaling activity, via direct or indirect interaction with the target

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution

A “pharmaceutical composition” refers to a formulation of a compound of the invention and a medium generally accepted in the art for the delivery of the biologically active compound to mammals. e.g., humans. Such a medium includes all pharmaceutically acceptable carriers, diluents or excipients thereof.

When introducing elements disclosed herein, the articles “a,” “an,” “the,” and “said” are intended to mean that there are one or more of the elements. The terms “comprising,” “having” and “including” are intended to be open-ended and mean that there may be additional elements other than the listed elements.

For example, provided herein are methods of treating various cancers in mammals (including humans and non-humans), comprising administering to a patient in need thereof a compound of the invention, or a pharmaceutically acceptable salt thereof. Such cancers include hematologic malignancies (leukemias, lymphomas, myelomas, myelodysplastic and myeloproliferative syndromes) and solid tumors (carcinomas such as oral, gall bladder, prostate, breast, lung, colon, pancreatic, renal, ovarian as well as soft tissue and osteosarcomas, and stromal tumors).

### Pharmaceutical Compositions

The invention provides a pharmaceutical composition comprising one or more compounds of the present invention. The pharmaceutical composition may include one or more additional active ingredients as described herein. The pharmaceutical composition may be administered for any of the disorders described herein.

The subject pharmaceutical compositions are typically formulated to provide a therapeutically effective amount of a compound of the present invention as the active ingredient. Where desired, the pharmaceutical compositions contain a compound of the present invention as the active ingredient and one or more pharmaceutically acceptable carriers or excipients, such as inert solid diluents and filers, diluents, including sterile aqueous solution and various organic solvents, permeation enhancers, solubilizers and adjuvants.

The pharmaceutical compositions can be administered alone or in combination with one or more other agents, which are also typically administered in the form of pharmaceutical compositions. Where desired, the subject compounds and other agent(s) may be mixed into a preparation or both components may be formulated into separate preparations to use them in combination separately or at the same time.

Methods include administration of a compound of the present invention by itself, or in combination as described herein, and in each case optionally including one or more suitable diluents, fillers, salts, disintegrants, binders, lubricants, glidants, wetting agents, controlled release matrices, colorants/flavouring, carriers, excipients, buffers, stabilizers, solubilizers, and combinations thereof.

Preparations of various pharmaceutical compositions are known in the art, e.g., Anderson, Philip O.: Knoben, James E.; Troutman, William G, eds., Handbook of Clinical Drug Data. Tenth Edition, McGraw-Hill, 2002: Pratt and Taylor, eds., Principles of Drug Action, Third Edition. Churchill Livingston, New York, 1990: Katzung, ed., Basic and Clinical Pharmacology, Ninth Edition, McGraw Hill, 2003: Goodman and Gilman, eds., The Pharmacological Basis of Therapeutics. Tenth Edition, McGraw Hill, 2001: Remingtons Pharmaceutical Sciences, 20th Ed., Lippincott Williams & Wilkins., 2000: Martindale, The Extra Pharmacopoeia, Thirty-Second Edition (The Pharmaceutical Press, London, 1999), all of which are incorporated by reference herein in their entirety.

The compounds or pharmaceutical composition of the present invention can be administered by any route that enables delivery of the compounds to the site of action, such as oral routes, intraduodenal routes, parenteral injection (including intravenous, intraarterial, subcutaneous, intramuscular, intravascular, intraperitoneal or infusion), topical administration (e.g. transdermal application), rectal administration, via local delivery by catheter or stent or through inhalation. The compounds can also be administered intraadiposally or intrathecally.

The compositions can be administered in solid, semi-solid, liquid or gaseous form, or may be in dried powder, such as lyophilized form. The pharmaceutical compositions can be packaged in forms convenient for delivery, including, for example, solid dosage forms such as capsules, sachets, cachets, gelatins, papers, tablets, capsules, suppositories, pellets, pills, troches, and lozenges. The type of packaging will generally depend on the desired route of administration. Implantable sustained release formulations are also contemplated, as are transdermal formulations.

### Method of Treatment

In further aspects, the present invention provides a use of a compound of the invention, of a pharmaceutically acceptable salt thereof, for the manufacture of a medicament for the treatment of cancer. In some embodiments, the present invention provides a use of a compound of the invention in the manufacture of a medicament for the treatment of any of cancer and/or neoplastic disorders.

A compound or composition described herein can be used to treat a neoplastic disorder. A “neoplastic disorder” is a disease or disorder characterized by cells that have the capacity for autonomous growth or replication, e.g., an abnormal state or condition characterized by proliferative cell growth. Exemplary neoplastic disorders include but are not limited to: carcinoma, sarcoma, metastatic disorders. Solid tumor such as oral, gall bladder, prostate, breast, lung, colon, pancreatic, renal, ovarian as well as soft tissue and osteosarcomas, and stromal tumors for e.g., tumors arising from prostate, brain, bone, colon, pancreas, lung, breast, ovarian, and liver origin, hematopoietic neoplastic disorders, e.g., leukemias, lymphomas, myelomas, myelodysplastic, myeloproliferative syndromes and other malignant plasma cell disorders, and metastatic tumors. Prevalent cancers include but not limited to: breast, prostate, colon, lung, liver, and pancreatic cancers. Treatment with the compound can be in an amount effective to ameliorate at least one symptom of the neoplastic disorder, e.g., reduced cell proliferation, reduced tumor mass, etc.

The disclosed methods are useful in the prevention and treatment of cancer, including for example, solid tumors, soft tissue tumors, and metastases thereof, as well as in familial cancer syndromes such as Li Fraumeni Syndrome, Familial Breast-Ovarian Cancer (BRCA1 or BRAC2 mutations) Syndromes, and others. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine.

Exemplary cancers including but not limited to tumors such as lung, prostate, breast, brain, skin, cervical carcinomas, testicular carcinomas, etc. More particularly, cancers that may be treated by the compositions and methods of the invention include, but are not limited, to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma): Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

Further preferred, the invention provides for methods for inhibiting KRAS activity in a cell, comprising contacting the cell in which inhibition of KRAS activity is desired with an effective amount of a compound of formula (A), (A-I), (A-II) and (A-III), pharmaceutically acceptable salts thereof or pharmaceutical compositions containing the compound or pharmaceutically acceptable salt thereof.

Further preferred, the invention provides for methods for inhibiting KRAS G12C activity in a cell, comprising contacting the cell in which inhibition of KRAS G12C activity is desired with an effective amount of a compound of formula (A), (A-I), (A-II) and (A-III), pharmaceutically acceptable salts thereof or pharmaceutical compositions containing the compound or pharmaceutically acceptable salt thereof.

In one embodiment, a cell in which inhibition of KRAS G12C activity is desired is contacted with an effective amount of a compound of formula (A), (A-I), (A-II) and (A-III) to negatively modulate the activity of KRAS G12C.

Further preferred, a therapeutically effective amount of pharmaceutically acceptable salt or pharmaceutical compositions containing a compound of formula (A), (A-I), (A-II) and (A-III), may be used.

By negatively modulating the activity of KRAS G12C, the methods described herein are designed to inhibit undesired cellular proliferation resulting from enhanced KRAS G12C activity within the cell. The cells may be contacted in a single dose or multiple doses in accordance with a particular treatment regimen to effect the desired negative modulation of KRAS G12C.

Further preferred, methods of treating cancer in a patient in need thereof, comprising administering to said patient a therapeutically effective amount of a compound of formula (A), (A-I), (A-II) and (A-III), pharmaceutically acceptable salts thereof or pharmaceutical compositions comprising the compound or pharmaceutically acceptable salts thereof are provided.

The compositions and methods provided herein may be used for the treatment of a KRAS associated cancer in a patient in need thereof, comprising administering to said patient a therapeutically effective amount of a compound of formula (A), (A-I), (A-II) and (A-III), pharmaceutically acceptable salts thereof or pharmaceutical compositions comprising the compound or pharmaceutically acceptable salts thereof are provided. In one embodiment, the KRAS G12C-associated cancer is lung cancer.

The compositions and methods provided herein may be used for the treatment of a KRAS G12C-associated cancer in a patient in need thereof, comprising administering to said patient a therapeutically effective amount of a compound of formula (A), (A-I), (A-II) and (A-III), pharmaceutically acceptable salts thereof or pharmaceutical compositions comprising the compound or pharmaceutically acceptable salts thereof are provided. In one embodiment, the KRAS G12C-associated cancer is lung cancer.

The compositions and methods provided herein may be used for the treatment of a wide variety of cancers including but not limited to tumors such as lung, prostate, breast, brain, skin, cervical carcinomas, testicular carcinomas, etc. More particularly, cancers that may be treated by the compositions and methods of the invention include, but are not limited, to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors. Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

Further preferred, the cancer is non-small cell lung cancer, colorectal cancer or pancreatic cancer.

Further preferred, the cancer is lung cancer or colorectal cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as in combination with other targeted agents or radiation or surgical intervention, either as an adjuvant prior to surgery or post-operatively.

The invention further provides herein a compound of formula (A), (A-I), (A-II) and (A-II), or a pharmaceutically acceptable salt or solvate thereof, or a pharmaceutical composition thereof as defined herein for use in therapy.

The invention further provides herein a compound of formula (A), (A-I), (A-II) and (A-III), or a pharmaceutically acceptable salt or solvate thereof or a pharmaceutical composition thereof as defined herein for use in the treatment of cancer.

The invention further provides herein is a compound of formula (A), (A-I), (A-II) and (A-III), or a pharmaceutically acceptable salt or solvate thereof for use in the inhibition of KRAS G12C.

The invention further provides herein is a compound of formula (A), (A-I), (A-II) and (A-III), or a pharmaceutically acceptable salt or solvate thereof or a pharmaceutical composition thereof as defined herein, for use in the treatment of a KRAS G12C-associated disease or disorder.

The invention further provides herein is the use of a compound of formula (A), (A-I), (A-I) and (A-III), or a pharmaceutically acceptable salt or solvate thereof, as defined herein in the manufacture of a medicament for the treatment of cancer.

The invention further provides herein is a use of a compound of formula (A), (A-I), (A-II) and (A-III), or a pharmaceutically acceptable salt or solvate thereof, as defined herein in the manufacture of a medicament for the inhibition of activity of KRAS G12C.

The invention further provides herein is the use of a compound of formula (A), (A-I), (A-II) and (A-III), or a pharmaceutically acceptable salt or solvate thereof, as defined herein, in the manufacture of a medicament for the treatment of a KRAS G12C-associated disease or disorder.

The invention further provides herein is a method for treating cancer in a patient in need thereof, the method comprising (a) determining that cancer is associated with a KRAS G12C mutation (e.g., a KRAS G12C-associated cancer) (e.g., as determined using a regulatory agency-approved, e.g., FDA-approved, assay or kit); and (b) administering to the patient a therapeutically effective amount of a compound of formula (A), (A-I), (A-II) and (A-III), or a pharmaceutically acceptable salt thereof, or a pharmaceutical composition thereof.

Metastases of the aforementioned cancers can also be treated or prevented in accordance with the methods described herein.

### Combination Therapies

In some embodiments, a compound described herein is administered together with an additional “second” therapeutic agent or treatment. The choice of second therapeutic agent may be made from any agent that is typically used in a monotherapy to treat the indicated disease or condition. As used herein, the term “administered together” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of any of the formulas described herein, an additional therapeutic agent, and a pharmaceutically acceptable carrier, adjuvant, or vehicle.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art. The additional agents may be administered separately, as part of a multiple dose regimen, from the compounds of this invention. Alternatively, those agents may be part of a single dosage form, mixed together with the compounds of this invention in a single composition.

The compounds, pharmaceutically acceptable salts thereof and pharmaceutical compositions comprising such compounds and salts also may be co-administered with other anti-neoplastic compounds, e.g., chemotherapy, or used in combination with other treatments, such as in combination with other targeted agents or radiation or surgical intervention, either as an adjuvant prior to surgery or post-operatively.

The compounds, pharmaceutically acceptable salts thereof and pharmaceutical compositions comprising such compounds comprising the step of administering simultaneously or sequentially to a subject in need thereof at least one other anti-cancer agent, anti-inflammatory agent, immunosuppressive agent, steroid, non-steroidal anti-inflammatory agent, antihistamine, analgesic, or a mixture thereof.

### Process for Preparation of the Compound of the Invention

The instant compounds of the present invention may be prepared by the following general process. The process provided herein can similarly be applied to synthesize all possible variation of the compound of the invention, and in particular compounds of formulas (A), (A-I), (A-II) and (A-III) as provided herein above with all intended modification or without any modification. Unless otherwise indicated, the variables such as R, R^{a}, R¹, A₁, A₂, Cy¹, Cy² and E used here in various intermediates or compounds of formulas are to be constructed to be the variables defined herein above in relation to compound of the invention and in particular to the compound of formula (A), (A-I), (A-II) and (A-III).

Synthetic Scheme A: This schematic representation of scheme aims to provide a possible route for the preparation of a compound of formula (A), (A-I), (A-II) and (A-III).

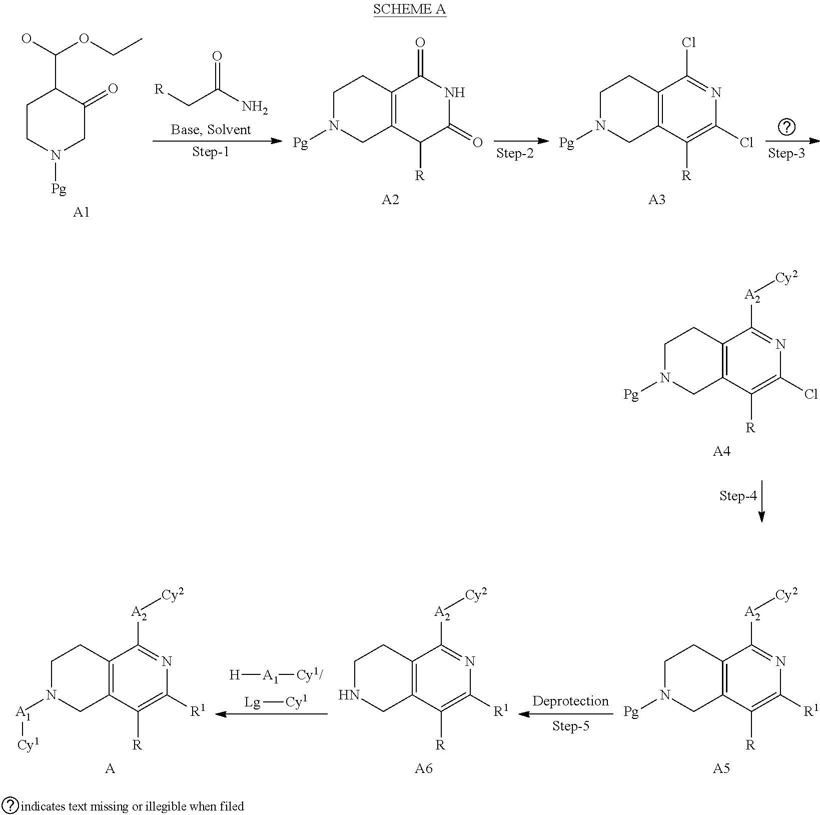

Intermediate of formula (A1) where in Pg is a suitable protecting group when reacted with an intermediate of formula R—CH₂—CONH₂, using a suitable base or reagent to get an intermediate of formula (A2). Intermediate of formula (A2) can then be subjected to chlorination using POCl₃ to get an intermediate of formula (A3) which on reacting with an intermediate f formula Lg-A₂-Cy² wherein Lg is a good leaving group using a suitable base to get an intermediate of formula (A4). Intermediate of formula (A4) is in turn can be coupled with an intermediate of formula Lg-R¹ wherein Lg is a good leaving group, using a suitable base to get an Intermediate of formula (A5). Intermediate of formula (A5). Intermediate (A5) can then be de-protected followed by coupling with a group Lg-At-Cy¹ under suitable conditions such as backward coupling or other suitable N-Alkylation or N-Arylation to provide a desired compound of formula (A)

### A. Scheme of Example 1 Provides for Illustrative Use of Scheme A as Below

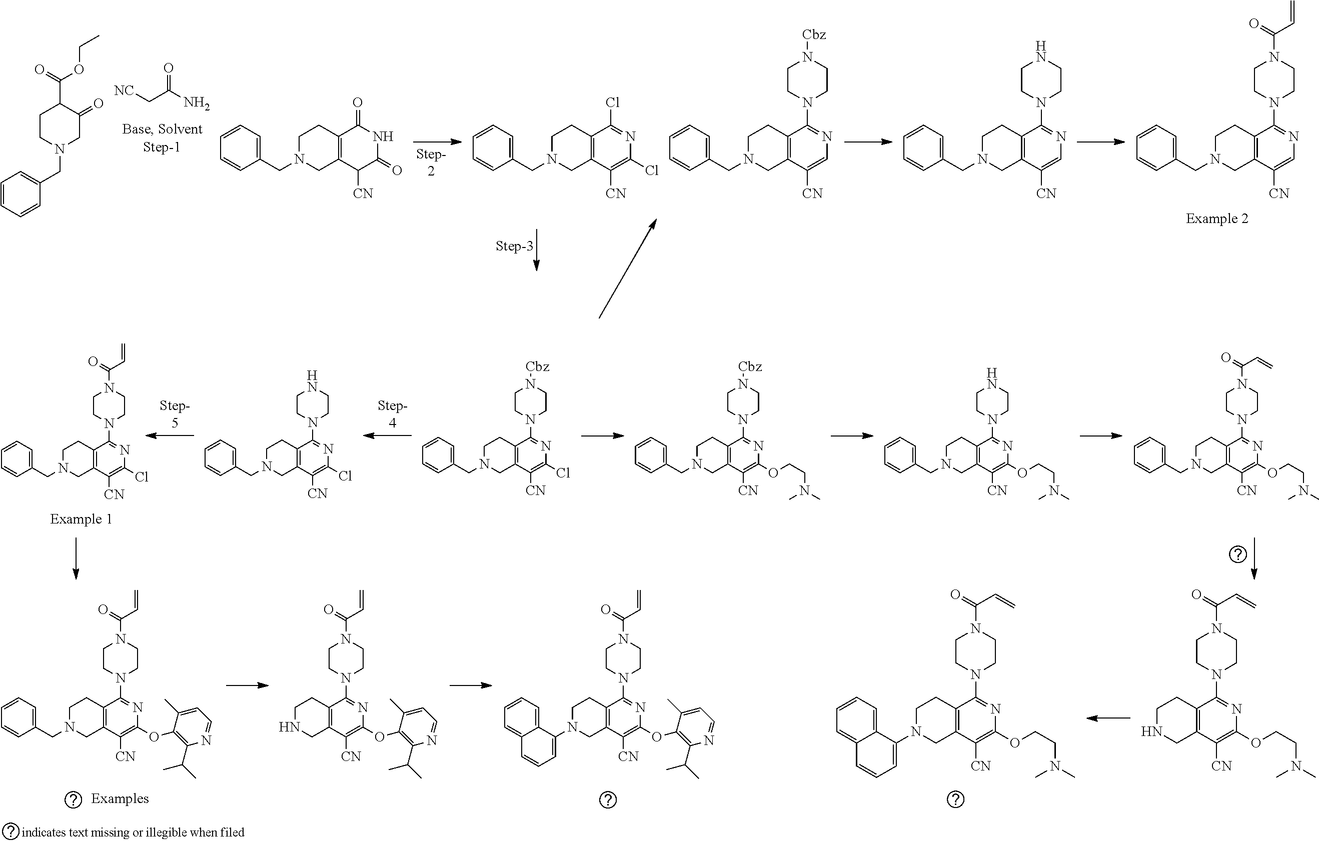

### B. Scheme Below Provides for Yet Another Illustrative Use of Scheme A to Prepare a Compound of Invention

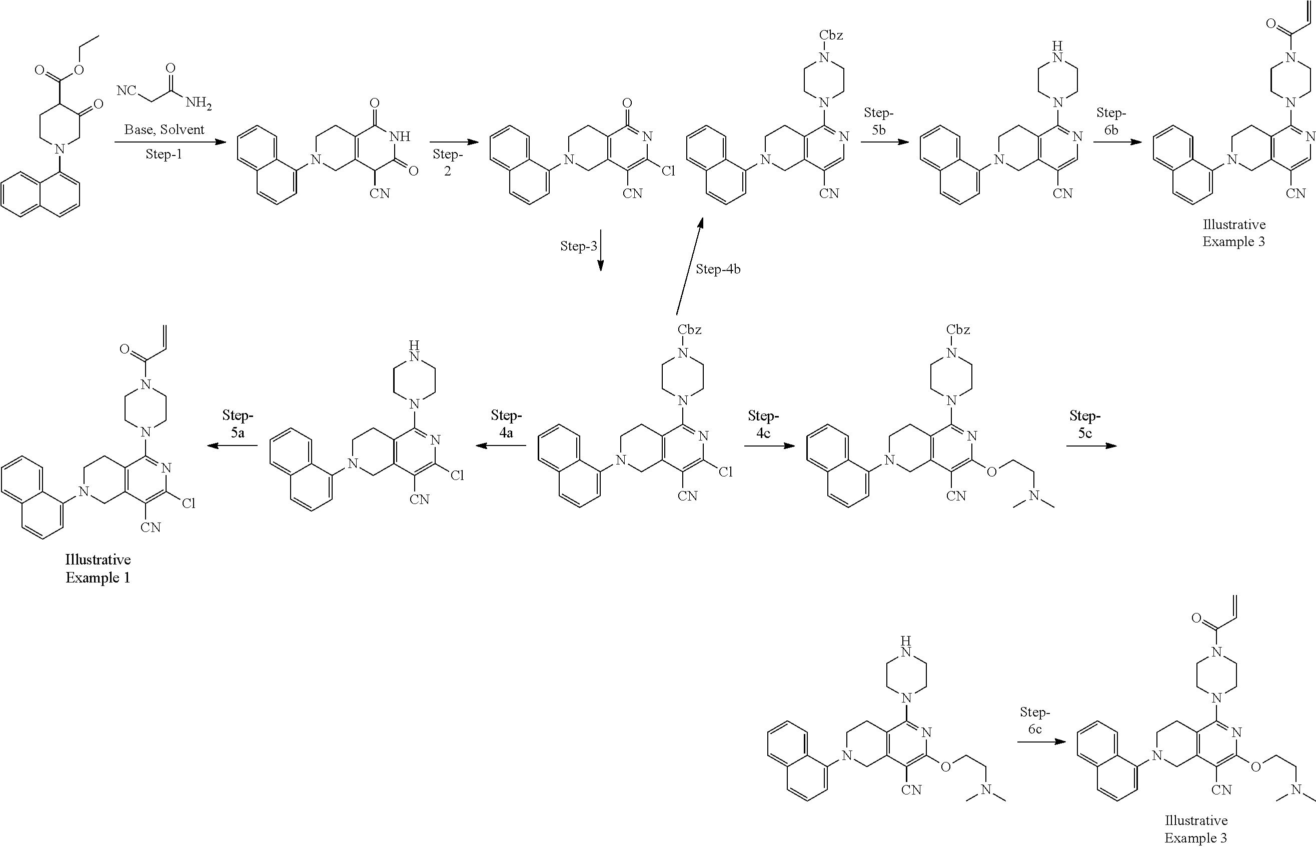

### C. Scheme Below Provides for Yet Another Illustrative Use of Scheme A to Prepare a Compound of Invention

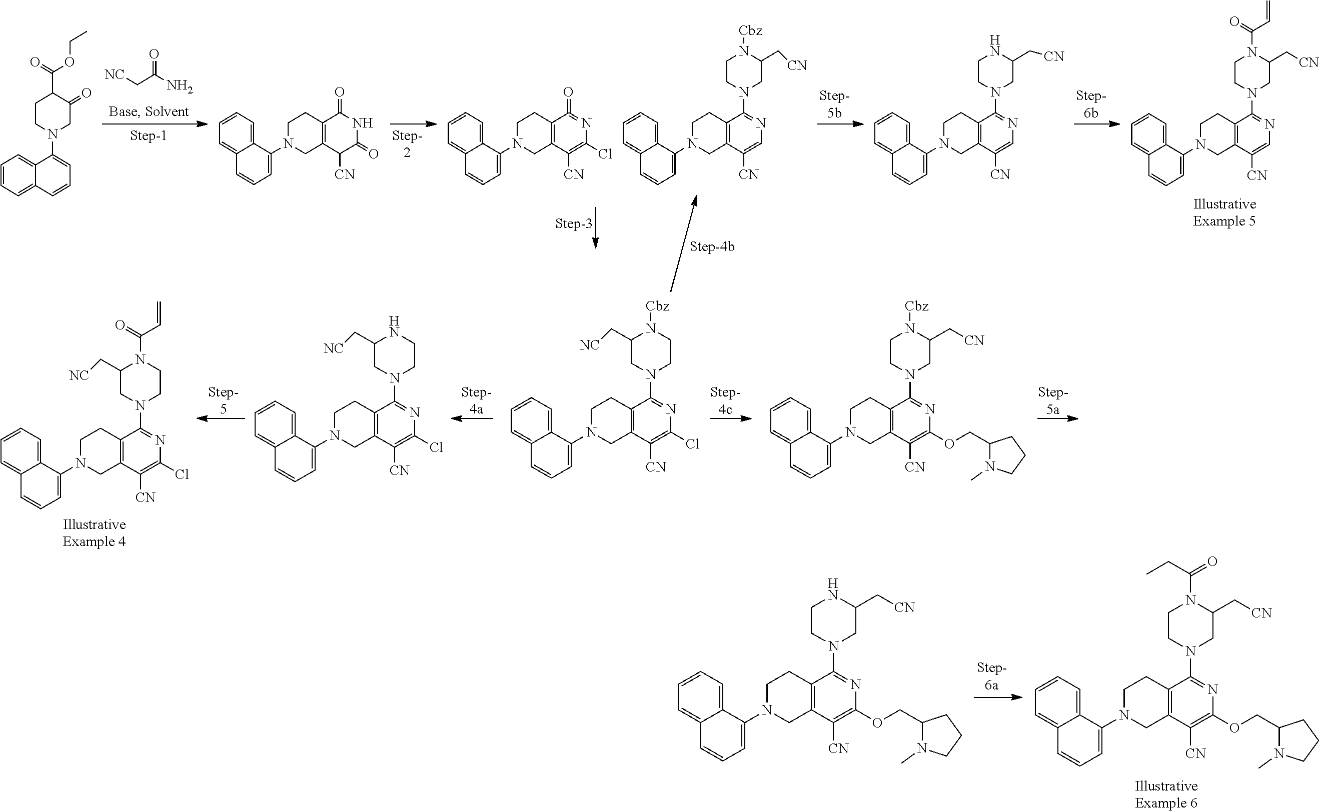

### EXPERIMENTAL DETAILS

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

### Part-A: Preparation of Intermediates

Intermediate 1: (ethyl 1-benzyl-3-oxopiperidine-4-carboxylate) In a reaction flask, (26 g, 80.00 mmol) of ethyl 4-(benzyl(2-methoxy-2-oxoethyl)amino)butanoate and (11 g, 160 mmol) of sodium ethoxide were added to 260 mL of toluene, heating reaction reflux 5 h, TLC monitoring of raw materials completely complete reaction, the reaction solution cooled to room temperature, add water 150 mL, with ethyl acetate 250 mL extraction reaction 2 times, the organic phase. The organic phase was washed with an amount of saturated brine solution and the organic phase was distilled off to give 14 g of oily compound. MS (m/z): 262.24 [M+H]⁺.

Intermediate 2: 6-benzyl-1,3-dioxo-1,2,3,4,5,6,7,8-octahydro-2,6-naphthyridine-4-carbo nitrile: In the 2-necked flask equipped with a mechanical stirrer, and a condenser was placed intermediate 1 (14 g, 50.00 mmol) in methanol (105 ml). The cyanoacetamide (4.5 g, 50.00 mmol) was added followed by addition of a potassium hydroxide solution in methanol (3.94 g, 50 mmol of KOH in 50 mL MeOH). The resulting mixture was stirred and heated to reflux. The product was started to form as a white solid after few minutes. The reaction mixture was refluxed for 4 hr and cooled to room temperature overnight. The white solid was collected and filter cake was washed with methanol (300 mL). The white solid was transferred into a 1 L single necked flask equipped with a mechanical stirrer with the aid of warm water (250 ml of 55° C.). The resulting mixture was stirred and acidified to pH=6 with acetic acid. After 1 hr stirring, the white solid was collected, washed with water, pressed well with a rubber dam and air dried in a hood overnight to give 10 g of the title compound. MS (m/z): 282.20 [M+H]⁺.

### Intermediate 3: 6-benzyl-1,3-dichloro-5,6,7,8-tetrahydro-2,6-naphthyridine-4-carbonitrile

Method I: Intermediate 2 (5 g, 10.00 mmol) and phosphorus oxychloride (20 mL). The vessel was sealed and heated at 180° C. for 4 hr, reaction mixture was concentrated under reduced pressure. The residue was dissolved in dichloromethane (500 ml) and added into a 1 L 3-necked flask equipped with a mechanical stirrer containing ice (about 45 g). The resulting mixture was basified to pH=9-10 by adding approximately 50 mL 20 percent aqueous sodium hydroxide solution. During this addition more ice was added to keep internal temperature under 30° C. The mixture was stirred for 15 min and then was filtered to remove a gel type particle. The organic layer was separated, washed with brine (50 mL), filtered through filter paper and concentrated under reduced pressure. (1.5 g) as an oil which solidified on standing. MS (m/z): 318.20 [M+H]⁺.

Method II: Intermediate 2 (20 gm g, 0.071 mol, salt free), tetramethylammonium chloride (9.3 g, 0.085 mol) and phosphorus oxychloride (67 mL, 0.71 mol) were taken in a 500 mL sealed vessel and heated at 120° C. for 8 hrs. The reaction was cooled to RT, slowly open the vessel. Reaction mixture was slowly poured into ice and stirred the reaction mixture for 30 min. Then the saturated solution of NaOH was added slowly to the cold and diluted RM to turn it alkaline (pH=8-10) and maintenance the temperature below 40° C. The brown colored solid was formed and it was filtrated over the filtration flask and dried over 2 hr we got Intermediate 3 (14 g). 1H-NMR (δ ppm, CDCl₃, 400 MHz): 7.36-7.30 (m, 5H), 3.81 (s, 2H), 3.75 (s, 2H), 2.84-2.82 (d, 2H, J=5.2 Hz), 2.80-2.78 (d, 2H, J=5.2 Hz). MS (m/z): 318.20 [M+H]⁺.

Intermediates 4A: benzyl-4-cyano-5,6,7,8-tetrahydro-2,6-naphthyridine-1,3-diyl bis(trifluoromethanesulfonate): Intermediate 2 (5 gram, 0.01 mole) and 2,6 lutidine (5.9 gram, 0.055 mole) were dissolved in DCM (200 ml) and cooled to 0° C. under nitrogen atmosphere, Triflic Anhydride (10 gram, 0.035) in DCM (25 ml) was then added dropwise over a period of 20 mins. The reaction was allowed to gradually warm to 25° C. over a period of 2 hr. The reaction mixture was concentrated in vacuo and the resulting oil was dissolved in ethyl acetate and the organic layer was washed thrice with 10% KHSO₄ followed by brine and the organic layer was concentrated in vacuo. The resulting solid was purified by column chromatography (100-2 (0) mesh silica gel, 0-40% ethyl acetate-hexane) to get Intermediate 4 (3.2 gm). 1H-NMR (δ ppm, CDCl₃, 400 MHz): 7.35 (m, 5H), 3.91 (s, 2H), 3.79 (s, 2H), 2.91 (t, 2H), 2.83 (m, 2H). MS (m/z): 545 [M+H]⁺

Intermediates 5: tert-butyl (1-(6-benzyl-3-chloro-4-cyano-5,6,7,8-tetrahydro-2,6-naphthyridin-1-yl)piperidin-4-yl)carbamate: tert-butyl piperidin-4-ylcarbamate (0.5 g, 16 mmol) was dissolved in DMF (5 ml), followed by addition of potassium carbonate (0.54 g, 30 mmol) in the reaction mixture and stirred at room temperature for 10 min. Intermediate 3 (0.5 g, 10 mmol) was added in reaction mixture and heated reaction at 80° C. for 20 minutes. The reaction completion was check by TLC, reaction was poured into water and ethyl acetate and separated. The organic layer was dried over sodium sulphate and concentrated under reduced pressure to get oily (0.3 g) compound, was used in next step without analytical data.

Intermediates 5A: Tert-butyl 4-(6-benzyl-4-cyano-3-(((trifluoromethyl)sulfonyl)oxy)-5,6,7,8-tetrahydro-2,6-naphthyridin-1-yl)piperazine-1-carboxylate: To solution of tert-butyl piperazine-1-carboxylate (0.46 gm, 0.001 mol) in THF (30 ml) was added DIPEA (1.2 ml, 0.03 mol) and stirred. Solution of Intermediate 4A (1.5 gm, 0.001 mol) in THF (10 ml) was added to the reaction mixture dropwise and stirred at room temperature for 2 hr. Reaction mixture was quenched with water and extracted by ethyl acetate. Separated organic layer, was washed with brine followed by drying over sodium sulphate. Organic layer was concentrated under reduce pressure to get the crude residue which was purified by column chromatography using ethyl acetate: hexane (25%) to get Intermediate 5A (1.25 gm). 1H-NMR (δ ppm, CDCl₃, 400 MHz): 7.36-7.35 (m, 4H), 7.32-7.30 (m, 1H), 3.82 (s, 2H), 3.72 (s, 2H), 3.52-3.51 (m, 4H), 3.43-3.41 (m, 4H), 2.68-2.63 (m, 4H). MS (m/z): 582.50 [M+H]⁺.

Intermediates 5B: Tert-butyl 4-(6-benzyl-4-cyano-3-(((trifluoromethyl)sulfonyl)oxy)-5,6,7,8-tetrahydro-2,6-naphthyridin-1-yl)piperazine-1-carboxylate: 2-(piperazin-2-yl) acetonitrile (0.920 gm, 0.0073 mol) and DIEPA (1.37 ml, 0.0073 mol) was dissolved in DCM (40 ml) and cooled to 0° C. under nitrogen atmosphere. Intermediates 4A (2.7 gm, 0.0049 mol) in DCM (10 ml) was added dropwise to the reaction mixture. The reaction was stirred at room temperature for 12 hr. To the above reaction mixture solution of (BOC)₂O in DCM (10 ml) was added dropwise at RT and stirred for another 12 hr. The reaction mixture was quenched with water and organic layer was separated and washed with brine and concentrated in vacuo to give oily residue which was purified by column chromatography (100-200 mesh silica gel) using 30% Ethyl acetate: hexane to get Intermediate 9B as white solid (1.7 gm). 1H-NMR (δ ppm, CDCl₃, 400 MHz): 7.38-7.33 (m, 5H), 4.62-4.60 (m, 1H), 4.06 (bs, 1H), 3.99-3.82 (m, 1H), 3.76-3.67 (m, 5H), 3.35-3.34 (m, 1H), 3.24-3.23 (m, 1H), 2.83-2.71 (m, 5H), 2.58-2.55 (m, 1H), 1.48 (s, 9H). MS (m/z): 621.22 [M+H]⁺.

Intermediates 5C: tert-butyl 4-(6-benzyl-3-chloro-4-cyano-5,6,7,8-tetrahydro-2,6-naphthyridin-1-yl)-2-(cyanomethyl)piperazine-1-carboxylate: 2-(piperazin-2-yl) acetonitrile (29.48 g, 0.23 mol) was added in 4 neck 3 lit RBF, in 1,4-dioxane (1000 ml), followed by addition of DIPEA (144 mL, 0.75 mol) in the above suspension and stirred at 0° C. for 10 min. Intermediate 3 (50 g, 0.15 mol) was added to the reaction mixture and stirred at 55° C. for 12 hrs. After formation of new polar spot reaction mixture was cooled to RT and Boc-anhydride (98 mL, 0.45 mol) was added to it and stirred at 55° C. for 2 hrs. After completion of reaction the RM was poured into cold water and extracted with ethyl acetate (1 lit×2). The organic layer was dried over sodium sulphate and concentrated under reduced pressure to get a crude residue. The crude was purified by column chromatography using 20-50% EtOAc in pet ether as mobile phase. The pure fractions were collected and concentrated under reduced pressure to affords Intermediate 5C (40 g) as white solid. 1H-NMR (δ ppm, CDC3, 400 MHz): 7.35-7.34 (m, 4H), 7.31-7.29 (m, 1H), 4.59 (bs, 11H), 3.90 (bs, 1H), 3.83-3.68 (m, 6H), 3.27-3.23 (m, 2H), 2.99-2.92 (m, 1H), 2.81-2.69 (m, 5H), 2.67-2.65 (m, 1H). MS (m/z): 507.2 [M+H]⁺.

Intermediates 5D: tert-butyl 4-(6-benzyl-4-cyano-3-(((trifluoromethyl)sulfonyl)oxy)-5,6,7,8-tetrahydro-2,6-naphthyridin-1-yl)-2-methylpiperazine-1-carboxylate: Was prepared using the process as described above for Intermediate 5A using 3-methyl piperazine-1-carboxylate (1.21 gm, 0.0065 mmol) and Intermediate 4 (3.0 gm, 0.0055 mol) to get Intermediate 5D (2.22 gm). MS (m/z): 596.24 [M+H]⁺.

Intermediates 5E: 6-benzyl-4-cyano-1-(2,6-dimethylmorpholino)-5,6,7,8-tetrahydro-2,6-naphthyridin-3-yl trifluoromethanesulfonate: Was prepared using the process as described above for Intermediate 5A using 2,6-dimethyl morpholine (1.25 gm, 0.00688 mmol) and Intermediate 4 (2.5 gm, 0.0045 mol) to get Intermediates 5E (1.8 gm). MS (m/z): 510.95 [M+H]⁺.

Intermediates 5F: tert-butyl (R)-4-(6-benzyl-4-cyano-3-(((trifluoromethyl)sulfonyl)oxy)-5,6,7,8-tetrahydro-2,6-naphthyridin-1-yl)-3-methylpiperazine-1-carboxylate: Was prepared using the process as described above for Intermediate 5A using tert-butyl (R) 3-methyl piperazine-1-carboxylate (1.21 gm, 0.0065 mmol) and Intermediate 4A (3.0 gm, 0.0055 mol) to get Intermediate 5F (2.22 gm). MS (m/z): 596.24 [M+H]⁺.

Intermediates 5G: tert-butyl (S)-4-(6-benzyl-4-cyano-3-(((trifluoromethyl)sulfonyl)oxy)-5,6,7,8-tetrahydro-2,6-naphthyridin-1-yl)-3-methylpiperazine-1-carboxylate: Was prepared using the process as described above for Intermediate 5A using tert-butyl (S) 3-methyl piperazine-1-carboxylate (1.21 gm, 0.0065 mmol) and Intermediate 4A (3.0 gm, 0.0055 mol) to get Intermediate 5G (2.22 gm). MS (m/z): 596.24 [M+H]⁺.

Intermediate 5H: tert-butyl 4-(6-benzyl-3-chloro-4-cyano-5,6,7,8-tetrahydro-2,6-naphthyridin-1-yl)piperazine-1-carboxylate: Was prepared using the process as described above for Intermediate 5A using N-Boc piperazine (1.1 gm) and Intermediate 3 (2.5 gm) to get Intermediate 5H (1.85 gm). 496.21 [M+H]⁺.

Intermediates 6: 1-(4-aminopiperidin-1-yl)-6-benzyl-3-chloro-5,6,7,8-tetrahydro-2,6-naphthyridine-4-carbonitrile: To a solution of Intermediate 5 (0.5 g) was dissolved in dioxane:HCl (10 ml), stirred reaction mixture for 1 hr at OC. After completion of the reaction, it was evaporated under reduced pressure to get oily compound, was dissolved in di ethyl ether to get solid, we used next step directly without analytical data.

Intermediates 6A1: Bert-butyl 4-(6-benzyl-4-cyano-3-hydroxy-5,6,7,8-tetrahydro-2,6-naphthyridin-1-yl)piperazine-1-carboxylate: Intermediate 5A (1.0 gm, 0.0018 mmol) was dissolved in THF (20 ml) and reaction mixture was stirred at RT, followed by addition of 10% NaOH (5 ml) and stirred the mixture for 30 min. The reaction mixture was extracted with ethyl acetate. The combined THF and ethyl acetate layer was dried over sodium sulphate and concentrated under vacuum to get Intermediate 6A1 as white solid (0.8 gm). MS (m/z): 450.12 [M+H]⁺.

Intermediates 6A2: Tert-butyl 4-(6-benzyl-4-cyano-3-hydroxy-5,6,7,8-tetrahydro-2,6-naphthyridin-1-yl)-2-(cyanomethyl)piperazine-1-carboxylate: Intermediate 5B (1.0 gm, 0.0018 mmol) was dissolved in THF (20 ml) and reaction mixture was stirred at RT, followed by addition of 10% NaOH (5 ml) and stirred the mixture for 30 min. The reaction mixture was extracted with ethyl acetate. The combined THF and ethyl acetate laver was dried over sodium sulphate and concentrated under vacuum to get Intermediate 6A2 as brown solid (0.8 gm). 1H-NMR (400 MHz)(CDCl3): 7.30-7.22 (m, 5H), 4.41 (bs, 1H), 4.13-4.11 (m, 1H), 3.75 (bs, 1H), 3.62 (bs, 2H), 3.51-3.47 (m, 2H), 3.33-3.22 (m, 3H), 2.68 (bs, 2H), 2.44-2.39 (m, 5H), 1.43 (s, 9H). MS: m/Z: 489.2 (M+1).

Intermediates 6B: tert-butyl 4-(6-benzyl-4-cyano-3-morpholino-5,6,7,8-tetrahydro-2,6-naphthyridin-1-yl)piperazine-1-carboxylate: K₂CO₃ (0.29 gm, 0.0021 mol) was added to a to solution of morpholine (0.112 ml, 0.0012 mol) in N-methyl Pyrrolidone (NMP, 4 ml) and stirred at RT. Intermediate 5A (0.5 gm, 0.00086 mol) in NMP was added to the reaction mixture dropwise followed by heating at 60° C. for 2 hr. Reaction mixture was quenched with water and extracted with ethyl acetate. Organic layer was washed with brine, dried over sodium sulphate and concentrated under vacuum to get crude residue which was purified by column chromatography using ethyl acetate: hexane (25%) to get Intermediate 6B (0.4 gm), 1H-NMR (δ ppm, CDCl3, 400 MHz): 7.38-7.34 (m, 4H), 7.30-7.7.29 (m, 1H), 3.81-3.79 (m, 4H), 3.76 (s, 2H), 3.70 (s, 2H), 3.61-3.58 (m, 4H), 3.51-3.47 (m, 4H), 3.28-3.26 (m, 4H), 2.58 (bs, 4H), 1.47 (s, 9H). MS (n/z): 519.22 [M+H]⁺.

Intermediates 6C: tert-butyl 4-(6-benzyl-4-cyano-3-(1,1-dioxidothiomorpholino)-5,6,7,8-tetrahydro-2,6-naphthyridin-1-yl)piperazine-1-carboxylate: Was prepared using the process as described above for Intermediate 6B using Thiomorpholine 1,1 dioxide (0.52 gm, 0.0051 mmol) and Intermediate 5B (1.5 gm, 0.0025 mol) to get Intermediates 6C as white solid (1.25 gm), 1H-NMR (8 ppm, CDCl₃, 400 MHz): 7.38-7.32 (m, 4H), 7.31-7.30 (m, 1H), 4.12-4.10 (m, 4H), 3.76 (m, 4H), 3.52-3.50 (m, 4H), 3.28-3.25 (m, 4H), 3.18-3.16 (m, 4H), 2.78 (bs, 4H), 1.47 (s, 9H). MS (n/z): 567.20 [M+H]⁺.

Intermediates 6D: tert-butyl 4-(6-benzyl-4-cyano-3-(4-methylpiperazin-1-yl)-5,6,7, -tetrahydro-2,6-naphthyridin-1-yl)piperazine-1-carboxylate: To solution of N-methyl piperazine (0.63 ml, 0.005 mol) in NMP (30 ml) was added DIPEA (2.0 ml, 0.01 mol) and stirred at RT. Intermediate 5A (2.2 gm, 0.003 mol) in NMP was added to the reaction mixture dropwise and heated at 60° C. for 2 hr. Reaction mixture was quenched with water and extracted with ethyl acetate. Organic layer was washed with brine, dried over sodium sulphate and concentrated under vacuum to get crude residue which was purified by column chromatography using ethyl acetate: hexane (25%) to get Intermediates 6D (1.0 gm), 1H-NMR (5 ppm, CDCl₃, 400 MHz): 7.38-7.32 (m, 4H), 7.29-7.28 (m, 1H), 3.76 (s, 2H), 3.70 (s, 2H), 3.50-3.49 (m, 4H), 3.39-3.36 (m, 4H), 3.27-3.23 (m, 4H), 2.84 (s, 3H), 2.55-2.53 (4H), 2.39-2.35 (2H), 2.02-2.01 (2H). MS (m/z): 532.50 [M+H]⁺.

Intermediates 6E: tert-butyl 4-(6-benzyl-4-cyano-3-(2-fluoropyridin-4-yl)-5,6,7,8-tetrahydro-2,6-naphthyridin-1-yl)piperazine-1-carboxylate: To solution of Intermediate 5A (1.5 gm, 0.0025 mol) and 3-fluoro 4-pyridine boronic acid (0.38 g, 0.0027 mol) in Dioxane: H₂O, K₂CO₃ (0.71 g, 0.0051 mol) was added and purged with N2 gas for 15 min. Tetrakis (0.13 g, 0.00012 mol) was added and again purged for 5 min with N2 gas, Reaction mixture was heated at 110° C. for 2 h. Reaction mixture was cooled to Rt and quenched with ice cold water and extracted with (3×50 ml) EtOAc, EtOAc layer was dried over Na2SO4 and concentrated under vacuum to get crude residue which was purified by column chromatography compound eluted at 30% EA in Hexane to get Intermediates 6E as white solid (0.2 gm), 1H-NMR (S ppm. CDCl₃, 400 MHz): 8.35-8.34 (m, 1H), 7.75-7.74 (m, 1H), 7.45 (s, 1H), 7.38-7.35 (m, 4H), 7.32-7.30 (m, 1H), 3.89 (s, 2H), 3.76 (s, 2H), 3.56 (bs, 4H), 3.39 (bs, 4H), 2.76 (bs, 2H), 2.69-2.68 (m, 2H), 1.48 (s, 9H). MS (m/z): 527[M+H]−.

Intermediates 6F: tert-butyl 4-(6-benzyl-4-cyano-3-(piperidin-1-yl)-5,6,7, -tetrahydro-2,6-naphthyridin-1-yl)piperazine-1-carboxylate, Was prepared using the process as described above for Intermediate 6D using piperidine (0.63 ml, 0.005 mol) and Intermediate 5A (2.2 gm, 0.003 mol) to get Intermediates 6F (1.0 gm), 1H-NMR (δ ppm, CDCl₃, 400 MHz): 7.38-7.35 (m, 4H), 7.34-7.32 (m, 1H), 3.75 (s, 2H), 3.69 (s, 2H), 3.58-3.51 (m, 4H), 3.49-3.48 (m, 4H), 3.26-3.24 (m, 4H), 2.57 (bs, 4H), 1.69-1.64 (bs, 6H), 1.47 (s, 9H). MS (m/z): 517 [M+H]⁺.

Intermediates 6G: tert-butyl 4-(6-benzyl-4-cyano-3-(2,& dimethylmorpholino)-5,6,7,8-tetrahydro-2,6-naphthyridin-1-yl)piperazine-1-carboxylate: Was prepared using the process as described above for Intermediate 6C using 2,6 dimorpholine (0.112 ml, 0.0012 mol) and Intermediate 5A (0.5 gm, 0.00086 mol) to get Intermediates 6G (0.4 gm), 1H-NMR (δ ppm, CDCl₃, 400 MHz): 7.36-7.35 (m, 5H), 4.11-4.08 (m, 2H), 3.76-3.70 (m, 6H), 3.51-3.48 (m, 4H), 3.26-3.24 (m, 4H), 2.69-2.67 (m, 2H), 2.58 (bs, 4H), 1.48 (s, 9H), 1.29 (s, 3H), 1.20 (s, 3H). MS (m/z): 547.72 [M+H]⁺.

Intermediates 6H: tert-butyl 4-(6-benzyl-4-cyano-3-(4-propionylpiperazin-1-yl)-5,6,7,8-tetrahydro-2,6-naphthyridin-1-yl)piperazine-1-carboxylate: To solution of piperazine (0.33 gm, 0.0028 mmol) in THF (15) was added DIPEA (1.29 ml, 0.0075 mmol and stirred at RT. Intermediate 5A (1.5 gm, 0.00258 mmol) in THF was added to the reaction mixture dropwise and heated at 80° C. for 2 hr. Reaction mixture was cooled at 0° C. and propionyl chloride (0.46 ml, 0.005 mmol) was added dropwise over a period of 10 min and stirred the reaction mixture for 1 hr. Reaction mixture was quenched with water and extracted with ethyl acetate. Organic layer was washed with brine, dried over sodium sulphate and concentrated under vacuum to get crude residue which was purified by column chromatography using ethyl acetate: hexane (25%) to get Intermediates 6H (1.2 gm), 1H-NMR (5 ppm, CDCl₃, 400 MHz): 7.38-7.26 (m, 5H), 3.76 (s, 2H), 3.70 (s, 4H), 3.55-3.54 (bs, 10H), 3.32-3.27 (m, 4H), 2.60 (bs, 4H), 2.40-2.30 (q, 2H), 1.47 (s, 9H), 1.90-1.54 (t, 3H). MS (m/z): 574.16 (M+H)⁺.

Intermediate 61 butyl 4-(6-benzyl-4-cyano-3-(4-(methylsulfonyl)piperazin-1-yl)-5,6,7,8-tetrahydro-2,6-naphthyridin-1-yl)piperazine-1-carboxylate: To solution of piperazine (0.33 gm, 0.0028 mmol) in THF (15 ml) was added DIPEA (1.29 ml, 0.0075 mmol) and stirred at RT. Tert-butyl 4-(6-benzyl-4-cyano-3-(((trifluoromethyl)sulfonyl)oxy)-5,6,7,8-tetrahydro-2,6-naphthyridin-1-yl)piperazine-1-carboxylate (1.5 gm, 0.00258 mmol) in THF was added to the above reaction mixture dropwise at same temp and heated at 80° C. for 2 hr. Reaction mixture was cooled at 0° C. and Methanesulfonyl chloride (0.32 ml, 0.005 mmol) was added to the reaction mixture dropwise over a period of 10 min and stirred for 1 hr. Reaction mixture was quenched with water and extracted with ethyl acetate, the organic layer was washed with brine and dried over sodium sulphate followed by evaporation to give crude product which was purified by column chromatography using ethyl acetate: hexane (25%) to get desired compound (1.2 gm), 1H-NMR (δ ppm, CDCl₃, 400 MHz): 7.38-7.26 (m, 5H), 3.76 (s, 2H), 3.70 (s, 4H), 3.55-3.54 (bs, 10H), 3.32-3.27 (m, 4H), 2.60 (bs, 4H), 2.40-2.30 (q, 2H), 1.47 (s, 9H), 1.90-1.54 (t, 3H). MS (m/z): 574.16 (M+H)⁺.

Intermediate 6J: Tert-butyl 4-(3-(4-acetylpiperazin-1-yl)-6-benzyl-4-cyano-5,6,7,8-tetrahydro-2,6-naphthyridin-1-yl)piperazine-1-carboxylate: To solution of piperazine (0.33 gm, 0.0028 mmol) in THF (15) was added DIPEA (1.29 ml, 0.0075 mmol) and stirred at RT. Tert-butyl 4-(6-benzyl-4-cyano-3-(((trifluoromethyl)sulfonyl)oxy)-5,6,7,8-tetrahydro-2,6-naphthyridin-1-yl)piperazine-1-carboxylate (1.5 gm, 0.00258 mmol) in THF was added in reaction mixture dropwise at same temp and it heated at 80° C. for 2 hr. The reaction completion was checked by TLC using ethyl acetate and hexane (30%). Reaction mixture was cooled at 0° C. and Acetyl chloride (0.22 ml, 0.005 mmol) was added in the reaction mixture over dropwise foe 10 min and stirred the reaction mixture for 1 hr. Reaction mixture was poured into water and ethyl acetate and separated organic layer, was washed with brine. Organic layer was dried over sodium sulphate its evaporated to crude product. The crude was purified by column chromatography using ethyl acetate: hexane (25%) to get Intermediates 6J (1.0 gm), 1H-NMR (δ ppm, CDCl₃, 400 MHz): 7.38-7.32 (m, 4H), 7.30-7.28 (m, 1H), 3.76-3.70 (m, 6H), 3.60 (s, 4H), 3.56-3.54 (m, 2H), 3.51-3.49 (m, 4H), 3.28-3.26 (m, 4H), 2.59 (bs, 4H), 2.13 (s, 3H), 1.47 (s, 9H). MS (m/z): 560.12 (M+H)⁺.

Intermediates 7: 1-(4-aminopiperidin-1-yl)-6-benzyl-5,6,7,8-tetrahydro-2,6-naphthyridine-4-carbonitrile: To a solution of tert-butyl (1-(6-benzyl-3-chloro-4-cyano-5,6,7,8-tetrahydro-2,6-naphthyridin-1-yl)piperidin-4-yl)carbamate (0.35 g) was dissolved in methanol (10 ml), triethylamine (20 ul) and Pd/C (0.35 g) and stirred reaction mixture for 12 hr at room temperature. After completion of the reaction, it was filtered over celite and mixture was evaporated to get oily compound (0.25 gm), we used next step directly without analytical data.

Intermediate 7A: Tert-butyl 4-(6-benzyl-4-cyano-3-(2,6-dimethylmorpholino)-5,6,7,8-tetrahydro-2,6-naphthyridin-1-yl)-2-(cyanomethyl)piperazine-1-carboxylate. Was prepared using the process as described above for Intermediate 6C using 2,6 dimorpholine (0.953 ml, 0.0051 mmol) and Intermediate 5C (3 gm, 0.0051 mmol) to get Intermediates 7A (2.6 gm), 1H-NMR (δ ppm, CDCl₃, 400 MHz): 7.38-7.34 (m, 4H), 7.30-7.28 (m, 1H), 4.68 (m, 2H), 4.13-4.07 (m, 3H), 3.84-3.76 (m, 4H), 3.67-3.61 (m, 4H), 3.13-3.08 (m, 2H), 2.77-26 (m, 3H), 2.65-2.64 (m, 3H), 2.48 (m, 2H), 1.48 (s, 9H), 1.29 (s, 3H), 1.20 (s, 3H). MS (m/z): 586.29 [M+H]⁺.

Intermediate 7B: tert-butyl 4-(6-benzyl-4-cyano-1-(2,6-dimethylmorpholino)-5,6,7,8-tetrahydro-2,6-naphthyridin-3-yl)piperazine-1-carboxylate: Was prepared using the process as described above for Intermediate 6C using tert-butyl piperazine-1-carboxylate (0.74 gm, 4.02 mmol) and Intermediate 5C (1.37 gm, 2.68 mmol) to get Intermediate 7B (1.2 gm), 1H-NMR (400 MHz): δ 7.34 (m, 4H), 7.30 (m, 1H), 4.22-4.17 (q, 2H), 3.64 (s, 2H), 3.20 (t, 2H), 2.81-2.76 (m, 1H), 2.60-2.58 (t, 2H), 2.40-2.31 (m, 2H), 1.26 (q, 3H). MS (m/z): 547.3 (M+1)+.

Intermediate 7C: tert-butyl 4-(6-benzyl-4-cyano-1-(2,6-dimethylmorpholino)-5,6,7,8-tetrahydro-2,6-naphthyridin-3-yl)-2-(cyanomethyl) piperazine-1-carboxylate: Was prepared using the process as described above for Intermediate 7B using 2-(piperazin-2-yl)acetonitrile dihydrochloride (1.6 gm, 13.1 mmol) and Intermediate 5E (4.4 gm, 8.6 mmol) to get Intermediate 7C (2.3 gm, 45.71%). MS (m/z): 547.3 (M+1).

Intermediates 7D: di-tert-butyl 4,4′-(6-benzyl-4-cyano-5,6,7,8-tetrahydro-2,6-naphthyridine-1,3-diyl)bis(piperazine-1-carboxylate): To a solution of tert-butyl piperazine-1-carboxylate (0.9 gm, 0.002 mol in THF (30 ml) was added DIPEA (1.2 ml, 0.03 mol and stirred at RT. Intermediate 5A (1.5 gm, 0.001 mol) in THF (10 ml) was added to the reaction mixture dropwise and heated to 80° C. for 2 hr. Reaction mixture was quenched with water and extracted with ethyl acetate. Organic layer, was washed with brine, dried over sodium sulphate and concentrated in vacuum to get a crude residue which was purified by column chromatography using ethyl acetate: hexane (25%) to get Intermediate 7D (1.25 gm). MS (m/z): 618.22 [M+H]⁺.

Intermediate 7E: Tert-butyl (R)-4-(6-benzyl-4-cyano-3-(4-methylpiperazin-1-yl)-5,6,7,8-tetrahydro-2,6-naphthyridin-1-yl)-3-methylpiperazine-1-carboxylate: Was prepared using the process as described above for Intermediate 5F using 1-Methyl piperazine (0.30 ml, 0.0027 mmol) (1.1 gm, 0.0018 mmol) to get Intermediates 7E (0.9 gm). MS (m/z): 546.23 (M+H)⁺.

Intermediate 7F: Tert-butyl (S)-4-(6-benzyl-4-cyano-3-(4-methylpiperazin-1-yl)-5,6,7,8-tetrahydro-2,6-naphthyridin-1-yl)-3-methylpiperazine-1-carboxylate: Was prepared using the process as described above for Intermediate 5A using 1-Methyl piperazine (0.30 ml, 0.0027 mmol) and Intermediate 5G (1.1 gm, 0.0018 mmol) to get Intermediate 7F as white solid (0.5 gm). MS (m/z): 546.23 (M+H)⁺.

Intermediate 7G: tert-butyl 4-(6-benzyl-4-cyano-3-(4-methylpiperazin-1-yl)-5,6,7,8-tetrahydro-2,6-naphthyridin-1-yl)-2-(cyanomethyl)piperazine-1-carboxylate: Was prepared using the process as described above for Intermediate 5C using 1-Methyl piperazine (0.30 ml, 0.0027 mol) (0.9 g, 0.0018 mmol) to get Intermediates 7G (0.9 gm), 71.35 (M+H)⁺.

Intermediate 8: tert-butyl 4-(6-benzyl-4-cyano-3-(2-morpholinoethoxy)-5,6,7,8-tetrahydro-2,6-naphthyridin-1-yl)piperazine-1-carboxylate: To a solution of Intermediate 5A (1.6 gm, 0.0035 mmol) in DMSO (20 mL) was added NaH (60% in mineral oil) (213 mg, 0.0089 mmol) and stirred at 0° C. for 20 minutes, 4 (2-chloroethyl morpholine. HCl) (1.3 gm, 0.0071 mmol) was added slowly and the reaction mixture was stirred at 80° C. for 3 hrs. The Reaction mixture was quenched with cold water (50 mL) and extracted with ethyl acetate (30 mL×2). The combined organic layer was washed with brine solution (30 mL), dried over Na2SO4 and evaporated under reduced pressure to get crude product which was purified through column chromatography using ethyl acetate and pet ether (50%) to get Intermediate 8 as white solid (1 gm), 1H-NMR (400 MHz): δ 7.37-7.34 (M, 4H), 7.32-7.30 (m, 1H), 4.49-4.46 (m, 4H), 3.77 (s, 2H), 3.73-3.69 (m, 6H), 3.51-3.50 (bs 4H), 3.29 (bs, 4H), 2.81-2.86 (m, 2H), 2.60 (bs, 8H), 1.47 (s, 9H). MS (m/z): 563.18 (M+1)⁺.

Intermediates 9A: tert-butyl 4-(6-benzyl-4-cyano-3-((methylsulfonyl)oxy)-5,6,7,8-tetrahydro-2,6-naphthyridin-1-yl)piperazine-1-carboxylate: To a solution of Intermediates 6A1 (2.1 gm, 0.0043 mol) in DCM (30 mL) was added TEA (1.5 mL, 0.0107 mol) at 0° C. Then methanesulphonyl chloride (0.5 mL, 0.0064 mol) solution in DCM (12 mL) was added slowly to the above solution and the reaction mixture was stirred at room temperature for 2 hrs. The reaction completion was checked by TLC using 40% Ethyl acetate and hexane as mobile phase. The reaction mixture was quenched into cold water and organic layer was separated. The aqueous layer was extracted with more DCM (20 mL×2) and the combined organic layer was washed with brine solution (20 mL). The organic layer was dried over Na2SO4 and concentrated under reduced pressure and temp. (30-35° C.) to get a crude residue which was purified by column chromatography (100-200 mesh silica gel) using 20-40% Ethyl acetate in hexane as mobile phase. The pure fractions were collected and concentrated to get Intermediate 9A (1.72 gm, 70.72%) as off-white solid, which used next reaction.

Intermediates 9B Tert-butyl 4-(6-benzyl-4-cyano-3-((methylsulfonyl)oxy)-5,6,7,8-tetrahydro-2,6-naphthyridin-1-yl)-2-(cyanomethyl)piperazine-1-carboxylate: Was prepared using the process as described above for Intermediates 9A, 1H-NMR (400 MHz) (CDCl3): 7.36-7.26 (m, 5H), 4.62 (bs, 1H), 4.13-4.09 (m, 1H), 3.90-3.81 (m, 2H), 3.78-3.67 (m, 4H), 3.44 (s, 3H), 2.29-3.25 (m, 2H), 3.05-2.98 (m, 1H), 2.79-2.68 (m, 5H), 2.61-2.58 (m, 1H) 1.50 (s, 9H). MS: m/Z; 567.2 (M+1).

Intermediate 10A: (tert-butyl 4-(6-benzyl-4-cyano-3-((1-methylpyrrolidin-2-yl)methoxy)-5,6,7,8-tetrahydro-2,6-naphthyridin-1-yl)piperazine-1-carboxylate): In a 100 mL pressure vessel, The suspension of Intermediate 5A (2.5 gm, 4.3 mmol), 1-methylpyrrolidine (0.99 gm, 8.6 mmol), Cs2CO3 (4.2 gm, 12.9 mmol) and ras BINAP (0.53 gm, 0.86 mmol) in Toluene (38 mL) was purged with N2 gas for 20 minutes. The Pd(OAc)2 (0.096 gm, 0.43 mmol) was added to the above reaction mixture and stirred at 120° C. for 3 hrs. The Reaction mixture was quenched with cold water (40 mL) and extracted with ethyl acetate (20 mL×2). The combined organic layer was washed with brine solution (30 mL), dried over Na2SO4 and evaporated under reduced pressure to get crude product which was purified through column chromatography using ethyl acetate and pet ether (30-100%) to get Intermediate 10A (1.2 gm, 50.93%), 1H-NMR (400 MHz): δ 7.38-7.26 (m, 5H), 4.324.23 (m, 2H), 3.78 (bs, 2H), 3.70 (bs, 2H), 3.52-3.43 (m, 4H), 3.31-3.28 (m, 4H), 3.11-3.07 (m, 1H), 2.70-2.66 (m, 1H), 2.60 (bs, 4H), 2.50 (s, 3H), 2.31-2.29 (m, 1H), 2.06-2.01 (m, 1H), 1.81-1.77 (m, 3H), 1.48 (s, 9H). MS (m/z): 547.3 (M+1), HPLC purity: 83.95%.

Intermediates 10B: tert-butyl 4-(6-benzyl-4-cyano-3-(((S)-1-methylpyrrolidin-2-yl)methoxy)-5,6,7,8-tetrahydro-2,6-naphthyridin-1-yl)2-(cyanomethyl)piperazine-1-carboxylate:

Method A: Was prepared using the process as described above for Intermediates 10A1H-NMR (400 MHz)(CDCl3): 7.37-7.29 (m, 5H), 4.62 (bs, 2H), 4.33-4.21 (m, 1H), 3.90-3.62 (m, 8H), 3.48-3.39 (m, 2H), 3.20-3.12 (m, 2H), 2.92-2.51 (m, 9H), 2.37-2.24 (m, 2H), 2.20-2.05 (m, 1H), 2.00-1.89 (m, 2H) 1.50 (s, 9H). MS: m/Z: 586.3 (M+1).

Method B: In 500 mL sealed tube, (S)-(1-methylpyrrolidin-2-yl)methanol (6.2 gm, 0.054 mol) was added followed by addition of Dry THF (150 ml) stirred the reaction mixture 0° C. NaH (60%) (1.74 gm, 0.072 mol) was added in reaction mixture and stirred the reaction same temperature over 30 min under the nitrogen. Intermediate 5 (18.5 g, 0.036 mol) was dissolved in dry THF (120 mL) added to the reaction mixture over dropwise and stirred at 85° C. for 12 hrs. After completion of reaction the RM was poured into saturated solution ammonium chloride (200 mL) and extracted with ethyl acetate (500 mL×2). The organic layer was dried over sodium sulphate and concentrated under reduced pressure to get a crude residue. The crude was purified by column chromatography using 3% DCM: Methanol as mobile phase. The pure fractions were collected and concentrated under reduced pressure to affords Intermediate 6 (14 g) as light-yellow solid, 1H-NMR (δ ppm, CDCl₃, 400 MHz): 7.37-7.32 (M, 4H), 7.31-7.29 (m, 1H), 4.31 (bs, 1H), 3.90 (bs, 1H), 3.67 (bs, 1H), 3.65-3.55 (m, 1H), 3.48-3.44 (m, 1H), 3.18-3.14 (m, 2H), 2.85-2.82 (m, 6H), 2.63 (s, 3H), 2.45 (m, 1H), 2.03-1.99 (m, 1H), 1.78.1.75 (m, 6H), 1.50 (s, 9H), MS (m/z): 586.34 [M+H]⁺.

Intermediate 11: tert-butyl4-(4-cyano-3-morpholino-5,6,7,8-tetrahydro-2,6-naphthyridin-1-yl)piperazine-1-carboxylate: In the 100 ml two neck RBF Intermediate 6B (0.9 gm, 1.59 mmol) in ethanol (18 mL) was purged with N2 gas for 20 minutes, followed by addition of Pd(OH)₂/C (20%, 0.27 gm, 0.3/w). The reaction mixture was stirred at 50° C. for 3 hrs under the H2 atmosphere. The reaction completion was checked by TLC using MeOH: DCM (5%). After completion of reaction, the mixture was filtered through celite bed under inert atmosphere and the filtrate was evaporated under reduced pressure to get a crude residue which was purified by column chromatography using 5% MeOH in DCM as mobile phase. The pure fractions were collected and concentrated to get the desired product (0.4 gm, 52.78%), 1H-NMR (400 MHz): δ 4.154.13 (m, 4H)), 3.55-3.53 (m, 4H), 3.29-3.26 (m, 4H), 3.19-3.17 (m, 4H), 3.14-3.10 (m, 2H), 3.09-3.06 (m, 2H), 2.64-2.62 (m, 2H), 2.07 (s, 1H), 1.49 (s, 9H) MS (m/z): 477.2 (M+1).

Intermediate 12: Tert-butyl 4-(4-cyano-3-(1,1-dioxidothiomorpholino)-5,6,7,8-tetrahydro-2,6-naphthyridin-1-yl)piperazine-1-carboxylate: Was prepared using the process as described above for Intermediate 11 using Intermediate 6C (0.9 gm, 0.0015 mmol) to get Intermediate 12 as white solid (0.6 gm), 1H-NMR (S ppm. CDCl₃, 400 MHz): 4.15-4.12 (m, 5H), 3.55-3.53 (M, 4H), 3.29-3.26 (m, 4H), 3.19-3.14 (m, 4H), 3.11-3.09 (m, 2H), 3.07-3.05 (m, 2H), 2.64-2.61 (m, 1H), 1.48 (s, 9H). MS (m/z): 478.02 [M+H]⁺.

Intermediate 13: tert-butyl 4-(4-cyano-3-(2-morpholinoethoxy)-5,6,7,8-tetrahydro-2,6-naphthyridin-1-yl)piperazine-1-carboxylate: Was prepared using the process as described above for Intermediate 11 using Intermediate 8 (1.0 gm, 0.0017 mmol) to get Intermediate 13 (0.3 gm). 1H-NMR (δ ppm, CDCl₃, 400 MHz): 4.49-4.46 (t, 2H), 4.11 (s, 4H), 3.73-3.71 (t, 4H), 3.54-3.52 (t, 4H), 3.30-3.81 (m, 4H), 3.01-2.99 (m, 2H), 2.91-2.78 (m, 2H), 2.61-2.59 (m, 4H), 2.50 (bs, 2H), 1.48 (s, 9H). MS (m/z): 473.56 [M+H]⁺.

Intermediate 14: tert-butyl 4-(4-cyano-3-(2,6-dimethylmorpholino)-5,6,7,8-tetrahydro-2,6-naphthyridin-1-yl)-2-(cyanomethyl)piperazine-1-carboxylate: Was prepared using the process as described above for Intermediate 11 using Intermediate 7A (2.6 gm, 0.0044 mmol) to get Intermediate 14 as white solid (2.2 gm). 1H-NMR (S ppm. CDCl₃, 400 MHz): 4.154.11 (m, 4H), 3.98 (s, 4H), 3.75-3.72 (m, 2H), 3.54-3.51 (m, 4H), 3.26-3.24 (m, 4H), 3.02-3.01 (m, 2H), 2.68-2.51 (m, 2H), 1.48 (s, 9H), 1.29 (s, 3H), 1.20 (s, 3H). MS (m/z): 457.12 [M+H]⁺.

Intermediate 15: (tert-butyl 4-(4-cyano-1-(2,6-dimethylmorpholino)-5,6,7,8-tetrahydro-2,6-naphthyridin-3-yl)piperazine-1-carboxylate): Was prepared using the process as described above for Intermediate 11 using Intermediate 7A (1.2 gm, 2.19 mmol) to get Intermediate 15 (0.4 gm, 40%). 1H-NMR (400 MHz): δ 4.15 (s, 2H)), 3.76-3.72 (m, 8H), 3.60-3.56 (m, 4H), 2.09-3.06 (m, 2H), 2.66-2.60 (m, 4H), 2.04 (s, 1H), 1.48 (s, 9H), 1.24 (s, 3H), 1.22 (s, 3H). MS (m/z): 457.3 (M+1).

Intermediate 16: (tert-butyl 4-(4-cyano-1-(2,6-dimethylmorpholino)-5,6,7,8-tetrahydro-2,6-naphthyridin-3-yl)-2-(cyanomethyl)piperazine-1-carboxylate): Was prepared using the process as described above for Intermediate 11 using Intermediate 7B (2.3 gm, 3.91 mmol) to get Intermediate 16 (0.9 gm, 46.46%). MS: m/z: 496.3 (M+1).

Intermediate 17: (tert-butyl 4-(4-cyano-3-((1-methylpyrrolidin-2-yl)methoxy)-5,6,7,8-tetrahydro-2,6-naphthyridin-1-yl)piperazine-1-carboxylate): Was prepared using the process as described above for Intermediate 11 using Intermediate 10A (1.2 gm, 2.19 mmol) to get Intermediate 17 (0.32 gm, 32.37%). MS (m/z): 457.2 (M+1).

Intermediates 18: tert-butyl 4-(4-cyano-3-(((S)-1-methylpyrrolidin-2-yl)methoxy)-5,6,7,8-tetrahydro-2,6-naphthyridin-1-yl)-2-(cyanomethyl)piperazine-1-carboxylat: In two neck 500 mL RBF, Intermediates 10B (10 gm, 0.017 mol) in Ethanol (200 mL) was degassed with N2 for 20 minutes. Then methanolic ammonia solution (12 mL) was added to the above solution, followed by addition of 6 gm Pd(OH)2/C (20%). The entire RM was stirred at 70° C. under the atmosphere of H2 gas for 24 hrs. The reaction completion was checked by TLC using 5% MeOH in DCM and 1 drop of methanolic ammonia. After completion of reaction, the mixture was filtered through celite bed under inert atmosphere and the filtrate was evaporated under reduced pressure to get the Intermediates 17 desired product of light-yellow solid (7.3 gm). 1H-NMR (400 MHz) (CDCl3): 4.814.78 (m, 1H), 4.61 (bs, 1H), 4.49-4.44 (m, 1H), 4.14-4.04 (m, 3H), 3.85-3.79 (m, 1H), 3.67-3.64 (m, 1H), 3.56-3.54 (m, 1H), 3.36-3.18 (m, 5H), 2.96-2.92 (m, 6H), 2.71-2.61 (m, 2H), 2.32-1.97 (m, 5H), 1.51 (s, 9H). MS (m/z): 496.3 (M+1).

Intermediate 18A: Di-tert-butyl4,4′-(4-cyano-5,6,7,8-tetrahydro-2,6-naphthyridine-1,3-diyl)bis(piperazine-1-carboxylate): To a solution of Intermediates 7D (1.0 gm, 0.001 mol) in ethanol was purged nitrogen gas for 20 min, after that added 5 drop of acetic acid, followed by addition of Pd₂(OH)₂ (0.3 gm) and the reaction mixture was heated at 50° C. for 12 hr. Reaction mixture was filtered over celite pad and filtrate, evaporated under reduced vacuum to get crude product. The crude was purified by column chromatography using Methanol: DCM (10%) to get Intermediate 26 as white solid (0.4 gm). 1H-NMR (δ ppm, CDCl₃, 400 MHz): 4.14 (s, 2H), 3.59-3.52 (m, 12H), 3.28-3.25 (m, 4H), 3.04-3.01 (m, 2H), 2.57-2.51 (m, 2H), 1.43 (s, 18H). MS (m/z): 528.25 [M+H]⁺.

Intermediate 18B: tert-butyl 4-(4-cyano-3-(4-methylpiperazin-1-yl)-5,6,7,8-tetrahydro-2,6-naphthyridin-1-yl)piperazine-1-carboxylate: Was prepared using the process as described above for Intermediate 18A using Intermediate 6D (1.0 gm, 0.001 mol) to get Intermediate 18B as white solid (0.4 gm). 1H-NMR (δ ppm, CDCl₃, 400 MHz): 4.08 (s, 2H), 3.69-3.67 (m, 4H), 3.53-3.48 (m, 4H), 3.28-3.27 (m, 4H), 3.00-2.98 (m, 2H), 2.60-2.58 (m, 4H), 2.55-2.53 (m, 2H), 2.37 (s, 3H), 1.48 (s, 9H). MS (m/z): 442 [M+H]⁺.

Intermediate 18C: tert-butyl 4-(4-cyano-3-(piperidin-1-yl)-5,6,7,8-tetrahydro-2,6-naphthyridin-1-yl)piperazine-1-carboxylate: Was prepared using the process as described above for Intermediate 18A using Intermediate 6F (0.6 gm, 0.0011 mol) to get Intermediate 18C as white solid (0.4 gm). MS (m/z): 428.45 [M+H]⁺.

Intermediate 18D: Tert-butyl4-(4-cyano-3-(2,6-dimethylmorpholino)-5,6,7,8-tetrahydro-2,6-naphthyridin-1-yl) piperazine-1-carboxylate: Was prepared using the process as described above for Intermediate 18A using Intermediate 6G (0.8 gm, 0.0018 mmol) to get Intermediate 18D (0.6 gm). 1H-NMR (δ ppm, CDCl₃, 400 MHz): 4.15-4.11 (m, 4H), 3.98 (s, 4H), 3.75-3.72 (m, 2H), 3.54-3.51 (m, 4H), 3.26-3.24 (m, 4H), 3.02-3.01 (m, 2H), 2.68-2.51 (m, 2H), 1.48 (s, 9H), 1.29 (s, 3H), 1.20 (s, 3H). MS (m/z): 457.12 [M+H]⁺.

Intermediate 18E: tert-butyl4-(4-cyano-3-(4-propionylpiperazin-1-yl)-5,6,7,8-tetrahydro-2,6-naphthyridin-1-yl)piperazine-1-carboxylate: Was prepared using the process as described above for Intermediate 18A using Intermediate 6H (1.0 gm, 0.0017 mmol) to get Intermediate 18E as white solid (0.6 gm). MS (m/z): 484.12 [M+H]⁺.

Intermediate 18F: tert-butyl 4-(4-cyano-3-(4-(methylsulfonyl)piperazin-1-yl)-5,6,7,8-tetrahydro-2,6-naphthyridin-1-yl)piperazine-1-carboxylate: Was prepared using the process as described above for Intermediate 18A using Intermediate 61 (1.0 gm, 0.0017 mmol) to get Intermediate 18F as white solid (0.6 gm). ¹H-NMR (δ ppm, CDCl₃, 400 MHz): 4.113 (s, 2H), 3.68-3.66 (m, 4H), 3.54-3.51 (m, 4H), 3.37-3.35 (m, 4H), 3.29-3.27 (m, 4H), 3.06-3.03 (m, 2H), 2.83 (s 3H), 2.61-2.58 (m, 2H), 1.48 (s, 9H). MS (m/z): 506.20 [M+H]⁺.

Intermediate 18G: tert-butyl 4-(3-(4-acetylpiperazin-1-yl)-4-cyano-5,6,7,8-tetrahydro-2,6-naphthyridin-1-yl)piperazine-1-carboxylate: Was prepared using the process as described above for Intermediate 26 using Intermediate 6J (0.8 gm, 0.0017 mmol) to get Intermediate 18G as white solid (0.6 gm). 1H-NMR (δ ppm, CDCl₃, 400 MHz): 4.11 (s, 2H), 3.75-3.74 (m, 2H), 3.64-3.59 (m, 6H), 3.58-3.52 (m, 4H), 3.29-3.27 (m, 4H), 3.04-3.03 (m, 2H), 2.58-2.56 (m, 2H), 2.13 (s, 3H), 1.48 (s, 9H). MS (m/z): 470.12 [M+H]⁺.

Intermediate 18H: tert-butyl 4-(4-cyano-5,6,7,8-tetrahydro-2,6-naphthyridin-1-yl)piperazine-1-carboxylate: Was prepared using the process as described above for Intermediate 11 using Intermediate 5H (0.7 gm, 0.0015 mol) to get Intermediate 18H (0.4 gm). 344.20 [M+H]⁺.

Intermediate 19A: tert-butyl 4-(6-(8-chloronaphthalen-1-yl)-4-cyano-3-morpholino-5,6,7,8-tetrahydro-2,6-naphthyridin-1-yl)piperazine-1-carboxylate: To a solution of Intermediate 11 (0.4 g, 0.00075 mmol) in 1,4-dioxane (10 ml) purged under nitrogen gas for 20 min, was added 1-Bromo-8-bromo naphthalene (0.3 gm, 0.0015 mmol), Sodium tert.butoxide (0.29 gm, 0.0030 mmol) and r-BINAP (0.093 gm, 0.00015 mmol) over a period of 10 min and stirred under Nitrogen atmosphere. Pd2(dba)3 (0.068 gm, 0.000075 mmol) was then added to the reaction mixture and heated at 130° C. for 16 hr. Reaction mixture was filtered over celite pad and filtrate, evaporated under reduced vacuum to get crude residue which was purified by column chromatography using MeOH: DCM (5%) to get Intermediate 19 A 1H-NMR (δ ppm, CDCl₃, 400 MHz): 7.77-7.75 (m, 1H), 7.64-7.62 (m, 1H), 7.53-7.51 (m, 1H), 7.46-7.42 (m, 1H), 7.31-7.32 (m, 1H), 7.23-7.21 (m, 1H), 4.58-4.53 (m, 1H), 3.87-3.81 (m, 1H), 3.64-3.62 (m, 4H), 3.59-3.54 (m, 4H), 3.52-3.49 (m, 2H), 3.23-3.22 (m, 2H), 3.15-3.05 (m, 2H), 2.60 (bs, 2H) 1.49 (s, 9H).

Intermediate 19B: Tert-butyl 4-(4-cyano-3-(1,1-dioxidothiomorpholino)-6-(naphthalen-1-yl)-5,6,7,8-tetrahydro-2,6-naphthyridin-1-yl)piperazine-1-carboxylate): Was prepared using the process as described above for Intermediate 19A using Intermediate 12 get Intermediate 19B as white solid (0.22 gm, 43.45%). 1H-NMR (400 MHz): δ 8.20-8.18 (m, 1H)), 7.89-7.87 (m, 1H), 7.64 (d, J=8.0 Hz, 1H), 7.53-7.50 (m, 2H), 7.46-7.43 (m, 1H), 7.19 (d, J=6.8 Hz, 1H), 4.40 (bs, 2H), 4.18-4.15 (m, 4H), 3.60-3.58 (m, 4H), 3.36 (bs, 6H), 3.22-3.19 (m, 4H), 2.88 (bs, 2H), 1.50 (s, 9H) MS (m/z): 603.2 (M+1).

Intermediate 19C: Tert-butyl 4-(4-cyano-3-(2,6-dimethylmorpholino)-6-(naphthalen-1-yl)-5,6,7,8-tetrahydro-2,6-naphthyridin-1-yl)-2-(cyanomethyl)piperazine-1-carboxylate: Was prepared using the process as described above for Intermediate 19A using Intermediate 14 (1 g, 0.0020 mmol), 1-Bromonapthene (0.59 ml, 0.004 mmol), cesium carbonate (1.97 gm, 0.0066 mmol), Ruphos (0.18 gm, 0.0004 mmol) and Pd2(dba)3 (0.184 gm, 0.000022 mmol) with toluene as a solvent at 110° C. for 3 hr to get Intermediate 19C as white sold (0.9 gm). 1H-NMR (δ ppm, CDCl₃, 400 MHz): 8.19-8.17 (m, 1H), 7.88-7.85 (m, 1H), 7.64-7.61 (m, 1H), 7.51-7.49 (m, 2H), 7.44-7.7.42 (m, 1H), 7.19-7.17 (m, 1H), 4.41 (bs, 1H), 4.40 (bs, 1H), 4.17-4.11 (m, 3H), 3.80-3.73 (m, 4H), 3.70 (bs, 1H), 2.25 (bs, 3H), 2.89-2.79 (m, 5H), 2.75-2.69 (m, 3H), 1.52 (s, 9H), 1.29 (s, 6H). MS (m/z): 622.22[M+H]⁺.

Intermediate 19D: tert-butyl 4-(4-cyano-3-(2-morpholinoethoxy)-6-(naphthalen-1-yl)-5,6,7,8-tetrahydro-2,6-naphthyridin-1-yl)piperazine-1-carboxylate: Was prepared using the process as described above for Intermediate 19A using Intermediate 13 (0.3 gm, 0.00063 mmol), 1-bromonaphthalene (0.18 mL, 0.001271 mmol), Ruphos (0.06 gm, 0.00012 mmol), Cs2CO3 (0.13 gm, 0.00031 mmol) and Pd2(dba)3 (0.058 gm, 0.000063 mmol) with toluene as a solvent and at 110° C. for 4 hrs to get Intermediate 19D as white solid (0.25 gm, 18.17%). 1H-NMR (400 MHz): δ 8.20-8.17 (m, 1H), 7.88-7.86 (m, 1H), 7.64-7.62 (m, 1H), 7.52-7.50 (m, 2H), 7.48-7.43 (m, 1H), 7.18-7.17 (m, 1H), 4.54 (bs, 2H), 4.41 (bs, 2H), 3.75 (bs, 4H), 3.58-3.57 (bs. 4H), 3.38 (bs, 5H), 2.85 (bs, 4H), 2.64 (bs, 4H), 1.49 (s, 9H). MS (m/z): 599.21 (M+1)

Intermediate 19E: tert-butyl 4-(6-(8-chloronaphthalen-1-yl)-4-cyano-3-(2-morpholinoethoxy)-5,6,7,8-tetrahydro-2,6-naphthyridin-1-yl)piperazine-1-carboxylate Was prepared using the process as described above for Intermediate 19A using Intermediate 13 (0.3 gm, 0.00063 mmol), 1-bromo-8-chloro naphthalene (0.36 mg, 0.001271 mmol), Ruphos (0.06 gm, 0.00012 mmol), Cs2CO3 (0.13 gm, 0.00031 mmol) and Pd2(dba)3 (0.058 gm, 0.000063 mmol) with toluene as a solvent and at 110° C. for 4 hrs to get Intermediate 19E as white solid (0.25 gm, 18.17%). 1H-NMR (400 MHz): δ 7.78-7.75 (m, 1H), 7.65-7.63 (m, 1H), 7.54-7.52 (m, 1H), 7.47-7.23 (m, 1H), 7.36-7.32 (m, 1H), 7.24-7.23 (m, 1H), 4.60-4.48 (m, 3H), 3.94-3.74 (m, 1H), 3.64-3.61 (m, 4H), 3.54-3.52 (m, 2H), 3.49-3.44 (m, 5H), 3.28-3.26 (m, 2H), 3.24-3.12 (m, 2H), 2.80-2.52 (m, 4H), 1.49 (s, 9H). MS (m/z): 634.20 (M+1)

Intermediate 19F: (tert-butyl 4-(4-cyano-1-(2,6-dimethylmorpholino)-6-(naphthalen-1-yl)-5,6,7,8-tetrahydro-2,6-naphthyridin-3-yl)piperazine-1-carboxylate)): Was prepared using the process as described above for Intermediate 19A using Intermediate 16 (0.4 gm, 0.88 mmol), 1-bromonaphthalene (0.25 mL, 1.75 mmol), xantphos (0.1 gm, 0.17 mmol), Cs2CO3 (0.86 gm, 2.63 mmol) and Pd2(dba)3 with toluene as a solvent and at 110° C. for 4 hrs to get Intermediate 19E (0.32 gm, 63.7%). 1H-NMR (400 MHz): δ 8.22-8.20 (m, 1H), 7.88-7.86 (m, 1H), 7.64-7.62 (m, 1H), 7.52-7.50 (m, 2H), 7.46-7.42 (m, 1H), 7.20-7.18 (m, 1H), 4.38 (bs, 2H)), 3.79-3.78 (m, 2H), 3.69-3.68 (m, 2H), 3.59-3.58 (m, 8H), 3.38-25 (m, 2H), 2.88-2.79 (m, 2H), 2.72-2.62 (m, 2H), 1.42 (s, 9H), 1.28 (s, 3H), 1.26 (s, 3H). MS (m/z): 583.3 (M+1).

Intermediate 19G: (tert-butyl 4-(4-cyano-1-(2,6-dimethylmorpholino)-6-(naphthalen-1-yl)-5,6,7,8-tetrahydro-2,6-naphthyridin-3-yl)-2-(cyanomethyl)piperazine-1-carboxylate): Was prepared using the process as described above for Intermediate 19A using Intermediate 16 (0.88 gm, 1.78 mmol), 1-bromonaphthalene (0.53 mL, 3.56 mmol), xantphos (0.2 gm, 0.36 mmol), Cs2CO3 (1.72 gm, 5.34 mmol) and Pd2(dba)3 (0.16 gm, 0.18 mmol) with toluene as a solvent and at 110° C. for 4 hrs to get Intermediate 19F as w % bite solid (0.2 gm, 18.17%). MS (m/z): 622.3 (M+1)

Intermediate 1911: (tert-butyl 4-(4-cyano-3-((1-methylpyrrolidin-2-yl)methoxy)-6-(naphthalen-1-yl)-5,6,7,8-tetrahydro-2,6-naphthyridin-1-yl)piperazine-1-carboxylate): Was prepared using the process as described above for Intermediate 19A using Intermediate 17 (0.32 gm, 0.71 mmol), 1-bromonaphthalene (0.21 mL, 1.42 mmol), RuPhos (0.066 gm, 0.14 mmol), Cs2CO3 (0.7 gm, 2.13 mmol) and Pd2(dba)3 (0.064 gm, 0.071 mmol) with toluene as a solvent and at 110° C. for 3 hrs to get Intermediate 19G (0.2 gm, 48.31%). 1H-NMR (400 MHz): δ 8.20-8.18 (m, 1H), 7.88-7.85 (m, 1H), 7.64-7.62 (m, 1H), 7.53-7.48 (m, 2H), 7.46-7.41 (m, 1H), 7.19-7.17 (m, 1H), 4.40-3.70 (m, 4H), 3.67 (bs, 4H), 3.30-3.12 (m, 5H), 3.11-2.87 (m, 1H), 2.75 (bs, 2H), 2.73-2.54 (m, 1H), 2.53 (s, 3H), 2.31-2.08 (m, 1H), 2.07-2.01 (m, 1H), 1.87-1.68 (m, 4H), 1.42 (s, 9H). MS (m/z): 583.3 (M+1).

Intermediates 191: tert-butyl 4-(6-(8-chloronaphthalen-1-yl)-4-cyano-3-(((S)-1-methylpyrrolidin-2-yl)methoxy)-5,6,7,8-tetrahydro-2,6-naphthyridin-1-yl)-2-(cyanomethyl)piperazine-1-carboxylate: In 500 mL sealed tube, Intermediate 18 (7 gm, 0.014 mol), 1-bromo-8-chloronaphthalene (5.01 gm, 0.027 mol), Xantphos (1.6 gm, 0.002 mol) and Cs2CO3 (13.8 gm, 0.042 mol) in Toluene (140 mL) was degassed with N2 for 20 minutes. Then Pd2(dba)3 (1.3 gm, 0.0014 mol) was added to the above solution and entire RM was stirred at 120° C. for 12 hrs. The reaction completion was checked by TLC using 5% MeOH in DCM and 1 drop of methanolic ammonia. After completion of reaction, the mixture was poured in cold water (20 mL) and extracted with ethyl acetate (30 mL×2). The organic layer was washed with brine solution (10 mL), dried over Na2SO4 and evaporated under reduced pressure to get a crude residue which was purified by column chromatography (100-200 mesh silica gel) using 3% MeOH in DCM as mobile phase. The pure fractions were collected and concentrated to get Intermediates 191 as the desired product (7.26 gm). 1H-NMR (400 MHz) (CDCl3): 7.77-7.75 (m, 1H), 7.66-7.62 (m, 1H), 7.53-7.51 (m, 1H), 7.48-7.42 (m, 1H), 7.40-7.32 (m, 11H), 7.28-7.18 (m, 1H), 4.59-4.55 (m, 2H), 4.34-4.30 (m, 2H), 3.90-3.77 (m, 1H), 3.60-3.55 (m, 2H), 3.34-3.31 (m, 1H), 3.19-3.13 (m, 11H), 3.09-3.03 (m, 4H), 2.73-2.64 (m, 3H), 2.53 (s, 3H), 2.33-2.31 (m, 1H), 2.06-2.03 (m, 1H), 1.85-1.78 (m, 5H), 1.51 (s, 9H). MS (m/z): 656.3 (M+1). HPLC: 99.01%.

Intermediates 19J: tert-butyl 4-(6-(3-(benzyloxy)naphthalen-1-yl)-4-cyano-3-(((S)-1-methylpyrrolidin-2-yl)methoxy)-5,6,7,8-tetrahydro-2,6-naphthyridin-1-yl)-2-(cyanomethyl)piperazine-1-carboxylate: Was prepared using the process as described above for Intermediate 19A using Intermediates 18 (1.4 gm, 0.0028 mol), 3-(benzyloxy)-1-bromonaphthalene (1.77 gm, 0.0056 mol), Xantphos (0.32 gm, 0.00056 mol), Cs2CO3 (2.7 gm, 0.0084 mol), Toluene (20 mL) and Pd2(dba)3 (0.26 gm, 0.00028 mol) to get the desired product of Intermediates 19J (1 gm, 49.06%). MS (m/z): 728.5 (M+1). HPLC: 80.75%.

Intermediate 19K: Di-tert-butyl 4,4′-(4-cyano-6-(naphthalen-1-yl)-5,6,7,8-tetrahydro-2,6-naphthyridine-1,3-diyl)bis(piperazine-1-carboxylate): To a solution of Intermediate 18A (0.4 g, 0.00075 mmol) in 1,4-dioxane (10 ml) purged under nitrogen gas for 20 min, was added 1-Bromonapthene (0.22 ml, 0.0015 mmol), Sodium tert.butoxide (0.29 gm, 0.0030 mmol) and r-BINAP (0.093 gm, 0.00015 mmol) over a period of 10 min and stirred under Nitrogen atmosphere. Pd2(dba)3 (0.068 gm, 0.000075 mmol) was then added to the reaction mixture and heated at 130° C. for 16 hr. Reaction mixture was filtered over celite pad and filtrate, evaporated under reduced vacuum to get crude residue which was purified by column chromatography using MeOH: DCM (5%) to get Intermediate 19K (0.34 gm). 1H-NMR (S ppm. CDCl₃, 400 MHz): 8.21-8.18 (m, 1H), 7.87-7.85 (m, 1H), 7.61-7.52 (m, 1H), 7.51-7.49 (m, 2H), 7.47-7.63 (m, 1H), 7.26-7.19 (m, 1H), 4.38 (s, 2H), 3.62-3.56 (m, 13H), 3.53 (bs, 5H), 2.85 (bs, 2H), 1.49 (s, 18H). MS (m/z): 554.37[M+H]⁺.

Intermediate 19L B: Di-tert-butyl 4,4′-(6-(benzo[b]thiophen-4-yl)-4-cyano-5,6,7,8-tetrahydro-2,6-naphthyridine-1,3-diyl)bis(piperazine-1-carboxylate) Was prepared using the process as described above for Intermediate 19K using Intermediate 18A (0.4 g, 0.00075 mmol) and 4-bromobenzo[b]thiophene (0.31 ml, 0.0015 mmol) to get Intermediate 19L (0.3 gm). 1H-NMR (δ ppm, CDCl3, 400 MHz): 7.62-7.59 (m, 1H), 7.45-7.44 (m, 2H), 7.32-7.26 (m, 1H), 6.99-6.97 (m, 11H), 4.41 (s, 2H), 3.62-3.55 (m, 11H), 3.45-3.32 (m, 7H), 2.82-2.79 (bs, 2H), 1.49 (s, 18H). MS (m/z): 660.12 [M+H]⁺.

Intermediate 19M: Tert-butyl 4-(4-cyano-3-(4-methylpiperazin-1-yl)-6-(naphthalen-1-yl)-5,6,7,8-tetrahydro-2,6-naphthyridin-1-yl)piperazine-1-carboxylate: Was prepared using the process as described above for Intermediate 19K using Intermediate 18B (0.4 g, 0.009 mmol), 1-Bromonapthene (0.26 ml, 0.0018 mol), Sodium tert.butoxide (0.348 gm, 0.0036 mol), r-BINAP (0.112 gm, 0.00018 mol) and Pd2(dba)3 (0.083 gm, 0.00009 mol) to get Intermediate 19M. (0.34 gm). 1H-NMR (o ppm, CDCl₃, 400 MHz): 8.21-8.19 (m, 1H), 7.787-7.85 (m, 1H), 7.62-7.60 (m, 1H), 7.52-7.47 (m, 2H), 7.45-7.41 (m, 1H), 7.19-7.17 (m, 1H), 4.38 (s, 2H), 3.72-3.69 (m, 4H), 3.58-3.55 (m, 4H), 3.55 (bs, 5H), 3.21 (s, 1H), 2.84 (bs, 2H), 2.57-2.55 (m, 4H), 2.35 (s, 3H), 1.49 (s, 9H). MS (m/z): 568.10[M+H]⁺.

Intermediate 19N: Tert-butyl 4-(4-cyano-6-(naphthalen-1-yl)-3-(piperidin-1-yl)-5,6,7,8-tetrahydro-2,6-naphthyridin-1-yl)piperazine-1-carboxylate: Was prepared using the process as described above for Intermediate 19K using Intermediate 18C (0.27 g, 0.0006 mmol), 1-Bromonapthene (0.18 ml, 0.0018 mol), Sodium tert.butoxide (0.243 gm, 0.00025 mol), r-BINAP (0.078 gm, 0.00016 mol) and Pd2(dba)3 (0.057 gm, 0.000063 mmol) to get Intermediate 19N as white solid (0.34 gm). 1H-NMR (δ ppm, CDCl₃, 400 MHz): 8.22-8.19 (m, 1H), 7.78-7.84 (m, 1H), 7.62-7.60 (m, 1H), 7.51-7.48 (m, 2H), 7.45-7.41 (M, 1H), 7.19-7.17 (m, 1H), 4.37 (bs, 2H), 3.63-3.62 (m, 4H), 3.58-3.55 (m, 4H), 3.33 (bs, 6H), 2.80 (bs, 2H), 1.49 (s, 9H), 1.23 (bs, 6H). MS (m/z): 553.10 [M+H]⁺.

Intermediate 19O: tert-butyl4-(4-cyano-3-(2,6-dimethylmorpholino)-6-(naphthalen-1-yl)-5,6,7,8-tetrahydro-2,6-naphthyridin-1-yl)piperazine-1-carboxylate Was prepared using the process as described above for Intermediate 19K using Intermediate 18D (0.5 g, 0.0010 mmol), 1-Bromonapthene (0.25 ml, 0.0020 mmol). Sodium tert.butoxide (0.38 gm, 0.004 mmol), r-BINAP (0.12 gm, 0.0002 mmol) and Pd2(dba)3 (0.09 gm, 0.00001 mmol) to get Intermediate 190 (0.4 gm). 1H-NMR (δ ppm, CDCl₃, 400 MHz): 8.21-8.19 (m, 1H), 7.87-7.85 (m, 1H), 7.62-7.60 (m, 1H), 7.51-7.49 (m, 2H), 7.45-7.41 (m, 1H), 7.26-7.17 (m, 1H), 4.38 (s, 2H), 4.17-4.14 (m, 2H), 3.80-3.72 (m, 2H), 3.69-3.57 (m, 4H), 3.56-3.33 (m, 6H), 2.89 (bs, 2H), 2.74-2.68 (m, 1H) 1.54 (s, 9H), 1.25 (s, 6H). MS (m/z): 584.12 [M+H]⁺.

Intermediate 19P: tert-butyl4-(4-cyano-6-(naphthalen-1-yl)-3-(4-propionylpiperazin-1-yl)-5,6,7,8-tetrahydro-2,6-naphthyridin-1-yl)piperazine-1-carboxylate: Was prepared using the process as described above for Intermediate 19K using Intermediate 18E (0.5 g, 0.0010 mmol), 1-Bromonapthene (0.25 ml, 0.0020 mmol), Sodium tert.butoxide (0.38 gm, 0.004 mmol), r-BINAP (0.12 gm, 0.0002 mmol) and Pd2(dba)3 (0.09 gm, 0.00001 mmol) to get Intermediate 19P (0.4 gm). 1H-NMR (δ ppm, CDCl₃, 400 MHz): 8.21-8.18 (m, 1H), 7.87-7.85 (m, 1H), 7.63-7.61 (m, 1H), 7.51-7.49 (m, 2H), 7.48-7.43 (m, 1H), 7.19-7.7 (m, 1H), 4.41 (s, 2H), 3.82 (s, 2H), 3.62-3.61 (m, 10H), 3.50-3.41 (m, 6H), 2.64 (bs, 2H), 2.50-2.48 (q, 2H), 1.48 (s, 9H), 1.30-1.25 (t, 3H) 1.25 (s, 6H). MS (m/z): 610.34 [M+H]⁺.

Intermediate 19Q: tert-butyl 4-(4-cyano-3-(4-(methylsulfonyl)piperazin-1-yl)-6-(naphthalen-1-yl)-5,6,7,8-tetrahydro-2,6-naphthyridin-1-yl)piperazine-1-carboxylate: Was prepared using the process as described above for Intermediate 19K using Intermediate 18F (0.6 g, 0.0011 mmol), 1-Bromonapthene (0.5 ml, 0.0022 mmol), Sodium tert.butoxide (0.42 gm, 0.0044 mmol), r-BINAP (0.13 gm, 0.00022 mmol) and Pd2(dba)3 (0.11 gm, 0.000011 mmol) to get Intermediate 19Q. as white solid (0.4 gm). 1H-NMR (δ ppm, CDCl₃, 400 MHz): 8.20-8.18 (m, 1H), 7.88-7.85 (m, 1H), 7.63-7.53 (m, 1H), 7.51-7.49 (m, 2H), 7.45-7.43 (m, 0.1H), 7.19-7.17 (m, 1H), 4.39 (bs. 2H), 3.71-3.69 (m, 4H), 3.59-3.56 (m, 4H), 3.48 (bs, 1H), 3.40-3.37 (m, 9H), 2.87 (bs, 2H), 2.82 (s, 3H), 1.48 (s, 9H).

Intermediate 19R: tert-butyl 4-(3-(4-acetylpiperazin-1-yl)-4-cyano-6-(naphthalen-1-yl)-5,6,7,8-tetrahydro-2,6-naphthyridin-1-yl)piperazine-1-carboxylate: Was prepared using the process as described above for Intermediate 19K using Intermediate 18G (0.6 g, 0.0011 mmol), 1-Bromonapthene (0.5 ml, 0.0022 mmol), Sodium tert.butoxide (0.42 gm, 0.0044 mmol), r-BINAP (0.13 gm, 0.00022 mmol) and Pd2(dba)3 (0.11 gm, 0.000011 mmol) to get Intermediate 19R as white sold (0.4 gm). 1H-NMR (δ ppm, CDCl3, 400 MHz): 8.22-8.18 (m, 1H), 7.87-7.85 (m, 1H), 7.63-7.61 (m, 1H), 7.51-7.49 (m, 2H), 7.45-7.2 (m, 1H), 7.19-7.17 (m, 1H), 4.34 (bs, 2H), 3.78-3.75 (m, 2H), 3.65-3.56 (m, 11H), 3.35 (bs, 5H), 2.68 (bs, 2H), 2.14 (s, 3H), 1.48 (s, 9H). MS (m/z): 596.20[M+H]⁺.

Intermediate 19S: tert-butyl 4-(6-(8-chloronaphthalen-1-yl)-4-cyano-1-(2,6-dimethylmorpholino)-5,6,7,8-tetrahydro-2,6-naphthyridin-3-yl)piperazine-1-carboxylate: Was prepared using the process as described above for Intermediate 19K using Intermediate 15 (0.6 g, 0.00131 mol), 1-Bromo-8-Chloro naphthalene (0.47 g, 0.0019 mol). Sodium tert.butoxide (0.44 gm, 0.0039 mol), r-BINAP (0.13 gm, 0.00013 mol) and Pd2(dba)3 (0.11 gm, 0.000013 mol) to get Intermediate 19S (0.4 gm). 618.29 [M+H]⁺.

Intermediate 19T: tert-butyl (S)-4-(6-(8-chloronaphthalen-1-yl)-4-cyano-3-((1-methylpyrrolidin-2-yl)methoxy)-5,6,7,8-tetrahydro-2,6-naphthyridin-1-yl)piperazine-1-carboxylate: Was prepared using the process as described above for Intermediate 19K using Intermediate 15 (0.6 g, 0.00131 mol), 1-Bromo-8-Chloro naphthalene (0.47 g, 0.0019 mol), Sodium tert.butoxide (0.44 g, 0.0039 mol), r-BINAP (0.13 g, 0.00013 mol) and Pd2(dba)3 (0.11 gm, 0.000013 mol) to get Intermediate 19S (0.35 g), 618.29 [M+H]⁺.

Intermediate 19U: tert-butyl (S)-4-(4-cyano-6-(3-hydroxynaphthalen-1-yl)-3-((1-methyl pyrrolidin-2-yl)methoxy)-5,6,7,8-tetrahydro-2,6-naphthyridin-1-yl)piperazine-1-carboxylate: Was prepared using the process as described above for Intermediate 19K using Intermediate 17 (0.6 g, 0.00131 mol), 3 Hydroxy-1-Bromo naphthalene (0.43 g, 0.0019 mol), Sodium tert.butoxide (0.44 g, 0.0039 mol), r-BINAP (0.13 g, 0.00013 mol) and Pd2(dba)3 (0.11 gm, 0.000013 mol) to get Intermediate 191 (0.38 g), 599.33 [M+H]+.

Intermediate 19V: tert-butyl (S)-4-(4-cyano-3-((1-methylpyrrolidin-2-yl)methoxy)-6-(quinazolin-4-yl)-5,6,7,8-tetrahydro-2,6-naphthyridin-1-yl)piperazine-1-carboxylate: Was prepared using the process as described above for Intermediate 19K using Intermediate 17 (0.6 g, 0.0013 mol), 4-bromoquinazoline (0.41 g, 0.0019 mol), Sodium tert.butoxide (0.44 gm, 0.0039 mol), r-BINAP (0.13 gm, 0.00019 mol) and Pd2(dba)3 (0.11 gm, 0.000013 mol) to get Intermediate 19V as off white solid (0.28 gm). 585.33 [M+H]⁺.

Intermediate 19W: tert-butyl (S)-4-(4-cyano-3-((1-methylpyrrolidin-2-yl)methoxy)-6-(quinolin-4-yl)-5,6,7,8-tetrahydro-2,6-naphthyridin-1-yl)piperazine-1-carboxylate: Was prepared using the process as described above for Intermediate 19K using Intermediate 17 (0.6 g, 0.0013 mol), 4-bromoquinoline (0.4 g, 0.0019 mol), Sodium tert.butoxide (0.44 gm, 0.0039 mol), r-BINAP (0.13 gm, 0.00013 mol) and Pd2(dba)3 (0.11 gm, 0.000013 mol) to get Intermediate 19W as off white solid (0.39 g), 584.50 [M+H]⁺.

Intermediate 19X: tert-butyl (S)-4-(4-cyano-6-(isoquinolin-1-yl)-3-((1-methylpyrrolidin-2-yl)methoxy)-5,6,7,8-tetrahydro-2,6-naphthyridin-1-yl)piperazine-1-carboxylate: Was prepared using the process as described above for Intermediate 19K using Intermediate 17 (0.6 g, 0.0013 mol), 1-bromoisoquinoline (0.4 g, 0.0019 mol), Sodium tert.butoxide (0.44 gm, 0.0039 mol), r-BINAP (0.13 gm, 0.00013 mol) and Pd2(dba)3 (0.11 gm, 0.000013 mol) to get Intermediate 19W as off white solid (0.39 g), 584.30 [M+H]⁺.

Intermediate 19Y: tert-butyl (S)-4-(4-cyano-3-((1-methylpyrrolidin-2-yl)methoxy)-6-(naphthalen-1-yl)-5,6,7,8-tetrahydro-2,6-naphthyridin-1-yl)piperazine-1-carboxylate: Was prepared using the process as described above for Intermediate 19K using Intermediate 17 (0.6 g, 0.0013 mol), 1-bromo naphthalene (0.4 g, 0.0019 mol), Sodium tert.butoxide (0.44 gm, 0.0039 mol), r-BINAP (0.13 gm, 0.00013 mol) and Pd2(dba)3 (0.11 gm, 0.000013 mol) to get Intermediate 19Y as off % white solid (0.41 gm). 583.20 [M+H]+.

Intermediate 19Z: tert-butyl 4-(4-cyano-3-(((S)-1-methylpyrrolidin-2-yl)methoxy)-6-(naphthalen-1-yl)-5,6,7,8-tetrahydro-2,6-naphthyridin-1-yl)-2-(cyanomethyl)piperazine-1-carboxylate: Was prepared using the process as described above for Intermediate 19K using Intermediate 18 (0.6 g, 0.0012 mol), 1-bromo naphthalene (0.37 g, 0.00018 mol), Sodium tert.butoxide (0.4 gm, 0.0036 mol), r-BINAP (0.075 g, 0.00012 mmol) and Pd2(dba)3 (0.11 gm, 0.000012 mol) to get Intermediate 19Z (0.36 gm). 622.35 [M+H]⁺.

Intermediate 20: tert-butyl 4-(4-cyano-3-morpholino-6-(naphthalen-1-yl)-5,6,7,8-tetrahydro-2,6-naphthyridin-1-yl)piperazine-1-carboxylate: Was prepared using the process as described above for Intermediate 19K using Intermediate 11 (0.5 g, 0.001 mol), 1-bromo naphthalene (0.36 g, 0.0017 mol), Sodium tert.butoxide (0.39 g, 0.0035 mol), r-BINAP (0.072 g, 0.0001 mol) and Pd2(dba)3 (0.11 gm, 0.00001 mol) to get Intermediate 20 (˜0.4 gm). 555.28 [M+H]⁺.

Intermediate 20A: tert-butyl 4-(4-cyano-6-(naphthalen-1-yl)-5,6,7,8-tetrahydro-2,6-naphthyridin-1-yl)piperazine-1-carboxylate: Was prepared using the process as described above for Intermediate 19K using Intermediate 18H (0.5 g, 0.0014 mol), 1-bromo naphthalene (0.45 g, 0.0021 mol), Sodium tert.butoxide (0.48 gm, 0.0043 mol), r-BINAP (0.09 g, 0.00014 mol) and Pd2(dba)3 (0.13 gm, 0.000014 mol) to get Intermediate 20A (0.38 gm). 470.25 [M+H]⁺.

### General Procedure for Deprotection (Method 20)

In Single neck RBF, a desired solution of Intermediate 19A to 19Z, 20 and 20A (qty as aforementioned mentioned above) in DCM was added Trifluroacetic acid (TFA) or dioxane in HCl at 0° C. for 1-2 hr. The reaction completion was checked by TLC using Methanol: DCM (5%) and 1 drop of methanolic ammonia. The solvent was evaporated under reduced pressure to get crude solid of deprotected compounds, which were used for next step without any purification or characterisation.

TABLE-3
Int 1

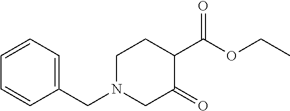 [SMILES: CCOC(=O)C1CCN(Cc2ccccc2)CC1=O]

Int 2

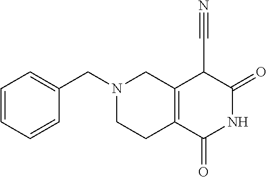 [SMILES: N#CC1C(=O)NC(=O)C2=C1CN(Cc1ccccc1)CC2]

Int 3

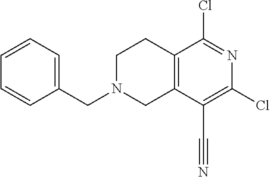 [SMILES: N#Cc1c(Cl)nc(Cl)c2c1CN(Cc1ccccc1)CC2]

Int 4A

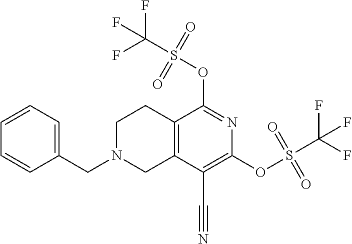 [SMILES: N#Cc1c(OS(=O)(=O)C(F)(F)F)nc(OS(=O)(=O)C(F)(F)F)c2c1CN(Cc1ccccc1)CC2]

Int 5

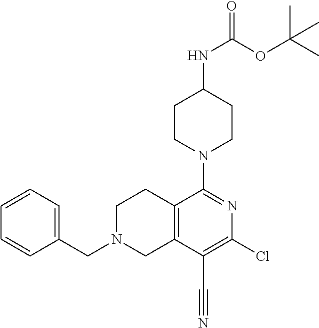 [SMILES: CC(C)(C)OC(=O)NC1CCN(c2nc(Cl)c(C#N)c3c2CCN(Cc2ccccc2)C3)CC1]

Int 5A

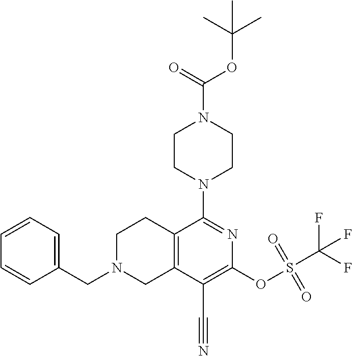 [SMILES: CC(C)(C)OC(=O)N1CCN(c2nc(OS(=O)(=O)C(F)(F)F)c(C#N)c3c2CCN(Cc2ccccc2)C3)CC1]

Int 5B

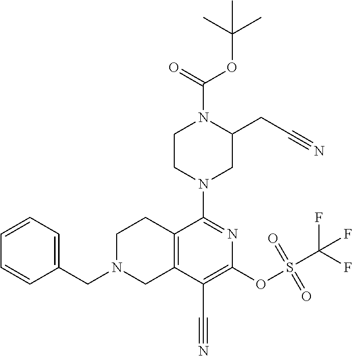 [SMILES: CC(C)(C)OC(=O)N1CCN(c2nc(OS(=O)(=O)C(F)(F)F)c(C#N)c3c2CCN(Cc2ccccc2)C3)CC1CC#N]

Int 5C

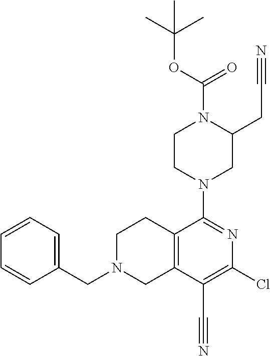 [SMILES: CC(C)(C)OC(=O)N1CCN(c2nc(Cl)c(C#N)c3c2CCN(Cc2ccccc2)C3)CC1CC#N]

Int 5D

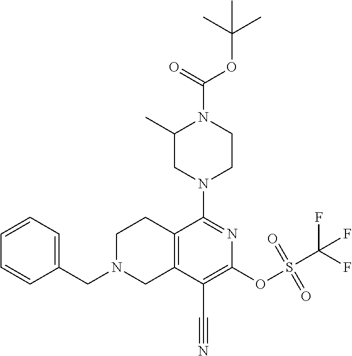 [SMILES: CC1CN(c2nc(OS(=O)(=O)C(F)(F)F)c(C#N)c3c2CCN(Cc2ccccc2)C3)CCN1C(=O)OC(C)(C)C]

Int 5E

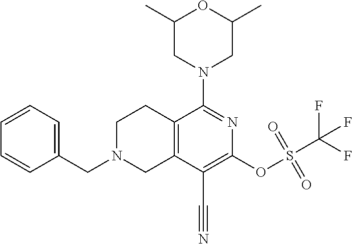 [SMILES: CC1CN(c2nc(OS(=O)(=O)C(F)(F)F)c(C#N)c3c2CCN(Cc2ccccc2)C3)CC(C)O1]

5F

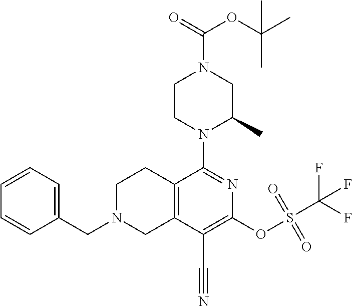 [SMILES: C[C@@H]1CN(C(=O)OC(C)(C)C)CCN1c1nc(OS(=O)(=O)C(F)(F)F)c(C#N)c2c1CCN(Cc1ccccc1)C2]

5G

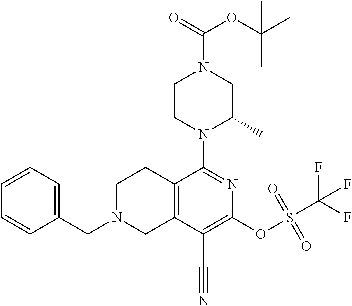 [SMILES: C[C@H]1CN(C(=O)OC(C)(C)C)CCN1c1nc(OS(=O)(=O)C(F)(F)F)c(C#N)c2c1CCN(Cc1ccccc1)C2]

5H

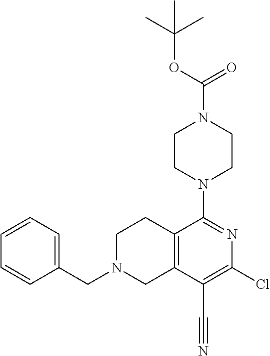 [SMILES: CC(C)(C)OC(=O)N1CCN(c2nc(Cl)c(C#N)c3c2CCN(Cc2ccccc2)C3)CC1]

Int 6

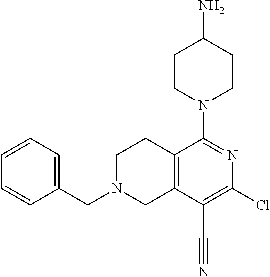 [SMILES: N#Cc1c(Cl)nc(N2CCC(N)CC2)c2c1CN(Cc1ccccc1)CC2]

Int 6A1

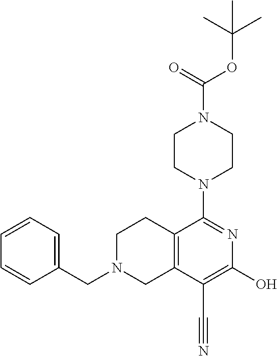 [SMILES: CC(C)(C)OC(=O)N1CCN(c2nc(O)c(C#N)c3c2CCN(Cc2ccccc2)C3)CC1]

Int 6A2

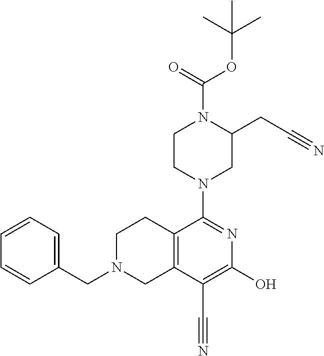 [SMILES: CC(C)(C)OC(=O)N1CCN(c2nc(O)c(C#N)c3c2CCN(Cc2ccccc2)C3)CC1CC#N]

Int 6B

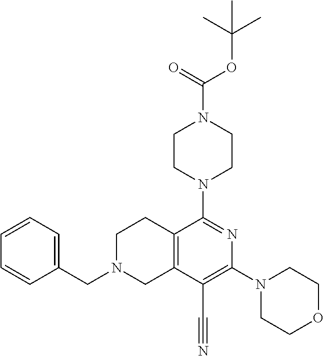 [SMILES: CC(C)(C)OC(=O)N1CCN(c2nc(N3CCOCC3)c(C#N)c3c2CCN(Cc2ccccc2)C3)CC1]

Int 6C

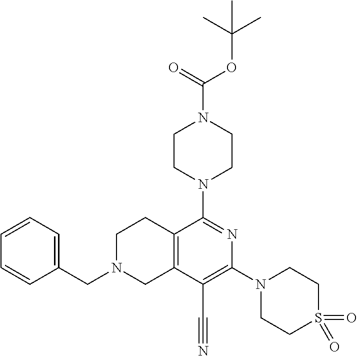 [SMILES: CC(C)(C)OC(=O)N1CCN(c2nc(N3CCS(=O)(=O)CC3)c(C#N)c3c2CCN(Cc2ccccc2)C3)CC1]

6D

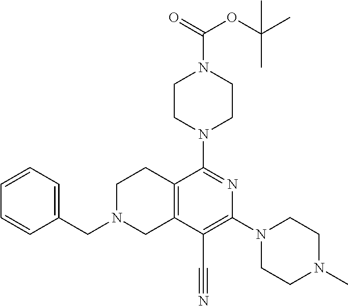 [SMILES: CN1CCN(c2nc(N3CCN(C(=O)OC(C)(C)C)CC3)c3c(c2C#N)CN(Cc2ccccc2)CC3)CC1]

6E

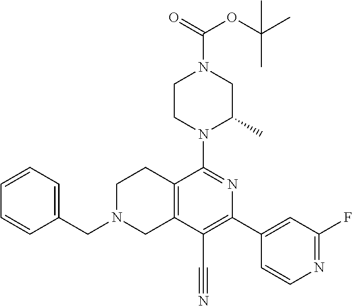 [SMILES: C[C@H]1CN(C(=O)OC(C)(C)C)CCN1c1nc(-c2ccnc(F)c2)c(C#N)c2c1CCN(Cc1ccccc1)C2]

6F

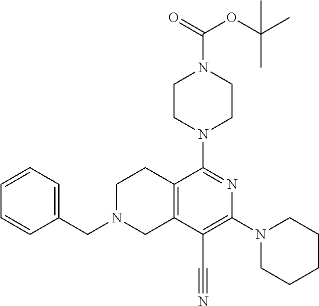 [SMILES: CC(C)(C)OC(=O)N1CCN(c2nc(N3CCCCC3)c(C#N)c3c2CCN(Cc2ccccc2)C3)CC1]

6G

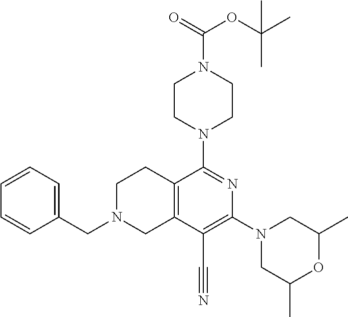 [SMILES: CC1CN(c2nc(N3CCN(C(=O)OC(C)(C)C)CC3)c3c(c2C#N)CN(Cc2ccccc2)CC3)CC(C)O1]

6H

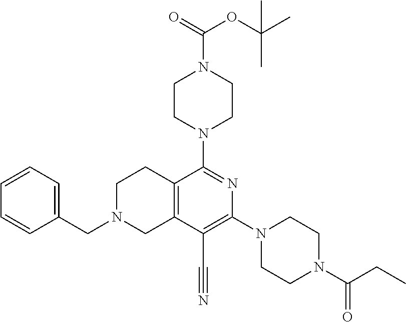 [SMILES: CCC(=O)N1CCN(c2nc(N3CCN(C(=O)OC(C)(C)C)CC3)c3c(c2C#N)CN(Cc2ccccc2)CC3)CC1]

6I

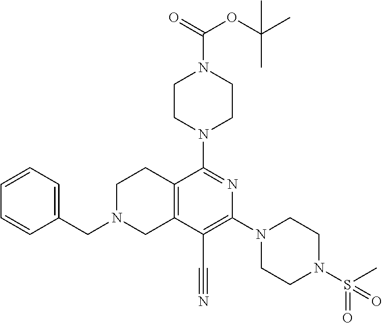 [SMILES: CC(C)(C)OC(=O)N1CCN(c2nc(N3CCN(S(C)(=O)=O)CC3)c(C#N)c3c2CCN(Cc2ccccc2)C3)CC1]

6J

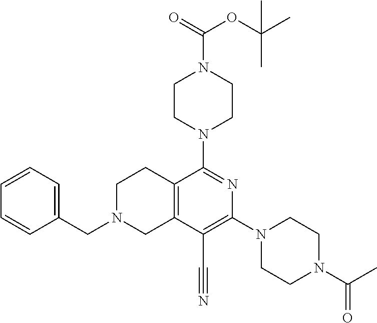 [SMILES: CC(=O)N1CCN(c2nc(N3CCN(C(=O)OC(C)(C)C)CC3)c3c(c2C#N)CN(Cc2ccccc2)CC3)CC1]

Int 7

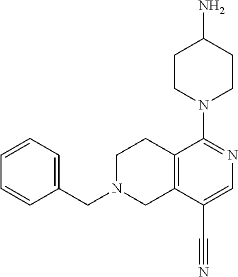 [SMILES: N#Cc1cnc(N2CCC(N)CC2)c2c1CN(Cc1ccccc1)CC2]

Int 7A

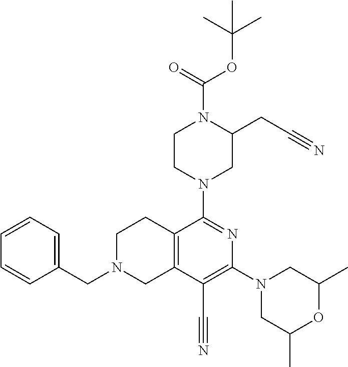 [SMILES: CC1CN(c2nc(N3CCN(C(=O)OC(C)(C)C)C(CC#N)C3)c3c(c2C#N)CN(Cc2ccccc2)CC3)CC(C)O1]

Int 7B

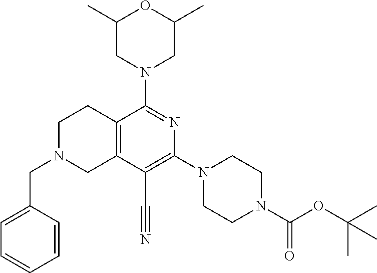 [SMILES: CC1CN(c2nc(N3CCN(C(=O)OC(C)(C)C)CC3)c(C#N)c3c2CCN(Cc2ccccc2)C3)CC(C)O1]

Int 7C

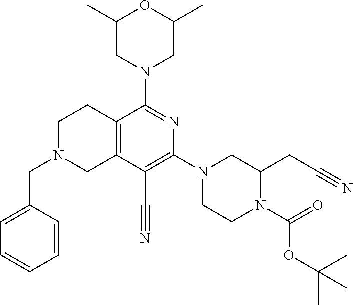 [SMILES: CC1CN(c2nc(N3CCN(C(=O)OC(C)(C)C)C(CC#N)C3)c(C#N)c3c2CCN(Cc2ccccc2)C3)CC(C)O1]

7D

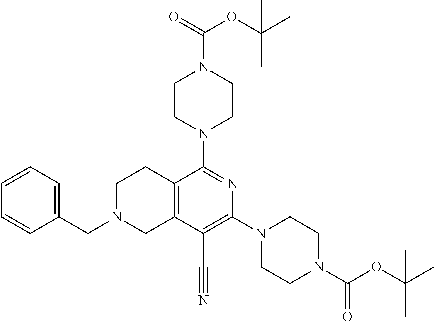 [SMILES: CC(C)(C)OC(=O)N1CCN(c2nc(N3CCN(C(=O)OC(C)(C)C)CC3)c3c(c2C#N)CN(Cc2ccccc2)CC3)CC1]

7E

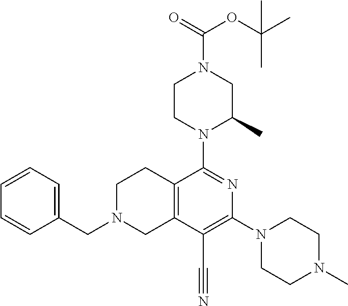 [SMILES: C[C@@H]1CN(C(=O)OC(C)(C)C)CCN1c1nc(N2CCN(C)CC2)c(C#N)c2c1CCN(Cc1ccccc1)C2]

7F

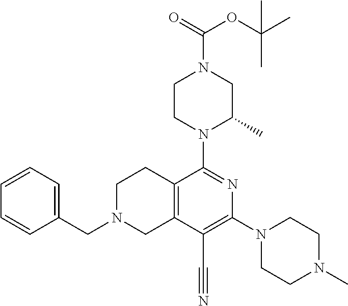 [SMILES: C[C@H]1CN(C(=O)OC(C)(C)C)CCN1c1nc(N2CCN(C)CC2)c(C#N)c2c1CCN(Cc1ccccc1)C2]

7G

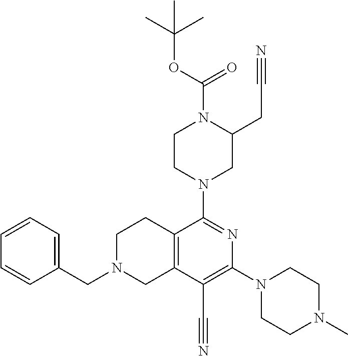 [SMILES: CN1CCN(c2nc(N3CCN(C(=O)OC(C)(C)C)C(CC#N)C3)c3c(c2C#N)CN(Cc2ccccc2)CC3)CC1]

Int 8

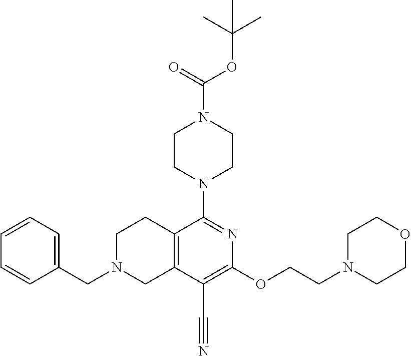 [SMILES: CC(C)(C)OC(=O)N1CCN(c2nc(OCCN3CCOCC3)c(C#N)c3c2CCN(Cc2ccccc2)C3)CC1]

Int 9A

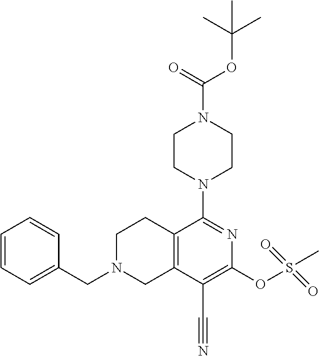 [SMILES: CC(C)(C)OC(=O)N1CCN(c2nc(OS(C)(=O)=O)c(C#N)c3c2CCN(Cc2ccccc2)C3)CC1]

Int 9B

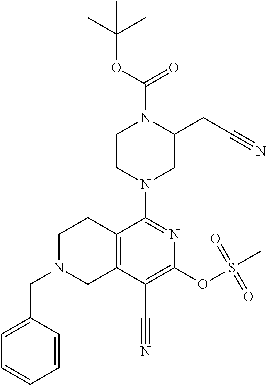 [SMILES: CC(C)(C)OC(=O)N1CCN(c2nc(OS(C)(=O)=O)c(C#N)c3c2CCN(Cc2ccccc2)C3)CC1CC#N]

Int 10A

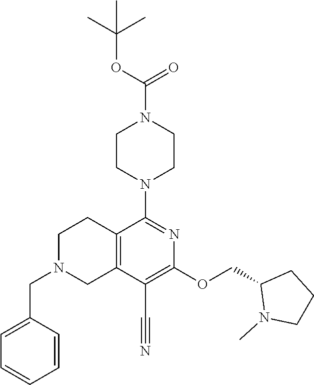 [SMILES: CN1CCC[C@H]1COc1nc(N2CCN(C(=O)OC(C)(C)C)CC2)c2c(c1C#N)CN(Cc1ccccc1)CC2]

Int 10B

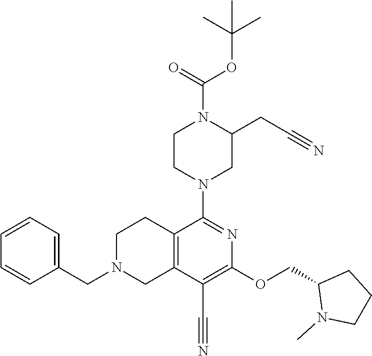 [SMILES: CN1CCC[C@H]1COc1nc(N2CCN(C(=O)OC(C)(C)C)C(CC#N)C2)c2c(c1C#N)CN(Cc1ccccc1)CC2]

Int 11

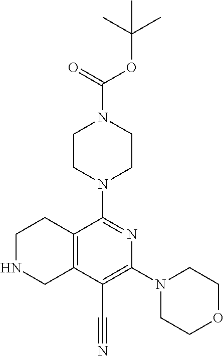 [SMILES: CC(C)(C)OC(=O)N1CCN(c2nc(N3CCOCC3)c(C#N)c3c2CCNC3)CC1]

Int 12

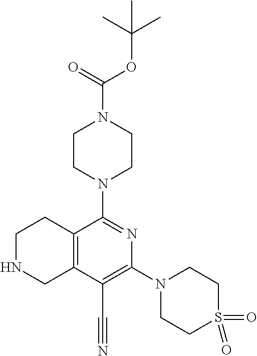 [SMILES: CC(C)(C)OC(=O)N1CCN(c2nc(N3CCS(=O)(=O)CC3)c(C#N)c3c2CCNC3)CC1]

Int 13/14

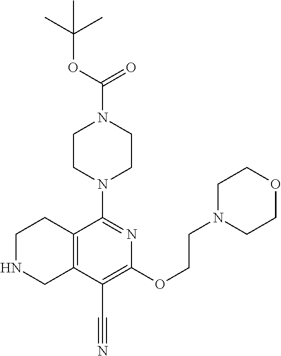 [SMILES: CC(C)(C)OC(=O)N1CCN(c2nc(OCCN3CCOCC3)c(C#N)c3c2CCNC3)CC1]

Int 14

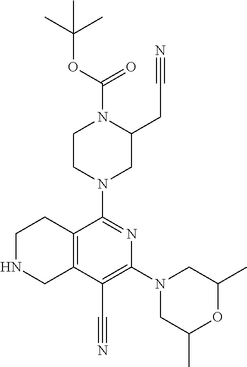 [SMILES: CC1CN(c2nc(N3CCN(C(=O)OC(C)(C)C)C(CC#N)C3)c3c(c2C#N)CNCC3)CC(C)O1]

Int 15

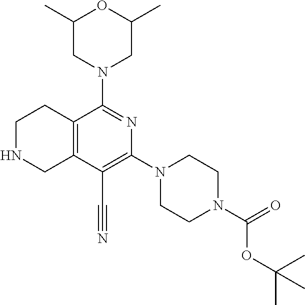 [SMILES: CC1CN(c2nc(N3CCN(C(=O)OC(C)(C)C)CC3)c(C#N)c3c2CCNC3)CC(C)O1]

Int 16

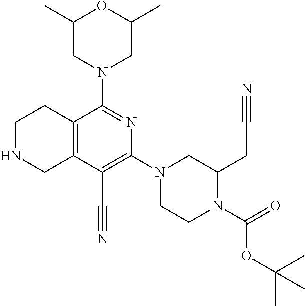 [SMILES: CC1CN(c2nc(N3CCN(C(=O)OC(C)(C)C)C(CC#N)C3)c(C#N)c3c2CCNC3)CC(C)O1]

Int 17

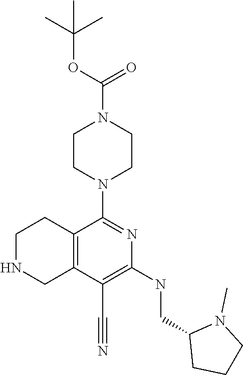 [SMILES: CN1CCC[C@@H]1CNc1nc(N2CCN(C(=O)OC(C)(C)C)CC2)c2c(c1C#N)CNCC2]

Int 18

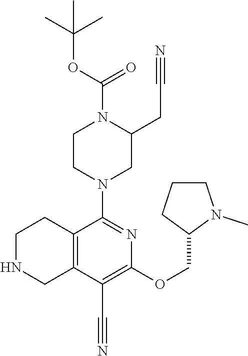

18A

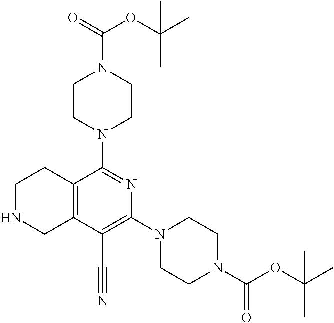 [SMILES: CC(C)(C)OC(=O)N1CCN(c2nc(N3CCN(C(=O)OC(C)(C)C)CC3)c3c(c2C#N)CNCC3)CC1]

18B

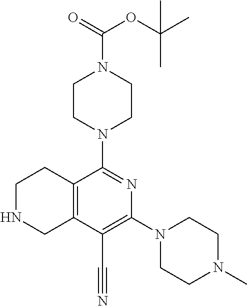 [SMILES: CN1CCN(c2nc(N3CCN(C(=O)OC(C)(C)C)CC3)c3c(c2C#N)CNCC3)CC1]

18C

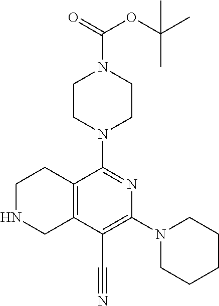 [SMILES: CC(C)(C)OC(=O)N1CCN(c2nc(N3CCCCC3)c(C#N)c3c2CCNC3)CC1]

18D

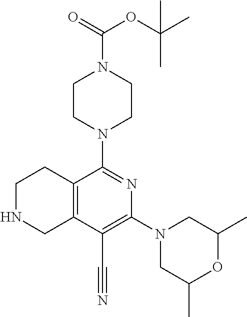 [SMILES: CC1CN(c2nc(N3CCN(C(=O)OC(C)(C)C)CC3)c3c(c2C#N)CNCC3)CC(C)O1]

18E

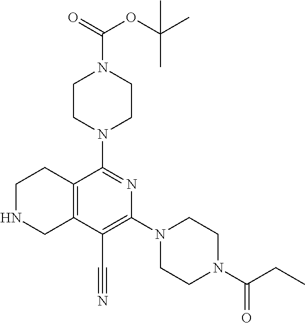 [SMILES: CCC(=O)N1CCN(c2nc(N3CCN(C(=O)OC(C)(C)C)CC3)c3c(c2C#N)CNCC3)CC1]

18F

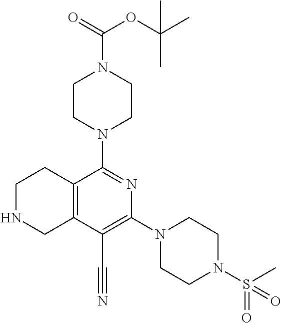 [SMILES: CC(C)(C)OC(=O)N1CCN(c2nc(N3CCN(S(C)(=O)=O)CC3)c(C#N)c3c2CCNC3)CC1]

18G

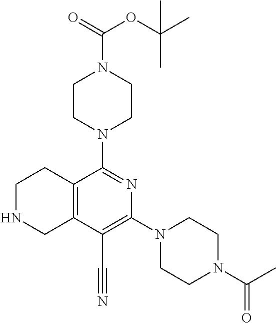 [SMILES: CC(=O)N1CCN(c2nc(N3CCN(C(=O)OC(C)(C)C)CC3)c3c(c2C#N)CNCC3)CC1]

Int 19A

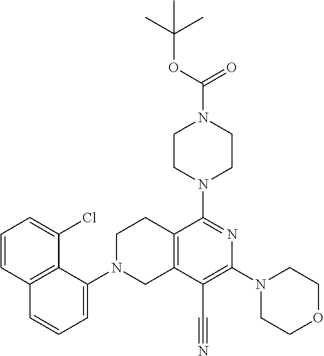 [SMILES: CC(C)(C)OC(=O)N1CCN(c2nc(N3CCOCC3)c(C#N)c3c2CCN(c2cccc4cccc(Cl)c24)C3)CC1]

18H

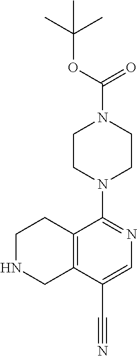 [SMILES: CC(C)(C)OC(=O)N1CCN(c2ncc(C#N)c3c2CCNC3)CC1]

Int 19B

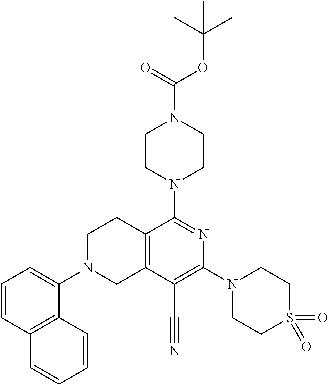 [SMILES: CC(C)(C)OC(=O)N1CCN(c2nc(N3CCS(=O)(=O)CC3)c(C#N)c3c2CCN(c2cccc4ccccc24)C3)CC1]

Int 19 C

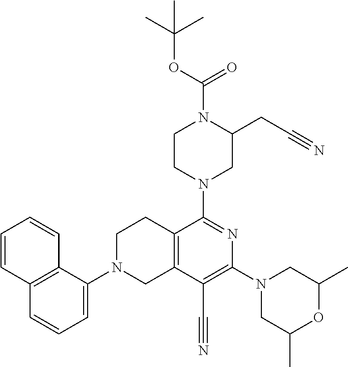 [SMILES: CC1CN(c2nc(N3CCN(C(=O)OC(C)(C)C)C(CC#N)C3)c3c(c2C#N)CN(c2cccc4ccccc24)CC3)CC(C)O1]

Int 19D

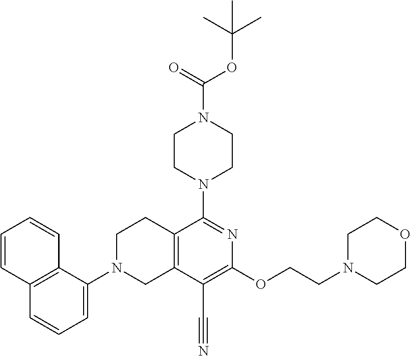 [SMILES: CC(C)(C)OC(=O)N1CCN(c2nc(OCCN3CCOCC3)c(C#N)c3c2CCN(c2cccc4ccccc24)C3)CC1]

Int 19E

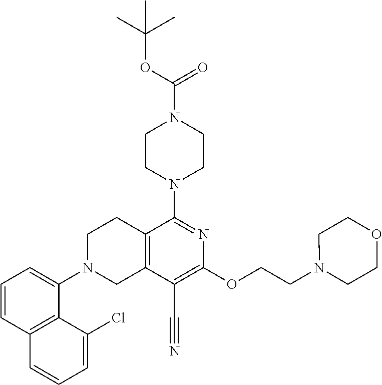 [SMILES: CC(C)(C)OC(=O)N1CCN(c2nc(OCCN3CCOCC3)c(C#N)c3c2CCN(c2cccc4cccc(Cl)c24)C3)CC1]

Int 19F

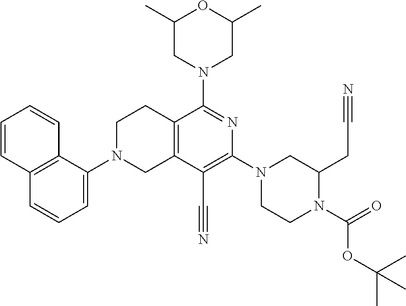 [SMILES: CC1CN(c2nc(N3CCN(C(=O)OC(C)(C)C)C(CC#N)C3)c(C#N)c3c2CCN(c2cccc4ccccc24)C3)CC(C)O1]

Int 19G

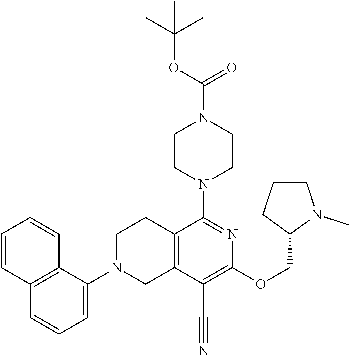 [SMILES: CN1CCC[C@H]1COc1nc(N2CCN(C(=O)OC(C)(C)C)CC2)c2c(c1C#N)CN(c1cccc3ccccc13)CC2]

Int 19H

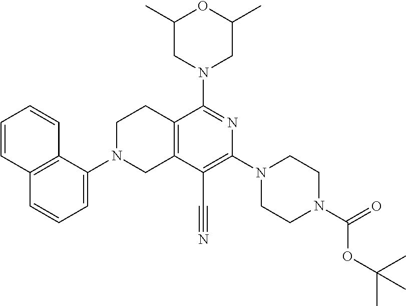 [SMILES: CC1CN(c2nc(N3CCN(C(=O)OC(C)(C)C)CC3)c(C#N)c3c2CCN(c2cccc4ccccc24)C3)CC(C)O1]

Int 19I

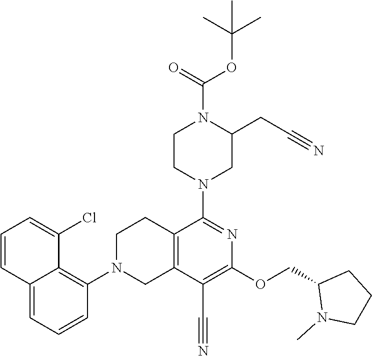 [SMILES: CN1CCC[C@H]1COc1nc(N2CCN(C(=O)OC(C)(C)C)C(CC#N)C2)c2c(c1C#N)CN(c1cccc3cccc(Cl)c13)CC2]

Int 19J

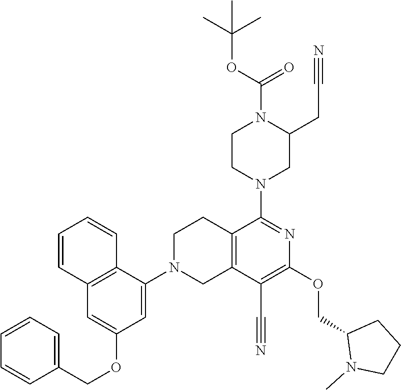 [SMILES: CN1CCC[C@H]1COc1nc(N2CCN(C(=O)OC(C)(C)C)C(CC#N)C2)c2c(c1C#N)CN(c1cc(OCc3ccccc3)cc3ccccc13)CC2]

19K

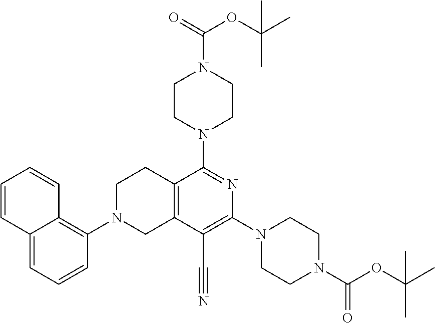 [SMILES: CC(C)(C)OC(=O)N1CCN(c2nc(N3CCN(C(=O)OC(C)(C)C)CC3)c3c(c2C#N)CN(c2cccc4ccccc24)CC3)CC1]

19L

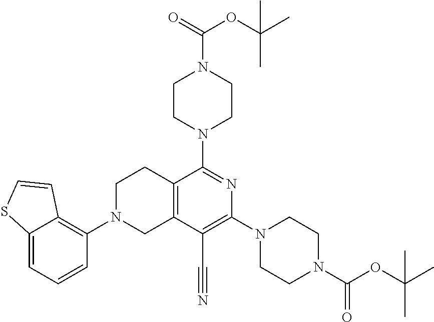 [SMILES: CC(C)(C)OC(=O)N1CCN(c2nc(N3CCN(C(=O)OC(C)(C)C)CC3)c3c(c2C#N)CN(c2cccc4sccc24)CC3)CC1]

19M

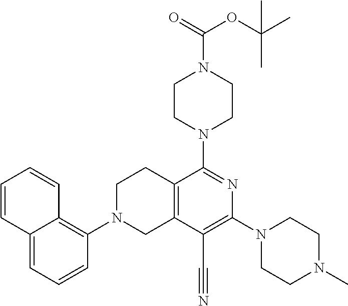 [SMILES: CN1CCN(c2nc(N3CCN(C(=O)OC(C)(C)C)CC3)c3c(c2C#N)CN(c2cccc4ccccc24)CC3)CC1]

19N

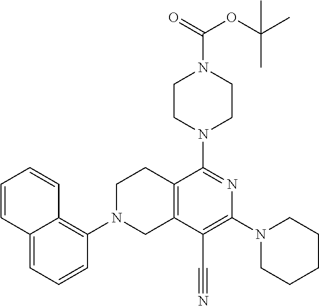 [SMILES: CC(C)(C)OC(=O)N1CCN(c2nc(N3CCCCC3)c(C#N)c3c2CCN(c2cccc4ccccc24)C3)CC1]

19O

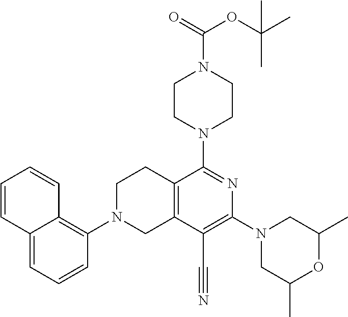 [SMILES: CC1CN(c2nc(N3CCN(C(=O)OC(C)(C)C)CC3)c3c(c2C#N)CN(c2cccc4ccccc24)CC3)CC(C)O1]

19P

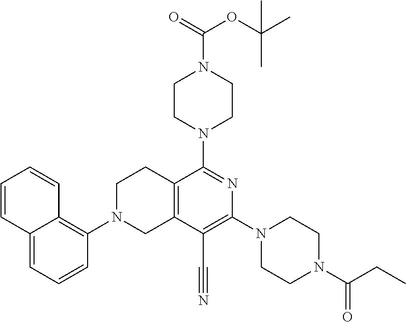 [SMILES: CCC(=O)N1CCN(c2nc(N3CCN(C(=O)OC(C)(C)C)CC3)c3c(c2C#N)CN(c2cccc4ccccc24)CC3)CC1]

19Q

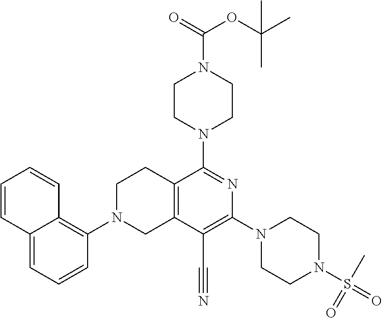 [SMILES: CC(C)(C)OC(=O)N1CCN(c2nc(N3CCN(S(C)(=O)=O)CC3)c(C#N)c3c2CCN(c2cccc4ccccc24)C3)CC1]

19R

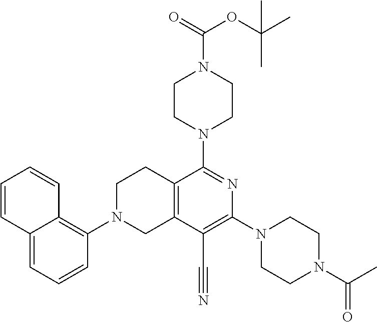 [SMILES: CC(=O)N1CCN(c2nc(N3CCN(C(=O)OC(C)(C)C)CC3)c3c(c2C#N)CN(c2cccc4ccccc24)CC3)CC1]

19S

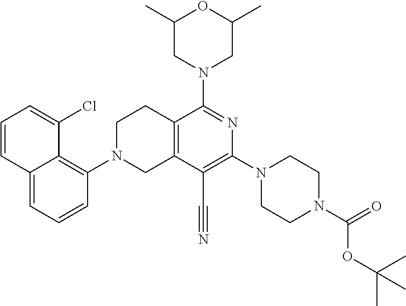 [SMILES: CC1CN(c2nc(N3CCN(C(=O)OC(C)(C)C)CC3)c(C#N)c3c2CCN(c2cccc4cccc(Cl)c24)C3)CC(C)O1]

19T

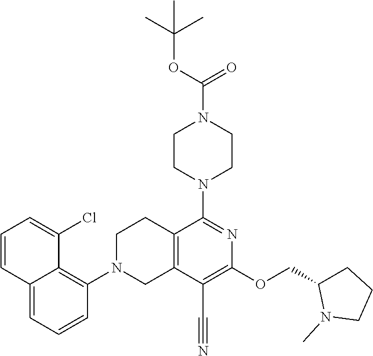 [SMILES: CN1CCC[C@H]1COc1nc(N2CCN(C(=O)OC(C)(C)C)CC2)c2c(c1C#N)CN(c1cccc3cccc(Cl)c13)CC2]

19U

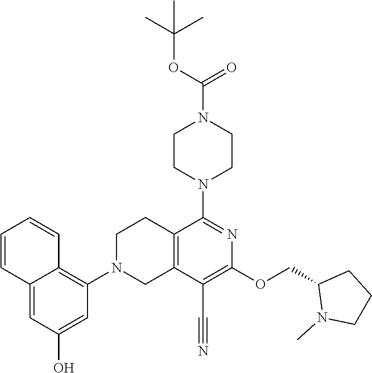 [SMILES: CN1CCC[C@H]1COc1nc(N2CCN(C(=O)OC(C)(C)C)CC2)c2c(c1C#N)CN(c1cc(O)cc3ccccc13)CC2]

19V

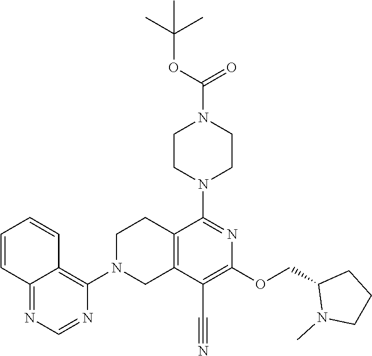 [SMILES: CN1CCC[C@H]1COc1nc(N2CCN(C(=O)OC(C)(C)C)CC2)c2c(c1C#N)CN(c1ncnc3ccccc13)CC2]

19W

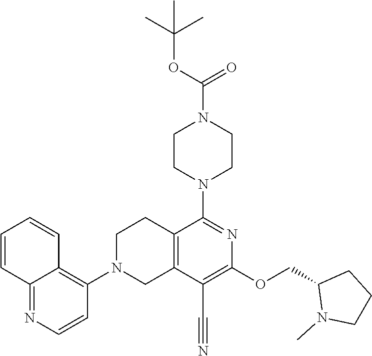 [SMILES: CN1CCC[C@H]1COc1nc(N2CCN(C(=O)OC(C)(C)C)CC2)c2c(c1C#N)CN(c1ccnc3ccccc13)CC2]

19X

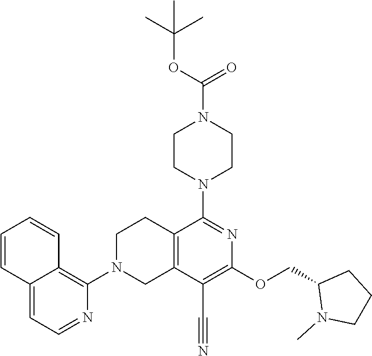 [SMILES: CN1CCC[C@H]1COc1nc(N2CCN(C(=O)OC(C)(C)C)CC2)c2c(c1C#N)CN(c1nccc3ccccc13)CC2]

19Y

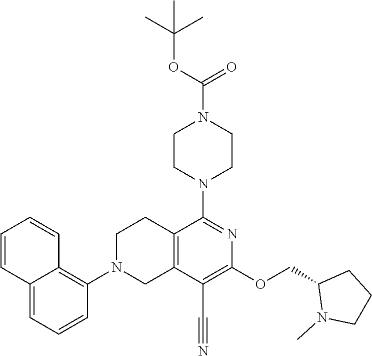 [SMILES: CN1CCC[C@H]1COc1nc(N2CCN(C(=O)OC(C)(C)C)CC2)c2c(c1C#N)CN(c1cccc3ccccc13)CC2]

19Z

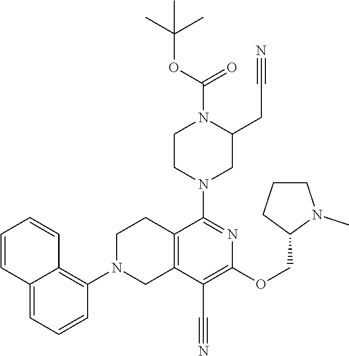 [SMILES: CN1CCC[C@H]1COc1nc(N2CCN(C(=O)OC(C)(C)C)C(CC#N)C2)c2c(c1C#N)CN(c1cccc3ccccc13)CC2]

20

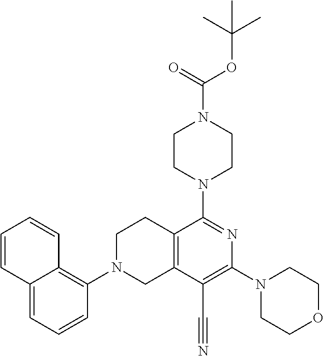 [SMILES: CC(C)(C)OC(=O)N1CCN(c2nc(N3CCOCC3)c(C#N)c3c2CCN(c2cccc4ccccc24)C3)CC1]

20A

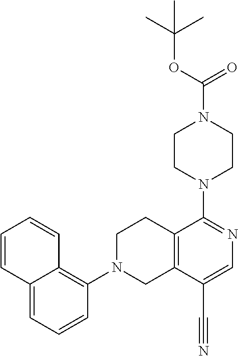 [SMILES: CC(C)(C)OC(=O)N1CCN(c2ncc(C#N)c3c2CCN(c2cccc4ccccc24)C3)CC1]

### Part-B—Preparation of Compounds

Example 1: 1-(4-acryloylpiperazin-1-yl)-3-(1,1-dioxidothiomorpholino)-6-(naphthalen-1-yl)-5,6,7,8-tetrahydro-2,6-naphthyridine-4-carbonitrile: In single Neck 100 mL RBF. deprotected intermediate from Method 20 (0.2 gm, 0.39 mmol) in DCM (5 mL) was added DIEPA (0.21 mL, 1.17 mmol) at 0° C. and stirred for 10 minutes. The acryloyl chloride (0.06 mL, 0.79 mmol) in DCM (3 ml) was added slowly to the above solution at 0° C. The reaction mixture was stirred at 0° C. for 40 minutes. The reaction completion was checked by TLC using MeOH: DCM (5%). The reaction mixture was quenched with saturated solution of NaHCO₃ (10 mL) and stirred the entire solution for 10 minutes then isolated the organic layer. The aqueous layer again extracted with DCM (10 mL×2) and the combined organic layers were washed with brine solution (10 mL), dried over Na2SO4 and concentrated under reduced pressure to get a crude residue which was purified by column chromatography using 2-3% MeOH in DCM as mobile phase. The pure fractions were collected and concentrated to get desired product (0.065 gm, 29.94%). 1H-NMR (δ ppm, CDCl₃, 400 MHz): δ 7.25 (m, 5H), 6.59 (dd, 1H), 6.34 (dd, 1H), 5.75 (dd, 1H), 4.39 (s, 2H), 3.82 (s, 3H), 3.74 (m, 7H), 3.24 (m, 4H), 3.08 (t, 2H), 2.54 (t, 4H), 2.35 (d, 3H), 1.25 (s, 1H), 1.19 (s, 1H). MS (m/z): 557.2 [M+H]. HPLC purity: 95.88%.

Example 2: 1-(4-acryloylpiperazin-1-yl)-6-(8-chloronaphthalen-1-yl)-3-morpholino-5,6,7,8-tetrahydro-2,6-naphthyridine-4-carbonitrile: Was prepared using the process as described above for Example 1 using Intermediate 19A to get 0.06 gm of 1-(4-acryloylpiperazin-1-yl)-6-(8-chloronaphthalen-1-yl)-3-morpholino-5,6,7,8-tetrahydro-2,6-naphthyridine-4-carbonitrile, 1H-NMR (δ ppm, CDCl₃, 400 MHz): δ 7.36-7.33 (m, 4H), 7.31-7.28 (m, 1H), 6.62-6.55 (m, 1H), 6.35-6.31 (m, 1H), 5.76-5.73 (m, 1H), 4.33-4.23 (m, 2H), 3.79-3.77 (bs, 4H), 3.70-3.66 (bs, 4H), 3.36 (bs, 4H), 3.12-3.08 (m, 1H), 2.72-2.68 (m, 1H), 2.61-2.59 (bs, 4H), 2.51 (bs, 3H), 2.34-2.30 (m, 1H), 2.06-2.01 (m, 1H), 1.83-1.67 (m, 3H). MS (n/z): 543.2 [M+H]⁺. HPLC purity: 96.69%.

Example 3: 1-(4-acryloylpiperazin-1-yl)-3-(2-morpholinoethoxy)-6-(naphthalen-1-yl)-5,6,7,8-tetrahydro-2,6-naphthyridine-4-carbonitrile: Was prepared using the process as described above for Example 1 using Intermediate 19D to get 0.17 gm of 1-(4-acryloylpiperazin-1-yl)-3-(2-morpholinoethoxy)-6-(naphthalen-1-yl)-5,6,7,8-tetrahydro-2,6-naphthyridine-4-carbonitrile, 1H-NMR (δ ppm, CDCl₃, 400 MHz): δ 7.36-7.33 (m, 4H), 7.31-7.28 (m, 1H), 6.62-6.55 (m, 1H), 6.35-6.31 (m, 1H), 5.76-5.73 (m, 1H), 4.33-4.23 (m, 2H), 3.79-3.77 (bs, 4H), 3.70-3.66 (bs, 4H), 3.36 (bs, 4H), 3.12-3.08 (m, 1H), 2.72-2.68 (m, 1H), 2.61-2.59 (bs, 4H), 2.51 (bs, 3H), 2.34-2.30 (m, 1H), 2.06-2.01 (m, 1H), 1.83-1.67 (m, 3H). MS (m/z): 553.3 [M+H]+. HPLC purity: 96.11%.

Example 4: 1-(4-acryloylpiperazin-1-yl)-6-(8-chloronaphthalen-1-yl)-3-(2-orpholinoethoxy)-5,6,7,8-tetrahydro-2,6-naphthyridine-4-carbonitrile: Was prepared using the process as described above for Example 1 using Intermediate 19E to get 0.1 gm of 1-(4-acryloylpiperazin-1-yl)-6-(8-chloronaphthalen-1-yl)-3-(2-morpholinoethoxy)-5,6,7,8-tetrahydro-2,6-naphthyridine-4-carbonitrile, 1H-NMR (δ ppm, CDCl₃, 400 MHz): δ 7.36-7.33 (m, 4H), 7.31-7.28 (m, 1H), 6.62-6.55 (m, 1H), 6.35-6.31 (m, 1H), 5.76-5.73 (m, 1H), 4.33-4.23 (m, 2H), 3.79-3.77 (bs, 4H), 3.70-3.66 (bs, 4H), 3.36 (bs. 4H), 3.12-3.08 (m, 1H), 2.72-2.68 (m, 1H), 2.61-2.59 (bs, 4H), 2.51 (bs, 3H), 2.34-2.30 (m, 1H), 2.06-2.01 (m, 1H), 1.83-1.67 (m, 3H). MS (m/z): 587.3 [M+H]⁺. HPLC purity: 96.11%.

Example 5: 3-(4-acryloylpiperazin-1-yl)-1-(2,6-dimethylmorpholino)-6-(naphthalen-1-yl)-5,6,7,8-tetrahydro-2,6-naphthyridine-4-carbonitrile: Was prepared using the process as described above for Example 1 using Intermediate 19H to get 0.12 gm of 3-(4-acryloylpiperazin-1-yl)-1-(2,6-dimethylmorpholino)-6-(naphthalen-1-yl)-5,6,7,8-tetrahydro-2,6-naphthyridine-4-carbonitrile, 1H-NMR (δ ppm, CDCl₃, 400 MHz): δ 7.36-7.33 (m, 4H), 7.31-7.28 (m, 1H), 6.62-6.55 (m, 1H), 6.35-6.31 (m, 1H), 5.76-5.73 (m, 1H), 4.33-4.23 (m, 2H), 3.79-3.77 (bs, 4H), 3.70-3.66 (bs, 4H), 3.36 (bs, 4H), 3.12-3.08 (m, 1H), 2.72-2.68 (m, 1H), 2.61-2.59 (bs, 4H), 2.51 (bs, 3H), 2.34-2.30 (m, 1H), 2.06-2.01 (m, 1H), 1.83-1.67 (m, 3H). MS (m/z): 537.3 [M+H]⁺. HPLC purity: 98.23%.

Example 6: 3-(4-acryloylpiperazin-1-yl)-6-(8-chloronaphthalen-1-yl)-1-(2,6-dimethylmorpholino)-5,6,7,8-tetrahydro-2,6-naphthyridine-4-carbonitrile: Was prepared using the process as described above for Example 1 to get 0.1 gm of 3-(4-acryloylpiperazin-1-yl)-6-(8-chloronaphthalen-1-yl)-1-(2,6-dimethylmorpholino)-5,6,7,8-tetrahydro-2,6-naphthyridine-4-carbonitrile, 1H-NMR (δ ppm, CDCl₃, 400 MHz): δ 7.36-7.33 (m, 4H), 7.31-7.28 (m, 1H), 6.62-6.55 (m, 1H), 6.35-6.31 (m, 1H), 5.76-5.73 (m, 1H), 4.33-4.23 (m, 2H), 3.79-3.77 (bs, 4H), 3.70-3.66 (bs, 4H), 3.36 (bs, 4H), 3.12-3.08 (m, 1H), 2.72-2.68 (m, 1H), 2.61-2.59 (bs, 4H), 2.51 (bs, 3H), 2.34-2.30 (m, 1H), 2.06-2.01 (m, 1H), 1.83-1.67 (m, 3H). MS (m/z): 571.3 [M+H]+. HPLC purity: 98.08%.

Example 7: 3-(4-acryloyl-3-(cyanomethyl)piperazin-1-yl)-1-(2,6-dimethylmorpholino)-6-(naphthalen-1-yl)-5,6,7,8-tetrahydro-2,6-naphthyridine-4-carbonitrile: Was prepared using the process as described above for Example 1 using Intermediate 19F to get desired product (0.04 gm, 17.03%). 1H-NMR (δ ppm, CDCl₃, 400 MHz): δ 7.25 (m, 5H), 6.59 (dd, 1H), 6.34 (dd, 1H), 5.75 (dd, 1H), 4.39 (s, 2H), 3.82 (s, 3H), 3.74 (m, 7H), 3.24 (m, 4H), 3.08 (t, 2H), 2.54 (1, 4H), 2.35 (d, 3H), 1.25 (s, 1H), 1.19 (s, 1H). MS (m/z): 576.2 [M+H]⁺. HPLC purity: 95.4%.

Example 8: (S)-1-(4-acryloylpiperazin-1-yl)-6-(8-chloronaphthalen-1-yl)-3-((1-methylpyrrolidin-2-yl)methoxy)-5,6,7,8-tetrahydro-2,6-naphthyridine-4-carbonitrile: Was prepared using the process as described above for Example 1 to get to afford (S)-1-(4-acryloylpiperazin-1-yl)-6-(8-chloronaphthalen-1-yl)-3-((1-methylpyrrolidin-2-yl)methoxy)-5,6,7,8-tetrahydro-2,6-naphthyridine-4-carbonitrile (0.05 gm, 12%) as off white solid. 1H-NMR (400 MHz) (CDCl3): 7.77 (d, J=6.8 Hz, 1H), 7.67-7.65 (m, 1H), 7.54-7.52 (m, 1H), 7.36-7.35 (m, 1H), 7.34-7.28 (m, 1H), 6.64-6.58 (m, 1H), 6.37-6.32 (m, 1H), 5.85-5.71 (m, 1H), 4.94-4.92 (m, 1H), 4.59-4.51 (m, 2H), 3.94-3.80 (m, 2H), 3.81-3.65 (m, 2H), 3.64-3.61 (m, 2H), 3.60-3.54 (m, 2H), 3.34-3.28 (m, 2H), 3.16-3.10 (m, 2H), 2.92 (s, 3H), 2.62-2.59 (m, 2H), 2.28-2.24 (m, 2H), 2.06-2.01 (m, 2H), 1.41-1.37 (m, 2H). MS (m/z): 571.2 (M+1). HPLC purity: 94.39%.

Example 9: (S)-1-(4-acryloylpiperazin-1-yl)-6-(3-hydroxynaphthalen-1-yl)-3-((1-methylpyrrolidin-2-yl)methoxy)-5,6,7,8-tetrahydro-2,6-naphthyridine-4-carbonitrile: Was prepared using the process as described above for Example 1 to get the desired product of (S)-1-(4-acryloylpiperazin-1-yl)-6-(3-hydroxynaphthalen-1-yl)-3-((1-methylpyrrolidin-2-yl)methoxy)-5,6,7,8-tetrahydro-2,6-naphthyridine-4-carbonitrile (0.035 gm) as white solid. 1H-NMR (400 MHz) (CDCl3): 7.98 (d, J=6.4 Hz, 1H), 7.65 (d, J=6.8 Hz, 1H), 7.42-7.40 (m, 1H), 7.32-7.29 (m, 1H), 6.90 (s, 1H), 6.74 (s, 1H), 6.59-6.53 (m, 1H), 6.35-6.32 (d, J=14 Hz, 1H), 5.75 (d, J=8.4 Hz, 1H), 4.48-4.44 (m, 1H), 4.33.4.30 (m, 2H), 4.21-4.18 (m, 1H), 3.78-3.61 (m, 4H), 3.36-3.33 (m, 5H), 3.18-3.15 (m, 1H), 2.83-2.79 (m, 2H), 2.62 (s, 3H), 2.10-2.06 (m, 2H), 1.82-1.81 (m, 1H), 1.78-1.74 (m, 2H), 1.44-1.41 (m, 2H). MS (m/z): 553.5 (M+1). HPLC purity: 94.12%.

Example 10: (S)-1-(4-(2-fluoroacryloyl)piperazin-1-yl)-3-((1-methylpyrrolidin-2-yl)methoxy)-6-(quinazolin-4-yl)-5,6,7,8-tetrahydro-2,6-naphthyridine-4-carbonitrile: To the 100 mL two neck RBF, a suspension of (S)-3-((1-methylpyrrolidin-2-yl)methoxy)-1-(piperazin-1-yl)-6-(quinazolin-4-yl)-5,6,7,8-tetrahydro-2,6-naphthyridine-4-carbonitrile hydrochloride (0.3 gm, 0.00062 mol) in ethyl acetate (1.8 mL) at 0° C. was added triethylamine (0.7 mL, 0.004% mol), 2-fluoroprop-2-enoic acid (0.11 gm, 0.00124 mol) and T3P (1.2 mL, 0.00186 mol, 50 wt. % in ethyl acetate). The mixture was warmed to 15° C. for 30 min and the reaction mixture was diluted with saturated aqueous sodium carbonate (10 mL). The aqueous phase was extracted with ethyl acetate (15 mL×2) and the combined organic layer was dried over anhydrous sodium sulfate, filtered, and concentrated to provide the crude residue. The residue was purified by prep-HPLC; column: Waters Xbridge C18 150×50 mm×10 μm, mobile phase: A [water (10 mM NH₄HCO₃)], B (ACN), B %: 42%-72%, 11.5 min); to afford (S)-1-(4-(2-fluoroacryloyl)piperazin-1-yl)-3-((1-methylpyrrolidin-2-yl)methoxy)-6-(quinazolin-4-yl)-5,6,7,8-tetrahydro-2,6-naphthyridine-4-carbonitrile (0.065 gm, 18.83%). MS (m/z): 557.2 (M+1).

Example 11: (S)-6-(8-chloronaphthalen-1-yl)-3-((1-methylpyrrolidin-2-yl)methoxy)-1-(4-(vinylsulfonyl)piperazin-1-yl)-5,6,7,8-tetrahydro-2,6-naphthyridine-4-carbonitrile: In Two neck 100 mL RBF, a suspension of (S)-6-(8-chloronaphthalen-1-yl)-3-((1-methylpyrrolidin-2-yl)methoxy)-1-(piperazin-1-yl)-5,6,7,8-tetrahydro-2,6-naphthyridine-4-carbonitrile hydrochloride (0.32 gm, 0.00062 mol) in DCM (4 mL) was added TEA (0.26 mL, 0.00186 mol) at 0° C. After 5 min, the solution of 2-chloroethanesulfonyl chloride (0.1 mL, 0.00124 mol) in DCM (8.8 mL) was added dropwise and stirred for at RT 12 hrs. The completion of reaction was checked by TLC using methanol: DCM (5%) and 1 drop of methanolic ammonia as mobile phase. The RM was concentrated under reduced pressure to get a crude mass which was purified by column chromatography (100-200 mesh silica gel) using 5-8% MeOH in DCM as mobile phase to afford (S)-6-(8-chloronaphthalen-1-yl)-3-((1-methylpyrrolidin-2-yl)methoxy)-1-(4-(vinylsulfonyl)piperazin-1-yl)-5,6,7,8-tetrahydro-2,6-naphthyridine-4-carbonitrile (0.04 gm, 10.6%) as white solid. 1H-NMR (400 MHz) (CDCl3): 7.79-7.76 (m, 1H), 7.67-7.64 (m, 1H), 7.57-7.44 (m, 2H), 7.38-7.33 (m, 1H), 7.28-7.26 (m, 1H), 6.49-6.47 (m, 1H), 6.33-6.29 (m, 1H), 6.15-6.11 (m, 1H), 5.21 (bs, 1H), 4.58-4.54 (m, 2H), 3.96-3.90 (m, 2H), 3.67-3.55 (m, 4H), 3.47-3.42 (m, 2H), 3.39-3.34 (m, 2H), 3.30-3.26 (m, 2H), 3.16-2.86 (m, 6H), 2.59-2.50 (m, 1H), 2.36-2.32 (m, 2H), 2.13-2.07 (m, 2H). MS (m/z): 607.4 (M+1). HPLC purity: 92.22%.

Example 12: 6-(8-chloronaphthalen-1-yl)-1-(4-(2-fluoroacryloyl)-3-methylpiperazin-1-yl)-3-(((S)-1-methylpyrrolidin-2-yl)methoxy)-5,6,7,8-tetrahydro-2,6-naphthyridine-4-carbonitrile: Was prepared using the process as described above for Example 10 to get crude product which was further purified by prep-HPLC; column: Sunfire C18 150×50 mm×10 μm, mobile phase: A [water (10 mM NH₄HCO₃)], B (ACN), B %: 42%-72%); to afford 6-(8-chloronaphthalen-1-yl)-1-((R)-4-(2-fluoroacryloyl)-3-methylpiperazin-1-yl)-3-(((S)-1-methyl pyrrolidin-2-yl)methoxy)-5,6,7,8-tetrahydro-2,6-naphthyridine-4-carbonitrile (0.16 gm, 22.4%). 1H-NMR (400 MHz) (CDCl3): 7.79-7.76 (m, 1H), 7.67-7.64 (m, 1H), 7.55-7.52 (m, 1H), 7.50-7.42 (m, 1H), 7.37-7.33 (m, 1H), 7.28-7.21 (m, 1H), 5.35-5.14 (m, 2H), 4.80-4.69 (m, 2H), 4.624.51 (m, 3H), 3.99-3.89 (m, 2H), 3.75-3.55 (m, 4H), 3.51-3.40 (m, 2H), 3.34-2.53 (m, 8H), 2.29-1.99 (m, 4H), 1.50-1.19 (m, 3H). MS (m/z): 603.2 (M+1). HPLC purity: 96.3% (considered with diastereomeric fractions).

Example 13: 1-(4-acryloyl-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile: Was prepared using the process as described above for Example 1 using Intermediate 19C to get 0.85 gm of 1-(4-acryloyl-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile. The racemic mixture (0.8 gm) was isolated by chiral separation using CHIRALCEL-ODH (4.6×250 mm) 5 μm and mobile phase; acetonitrile (100%) to provide 0.33 gm & 0.36 gm of isomers 1 and isomer 2 respectively as example 14 and example 15. 1H-NMR (δ ppm, CDCl₃, 400 MHz): δ 7.36-7.33 (m, 4H), 7.31-7.28 (m, 1H), 6.62-6.55 (m, 1H), 6.35-6.31 (m, 1H), 5.76-5.73 (m, 1H), 4.33-4.23 (m, 2H), 3.79-3.77 (bs, 4H), 3.70-3.66 (bs, 4H), 3.36 (bs, 4H), 3.12-3.08 (m, 1H), 2.72-2.68 (m, 1H), 2.61-2.59 (bs, 4H), 2.51 (bs, 3H), 2.34-2.30 (m, 1H), 2.06-2.01 (m, 1H), 1.83-1.67 (m, 3H). MS (m/z): 576.2 [M+H]⁺. HPLC purity: 96.72%.

Example 14: 1—((S)-4-acryloyl-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile: 1H-NMR (δ ppm, CDCl₃, 400 MHz): δ 7.36-7.33 (m, 4H), 7.31-7.28 (m, 1H), 6.62-6.55 (m, 1H), 6.35-6.31 (m, 1H), 5.76-5.73 (m, 1H), 4.334.23 (m, 2H), 3.79-3.77 (bs, 4H), 3.70-3.66 (bs, 4H), 3.36 (bs, 4H), 3.12-3.08 (m, 1H), 2.72-2.68 (m, 1H), 2.61-2.59 (bs, 4H), 2.51 (bs, 3H), 2.34-2.30 (m, 1H), 2.06-2.01 (m, 1H), 1.83-1.67 (m, 3H). MS (m/z): 576.2 [M+H]⁺. HPLC purity: 97.62%.

Example 15: 1—((R)-4-acryloyl-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile: 1H-NMR (δ ppm, CDCl₃, 400 MHz): δ 7.36-7.33 (m, 4H), 7.31-7.28 (m, 1H), 6.62-6.55 (m, 1H), 6.35-6.31 (m, 1H), 5.76-5.73 (m, 1H), 4.33-4.23 (m, 2H), 3.79-3.77 (bs, 4H), 3.70-3.66 (bs, 4H), 3.36 (bs, 4H), 3.12-3.08 (m, 1H), 2.72-2.68 (m, 1H), 2.61-2.59 (bs, 4H), 2.51 (bs, 3H), 2.34-2.30 (m, 1H), 2.06-2.01 (m, 1H), 1.83-1.67 (m, 3H). MS (m/z): 576.2 [M+H]⁺. HPLC purity: 97.31%.

Example 16: 1-(4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile: Was prepared using the process as described above for Example 1 to get The pure fractions were collected and concentrated to get the desired product (0.61 gm, 34.51%). 1H-NMR (400 MHz) (CDCl3): 8.20-8.17 (m, 1H), 7.88-7.86 (m, 1H), 7.64 (d, J=8.4 Hz, 1H), 7.53-7.51 (m, 2H), 7.50-7.44 (m, 1H), 7.18 (d, J=7.2 Hz, 1H), 6.64-6.59 (m, 1H), 6.42-6.35 (m, 1H), 5.84-5.77 (m, 1H), 4.94-4.91 (m, 2H), 4.494.41 (m, 4H), 4.03-3.95 (m, 2H), 3.86-3.83 (m, 1H), 3.58-3.31 (m, 7H), 3.02-2.98 (m, 5H), 2.25-2.23 (m, 2H), 2.03-1.98 (m, 3H), 1.41-1.29 (m, 1H). MS (m/z): 576.25 (M+1). HPLC purity: 95.04%.

Example 17: 1—((S)-4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile: 1-((S)-4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile (0.13 gm) was isolated by chiral separation using CHIRALCEL-ODH (4.6×250 mm) 5 μm and mobile phase: acetonitrile (100%) from the racemic mixture of 1-(4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile (0.57 gm). 1H-NMR (δ ppm, CDCl3, 400 MHz): δ 8.19-8.17 (m, 1H), 7.88-7.86 (m, 1H), 7.64 (d, J=6 Hz, 1H), 7.52-7.51 (m, 2H), 7.46-7.43 (m, 1H), 7.18 (d, J=6 Hz, 1H), 6.61 (bs, 1H), 6.40 (d, J=12.4 Hz, 1H), 5.83 (d, J=8.4 Hz, 1H), 5.09 (bs, 1H), 4.86 (bs, 2H), 4.51-4.43 (m, 4H), 4.01-3.84 (m, 4H), 3.59-3.35 (m, 4H), 3.23-3.11 (m, 2H), 3.01-2.96 (m, 2H), 2.84 (s, 3H), 2.27-2.16 (m, 2H), 2.03-1.99 (m, 2H), 1.41-1.36 (m, 1H). MS (m/z): 576.5 [M+H]⁺. HPLC purity: 96.8%.

Example 18: 1—((R)-4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile: 1-((R)-4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile (0.125 gm) was isolated by chiral separation using CHIRALCEL-ODH (4.6×250 mm) 5 μm and mobile phase, acetonitrile (100%) from racemic mixture of 1-(4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile (0.57 gm). 1H-NMR (δ ppm, CDCl₃, 400 MHz): δ 8.19-8.17 (m, 1H), 7.88-7.86 (m, 1H), 7.64 (d, J=6 Hz, 1H), 7.52-7.50 (m, 2H), 7.44-7.43 (m, 1H), 7.18 (d, J=6 Hz, 1H), 6.60 (bs, 1H), 6.40 (d, J=13.2 Hz, 1H), 5.83 (d, J=8.4 Hz, 1H), 5.11-5.05 (m, 1H), 4.84-4.80 (m, 1H), 4.53-4.39 (m, 4H), 3.98-3.95 (m, 1H), 3.84-3.82 (m, 1H), 3.63 (bs, 2H), 3.36-3.33 (m, 4H), 3.11-3.10 (m, 2H), 3.02-2.95 (m, 2H), 2.86 (s, 3H), 2.78-2.75 (m, 1H), 2.26-2.14 (m, 2H), 2.02-1.98 (m, 2H), 1.37-1.32 (m, 1H). MS (m/z): 576.5 [M+H]⁺. HPLC purity: 93.99%.

Example 19: 1-(4-acryloyl-3-(cyanomethyl)piperazin-1-yl)-6-(3-hydroxynaphthalen-1-yl)-3-(((S)-1-methylpyrrolidin-2-yl)methoxy)-5,6,7,8-tetrahydro-2,6-naphthyridine-4-carbonitrile: Was prepared using the process as described above for Example 1 using Intermediate 19J to get 1-(4-acryloyl-3-(cyanomethyl)piperazin-1-yl)-6-(3-hydroxynaphthalen-1-yl)-3-(((S)-1-methyl pyrrolidin-2-yl)methoxy)-5,6,7,8-tetrahydro-2,6-naphthyridine-4-carbonitrile, was further purified by prep-HPLC; column: Sunfire C18 150×50 mm×10 μm, mobile phase: A [water (10 mM NH₄HCO₃)], B (ACN), B %: 42%-72%) to afford 1-(4-acryloyl-3-(cyanomethyl)piperazin-1-yl)-6-(3-hydroxynaphthalen-1-yl)-3-(((S)-1-methyl pyrrolidin-2-yl)methoxy)-5,6,7,8-tetrahydro-2,6-naphthyridine-4-carbonitrile (0.05 gm) as white solid, 1H-NMR (400 MHz) (CDCl3): 8.00-7.96 (m, 1H), 7.64 (m, J=6 Hz, 1H), 7.42-7.39 (m, 1H), 7.32-7.29 (m, 1H), 6.90 (s, 1H), 6.70 (s, 1H), 6.55 (bs, 1H), 6.37 (d, J=13.2 Hz, 1H), 5.81 (d, J=8.4 Hz, 1H), 5.01-4.92 (m, 1H), 4.594.58 (m, 1H), 4.34-4.26 (m, 3H), 3.85-3.82 (m, 1H), 3.76-3.64 (m, 2H), 3.44-3.24 (m, 4H), 2.95-2.79 (m, 5H), 2.71 (s, 3H), 2.49 (bs, 2H), 2.10-1.99 (m, 3H), 1.88-1.82 (m, 2H), 1.62-1.60 (m, 1H). MS (m/z): 592.5 (M+1). HPLC purity: 98.62%.

Example 20: 1-(3-(cyanomethyl)-4-(2-fluoroacryloyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile: Was prepared using the process as described above for Example 1 to get to provide the crude residue. The residue was purified by prep-HPLC; column: Sunfire C18 150×50 mm×10 μm, mobile phase: A [water (10 mM NH₄HCO₃)], B (ACN), B %: 42%-72%); to afford 1-(3-(cyanomethyl)-4-(2-fluoroacryloyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile (0.09 gm, 26.6%). 1H-NMR (400 MHz) (CDCl3); 8.19-8.17 (m, 1H), 7.88-7.85 (m, 1H), 7.64 (d, J=8.0 Hz, 1H), 7.54-7.49 (m, 2H), 7.46-7.42 (m, 1H), 7.18 (d, J=7.6 Hz, 1H), 5.48-5.36 (m, 1H), 5.28-5.23 (m, 1H), 4.92 (bs, 2H), 4.50-4.43 (m, 3H), 4.09-3.95 (m, 2H), 3.84-3.81 (m, 1H), 3.56-3.31 (m, 6H), 3.09-2.71 (m, 8H), 2.31-2.15 (m, 2H), 2.06-1.97 (m, 2H), 1.43-1.41 (m, 1H). MS (m/z): 594.4 (M+1). HPLC purity: 95.35%.

Example 21: (S)-1-(4-acryloylpiperazin-1-yl)-6-(isoquinolin-4-yl)-3-((1-methylpyrrolidin-2-yl)methoxy)-5,6,7,8-tetrahydro-2,6-naphthyridine-4-carbonitrile: Was prepared using the process as described above for Example 1 to get to get a crude residue. The residue was purified by prep-HPLC; column: Sunfire C18 150×50 mm×10 μm, mobile phase: A [water (10 mM NH₄HCO₃)₁, B (ACN), B %: 42%-72%); to afford (S)-1-(4-acryloylpiperazin-1-yl)-6-(isoquinolin-4-yl)-3-((1-methylpyrrolidin-2-yl)methoxy)-5,6,7,8-tetrahydro-2,6-naphthyridine-4-carbonitrile (0.04 gm, 12%). 1H-NMR (400 MHz) (CDCl3): 9.05 (s, 1H), 8.28 (m, 1H), 8.13 (d, J=6.4 Hz, 1H), 8.01 (d, J=6.4 Hz, 1H), 7.76-7.73 (m, 1H), 7.66-7.63 (m, 1H), 6.64-6.58 (m, 1H), 6.35 (d, J=14.0 Hz, 1H), 5.77 (d, J=8.8 Hz, 1H), 4.47-4.33 (m, 4H), 3.84-3.73 (m, 4H), 3.49-3.44 (m, 6H), 3.19 (bs, 1H), 2.87-2.80 (m, 3H), 2.58 (s, 3H), 2.39-2.31 (m, 1H), 2.09-2.04 (m, 2H), 2.03-1.92 (m, 2H). MS (m/z): 538.5 (M+1). HPLC purity: 97.64%.

Example 22: (S)-1-(4-acryloylpiperazin-1-yl)-3-((1-methylpyrrolidin-2-yl)methoxy)-6-(quinolin-8-yl)-5,6,7,8-tetrahydro-2,6-naphthyridine-4-carbonitrile: Was prepared using the process as described above for Example 1 to get crude residue. The residue was purified by prep-HPLC; column: Sunfire C18 150×50 mm×10 μm, mobile phase: A [water (10 mM NH₄HCO₃)], B (ACN), B %: 42%-72%); to afford (S)-1-(4-acryloylpiperazin-1-yl)-6-(isoquinolin-4-yl)-3-((1-methylpyrrolidin-2-yl)methoxy)-5,6,7,8-tetrahydro-2,6-naphthyridine-4-carbonitrile (0.04 gm, 12%). 1H-NMR (400 MHz)(CDCl3): 9.05 (s, 1H), 8.28 (m, 1H), 8.13 (d, J=6.4 Hz, 1H), 8.01 (d, J=6.4 Hz, 1H), 7.76-7.73 (m, 1H), 7.66-7.63 (m, 1H), 6.64-6.58 (m, 1H), 6.35 (d, J=14.0 Hz, 1H), 5.77 (d, J=8.8 Hz, 1H), 4.47-4.33 (m, 4H), 3.84-3.73 (m, 4H), 3.49-3.44 (m, 6H), 3.19 (bs, 1H), 2.87-2.80 (m, 3H), 2.58 (s, 3H), 2.39-2.31 (m, 11H), 2.09-2.04 (m, 2H), 2.03-1.92 ((m, 2H). MS (m/z): 538.5 (M+1). HPLC purity: 97.64%.

Example 23: 1-(4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile: To Intermediate 9 (8 gm, 0.014 mol) in DCM (80 ml) was added DIPEA (5.1 ml, 0.028 mol) at 0° C. After 5 min, the solution of acryloyl chloride (1.7 mL, 0.021 moles) in DCM (15 mL) was added dropwise and stirred for 30 min at 0° C. The completion of reaction was checked by TLC using methanol: DCM (5%) as mobile phase. Saturated solution of NaHCO₃ (20 mL) was added to the reaction mixture and stirred for more 15 min then extracted with DCM (20 ml×3). The organic layer was washed with NaHCO₃ (10 mL), dried over Na2SO4 and evaporated under reduced pressure to get, was purified by prep-HPLC; column: Tri Art C18*250*20*mm*5 um mobile phase: 0.1% NH3 in H2O B: ACN(25:75) to afford 1-(4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile (1.2 g). ¹H-NMR (400 MHz) (CDCl3): 7.77-7.75 (m, 1H), 7.66-7.62 (m, 1H), 7.53-7.51 (m, 1H), 7.46-7.41 (m, 1H), 7.36-7.32 (m, 1H), 7.28-7.19 (m, 11H), 6.59 (bs, 1H), 6.37 (m, 1H), 5.82 (m, 1H), 4.65-4.54 (m, 1H), 4.32-4.12 (m, 2H), 3.90-3.82 (m, 4H), 3.62-3.52 (m, 2H), 3.45-3.47 (m, 1H), 2.70-2.62 (m, 4H), 2.52-2.42 (m, 5H), 2.34 (s, 3H), 1.87-1.85 (m, 1H), 1.82-1.78 (m, 1H), 1.76-1.72 (m, 6H). MS (m/z): 610.4 (M+1). HPLC purity: 97.16%.

Racemic mixture of 1-(4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile (1.2 gm) was subjected by chiral separation using CHIRALCEL-ODH (4.6×250 mm) 5 μm and mobile phase; acetonitrile (100%): 0.1% DEA to get and 1-((R)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile (Isomer 1) (0.51 gm) and 1-((S)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile (Isomer 2) (0.49 gm)

Example 24: 1—((R)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile (Isomer 1): 1H-NMR (δ ppm, CDCl₃, 400 MHz): δ 7.78-7.75 (m, 1H), 7.67-7.63 (m, 1H), 7.53 (d, J=7.6 Hz, 1H), 7.52-7.51 (m, 2H), 7.49-7.41 (m, 1H), 7.37-7.33 (m, 1H), 7.29-7.26 (m, 1H), 6.63-6.56 (m, 1H), 6.39 (d, J=16.8 Hz, 1H), 5.83 (d, J=11.2 Hz, 1H), 5.12-5.10 (m, 1H), 4.64-4.56 (m, 1H), 4.38-4.29 (m, 2H), 4.01-3.83 (m, 3H), 3.68-3.59 (m, 2H), 3.41-3.38 (m, 1H), 3.22-3.05 (m, 4H), 2.94-2.84 (m, 11H), 2.75-2.62 (m, 3H), 2.54 (s, 3H), 2.35-2.33 (m, 1H), 2.09-2.02 (m, 1H), 1.88-1.73 (m, 1H). MS (m/z): 610.4 [M+H]⁺. HPLC purity: 97.6%. chiral purity: 99.01%.

Example 25: 1—((S)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile (Isomer 2) (Cpd A): 1H-NMR (δ ppm, CDCl₃, 400 MHz): δ 7.78-7.76 (m, 1H), 7.67-7.63 (m, 1H), 7.53 (d, J=7.6 Hz, 1H), 7.49-7.42 (m, 1H), 7.37-7.33 (m, 1H), 7.29-7.26 (m, 1H), 6.59-6.56 (m, 1H), 6.39 (d, J=16.8 Hz, 1H), 5.83 (d, J=10.4 Hz, 1H), 5.12 (bs, 1H), 4.64-4.56 (m, 1H), 4.42-4.29 (m, 2H), 4.01-3.83 (m, 3H), 3.69-3.59 (m, 1H), 3.42-3.38 (m, 1H), 3.23-3.04 (m, 4H), 2.91-2.77 (m, 2H), 2.71-2.67 (m, 1H), 2.55 (s, 3H), 2.40-2.33 (m, 1H), 2.10-2.03 (m, 2H), 2.01-1.73 (m, 5H). MS (m/z): 610.4 [M+H]⁺. HPLC purity: 98.9%, chiral purity: 99.6%

Example 26: 6-(8-chloronaphthalen-1-yl)-1-(3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile: To a suspension of 6-(8-chloronaphthalen-1-yl)-1-(3-(cyanomethyl)piperazin-1-yl)-3-(((S)-1-methylpyrrolidin-2-yl)methoxy)-5,6,7,8-tetrahydro-2,6-naphthyridine-4-carbonitrile hydrochloride (1.8 gm, 0.00324 mol) in ethyl acetate (10.8 mL) at 0° C. was added triethylamine (3.6 mL, 0.02592 mol), 2-fluoroprop-2-enoic acid (0.58 gm, 0.00647 mol) and T3P (6.2 mL, 0.00972 mol, 50 wt. % in ethyl acetate). The mixture was warmed to 15° C. for 30 min and the reaction mixture was diluted with saturated aqueous sodium carbonate (40 mL). The aqueous phase was extracted with ethyl acetate (100 mL×2) and the combined organic layer was dried over anhydrous sodium sulfate, filtered and concentrated to provide the crude residue. The residue was purified by prep-HPLC; column: Sunfire C 18 150×50 mm-10 μm, mobile phase: A: water (10 mM NH4HCO3), B: ACN, B %: 25%-55%, to afford 6-(8-chloronaphthalen-1-yl)-1-((S)-(3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile (0.42 gm, 20.64%).

Example 27: 6-(8-chloronaphthalen-1-yl)-1-((S)-3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile: 6-(8-chloronaphthalen-1-yl)-1-((S)-3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile (0.075 gm, chiral purity: 99%) was isolated by chiral separation using CHIRALCEL-ODH (4.6×250 mm) 5 μm and mobile phase: acetonitrile (100%) from the racemic mixture of 6-(8-chloronaphthalen-1-yl)-1-(3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile (0.42 gm). 1H-NMR (δ ppm, CDCl₃, 400 MHz): δ 7.79-7.76 (m, 1H), 7.67-7.63 (m, 1H), 7.53 (d, J=7.2 Hz, 1H), 7.49.7.42 (m, 1H), 7.37-7.33 (m, 1H), 7.29-7.26 (m, 1H), 5.48-5.36 (m, 1H), 5.28-5.23 (m, 1H), 4.64-4.56 (m, 1H), 4.38-4.27 (m, 2H), 4.09-3.81 (m, 3H), 3.67-3.59 (m, 2H), 3.51-3.45 (m, 1H), 3.41-3.36 (m, 1H), 3.29-3.10 (m, 4H), 2.99-2.89 (m, 2H), 2.74-2.70 (m, 2H), 2.53 (s, 3H), 2.36-2.29 (m, 1H), 2.10-1.98 (m, 1H), 1.89-1.61 (m, 3H), 1.29-1.23 (m, 1H). MS (m/z): 628.3 [M+H]+. HPLC purity: 97.6%.

Example 28: 6-(8-chloronaphthalen-1-yl)-1-((R)-3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile: 6-(8-chloronaphthalen-1-yl)-1-((R)-3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile (0.095 gm, chiral purity: 99%) was isolated by chiral separation using CHIRALCEL-ODH (4.6×250 mm) 5 μm and mobile phase: acetonitrile (100%) from the racemic mixture of 6-(8-chloronaphthalen-1-yl)-1-(3-(cyanomethyl)-4-(2-fluoroacryloyl)piperazin-1-yl)-3-(((S)-1-methylpyrrolidin-2-yl) methoxy)-5,6,7,8-tetrahydro-2,6-naphthyridine-4-carbonitrile (0.42 gm). 1H-NMR (δ ppm, CDCl₃, 400 MHz): δ 7.79-7.76 (m, 1H), 7.67-7.63 (m, 1H), 7.53 (d, J=7.2 Hz, 1H), 7.49.7.42 (m, 1H), 7.37-7.33 (m, 1H), 7.29-7.26 (m, 1H), 5.48-5.36 (m, 1H), 5.28-5.23 (m, 1H), 4.64-4.56 (m, 1H), 4.38-4.27 (m, 2H), 4.09-3.81 (m, 3H), 3.67-3.59 (m, 2H), 3.51-3.45 (m, 1H), 3.41-3.36 (m, 1H), 3.29-3.10 (m, 4H), 2.99-2.89 (m, 2H), 2.74-2.70 (m, 2H), 2.53 (s, 3H), 2.36-2.29 (m, 1H), 2.10-1.98 (m, 1H), 1.89-1.61 (m, 3H), 1.29-1.23 (m, 1H). MS (m/z): 628.3 [M+H]⁺. HPLC purity: 97.6%.

Example 29: 1-(4-acryloylpiperazin-1-yl)-6-benzyl-3-chloro-5,6,7,8-tetrahydro-2,6-naphthyridine-4-carbonitrile: To a solution of intermediate 4 (0.2 g, 0.5 mmol) was dissolved in dry DCM (10 ml), followed by addition of DIPEA (0.3 g, 1.5 mmol) and stirred reaction mixture for 20 min at 0° C. Acryloyl chloride (0.053 g, 0.6 mmol) was added dropwise in the reaction mixture and stirred same temperature for 30 min. The reaction mixture was diluted with DCM and washed with water. The organic layer was dried over sodium sulphate and concentrated under reduced pressure to afford the title solid compound, yield (20 mg). 1H-NMR (δ ppm, CDCl₃, 400 MHz): 7.36-7.29 (m, 5H), 6.60-6.54 (m, 1H), 6.34-6.30 (m, 1H), 5.75-5.73 (m, 1H), 3.80 (s, 4H), 3.68-3.65 (m, 4H), 3.43-3.41 (m, 4H), 2.67-2.59 (m, 4H), MS (m/z): 422.19 [M+H]⁺. HPLC purity: 97.38%.

Example 30: N-(1-(6-benzyl-3-chloro-4-cyano-5,6,7,8-tetrahydro-2,6-naphthyridin-1-yl)piperidin-4-yl)acrylamide: To a solution of intermediate 7 (0.2 g, 52 mmol) was dissolved in dry DCM (10 ml), followed by addition of DIPEA (0.23 g, 157 mmol) and stirred reaction mixture for 20 min at 0° C. Acryloyl chloride (0.2 g, 62 mmol) was added dropwise in the reaction mixture and stirred same temperature for 30 min. The reaction mixture was diluted with DCM and washed with water. The organic layer was dried over sodium sulphate and concentrated under reduced pressure to afford the title solid compound, yield (25 mg). 1H-NMR (δ ppm, CDCl₃, 400 MHz): 8.32 (s, 1H), 7.36-7.34 (m, 5H), 6.31-6.27 (m, 1H), 6.09-6.03 (m, 1H), 5.67-5.65 (m, 1H), 5.43-5.41 (d, 1H), 4.41-4.08 (m, 1H), 3.80 (s, 2H), 3.75-3.71 (m, 4H), 3.06-3.01 (m, 2H), 2.70-2.62 (m, 4H), 2.08-2.03 (m, 2H), MS (m/z): 402.21 [M+H]⁺. HPLC purity: 97.00%.

Example 31: N-(1-(6-benzyl-4-cyano-5,6,7,8-tetrahydro-2,6-naphthyridin-1-yl)piperidin-4-yl)acrylamide: To a solution of intermediate 6 (0.12 g, 31 mmol) was dissolved in dry DCM (10 ml), followed by addition of DIPEA (0.17 g, 91 mmol) and stirred reaction mixture for 20 min at 0° C. Acryloyl chloride (0.033 g, 35 mmol) was added dropwise in the reaction mixture and stirred same temperature for 30 min. The reaction mixture was diluted with DCM and washed with water. The organic layer was dried over sodium sulphate and concentrated under reduced pressure to afford the title solid compound, yield (25 mg). ¹H-NMR (δ ppm, CDCl₃, 400 MHz): 7.36-7.27 (m, 5H), 6.30-6.27 (m, 1H), 6.08-6.03 (m, 1H), 5.67-5.65 (m, 1H), 5.43-5.41 (d, 1H), 4.14-4.10 (m, 1H), 3.80 (s, 4H), 3.78-3.68 (m, 2H), 3.09-3.04 (m, 2H), 2.64-2.61 (m, 5H), 2.21-2.02 (m, 3H), MS (m/z): 436.12 [M+H]⁺. HPLC purity: 96.25%.

Example 32: 1,3-bis(4-acryloylpiperazin-1-yl)-6-benzyl-5,6,7,8-tetrahydro-2,6-naphthyridine-4-carbonitrile: Was prepared using the process as described above for Example 1 using Intermediate 7D to get 20 mg of 1,3-bis(4-acryloylpiperazin-1-yl)-6-benzyl-5,6,7,8-tetrahydro-2,6-naphthyridine-4-carbonitrile ¹H-NMR (5 ppm, CDCl3, 400 MHz): 7.38-7.26 (m, 5H), 6.57-6.53 (m, 2H), 6.34-6.31 (m, 2H), 5.75-5.73 (m, 2H), 3.81-3.61 (m, 9H), 3.33 (s, 4H), 2.63 (s, 4H), 1.53-1.50 (m, 2H), 1.42-1.41 (m, 2H), 1.29-1.23 (m, 4H) 526.27 [M+H]⁺. HPLC Purity: 97.67%

Example 33 1-(4-acryloylpiperazin-1-yl)-6-benzyl-4-cyano-5,6,7,8-tetrahydro-2,6-naphthyridin-3-yl acrylate: Was prepared using the process as described above for Example 1 using Intermediate 6A1 to get 50 mg of 1-(4-acryloylpiperazin-1-yl)-6-benzyl-4-cyano-5,6,7,8-tetrahydro-2,6-naphthyridin-3-yl acrylate. 1H-NMR (δ ppm, CDCl3, 500 MHz): δ 7.37-7.33 (m, 4H), 7.32-7.29 (m, 1H), 6.70-6.66 (M, 1H), 6.60-6.53 (m, 1H), 6.37-6.29 (m, 2H), 6.12-6.10 (m, 1H), 5.75-5.72 (m, 1H), 3.83 (s, 2H), 3.77 (bs, 2H), 3.73 (s, 2H), 3.66 (bs, 2H), 3.40 (bs, 4H), 2.70-2.66 (m, 4H). MS (m/z): 458.25 [M+H]⁺. HPLC purity: 96.00%.

Example 34: 1-(4-acryloylpiperazin-1-yl)-6-benzyl-3-morpholino-5,6,7, -tetrahydro-2,6-naphthyridine-4-carbonitrile: Was prepared using the process as described above for Example 1 using Intermediate 6B to get 70 mg of 1-(4-acryloylpiperazin-1-yl)-6-benzyl-3-morpholino-5,6,7,8-tetrahydro-2,6-naphthyridine-4-carbonitrile. 1H-NMR (δ ppm, CDCl3, 400 MHz): δ 7.36 (m, 4H), 7.30 (m, 1H), 6.58 (m, 1H), 6.33 (m, 1H), 5.74 (m, 1H), 3.81 (m, 4H), 3.77 (s, 4H), 3.71 (m, 2H), 3.66 (s, 2H), 3.60 (m, 4H), 3.34 (s, 4H), 2.60 (s, 4H). MS (m/z): 473 [M+H]⁺. HPLC purity: 99.75%.

Example 35: 1-(4-acryloylpiperazin-1-yl)-6-benzyl-3-(4-methylpiperazin-1-yl)-5,6,7,8-tetrahydro-2,6-naphthyridine-4-carbonitrile: Was prepared using the process as described above for Example 1 using Intermediate 6D to get 30 mg of 1-(4-acryloylpiperazin-1-yl)-6-benzyl-3-(4-methylpiperazin-1-yl)-5,6,7,8-tetrahydro-2,6-naphthyridine-4-carbonitrile. ¹H-NMR (5 ppm. CDCl₃, 400 MHz): 7.39-7.33 (m, 4H), 7.30-7.27 (m, 1H), 6.61-6.55 (m, 1H), 6.34-6.30 (m, 1H), 5.74-5.71 (m, 1H), 3.76 (s, 4H), 3.70 (s, 2H), 3.66-3.64 (m, 6H), 3.33 (bs, 4H), 2.59 (bs, 4H), 2.55-2.53 (m, 4H), 2.34 (s, 3H). HPLC Purity: 99.06%

Example 36: 1-(4-acryloyl-3-(cyanomethyl)piperazin-1-yl)-6-benzyl-3-(4-methylpiperazin-1-yl)-5,6,7,8-tetrahydro-2,6-naphthyridine-4-carbonitrile: Was prepared using the process as described above for Example 1 to get 70 mg of 1-(4-acryloyl-3-(cyanomethyl)piperazin-1-yl)-6-benzyl-3-(4-methylpiperazin-1-yl)-5,6,7,8-tetrahydro-2,6-naphthyridine-4-carbonitrile. 1H-NMR (δ ppm, CDCl₃, 400 MHz): δ 7.33 (m, 5H), 6.57 (m, 1H), 6.35 (m, 1H), 5.79 (m, 1H), 3.86 (m, 1H), 3.77 (m, 2H), 3.66 (m, 7H), 3.53 (s, 1H), 3.22 (s, 1H), 2.91 (q, 2H), 2.73 (m, 2H), 2.67 (m, 2H), 2.60 (m, 4H), 2.52 (s, 2H), 2.37 (s, 3H). MS (m/z): 525 [M+H]⁺. HPLC purity: 97.00%.

Example 37: 6-(1-naphthoyl)-1-(4-acryloyl-3-(cyanomethyl)piperazin-1-yl)-3-(4-methylpiperazin-1-yl)-5,6,7,8-tetrahydro-2,6-naphthyridine-4-carbonitrile: Was prepared using the process as described above for Example 1 to get 40 mg 6-(1-naphthoyl)-1-(4-acryloyl-3-(cyanomethyl)piperazin-1-yl)-3-(4-methylpiperazin-1-yl)-5,6,7,8-tetrahydro-2,6-naphthyridine-4-carbonitrile. 1H-NMR (δ ppm, CDCl₃, 400 MHz): 7.93-7.80 (m, 3H), 7.54-7.32 (m, 4H), 6.59-6.51 (m, 1H), 6.42-6.34 (m, 1H), 5.84-5.78 (m, 1H), 4.44-4.42 (m, 2H), 3.85-3.83 (m, 1H), 3.77-3.67 (m, 4H), 3.64-3.61 (m, 4H), 3.29-3.21 (m, 2H), 2.95-2.77 (m, 4H), 2.61-2.57 (m, 1H), 2.54-2.49 (s, 4H), 2.47-2.46 (m, 1H), 2.30 (s, 3H). HPLC Purity: 88.73

Example 38: (S)-1-(4-acryloyl-2-methylpiperazin-1-yl)-6-benzyl-3-(4-methylpiperazin-1-yl)-5,6,7,8-tetrahydro-2,6-naphthyridine-4-carbonitrile: Was prepared using the process as described above for Example 1 using Intermediate 7F to get e 95 mg of (S)-1-(4-acryloyl-2-methylpiperazin-1-yl)-6-benzyl-3-(4-methylpiperazin-1-yl)-5,6,7,8-tetrahydro-2,6-naphthyridine-4-carbonitrile 1H-NMR (δ ppm, CDCl₃, 400 MHz): δ 7.39-7.27 (m, 5H), 6.65-6.51 (m, 1H), 6.36-6.30 (m, 1H), 5.76-5.71 (m, 1H), 4.00-4.04 (m, 1H), 3.86-3.80 (m, 1H), 3.77-3.74 (m, 3H), 3.66-3.61 (m, 4H), 3.53-3.43 (m, 2H), 3.37-3.25 (m, 1H), 2.64-2.55 (m, 8H), 2.35 (s, 3H), 2.12-1.88 (m, 3H), 1.24 (s, 3H). MS (m/z): 500.02 [M+H]⁺. HPLC purity: 95%.

Example 39: (R)-1-(4-acryloyl-2-methylpiperazin-1-yl)-6-benzyl-3-(4-methylpiperazin-1-yl)-5,6,7,8-tetrahydro-2,6-naphthyridine-4-carbonitrile: Was prepared using the process as described above for Example 1 using Intermediate 7E to get 114 mg of (R)-1-(4-acryloyl-2-methylpiperazin-1-yl)-6-benzyl-3-(4-methylpiperazin-1-yl)-5,6,7,8-tetrahydro-2,6-naphthyridine-4-carbonitrile. 1H-NMR (δ ppm, CDCl₃, 500 MHz): δ 7.39-7.35 (m, 4H), 7.33-7.28 (m, 1H), 6.64-6.50 (m, 1H), 6.35-6.30 (m, 1H), 5.77-5.71 (m, 1H), 4.39-4.36 (m, 1H), 4.16-4.13 (m, 1H), 3.99-3.97 (m, 2H), 3.83-3.64 (m, 4H), 3.62-3.35 (m, 4H), 3.31-3.17 (m, 3H), 2.67-2.65 (m, 4H), 2.62-2.53 (m, 4H), 2.40 (s, 3H), 1.15-1.14 (m, 3H). MS (m/z): 500 [M+H]+. HPLC purity: 96.42%.

Example 40: 1-(4-acryloylpiperazin-1-yl)-6-benzyl-3-hydroxy-5,6,7,8-tetrahydro-2,6-naphthyridine-4-carbonitrile: Was prepared using the process as described above for Example 1 using Intermediate 6A1 to get 30 mg of 1-(4-acryloylpiperazin-1-yl)-6-benzyl-3-hydroxy-5,6,7,8-tetrahydro-2,6-naphthyridine-4-carbonitrile. 1H-NMR (δ ppm, CDCl₃, 500 MHz): δ 12.13 (bs, 1H), 7.38-7.34 (m, 4H), 7.32-7.27 (m, 1H), 6.64-6.58 (m, 1H), 6.34-6.31 (m, 1H), 5.77-5.74 (m, 1H), 3.78 (bs, 4H), 3.75 (s, 2H), 3.65 (s, 2H), 3.53 (bs, 2H), 3.42 (bs, 2H), 2.59-2.54 (m, 4H). MS (m/z): 404 [M+H]⁺. HPLC purity: 99.79%.

Example 41: 1-(4-acryloylpiperazin-1-yl)-3-(4-methylpiperazin-1-yl)-6-(naphthalen-1-yl)-5,6,7,8-tetrahydro-2,6-naphthyridine-4-carbonitrile: Was prepared using the process as described above for Example 1 using Intermediate 19M to get 113 mg of 1-(4-acryloylpiperazin-1-yl)-3-(4-methylpiperazin-1-yl)-6-(naphthalen-1-yl)-5,6,7,8-tetrahydro-2,6-naphthyridine-4-carbonitrile. 1H-NMR (δ ppm, CDCl₃, 500 MHz): δ 8.21-8.19 (m, 1H), 7.88-7.86 (m, 1H), 7.62 (d, J=8.0 Hz, 1H), 7.52-7.49 (m, 2H), 7.45-7.42 (m, 1H), 7.19-7.18 (d, J=7.0 Hz, 1H), 6.64-6.58 (m, 1H), 6.42-6.33 (m, 2H), 6.17-6.12 (m, 1H), 5.86-5.74 (m, 2H), 4.39 (bs, 2H), 3.84 (bs, 2H), 3.75-3.71 (m, 6H), 3.42 (bs, 6H), 2.87 (bs, 2H), 2.65-2.63 (m, 4H), 2.39 (s, 3H). MS (m/z): 523 [M+H]+. HPLC purity: 95.75%.

Example 42: 1-(4-acryloylpiperazin-1-yl)-6-benzyl-3-(piperidin-1-yl)-5,6,7,8-tetrahydro-2,6-naphthyridine-4-carbonitrile: Was prepared using the process as described above for Example 1 using Intermediate 6F to get 65 mg 0.065 gm of 1-(4-acryloylpiperazin-1-yl)-6-benzyl-3-(piperidin-1-yl)-5,6,7,8-tetrahydro-2,6-naphthyridine-4-carbonitrile. 1H-NMR (δ ppm, CDCl₃, 500 MHz): δ 7.39-7.33 (m, 4H), 7.30-7.28 (m, 1H), 6.61-6.55 (m, 1H), 6.34-6.29 (m, 1H), 5.73-5.71 (m, 1H), 3.76 (bs, 4H), 3.70 (s, 2H), 3.65 (bs, 2H), 3.58 (bs, 4H), 3.32 (bs. 4H), 2.59 (bs, 4H), 1.65 (bs, 6H). MS (nm/z): 471 [M+H]⁺. HPLC purity: 99.53%.

Example 43: 1-(4-acryloylpiperazin-1-yl)-6-benzyl-3-(2-fluoropyridin-4-yl)-5,6,7,8-tetrahydro-2,6-naphthyridine-4-carbonitrile: Was prepared using the process as described above for Example 1 using Intermediate 6E to get 110 mg of 1-(4-acryloylpiperazin-1-yl)-6-benzyl-3-(2-fluoropyridin-4-yl)-5,6,7,8-tetrahydro-2,6-naphthyridine-4-carbonitrile. 1H-NMR (δ ppm, CDCl₃, 400 MHz): δ 8.35 (d, J=5.6 Hz, 1H), 7.76-7.73 (m, 1H), 7.44 (bs, 1H), 7.40-7.35 (m, 4H), 7.33-7.23 (m, 1H), 6.63-6.56 (m, 1H), 6.36-6.31 (m, 1H), 5.77-5.74 (m, 1H), 3.91-3.72 (m, 8H), 3.49-3.44 (m, 4H), 2.80-2.78 (m, 2H), 2.71-2.69 (m, 2H). MS (m/z): 483 [M+H]⁺. HPLC purity: 99.8%.

Example 44: 1-(4-acryloylpiperazin-1-yl)-6-(naphthalen-1-yl)-3-(piperidin-1-yl)-5,6,7,8-tetrahydro-2,6-naphthyridine-4-carbonitrile: Was prepared using the process as described above for Example 1 using Intermediate 19N to get 150 mg of 1-(4-acryloylpiperazin-1-yl)-6-(naphthalen-1-yl)-3-(piperidin-1-yl)-5,6,7,8-tetrahydro-2,6-naphthyridine-4-carbonitrile. 1H-NMR (o ppm. CDCl₃, 400 MHz): δ 8.21 (m, 1H), 7.86 (m, 1H), 7.62 (m, 1H), 7.51 (m, 2H), 7.43 (m, 1H), 7.19 (m, 1H), 6.61 (m, 1H), 6.34 (s, 1H), 5.75 (m, 1H), 4.38 (s, 2H), 3.84 (s, 2H), 3.72 (s, 2H), 3.63 (m, 4H), 3.37 (m, 6H), 2.83 (d, 2H), 1.68 (m, 6H), MS (m/z): 507[M+H]⁺. HPLC purity: 98.89%.

Example 45: 1-(4-acryloylpiperazin-1-yl)-6-benzyl-3-((1-methylpyrrolidin-2-yl)methoxy)-5,6,7,8-tetrahydro-2,6-naphthyridine-4-carbonitrile: Was prepared using the process as described above for Example 1 using Intermediate 10A to get 52 mg of 1-(4-acryloylpiperazin-1-yl)-6-benzyl-3-((1-methylpyrrolidin-2-yl)methoxy)-5,6,7,8-tetrahydro-2,6-naphthyridine-4-carbonitrile. 1H-NMR (δ ppm, CDCl₃, 400 MHz): δ 7.36-7.33 (m, 4H), 7.31-7.28 (m, 1H), 6.62-6.55 (m, 1H), 6.35-6.31 (m, 1H), 5.76-5.73 (m, 1H), 4.33-4.23 (m, 2H), 3.79-3.77 (bs, 4H), 3.70-3.66 (bs, 4H), 3.36 (bs, 4H), 3.12-3.08 (m, 1H), 2.72-2.68 (m, 1H), 2.61-2.59 (bs, 4H), 2.51 (bs, 3H), 2.34-2.30 (m, 1H), 2.06-2.01 (m, 1H), 1.83-1.67 (m, 3H). MS (m/z): 502 [M+H]⁺. HPLC purity: 96.86%.

Example 46: 1,3-bis(4-acryloylpiperazin-1-yl)-6-(naphthalen-1-yl)-5,6,7,8-tetrahydro-2,6-naphthyridine-4-carbonitrile: Was prepared using the process as described above for Example 1 using Intermediate 19K to get 85 mg of 1,3-bis(4-acryloylpiperazin-1-yl)-6-(naphthalen-1-yl)-5,6,7,8-tetrahydro-2,6-naphthyridine-4-carbonitrile. 1H-NMR (o ppm, CDCl₃, 400 MHz): 8.21-8.18 (m, 1H), 7.88-7.85 (m, 1H). 7.64-7.62 (m, 1H), 7.53-7.48 (m, 2H), 7.45-7.41 (m, 1H), 7.19-7.17 (m, 1H), 6.63-6.56 (m, 2H), 6.37-6.31 (m, 2H), 5.77-5.73 (m, 2H), 4.40 (s, 2H), 3.84 (bs, 4H), 3.72 (bs, 4H), 3.68-3.66 (m, 4H), 3.50-3.35 (m, 6H), 2.88 (bs, 2H). 562 [M+H]⁺. HPLC Purity; 98.56%

Example 47: 1,3-bis(4-acryloylpiperazin-1-yl)-6-(benzo[b]thiophen-4-yl)-5,6,7,8-tetrahydro-2,6-naphthyridine-4-carbonitrile: Was prepared using the process as described above for Example 1 using Intermediate 19L to get 60 mg of 1,3-bis(4-acryloylpiperazin-1-yl)-6-(benzo[b]thiophen-4-yl)-5,6,7,8-tetrahydro-2,6-naphthyridine-4-carbonitrile. 1H-NMR (δ ppm, CDCl₃, 400 MHz): 7.63-7.61 (m, 1H), 7.45-7.44 (m, 2H), 7.33-7.29 (m, 1H), 6.99-6.97 (m, 1H), 6.63-6.56 (m, 2H), 6.37-6.31 (m, 2H), 5.77-5.72 (m, 2H), 4.43 (s, 2H), 3.88 (bs, 4H), 3.72-3.66 (m, 8H), 3.43-3.40 (m, 6H), 2.84-2.82 (m, 2H), 568.41 [M+H]1. HPLC Purity: 99.38%

Example 48: 1-(4-acryloylpiperazin-1-yl)-6-(naphthalen-1-yl)-3-(4-propionylpiperazin-1-yl)-5,6,7,8-tetrahydro-2,6-naphthyridine-4-carbonitrile: Was prepared using the process as described above for Example 1 using Intermediate 19P to get 55 mg of 1-(4-acryloylpiperazin-1-yl)-6-(naphthalen-1-yl)-3-(4-propionylpiperazin-1-yl)-5,6,7,8-tetrahydro-2,6-naphthyridine-4-carbonitrile. 1H-NMR (δ ppm, CDCl₃, 400 MHz): 8.21-8.18 (m, 1H), 7.88-7.65 (m, 1H), 7.64-7.62 (m, 1H), 7.53-7.49 (m, 2H), 7.45-7.41 (m, 1H), 7.21-7.18 (m, 1H), 6.64-6.57 (m, 1H), 6.37-6.32 (m, 1H), 5.77-5.74 (m, 1H), 4.40 (s, 2H), 3.83-3.72 (m, 6H), 3.65-3.63 (m, 6H), 3.41 (bs, 4H), 3.40-3.35 (m, 2H), 2.87 (s, 2H), 1.20-1.16 (m, 3H), 564.11[M+H]⁺. HPLC Purity: 97.59%

Example 49: 3-(4-acryloylpiperazin-1-yl)-6-(naphthalen-1-yl)-1-(4-propionylpiperazin-1-yl)-5,6,7,8-tetrahydro-2,6-naphthyridine-4-carbonitrile; Step 1: tert-butyl 4-(4-cyano-6-(naphthalen-1-yl)-1-(4-propionylpiperazin-1-yl)-5,6,7,8-tetrahydro-2,6-naphthyridin-3-yl) piperazine-1-carboxylate was prepared using the similar process as described above for Example 41 with necessary variation in the starting material and reactants to get 150 mg of tilted compound. 609.34 [M+H]⁺.

Step 2: Was prepared using the process as described above for Example 1 (using the intermediate of step 1) to get 85 mg of 3-(4-acryloylpiperazin-1-yl)-6-(naphthalen-1-yl)-1-(4-propionylpiperazin-1-yl)-5,6,7,8-tetrahydro-2,6-naphthyridine-4-carbonitrile. 1H-NMR (8 ppm, CDCl3, 400 MHz): 8.21-8.18 (m, 1H), 7.88-7.86 (m, 1H), 7.64-7.62 (m, 1H), 7.53-7.49 (m, 2H), 7.46-7.42 (m, 1H), 7.19-7.18 (m, 11H), 6.63-6.56 (m, 11H), 6.36-6.31 (m, 11H), 5.76-5.73 (m, 1H), 4.40 (s, 2H), 3.84-3.72 (m, 6H), 3.67-3.62 (m, 6H), 3.40-3.36 (m, 6H), 2.88-2.87 (m, 2H), 2.43-2.38 (m, 2H). 1.22-1.17 (m, 3H). 564.52 [M+H]⁺. HPLC Purity: 97.49%

Example 50: 1-(4-acryloylpiperazin-1-yl)-3-morpholino-6-(naphthalen-1-yl)-5,6,7,8-tetrahydro-2,6-naphthyridine-4-carbonitrile: Was prepared using the process as described above for Example 1 to get 80 mg of 1-(4-acryloylpiperazin-1-yl)-3-morpholino-6-(naphthalen-1-yl)-5,6,7,8-tetrahydro-2,6-naphthyridine-4-carbonitrile. 1H-NMR (δ ppm, CDCl₃, 400 MHz): 8.21-8.18 (m, 1H), 7.88-7.84 (m, 1H), 7.63-7.61 (m, 1H), 7.53-7.49 (m, 2H), 7.45-7.41 (m, 11H), 7.19-7.17 (m, 1H), 6.64-6.57 (m, 1H), 6.37-6.32 (m, 1H), 5.77-5.74 (m, 1H), 4.39 (s, 2H), 3.84-3.82 (m, 6H), 3.72-3.64 (m, 6H), 3.41 (bs, 4H), 2.88 (s, 2H). 509.44[M+H]⁺. HPLC Purity: 95.74%

Example 51: 3-(4-acetylpiperazin-1-yl)-1-(4-acryloylpiperazin-1-yl)-6-(naphthalen-1-yl)-5,6,7,8-tetrahydro-2,6-naphthyridine-4-carbonitrile: Was prepared using the process as described above for Example 1 using Intermediate 19R to get 75 mg of 3-(4-acetylpiperazin-1-yl)-1-(4-acryloylpiperazin-1-yl)-6-(naphthalen-1-yl)-5,6,7,8-tetrahydro-2,6-naphthyridine-4-carbonitrile. 1H-NMR (δ ppm, CDCl₃, 400 MHz): 8.21-8.18 (m, 1H), 7.88-7.86 (m, 1H), 7.64-7.62 (m, 1H), 7.52-7.50 (m, 2H), 7.45-7.42 (m, 1H), 7.19-7.17 (m, 1H), 6.64-6.57 (m, 1H), 6.37-6.32 (m, 1H), 5.77-5.74 (m, 1H), 4.40 (s, 2H), 3.78-3.75 (m, 2H), 3.67-3.65 (m, 4H), 3.62-3.60 (m, 6H), 3.40 (bs, 6H), 2.88 (s, 2H), 2.14 (m, 3H). 550.51 [M+H]⁺. HPLC Purity: 98.69%

Example 52: 1-(4-acryloylpiperazin-1-yl)-3-(2,6-dimethylmorpholino)-6-(naphthalen-1-yl)-5,6,7,8-tetrahydro-2,6-naphthyridine-4-carbonitrile: Was prepared using the process as described above for Example 1 using Intermediate 190 to get 60 mg of 1-(4-acryloylpiperazin-1-yl)-3-(2,6-dimethylmorpholino)-6-(naphthalen-1-yl)-5,6,7,8-tetrahydro-2,6-naphthyridine-4-carbonitrile. 1H-NMR (δ ppm, CDCl₃, 400 MHz): 8.22-8.19 (m, 1H), 7.88-7.84 (m, 1H), 7.63-7.61 (m, 1H), 7.53-7.49 (m, 2H), 7.48-7.41 (m, 1H), 7.19-7.17 (m, 1H), 6.66-6.58 (m, 1H), 6.37-6.32 (m, 1H), 5.77-5.74 (m, 1H), 4.39 (s, 2H), 4.17-4.14 (m, 2H), 3.84-3.81 (m, 2H), 3.79-3.72 (m, 5H), 3.39 (bs, 5H), 2.88 (s, 2H), 2.74-2.68 (m, 2H), 1.25-1.20 (m, 6H), 535.35[M+H]⁺. HPLC Purity: 99.19%

Example 53: 1-(4-acryloylpiperazin-1-yl)-3-(4-(methylsulfonyl)piperazin-1-yl)-6-(naphthalen-1-yl)-5,6,7,8-tetrahydro-2,6-naphthyridine-4-carbonitrile: Was prepared using the process as described above for Example 1 using Intermediate 19Q to get 61 mg of 1-(4-acryloylpiperazin-1-yl)-3-(4-(methylsulfonyl)piperazin-1-yl)-6-(naphthalen-1-yl)-5,6,7,8-tetrahydro-2,6-naphthyridine-4-carbonitrile 1H-NMR (δ ppm, CDCl₃, 400 MHz): 8.20-8.18 (m, 1H), 7.88-7.86 (m, 1H), 7.64-7.62 (m, 1H), 7.52-7.50 (m, 2H), 7.46-7.42 (m, 1H), 7.19-7.17 (m, 1H), 6.65-6.58 (m, 1H), 6.38-6.33 (m, 1H), 5.78-5.75 (m, 1H), 4.40 (s, 2H), 3.84-3.73 (m, 2H), 3.71-3.68 (m, 6H), 3.49 (bs, 4H), 3.43-3.378 (m, 6H), 2.88 (s, 2H), 2.86-2.83 (s, 2H), 586.07[M+H]⁺. HPLC Purity: 98.43%

Example 54: 1-(4-acryloylpiperazin-1-yl)-3-(4-ethylpiperazin-1-yl)-6-(naphthalen-1-yl)-5,6,78-tetrahydro-2,6-naphthyridine-4-carbonitrile; Step 1: tert-butyl 4-(4-cyano-3-(4-ethylpiperazin-1-yl)-6-(naphthalen-1-yl)-5,6,7,8-tetrahydro-2,6-naphthyridin-1-yl)piperazine-1-carboxylate was prepared using the process as described above for Example 41 using Intermediate 5A and 1-ethyl piperazine instead of 1-methyl piperazine to get 120 mg of desired intermediate. 581.35 [M+H]⁺.

Step 2: Was prepared using the process as described above for Example 1 (using the intermediate of step 1) to get 45 mg to get 1-(4-acryloylpiperazin-1-yl)-3-(4-ethylpiperazin-1-yl)-6-(naphthalen-1-yl)-5,6,7,8-tetrahydro-2,6-naphthyridine-4-carbonitrile. 1H-NMR (δ ppm, CDCl₃, 400 MHz): 8.20-8.18 (m, 1H), 7.87-7.86 (m, 1H), 7.63-7.62 (m, 1H), 7.52-7.48 (m, 2H), 7.45-7.42 (m, 1H), 7.19-7.17 (m, 1H), 6.64-6.58 (m, 1H), 6.41-6.33 (m, 1H), 5.77-5.75 (m, 1H), 4.39 (s, 2H), 3.84-3.72 (m, 9H), 3.49-3.43 (m, 7H), 2.88 (s, 4H), 2.80-2.78 (m, 2H), 1.33-1.23 (m, 3H). 536.13[M+H]⁺. HPLC Purity: 98.10%

Example 55: (S)-1-(4-acryloylpiperazin-1-yl)-3-((1-methylpyrrolidin-2-yl)methoxy)-6-(naphthalen-1-yl)-5,6,7,8-tetrahydro-2,6-naphthyridine-4-carbonitrile: Was prepared using the process as described above for Example 1 to get 50 mg of (S)-1-(4-acryloylpiperazin-1-yl)-3-((1-methylpyrrolidin-2-yl)methoxy)-6-(naphthalen-1-yl)-5,6,7,8-tetrahydro-2,6-naphthyridine-4-carbonitrile. 1H-NMR (δ ppm, CDCl₃, 400 MHz): δ 8.20-8.18 (m, 1H), 7.88-7.85 (m, 1H), 7.64-7.61 (m, 1H), 7.53-7.48 (m, 2H), 7.45-7.41 (m, 1H), 7.18-7.17 (m, 1H), 6.64-6.57 (m, 1H), 6.37-6.33 (m, 1H), 5.78-5.75 (m, 1H), 4.41 (bs, 2H), 4.34-4.27 (m, 2H), 3.84-3.73 (m, 4H), 3.44-3.34 (m, 6H), 3.12-3.08 (m, 1H), 2.88 (bs, 2H), 2.73-2.70 (m, 1H), 2.52 (s, 3H), 2.35-2.28 (m, 1H), 2.08-2.03 (m, 1H), 1.84-1.69 (m, 3H). MS (m/z): 537.24 [M+H]⁺. HPLC purity: 97.91%.

Example 56: 1-(4-acryloylpiperazin-1-yl)-6-(naphthalen-1-yl)-5,6,7,8-tetrahydro-2,6-naphthyridine-4-carbonitrile: Was prepared using the process as described above for Example 1 to get 60 mg of 1-(4-acryloylpiperazin-1-yl)-6-(naphthalen-1-yl)-5,6,7,8-tetrahydro-2,6-naphthyridine-4-carbonitrile. 1H-NMR (S ppm. CDCl₃, 400 MHz): δ 8.42 (s, 1H), 8.20-8.18 (m, 1H), 7.89-7.86 (m, 1H), 7.66-7.64 (m, 1H), 7.53-7.52 (m, 2H), 7.51-7.45 (m, 1H), 7.21-7.19 (m, 1H), 6.64-6.59 (m, 1H), 6.37-6.33 (m, 1H), 5.77-5.75 (m, 1H), 4.45 (bs, 2H), 3.86-3.75 (m, 4H), 3.45 (bs, 6H), 2.99 (bs, 2H). MS (m/z): 424.13 [M+H]⁺. HPLC purity: 99.08%.

Example 57: 1-(4-(2-fluoroacryloyl)piperazin-1-yl)-6-(naphthalen-1-yl)-3-(4-propionylpiperazin-1-yl)-5,6,7,8-tetrahydro-2,6-naphthyridine-4-carbonitrile: To a solution of 6-(naphthalen-1-yl)-1-(piperazin-1-yl)-3-(4-propionylpiperazin-1-yl)-5,6,7,8-tetrahydro-2,6-naphthyridine-4-carbonitrile hydrochloride (0.30 g, 0.54 mmol) in ethyl acetate (19 mL) at 0° C., was added triethylamine (0.62 mL, 4.4 mmol), 2-fluoroprop-2-enoic acid (0.086 mL, 1.1 mmol) and T3P (0.98 mL, 1.65 mmol, 50 wt. % in ethyl acetate). The mixture was warmed to 15° C. for 30 min and the reaction mixture was diluted with saturated solution of sodium carbonate (20 mL). The aqueous phase was extracted with ethyl acetate (2×20 mL) and the combined organic layer was dried over Na2SO4, filtered and concentrated to provide the crude residue. The residue was purified by column chromatography using 4% MeOH in DCM. The pure fractions were concentrated under reduced pressure to give 0.06 gm of 1-(4-(2-fluoroacryloyl)piperazin-1-yl)-6-(naphthalen-1-yl)-3-(4-propionylpiperazin-1-yl)-5,6,7,8-tetrahydro-2,6-naphthyridine-4-carbonitrile. 1H-NMR (δ ppm, CDCl₃, 400 MHz): δ 8.20-8.18 (m, 1H), 7.88-7.86 (m, 1H), 7.66-7.64 (m, 1H), 7.52-7.49 (m, 2H), 7.46-7.42 (m, 1H), 7.20-7.18 (m, 1H), 5.39-5.16 (m, 2H), 4.40 (bs, 2H), 3.77 (bs. 6H), 3.66-3.63 (m, 6H), 3.43 (bs, 6H), 2.88 (bs, 2H), 2.42-2.36 (m, 2H), 1.18-1.68 (m, 3H). MS (m/z): 582.26 [M+H]⁺. HPLC purity: 98.33%.

Other intermediates required to prepare any of the above examples and other illustrated compounds can be synthesized using the general and specific process as described herein above with all possible variation required to synthesize the said intermediates and compounds as envisaged herein.

### Part C: Biological Activity

The biological and/or pharmacological properties of the compounds of this invention may be confirmed by variety of assays. The biological and/or pharmacological assays which can be carried out with the compounds according to the invention and/or their pharmaceutically acceptable salts is exemplified below.

### Test 1: In Vitro Cell Proliferation Assay in NCI-H358 and MIAPACA-2 Cell Lines

Growth inhibition assays is to be carried out using 10% FBS supplemented media. Cells are to be seeded at a desired concentration of 1,500-6,000 cells/well in a 96-well plate. Test compounds at a concentration range from 1 to 30 uM will be added after 24 hours. Growth will be assessed using the CCK-8 kit for measuring reduction at 0 h (prior to the addition of the test compound) and 72 hours after the addition of test compound. Absorbance read on a BIO-RAD iMark Microplate or any equivalent microplate reader at a wavelength of 450 nm. Data was analysed and percent inhibition due to the test compound compared to the control is calculated accordingly.

TABLE 4

| % Inhibition of | |
|---|---|
| Cell Proliferation | |
| MiaPaca | H358 |

| Examples | TC1 | TC2 | TC1 | TC2 |
|---|---|---|---|---|
| Ex 1 | — | D | — | — |
| Ex 8 | B | D | — | — |
| Ex 9 | D | D | — | — |
| Ex 10 | D | D | — | — |
| Ex 11 | D | D | — | — |
| Ex 12 | C | D | — | — |
| Ex 13 | D | D | — | — |
| Ex 14 | D | D | — | — |
| Ex 15 | D | D | — | — |
| Ex 17 | D | D | — | — |
| Ex 18 | D | D | — | — |
| Ex 19 | D | D | — | — |
| Ex 20 | C | D | — | — |
| Ex 21 | C | C | — | — |
| Ex 23 | D | D | D | D |
| Ex 24 | D | D | D | D |
| Ex 25 | D | D | D | D |
| Ex 26 | — | — | B | D |
| Ex 27 | — | — | C | D |
| Ex 28 | D | D | C | D |
| Ex 32 | — | — | — | C |
| Ex 33 | — | — | — | A |
| Ex 34 | — | — | — | B |
| Ex 35 | — | — | — | B |
| Ex 36 | — | — | — | B |
| Ex 37 | — | — | — | D |
| Ex 38 | — | — | — | A |
| Ex 39 | — | — | — | B |
| Ex 40 | — | — | — | A |
| Ex 41 | — | — | — | B |
| Ex 42 | — | — | — | B |
| Ex 43 | — | — | — | B |
| Ex 44 | — | — | — | A |
| Ex 45 | — | — | — | A |
| Ex 46 | — | — | — | D |
| Ex 47 | — | — | — | D |
| Ex 48 | — | — | — | D |
| Ex 49 | — | — | — | D |
| Ex 50 | — | — | — | D |
| Ex 51 | — | — | — | C |
| Ex 52 | — | D | — | D |
| Ex 53 | — | D | — | D |
| Ex 54 | — | D | — | D |
| Ex 55 | — | D | — | D |
| Ex 56 | — | — | — | A |
| Ex 57 | — | — | — | D |

A = ≤25%;
B = >25 to ≤50;
C = >50 to ≤75;
D = >75% to 100%
TC1: Test Concentration of 3 uM & TC2: Test Concentration of 10 uM

### Test 2: In Vitro Cell Proliferation Assay in KRASG12 Cell Lines

Growth inhibition assays was carried out using 10% FBS supplemented media. Cells were seeded at a desired concentration of 1000-6000 cells/well in a well plate with desired number of wells. Test compounds at a desired concentration range were added after 24 hours. Growth was assessed using the Cell Titer-Glo (CTG. Cell Signaling) for measuring reduction at 0 h (prior to the addition of the test compound) and 3 day (2D) or 7 day (3D) after the addition of test compound. Absorbance read on a BIO-RAD iMark Microplate or any equivalent microplate reader at a predetermined a wavelength for e.g. 450 nm. Data was be analysed and percent inhibition and/or IC50 for each test compound is calculated accordingly. Results: Data provide for a representative Cpd A across various cell lines including selectivity

TABLE 5

| 3 D Cell Proliferation Assay | Cell Line | 3D IC50 |
|---|---|---|
| Effect of Cpd A KRAS G12 | NCI-H1373 | [symbol] [symbol] [symbol] |
| C Mutant Cell Line | NCI-H1792 | [symbol] [symbol] |
| | NCI-H23 | [symbol] [symbol] [symbol] |
| | Calu-1 | [symbol] [symbol] [symbol] |
| | SW-837 | [symbol] [symbol] [symbol] |
| | SW1463 | [symbol] [symbol] [symbol] [symbol] |
| | eCT26 | [symbol] [symbol] [symbol] [symbol] |
| | CO-04-0070* | [symbol] [symbol] [symbol] |
| Effect of Cpd B KRAS G12 | SW1463 | [symbol] |
| C Mutant Cell Line | eCT26 | [symbol] [symbol] |
| | CO-04-0070* | [symbol] [symbol] [symbol] |

*PDC; e: engineered for KRASG12C mutation [symbol]
= >1000 nM; [symbol] [symbol]
= <1000 to >200 nm; [symbol] [symbol] [symbol]
= <200 to >100 nm; [symbol] [symbol] [symbol] [symbol]
= <100 nM

TABLE 6
2D Cell Proliferation Assay

| | Cell Line | Fold Selectivity# |
|---|---|---|
| NON- KRAS | A549 | ΩΩΩ |
| G12C Mutant | HCT116 | Ω |
| Cell Line | | |

#Over KRAS G12C Mutant Cell Line
Ω = >100 fold;
ΩΩΩ >600 fold

### Test 3: Nucleotide Exchange Assay

### HTRF-Based Nucleotide Exchange Assay Detecting GTP Binding to K-Ras

A human KRAS G12C protein (corresponding to amino acid 2-169) was mixed with a a-GST Tb antibody (1.5× solution) and 10 uL of the solution was added to the reaction wells). Compounds (each 10 concentrations or any other concentration of choice at 3-fold or fold of choice for serial dilutions with a starting concentration of ˜300 um or 100 μM or 50 uM) were then delivered to the reaction wells using acoustic dispenser (Echo, Labcyte) and incubated with the Kras/aGST-Tb antibody for 1-hour at room temperature. After 1-hour incubation 5 μL of SOS1/GTP solution (SOS1—(corresponding to amino acid 564-1049) and GDP-DY-647P1 prepared using reaction buffer-20 mM Hepes, pH7.4, 150 mM Nacl, 5 mM MgCl2, 1 mM DTT, 0.05% BSA, 0.0025% NP40) was added to reaction wells to initiate the exchange reaction. HTRF based SOS1 mediated exchange of GDP to GTP was measured on a microplate reader PEHRAstar (BMG Labtech) at an excitation wavelength of 337 nm and emission wavelengths of 665 and 620 nm. No-SOS1 reaction or highest control compound concentration was used as blank and % inhibition was calculated and/or IC50 was determined using Sigmoidal dose response (variable slope) equation. Representative compound Cpd A showed a IC50 of <10 nM.

Test 4: Protein Protein Interaction Assay (PPI Assay): HTRF Based PPI Assay Detecting Binding of KRAS to cRAF Protein (KRAS:cRAF)

5 μL of 3× Human KRAS G12C protein (b-Kras G12C (GppNHp); corresponding to amino acid 2-169) was delivered to assay wells. Compounds (tested in 10-concentration IC50 mode with 3-fold serial dilution at a starting concentration of 10 μM in duplicate) were added to assay wells using acoustic technology (ECHO, Labcyte) followed by 30-minute incubation at room temperature. Following incubation 5 uL of 3× cRAF protein (GST-cRAF corresponding to amino acid 2-303)) was added to assay wells. After 30 minutes of incubation 5 uL of 3× detection mix containing MAb Anti GST-Tb (Cisbio 61GSTTLB) and Streptavidin-XL665 (Cisbio 610SAXLB) cryptate is added to assay wells. After 60-120 minutes of incubation HTRF based signal was measured on a microplate reader PEHRAstar (BMG Labtech) at an excitation wavelength of 337 nm and emission wavelengths of 665 and 620 nm. IC50 was determined using Sigmoidal dose response (variable slope) equation when the activities at the highest concentration of compounds were less than 65%.

### Test 5: Biomarker Evaluation in NCI-H358 and MIA PaCa-2 Cells Using Western Blotting

Protein lysate were prepared using RIPA lysis buffer representing both the control and test samples. In the instant invention Protein lysate were prepared using NCI-H358 and MIA PaCa-2 cells treated with representative example of the invention over time course using a 9 point concentration-response for measuring modulation of pERK. Total Protein was estimated by Bradford method and absorbance is measured at 595 nm using Bio-Rad imark reader. The total proteins isolated were separated on a 10% SDS PAGE electrophoresis and transferred on to a Nitrocellulose membrane. After transfer, the membrane was blocked using 5% BSA prepared in PBST (0.1% tween-20) for 1 hr at room temperature and washed with 1×PBS and PBST. Membrane was then probed with Rabbit Monoclonal Primary antibody such as pERK (MA5-15173), ERK(MA5-15134), procured from Invitrogen™. ThermoFisher Scientific USA. Primary antibody (1:2000 dilution) prepared in 5% BSA, 0.1% tween-20 solution for overnight at 4° C. Following incubation with primary antibody membrane were washed thrice with 1×PBS and PBST followed by incubation with Goat Anti-Rabbit Secondary IgG HRP conjugated (from Invitrogen™) at (1:10,000 dilutions, prepared in 3% skim milk, 0.1% tween-20) for 1 hr at room temperature. Following incubation, the blot is washed and developed using G-biosciences femto LUCENT™ PLUS-HRP Chemiluminescent reagent in the Bio-Rad Chemidoc™ Imager system. The resulted bands on the blot were quantified using the Image J software.

Results: Representative compound tested demonstrated a dose and time dependent modulation of pERK, downstream biomarker representing KRAS signaling.

TABLE 7

| BIOMARKER | IC50 (nM) | pERK/β-actin | | | pERK/ERK | | |
|---|---|---|---|---|---|---|---|
| EVALUATION | Time | T1 | T2 | T3 | T1 | T2 | T3 |
| KRAS G12 C | NCI-H358 | Ω | Ω Ω Ω Ω | Ω Ω | Ω | Ω Ω Ω | Ω Ω Ω |
| Mutant Cell Line | MIA PaCa-2 | Ω Ω | Ω Ω Ω | Ω Ω | Ω | Ω Ω Ω | Ω Ω Ω |

Ω Ω Ω Ω = <5 nm;
Ω Ω Ω = >5 nm to 25 nM;
Ω Ω = >25 to 50 nM;
Ω = >50 to 100 nM
T1 = 3 h;
T2 = 6 h;
T3 = 24 h

### Test 6: Anti-Tumor Effect of Compound of the Invention in Female Balb/c Nude Mice Bearing NCI-H358 Human Non-Small Cell Lung Cancer Xenograft

NCI-H358 Xenograft was used to test representative compound of the invention where in NCI-H358 cells were inoculated in a mice model representing Lung Cancer with two different cohorts/group comprising of Control (Vehicle treated, GI) and two treatment cohorts/group namely Paclitaxel as standard (10 mg/kg Q3D, IV, G3) and test compound (100 mg/kg QD, Cpd A, G3) with each cohorts/group. Treatment was initiated when maximum tumor growth reached >200 mm3 and continued till the Vehicle are reached a maximum tumor volume of 2000 mm3.

### Results:

Test compound showed a Tumor Growth inhibition (TGI) of >65% with statistically significant (P<0.01\hat{ }) reduction in tumor volume noticed from day 3 onwards (n=5). Test compound showed statistically significant reduction in tumour volume as compared to Paclitaxel@ 10 mg/kg Q3DX3 Weeks, IV. No significant effect was seen on overall body weight over the 21-day study period.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance

1. (canceled)

2. A compound of formula (A-I) 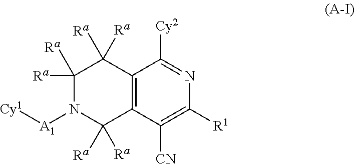
or a tautomer thereof, isotope thereof, prodrug thereof, N-oxide thereof, a pharmaceutically acceptable ester thereof, a pharmaceutically acceptable salt thereof, or a stereoisomer thereof, wherein
A₁ is absent or is independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₚ—, —O—, —S—, —S(═O)ₚ—, —C(═O)—, —NR^{x}—, —CO—NR^{x}— and —NR^{x}—CO—;
Cy¹ is selected form a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
Cy² is selected from cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl which is optionally substituted by E;
E is a group capable of forming a covalent bond or an “Electrophile” or “electrophilic moiety capable of forming a covalent bond;
R¹ at each occurrence is independently selected from hydrogen, hydroxy, halogen, substituted or unsubstituted amino, substituted or unsubstituted alkyl, substituted or unsubstituted alkoxy, substituted or unsubstituted alkenyl, substituted or unsubstituted alkynyl, substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl, C(═O)OR^{b}, —C(═O)R^{b}, —C(═S)R^{b}, —C(═O)NR^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)N^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
R^{a} at each occurrence is independently selected from hydrogen, hydroxy, halogen, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}R^{c}, —NR^{b}—OR^{c}, —NR^{b}C(═O)R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z} or any two R^{a} may be joined to a form a substituted or unsubstituted saturated or unsaturated 3-6 member ring, which may optionally include heteroatoms which may be same or different and are selected from O, NR^{a} or S or; any two R^{a} attached to the same carbon atom may be joined to a form a Oxo (C═O), Imino (═NR^{b}), C═S(O)ₚ, or substituted or unsubstituted saturated or unsaturated 3-6 member ring, which may optionally include heteroatoms which may be same or different and are selected from O, NR^{x} or S;
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{x} and R^{y} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio(═S) or imino(═NR^{x}) group; and
each occurrence of p is independently 0, 1 or 2, with the proviso that,
when Cy² is Morpholine, Piperidine-1-yl or 3,3-dimethylpiperidine-1-yl and A₁-Cy¹ is Benzyl or Cyclohexane then R¹ cannot be—Chloro, Morpholine, Piperidine-1-yl, N-benzyl amine, or N-cyclohexyl amine or −3 amino allyl.

3. (canceled)

4. (canceled)

5. (canceled)

6. (canceled)

7. (canceled)

8. (canceled)

9. (canceled)

10. (canceled)

11. The compound of claim 2, wherein Cy¹ is selected form a cyclic group selected from a group consisting of substituted or unsubstituted heterocyclyl, substituted or unsubstituted aryl, substituted or unsubstituted arylalkyl, substituted or unsubstituted heteroaryl, and substituted or unsubstituted heteroarylalkyl.

12. The compound of claim 2, wherein Cy¹ is selected form a cyclic group selected from substituted or unsubstituted aryl and substituted or unsubstituted heteroaryl.

13. The compound of claim 2, wherein Cy¹ is selected form the group consist of:

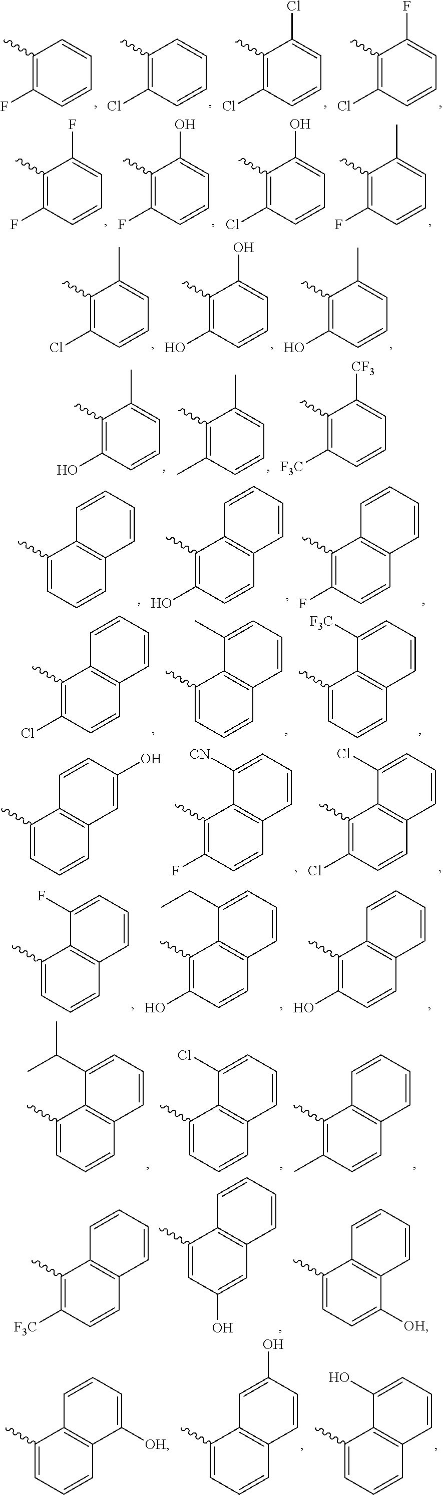

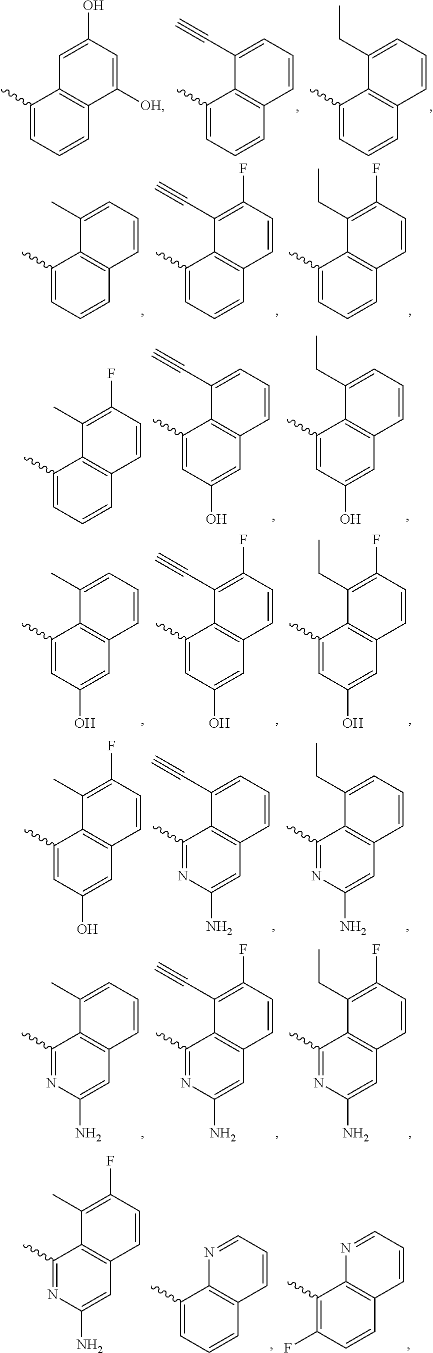

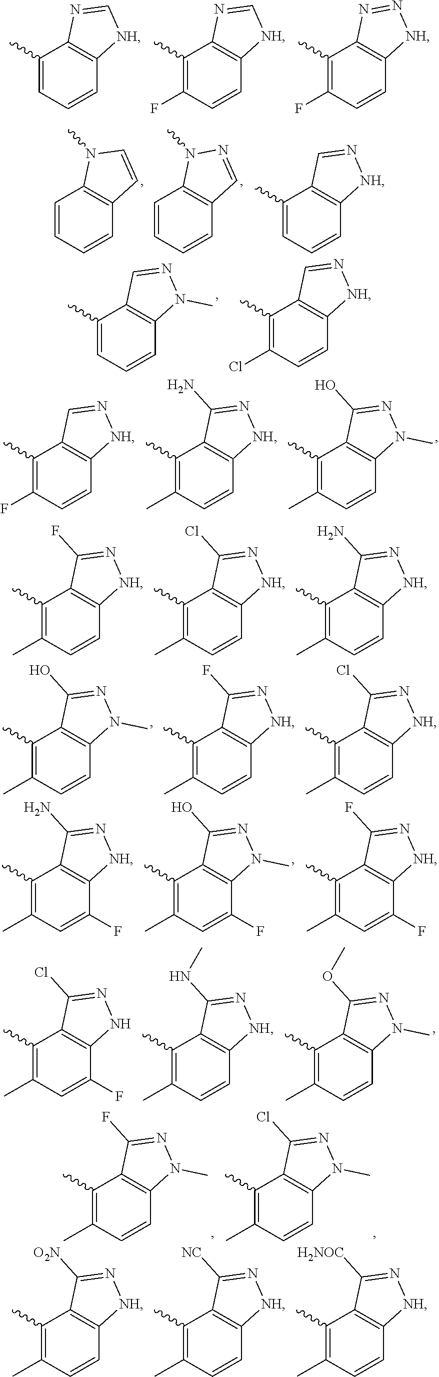

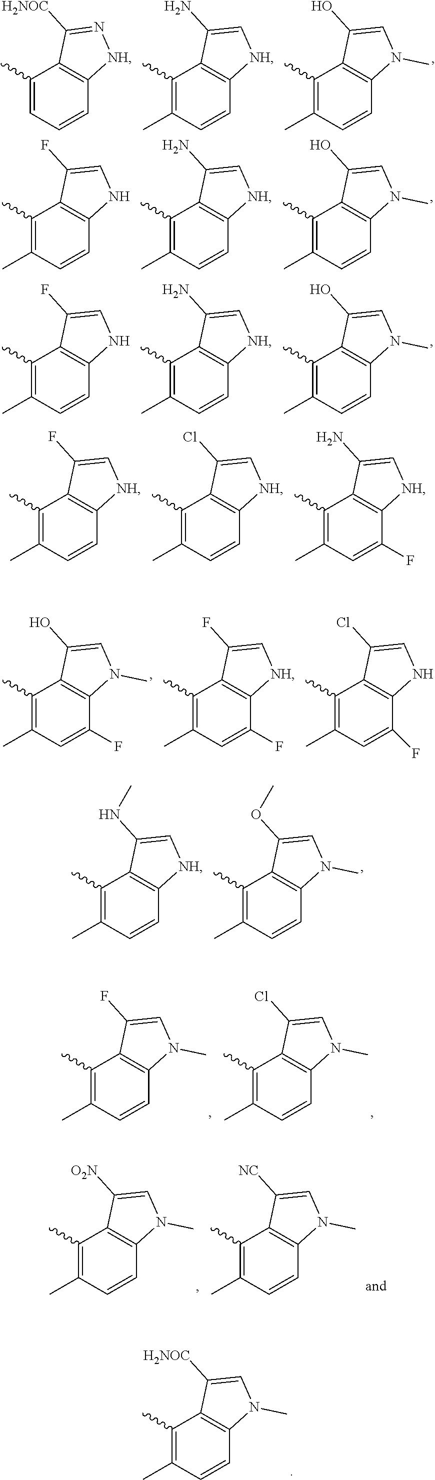

14. (canceled)

15. The compound of claim 2, wherein Cy² is selected from cyclic group selected from substituted or unsubstituted heterocyclyl and substituted or unsubstituted heterocyclealkyl, wherein each group is optionally further substituted with group E.

16. The compound of claim 2, wherein Cy² is selected form the group consisting of: 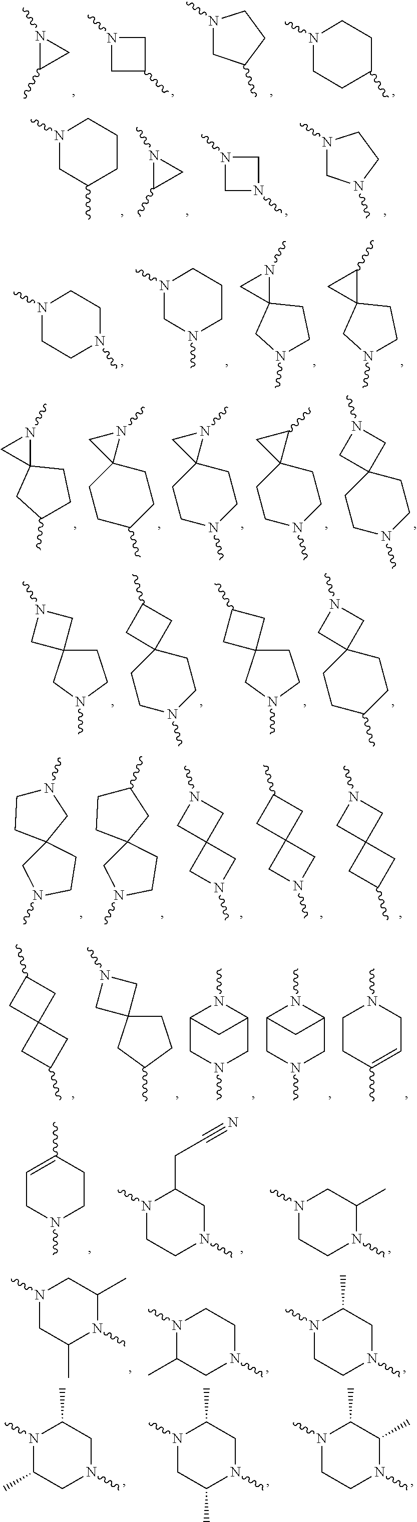 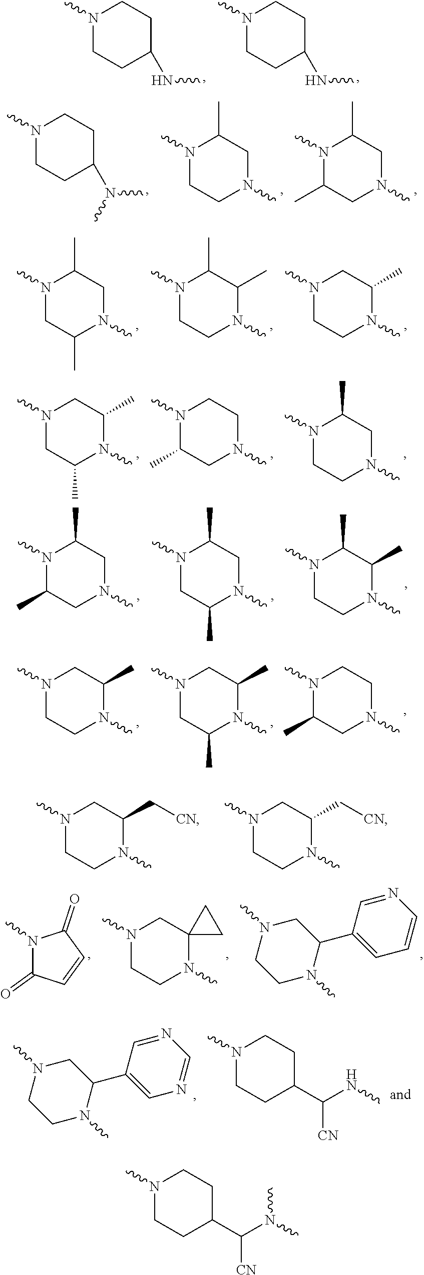
substituted with E a group capable of forming a covalent bond.

17. The compound of claim 2, wherein E is selected from the group consisting of:

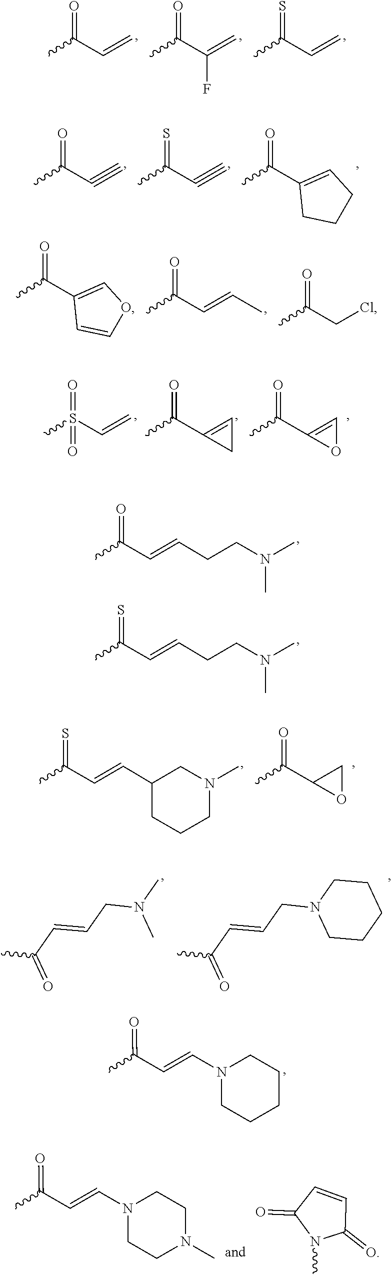

18. (canceled)

19. The compound of claim 2, wherein A₂-Cy²-E is selected from the group consisting of:

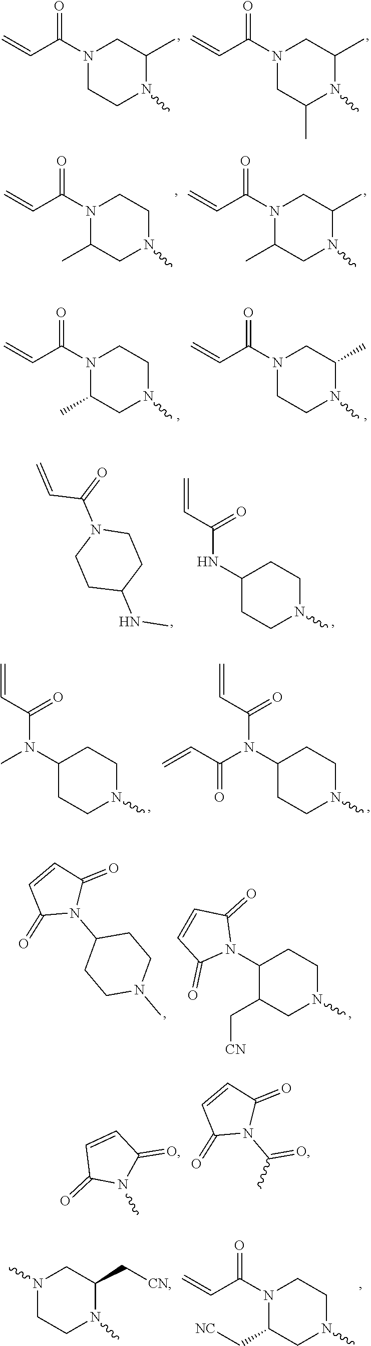

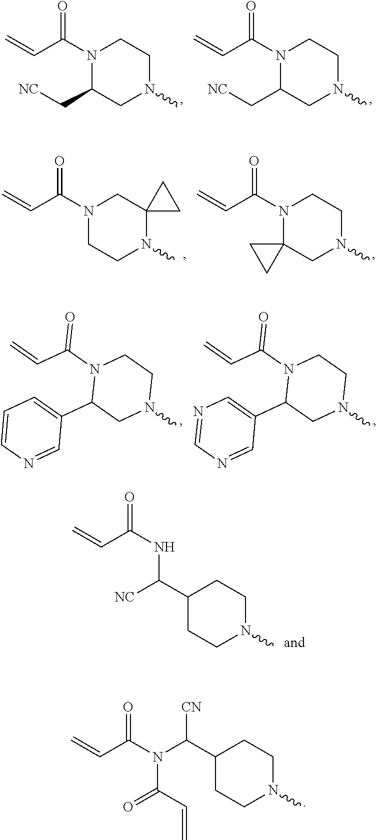

20. (canceled)

21. (canceled)

22. The compound of claim 2, wherein
i) R¹ is selected from hydrogen, halogen, OR^{b}, S—R^{b}, —S(═O)pR^{b}—, —C(═O)—R^{b}, —NR^{b}R^{c}, —CO—NR^{b}R^{c}— and —NR^{b}—CO—R^{c}; or
ii) R¹ is —O—R^{b} or
iii) R¹ is —NR^{b}R^{c}
iv) R¹ is independently selected from, substituted or unsubstituted alkyl, substituted or unsubstituted alkoxy, substituted or unsubstituted heterocyclyl, substituted or unsubstituted heterocyclylalkyl, substituted or unsubstituted heteroaryl, substituted or unsubstituted heteroarylalkyl or
v) R¹ is independently selected from substituted or unsubstituted alkyl, substituted or unsubstituted heterocyclyl, substituted or unsubstituted heterocyclylalkyl and substituted or unsubstituted heteroaryl.

23. (canceled)

24. (canceled)

25. (canceled)

26. (canceled)

27. The compound of claim 2, wherein R¹ is independently selected from the group consisting of:

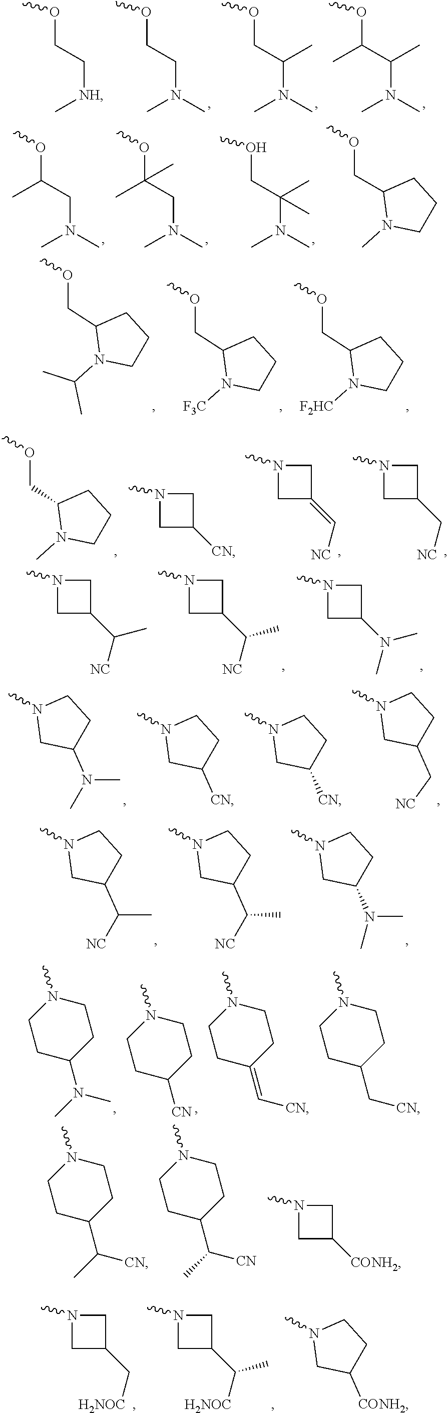

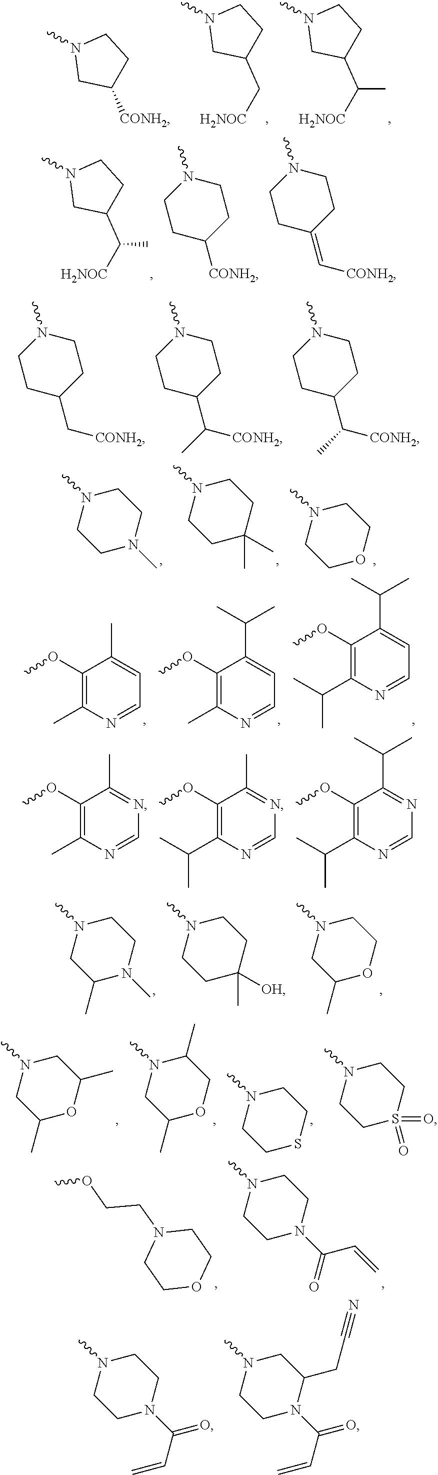

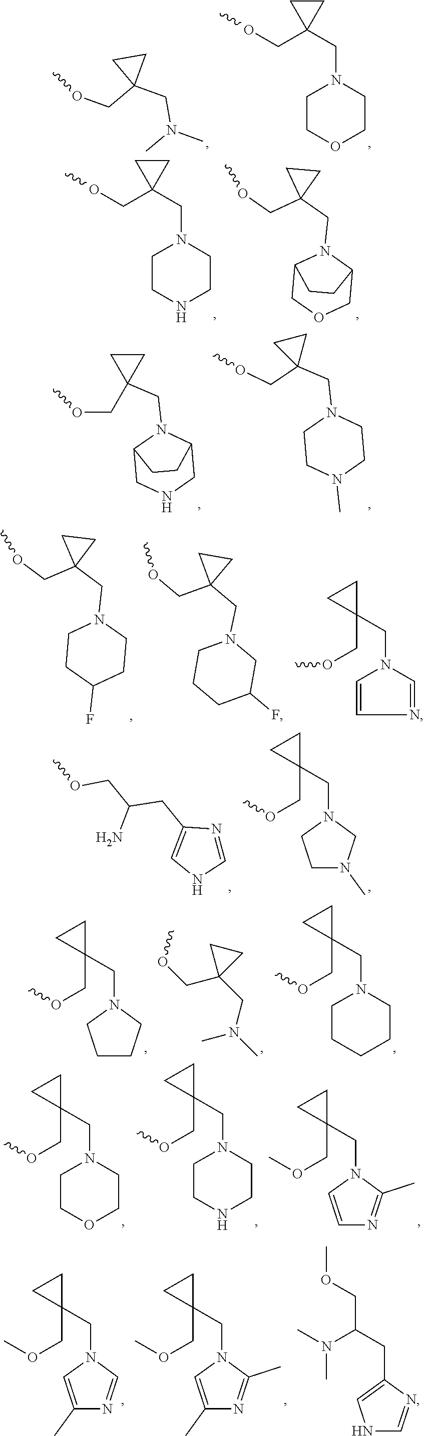

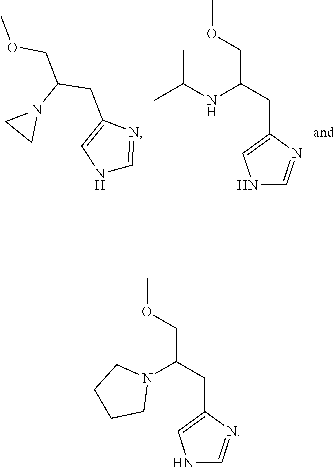

28. (canceled)

29. (canceled)

30. The compound of claim 2, having a compound of formula (A-III) 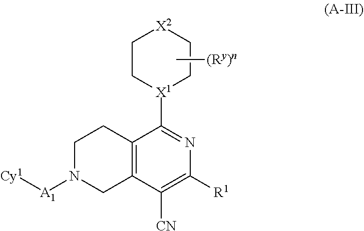
or a tautomer thereof, isotope thereof, prodrug thereof, N-oxide thereof, a pharmaceutically acceptable ester thereof, a pharmaceutically acceptable salt thereof, or a stereoisomer thereof, wherein
A₁ is absent or substituted or unsubstituted alkyl;
Cy¹ is selected from substituted or unsubstituted aryl or substituted or unsubstituted heteroaryl;
R¹ is selected from hydrogen, halogen, substituted or unsubstituted alkyl, —NR^{b}R^{c} or —OR^{b};
each occurrence of R^{b} and R^{c} are independently selected from hydrogen, substituted or unsubstituted alkyl, or variables of R^{b} and R^{c} together with the nitrogen which they attached can form a substituted or unsubstituted heterocyclic ring;
X¹ is C or N;
X² is selected from —N-E or CH₂-E, O or S;
E is a group capable of forming a covalent bond or an “Electrophile” or “electrophilic moiety capable of forming a covalent bond.
R^{y} is selected from hydrogen, halogen, substituted or unsubstituted alkyl; and
n is 0, 1, 2, 3, 4, 5, 6, 7 or 8.

31. (canceled)

32. The compound of claim 30, wherein A₁ is absent.

33. The compound of claim 30, wherein
i) Cy¹ is selected from substituted or unsubstituted aryl; or
ii) Cy¹ is selected from phenyl or naphthalene, optionally substituted with halogen, hydroxy or substituted or unsubstituted alkyl; or
iii) Cy¹ is selected from quinoline or quinazoline, optionally substituted with halogen, hydroxy or substituted or unsubstituted alkyl.

34. (canceled)

35. (canceled)

36. The compound of claim 30, wherein R¹ is selected from hydrogen, halogen, 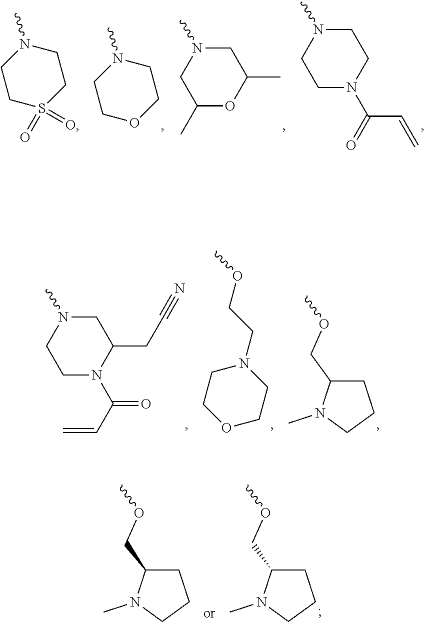
X¹ is N and
X² is selected from —N-E or CH₂-E or O, wherein E is selected from

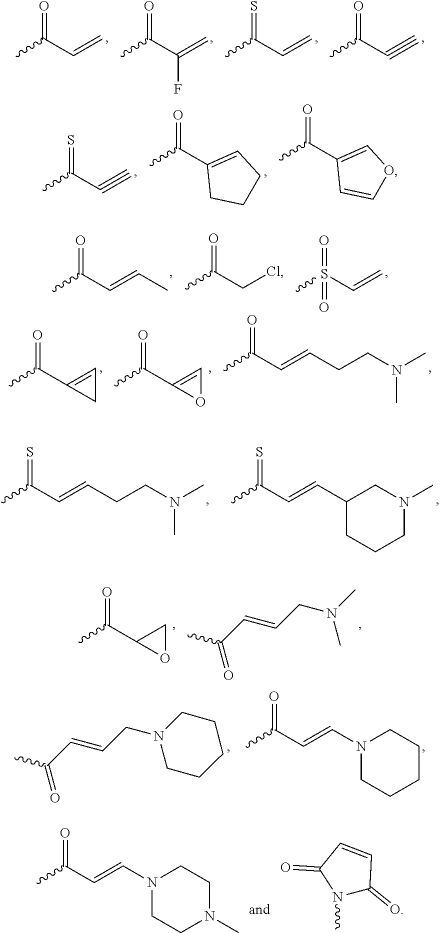

37. (canceled)

38. (canceled)

39. (canceled)

40. The compound of claim 30, wherein
A₁ is absent or methyl;
Cy¹ is selected from substituted or unsubstituted aryl;
R¹ is selected from hydrogen, halogen, 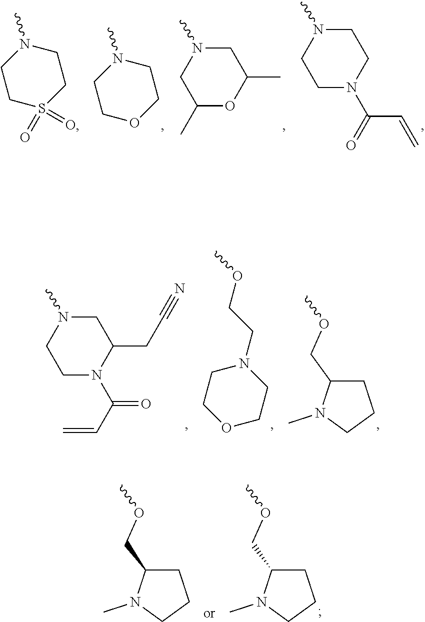
X¹ is N; and
X² is selected from —N-E or CH₂-E or O, wherein E is selected from the group consisting of:

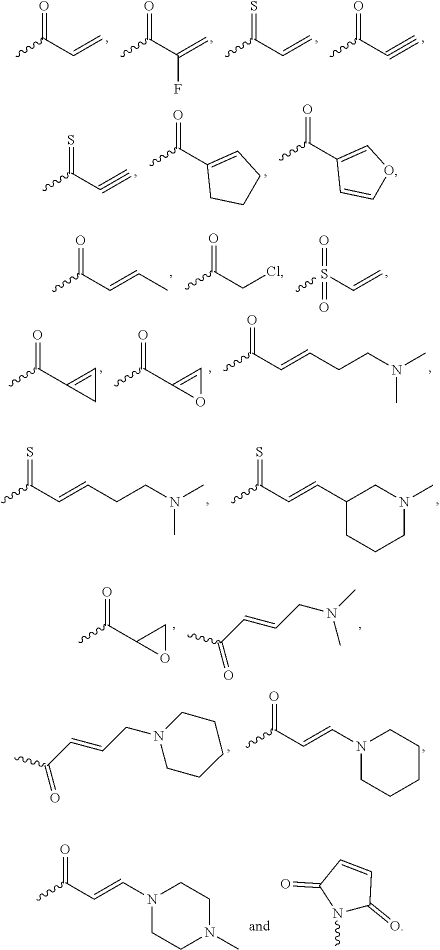

41. The compound of claim 2 selected from the group consisting of:
1-(4-acryloylpiperazin-1-yl)-3-(1,1-dioxidothiomorpholino)-6-(naphthalen-1-yl)-5,6,7,8-tetrahydro-2,6-naphthyridine-4-carbonitrile;
1-(4-acryloylpiperazin-1-yl)-6-(8-chloronaphthalen-1-yl)-3-morpholino-5,6,7,8-tetrahydro-2,6-naphthyridine-4-carbonitrile;
1-(4-acryloylpiperazin-1-yl)-3-(2-morpholinoethoxy)-6-(naphthalen-1-yl)-5,6,7,8-tetrahydro-2,6-naphthyridine-4-carbonitrile;
1-(4-acryloylpiperazin-1-yl)-6-(8-chloronaphthalen-1-yl)-3-(2-orpholinoethoxy)-5,6,7,8-tetrahydro-2,6-naphthyridine-4-carbonitrile;
3-(4-acryloylpiperazin-1-yl)-1-(2,6-dimethylmorpholino)-6-(naphthalen-1-yl)-5,6,7,8-tetrahydro-2,6-naphthyridine-4-carbonitrile;
3-(4-acryloylpiperazin-1-yl)-6-(8-chloronaphthalen-1-yl)-1-(2,6-dimethylmorpholino)-5,6,7,8-tetrahydro-2,6-naphthyridine-4-carbonitrile;
3-(4-acryloyl-3-(cyanomethyl)piperazin-1-yl)-1-(2,6-dimethylmorpholino)-6-(naphthalen-1-yl)-5,6,7,8-tetrahydro-2,6-naphthyridine-4-carbonitrile;
(S)-1-(4-acryloylpiperazin-1-yl)-6-(8-chloronaphthalen-1-yl)-3-((1-methylpyrrolidin-2-yl)methoxy)-5,6,7,8-tetrahydro-2,6-naphthyridine-4-carbonitrile;
(S)-1-(4-acryloylpiperazin-1-yl)-6-(3-hydroxynaphthalen-1-yl)-3-((1-methylpyrrolidin-2-yl)methoxy)-5,6,7,8-tetrahydro-2,6-naphthyridine-4-carbonitrile;
(S)-1-(4-(2-fluoroacryloyl)piperazin-1-yl)-3-((1-methylpyrrolidin-2-yl)methoxy)-6-(quinazolin-4-yl)-5,6,7,8-tetrahydro-2,6-naphthyridine-4-carbonitrile;
(S)-6-(8-chloronaphthalen-1-yl)-3-((1-methylpyrrolidin-2-yl)methoxy)-1-(4-(vinylsulfonyl)piperazin-1-yl)-5,6,7,8-tetrahydro-2,6-naphthyridine-4-carbonitrile;
6-(8-chloronaphthalen-1-yl)-1-(4-(2-fluoroacryloyl)-3-methylpiperazin-1-yl)-3-(((S)-1-methylpyrrolidin-2-yl)methoxy)-5,6,7,8-tetrahydro-2,6-naphthyridine-4-carbonitrile.
1-(4-acryloyl-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile;
1-((S)-4-acryloyl-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile;
1-((R)-4-acryloyl-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile;
1-(4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile;
1-((S)-4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile;
1-((R)-4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile;
1-(4-acryloyl-3-(cyanomethyl)piperazin-1-yl)-6-(3-hydroxynaphthalen-1-yl)-3-(((S)-1-methylpyrrolidin-2-yl)methoxy)-5,6,7,8-tetrahydro-2,6-naphthyridine-4-carbonitrile;
1-(3-(cyanomethyl)-4-(2-fluoroacryloyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile;
(S)-1-(4-acryloylpiperazin-1-yl)-6-(isoquinolin-4-yl)-3-((1-methylpyrrolidin-2-yl)methoxy)-5,6,7,8-tetrahydro-2,6-naphthyridine-4-carbonitrile;
(S)-1-(4-acryloylpiperazin-1-yl)-3-((1-methylpyrrolidin-2-yl)methoxy)-6-(quinolin-8-yl)-5,6,7,8-tetrahydro-2,6-naphthyridine-4-carbonitrile;
1-(4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
1-((R)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
1-((S)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
6-(8-chloronaphthalen-1-yl)-1-(3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile;
6-(8-chloronaphthalen-1-yl)-1-((S)-3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile;
6-(8-chloronaphthalen-1-yl)-1-((R)-3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile;
1-(4-acryloylpiperazin-1-yl)-6-benzyl-3-chloro-5,6,7,8-tetrahydro-2,6-naphthyridine-4-carbonitrile;
N-(1-(6-benzyl-3-chloro-4-cyano-5,6,7,8-tetrahydro-2,6-naphthyridin-1-yl)piperidin-4-yl)acrylamide;
N-(1-(6-benzyl-4-cyano-5,6,7,8-tetrahydro-2,6-naphthyridin-1-yl)piperidin-4-yl)acrylamide;
1,3-bis(4-acryloylpiperazin-1-yl)-6-benzyl-5,6,7,8-tetrahydro-2,6-naphthyridine-4-carbonitrile;
1-(4-acryloylpiperazin-1-yl)-6-benzyl-4-cyano-5,6,7,8-tetrahydro-2,6-naphthyridin-3-yl acrylate;
1-(4-acryloylpiperazin-1-yl)-6-benzyl-3-morpholino-5,6,7,8-tetrahydro-2,6-naphthyridine-4-carbonitrile;
1-(4-acryloylpiperazin-1-yl)-6-benzyl-3-(4-methylpiperazin-1-yl)-5,6,7,8-tetrahydro-2,6-naphthyridine-4-carbonitrile;
1-(4-acryloyl-3-(cyanomethyl)piperazin-1-yl)-6-benzyl-3-(4-methylpiperazin-1-yl)-5,6,7,8-tetrahydro-2,6-naphthyridine-4-carbonitrile;
6-(1-naphthoyl)-1-(4-acryloyl-3-(cyanomethyl)piperazin-1-yl)-3-(4-methylpiperazin-1-yl)-5,6,7,8-tetrahydro-2,6-naphthyridine-4-carbonitrile;
(S)-1-(4-acryloyl-2-methylpiperazin-1-yl)-6-benzyl-3-(4-methylpiperazin-1-yl)-5,6,7,8-tetrahydro-2,6-naphthyridine-4-carbonitrile;
(R)-1-(4-acryloyl-2-methylpiperazin-1-yl)-6-benzyl-3-(4-methylpiperazin-1-yl)-5,6,7,8-tetrahydro-2,6-naphthyridine-4-carbonitrile;
1-(4-acryloylpiperazin-1-yl)-6-benzyl-3-hydroxy-5,6,7,8-tetrahydro-2,6-naphthyridine-4-carbonitrile;
1-(4-acryloylpiperazin-1-yl)-3-(4-methylpiperazin-1-yl)-6-(naphthalen-1-yl)-5,6,7,8-tetrahydro-2,6-naphthyridine-4-carbonitrile;
1-(4-acryloylpiperazin-1-yl)-6-benzyl-3-(piperidin-1-yl)-5,6,7,8-tetrahydro-2,6-naphthyridine-4-carbonitrile;
1-(4-acryloylpiperazin-1-yl)-6-benzyl-3-(2-fluoropyridin-4-yl)-5,6,7,8-tetrahydro-2,6-naphthyridine-4-carbonitrile;
1-(4-acryloylpiperazin-1-yl)-6-(naphthalen-1-yl)-3-(piperidin-1-yl)-5,6,7,8-tetrahydro-2,6-naphthyridine-4-carbonitrile;
1-(4-acryloylpiperazin-1-yl)-6-benzyl-3-((1-methylpyrrolidin-2-yl)methoxy)-5,6,7,8-tetrahydro-2,6-naphthyridine-4-carbonitrile;
1,3-bis(4-acryloylpiperazin-1-yl)-6-(naphthalen-1-yl)-5,6,7,8-tetrahydro-2,6-naphthyridine-4-carbonitrile;
1,3-bis(4-acryloylpiperazin-1-yl)-6-(benzo[b]thiophen-4-yl)-5,6,7,8-tetrahydro-2,6-naphthyridine-4-carbonitrile;
1-(4-acryloylpiperazin-1-yl)-6-(naphthalen-1-yl)-3-(4-propionylpiperazin-1-yl)-5,6,7,8-tetrahydro-2,6-naphthyridine-4-carbonitrile;
3-(4-acryloylpiperazin-1-yl)-6-(naphthalen-1-yl)-1-(4-propionylpiperazin-1-yl)-5,6,7,8-tetrahydro-2,6-naphthyridine-4-carbonitrile;
1-(4-acryloylpiperazin-1-yl)-3-morpholino-6-(naphthalen-1-yl)-5,6,7,8-tetrahydro-2,6-naphthyridine-4-carbonitrile;
3-(4-acetylpiperazin-1-yl)-1-(4-acryloylpiperazin-1-yl)-6-(naphthalen-1-yl)-5,6,7,8-tetrahydro-2,6-naphthyridine-4-carbonitrile;
1-(4-acryloylpiperazin-1-yl)-3-(2,6-dimethylmorpholino)-6-(naphthalen-1-yl)-5,6,7,8-tetrahydro-2,6-naphthyridine-4-carbonitrile;
1-(4-acryloylpiperazin-1-yl)-3-(4-(methylsulfonyl)piperazin-1-yl)-6-(naphthalen-1-yl)-5,6,7,8-tetrahydro-2,6-naphthyridine-4-carbonitrile;
1-(4-acryloylpiperazin-1-yl)-3-(4-ethylpiperazin-1-yl)-6-(naphthalen-1-yl)-5,6,7,8-tetrahydro-2,6-naphthyridine-4-carbonitrile;
(S)-1-(4-acryloylpiperazin-1-yl)-3-((1-methylpyrrolidin-2-yl)methoxy)-6-(naphthalen-1-yl)-5,6,7,8-tetrahydro-2,6-naphthyridine-4-carbonitrile;
1-(4-acryloylpiperazin-1-yl)-6-(naphthalen-1-yl)-5,6,7,8-tetrahydro-2,6-naphthyridine-4-carbonitrile;
1-(4-(2-fluoroacryloyl)piperazin-1-yl)-6-(naphthalen-1-yl)-3-(4-propionylpiperazin-1-yl)-5,6,7,8-tetrahydro-2,6-naphthyridine-4-carbonitrile; and
a pharmaceutically acceptable salts thereof.

42. A compound selected from the group consisting of:
1-(4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
1-((R)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
1-((S)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
6-(8-chloronaphthalen-1-yl)-1-(3-(cyanomethyl)-4-(2-fluoroacryloyl)piperazin-1-yl)-3-(((S)-1-methylpyrrolidin-2-yl)methoxy-5,6,7,8-tetrahydro-2,6-naphthyridine-4-carbonitrile;
6-(8-chloronaphthalen-1-yl)-1-((S)-3-(cyanomethyl)-4-(2-fluoroacryloyl)piperazin-1-yl)-3-(((S)-1-methylpyrrolidin-2-yl)methoxy-5,6,7,8-tetrahydro-2,6-naphthyridine-4-carbonitrile; and
6-(8-chloronaphthalen-1-v)-1-((R)-3-(cyanomethyl)-4-(2-fluoroacryloyl)piperazin-1-yl)-3-(((S)-1-methylpyrrolidin-2-yl)methoxy-5,6,7,8-tetrahydro-2,6-naphthyridine-4-carbonitrile; and
a pharmaceutically acceptable salt thereof.

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. A Pharmaceutical composition comprising a compound of claim 2, or a pharmaceutically acceptable salt thereof, and a pharmaceutically acceptable carrier.

55. A method for treating cancer in a subject in need thereof, the method comprising administering to a subject in need thereof a therapeutically effective amount of a compound of claim 2, or a pharmaceutically acceptable salt thereof.

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. A compound selected from the group consisting of:
1-(4-acryloylpiperazin-1-yl)-3-(1,1-dioxidothiomorpholino)-6-(naphthalen-1-yl)-5,6,7,8-tetrahydro-2,6-naphthyridine-4-carbonitrile;
1-(4-acryloylpiperazin-1-yl)-6-(8-chloronaphthalen-1-yl)-3-morpholino-5,6,7,8-tetrahydro-2,6-naphthyridine-4-carbonitrile;
1-(4-acryloylpiperazin-1-yl)-3-(2-morpholinoethoxy)-6-(naphthalen-1-yl)-5,6,7,8-tetrahydro-2,6-naphthyridine-4-carbonitrile;
1-(4-acryloylpiperazin-1-yl)-6-(8-chloronaphthalen-1-yl)-3-(2-orpholinoethoxy)-5,6,7,8-tetrahydro-2,6-naphthyridine-4-carbonitrile;
3-(4-acryloylpiperazin-1-yl)-1-(2,6-dimethylmorpholino)-6-(naphthalen-1-yl)-5,6,7,8-tetrahydro-2,6-naphthyridine-4-carbonitrile;
3-(4-acryloylpiperazin-1-yl)-6-(8-chloronaphthalen-1-yl)-1-(2,6-dimethylmorpholino)-5,6,7,8-tetrahydro-2,6-naphthyridine-4-carbonitrile;
3-(4-acryloyl-3-(cyanomethyl)piperazin-1-yl)-1-(2,6-dimethylmorpholino)-6-(naphthalen-1-yl)-5,6,7,8-tetrahydro-2,6-naphthyridine-4-carbonitrile;
(S)-1-(4-acryloylpiperazin-1-yl)-6-(8-chloronaphthalen-1-yl)-3-((1-methylpyrrolidin-2-yl)methoxy)-5,6,7,8-tetrahydro-2,6-naphthyridine-4-carbonitrile;
(S)-1-(4-acryloylpiperazin-1-yl)-6-(3-hydroxynaphthalen-1-yl)-3-((1-methylpyrrolidin-2-yl)methoxy)-5,6,7,8-tetrahydro-2,6-naphthyridine-4-carbonitrile;
(S)-1-(4-(2-fluoroacryloyl)piperazin-1-yl)-3-((1-methylpyrrolidin-2-yl)methoxy)-6-(quinazolin-4-yl)-5,6,7,8-tetrahydro-2,6-naphthyridine-4-carbonitrile;
(S)-6-(8-chloronaphthalen-1-yl)-3-((1-methylpyrrolidin-2-yl)methoxy)-1-(4-(vinylsulfonyl)piperazin-1-yl)-5,6,7,8-tetrahydro-2,6-naphthyridine-4-carbonitrile;
6-(8-chloronaphthalen-1-yl)-1-(4-(2-fluoroacryloyl)-3-methylpiperazin-1-yl)-3-(((S)-1-methylpyrrolidin-2-yl)methoxy)-5,6,7,8-tetrahydro-2,6-naphthyridine-4-carbonitrile;
1-(4-acryloyl-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile;
1-((S)-4-acryloyl-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile;
1-((R)-4-acryloyl-3-(cyanomethyl)piperazin-1-yl)-3-(2,6-dimethylmorpholino)-6-(naphthalen-1-yl)-5,6,7,8-tetrahydro-2,6-naphthyridine-4-carbonitrile;
1-(4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile;
1-((S)-4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile;
1-((R)-4-acryloyl-3-(cyanomethyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile;
1-(4-acryloyl-3-(cyanomethyl)piperazin-1-yl)-6-(3-hydroxynaphthalen-1-yl)-3-(((S)-1-methylpyrrolidin-2-yl)methoxy)-5,6,7,8-tetrahydro-2,6-naphthyridine-4-carbonitrile;
1-(3-(cyanomethyl)-4-(2-fluoroacryloyl)piperazin-1-yl)-3-(((S)-1-methylpyrrolidin-2-yl)methoxy)-6-(naphthalen-1-yl)-5,6,7,8-tetrahydro-2,6-naphthyridine-4-carbonitrile;
(S)-1-(4-acryloylpiperazin-1-yl)-6-(isoquinolin-4-yl)-3-((1-methylpyrrolidin-2-yl)methoxy)-5,6,7,8-tetrahydro-2,6-naphthyridine-4-carbonitrile;
(S)-1-(4-acryloylpiperazin-1-yl)-3-((1-methylpyrrolidin-2-yl)methoxy)-6-(quinolin-8-yl)-5,6,7,8-tetrahydro-2,6-naphthyridine-4-carbonitrile;
1-(4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
1-((R)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
1-((S)-4-acryloyl-3-(cyanomethyl)piperazin-1-yl)-6-(8-chloronaphthalen-1-yl)-3-(((S)-1-methylpyrrolidin-2-yl)methoxy)-5,6,7,8-tetrahydro-2,6-naphthyridine-4-carbonitrile;
6-(8-chloronaphthalen-1-yl)-1-(3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile;
6-(8-chloronaphthalen-1-yl)-1-((S)-3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile;
6-(8-chloronaphthalen-1-yl)-1-((R)-3-(cyanomethyl)-4-(2-fluoroacryloyl)piperazin-1-yl)-3-(((S)-1-methylpyrrolidin-2-yl)methoxy)-5,6,7,8-tetrahydro-2,6-naphthyridine-4-carbonitrile;
1-(4-acryloylpiperazin-1-yl)-3-(4-methylpiperazin-1-yl)-6-(naphthalen-1-yl)-5,6,7,8-tetrahydro-2,6-naphthyridine-4-carbonitrile;
1-(4-acryloylpiperazin-1-yl)-6-(naphthalen-1-yl)-3-(piperidin-1-yl)-5,6,7,8-tetrahydro-2,6-naphthyridine-4-carbonitrile;
1,3-bis(4-acryloylpiperazin-1-yl)-6-(naphthalen-1-yl)-5,6,7,8-tetrahydro-2,6-naphthyridine-4-carbonitrile;
1,3-bis(4-acryloylpiperazin-1-yl)-6-(benzo[b]thiophen-4-yl)-5,6,7,8-tetrahydro-2,6-naphthyridine-4-carbonitrile;
1-(4-acryloylpiperazin-1-yl)-6-(naphthalen-1-yl)-3-(4-propionylpiperazin-1-yl)-5,6,7,8-tetrahydro-2,6-naphthyridine-4-carbonitrile;
3-(4-acryloylpiperazin-1-yl)-6-(naphthalen-1-yl)-1-(4-propionylpiperazin-1-yl)-5,6,7,8-tetrahydro-2,6-naphthyridine-4-carbonitrile;
1-(4-acryloylpiperazin-1-yl)-3-morpholino-6-(naphthalen-1-yl)-5,6,7,8-tetrahydro-2,6-naphthyridine-4-carbonitrile;
3-(4-acetylpiperazin-1-yl)-1-(4-acryloylpiperazin-1-yl)-6-(naphthalen-1-yl)-5,6,7,8-tetrahydro-2,6-naphthyridine-4-carbonitrile;
1-(4-acryloylpiperazin-1-yl)-3-(2,6-dimethylmorpholino)-6-(naphthalen-1-yl)-5,6,7,8-tetrahydro-2,6-naphthyridine-4-carbonitrile;
1-(4-acryloylpiperazin-1-yl)-3-(4-(methylsulfonyl)piperazin-1-yl)-6-(naphthalen-1-yl)-5,6,7,8-tetrahydro-2,6-naphthyridine-4-carbonitrile;
1-(4-acryloylpiperazin-1-yl)-3-(4-ethylpiperazin-1-yl)-6-(naphthalen-1-yl)-5,6,7,8-tetrahydro-2,6-naphthyridine-4-carbonitrile;
(S)-1-(4-acryloylpiperazin-1-yl)-3-((1-methylpyrrolidin-2-yl)methoxy)-6-(naphthalen-1-yl)-5,6,7,8-tetrahydro-2,6-naphthyridine-4-carbonitrile;
1-(4-acryloylpiperazin-1-yl)-6-(naphthalen-1-yl)-5,6,7,8-tetrahydro-2,6-naphthyridine-4-carbonitrile;
1-(4-(2-fluoroacryloyl)piperazin-1-yl)-6-(naphthalen-1-yl)-3-(4-propionylpiperazin-1-yl)-5,6,7,8-tetrahydro-2,6-naphthyridine-4-carbonitrile; and
a pharmaceutically acceptable salt thereof.
